SEQUENCE LISTING (59 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 5 - protein, 114 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 6 - protein, 231 aa, synthetic construct
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 7 - protein, 231 aa, synthetic construct
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 8 - protein, 231 aa, synthetic construct
EPKSSDKTHTCPPCPAPPVAGPSVFLFPPTPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 9 - protein, 349 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPG

SEQ ID NO: 10 - protein, 300 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 11 - protein, 350 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 12 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 231 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKQPREPQVYTLPPSREEMTKNQVSLTCLVKGFYPSDIAVEWESNGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 300 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPTPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 17 - protein, 350 aa, synthetic construct
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 18 - protein, 301 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 350 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 20 - protein, 301 aa, synthetic construct; identical to SEQ ID NO: 18

SEQ ID NO: 21 - protein, 350 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 22 - protein, 301 aa, synthetic construct; identical to SEQ ID NO: 18

SEQ ID NO: 23 - protein, 350 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 24 - protein, 301 aa, synthetic construct; identical to SEQ ID NO: 18

SEQ ID NO: 25 - protein, 345 aa, synthetic construct
DWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 26 - protein, 296 aa, synthetic construct
ITCPPPMSVEHADIWVKSYSLYSRERYICNSGFKRKAGTSSLTECVLNKATNVAHWTTPSLKCIREPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 345 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 28 - protein, 296 aa, synthetic construct; identical to SEQ ID NO: 26

SEQ ID NO: 29 - protein, 350 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 30 - protein, 301 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 31 - protein, 350 aa, synthetic construct
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 32 - protein, 301 aa, synthetic construct; identical to SEQ ID NO: 30

SEQ ID NO: 33 - protein, 350 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 34 - protein, 301 aa, synthetic construct; identical to SEQ ID NO: 30

SEQ ID NO: 35 - protein, 345 aa, synthetic construct
DWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 36 - protein, 296 aa, synthetic construct
ITCPPPMSVEHADIWVKSYSLYSRERYICNSGFKRKAGTSSLTECVLNKATNVAHWTTPSLKCIREPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 37 - protein, 345 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 38 - protein, 296 aa, synthetic construct; identical to SEQ ID NO: 36

SEQ ID NO: 39 - protein, 25 aa, synthetic construct
  SITE 1..25: note = "This sequence may encompass 1-5 'Gly Gly Gly Gly Ser'repeating units"
GGGGSGGGGSGGGGSGGGGSGGGGS

SEQ ID NO: 40 - protein, 25 aa, synthetic construct
  SITE 1..25: note = "This sequence may encompass 1-5 'Ser Ser Ser Ser Gly'repeating units"
SSSSGSSSSGSSSSGSSSSGSSSSG

SEQ ID NO: 41 - protein, 25 aa, synthetic construct
  SITE 1..25: note = "This sequence may encompass 1-5 'Gly Ser Ser Gly Gly'repeating units"
GSSGGGSSGGGSSGGGSSGGGSSGG

SEQ ID NO: 42 - protein, 25 aa, synthetic construct
  SITE 1..25: note = "This sequence may encompass 1-5 'Gly Gly Ser Gly Gly'repeating units"
GGSGGGGSGGGGSGGGGSGGGGSGG

SEQ ID NO: 43-44 - skipped (2 entries)

SEQ ID NO: 45 - protein, 6 aa, synthetic construct
TVAAPS

SEQ ID NO: 46 - skipped

SEQ ID NO: 47 - protein, 7 aa, synthetic construct
ASTSGPS

SEQ ID NO: 48 - protein, 18 aa, synthetic construct
KESGSVSSEQLAQFRSLD

SEQ ID NO: 49 - protein, 14 aa, synthetic construct
EGKSSGSGSESKST

SEQ ID NO: 50 - protein, 6 aa, synthetic construct
GGGGGG

SEQ ID NO: 51 - protein, 8 aa, synthetic construct
GGGGGGGG

SEQ ID NO: 52 - protein, 12 aa, synthetic construct
GSAGSAAGSGEF

SEQ ID NO: 53 - protein, 5 aa, synthetic construct
GGGGS

SEQ ID NO: 54 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 55 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 56 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 57 - protein, 350 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 58 - protein, 301 aa, synthetic construct; identical to SEQ ID NO: 30

SEQ ID NO: 59 - protein, 301 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPTPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

(19) United States
(12) Patent Application Publication
(10) Pub. No.: US 2024/0216473 A1
(43) Pub. Date: Jul. 4, 2024

(54) IL15/IL15R ALPHA HETERODIMERIC FC-FUSION PROTEINS FOR THE EXPANSION OF NK CELLS IN THE TREATMENT OF SOLID TUMOURS

(71) Applicants: Genentech, Inc., South San Francisco, CA (US); Xencor, Inc., Pasadena, CA (US)

(72) Inventors: Shomyseh Sanjabi, South San Francisco, CA (US); Vittal Shivva, South San Francisco, CA (US); Alexander Joachim Paul Ungewickell, South San Francisco, CA (US); Rajbharan Yadav, South San Francisco, CA (US)

(21) Appl. No.: 18/431,350

(22) Filed: Feb. 2, 2024

Related U.S. Application Data
(63) Continuation of application No. PCT/US2022/074453, filed on Aug. 3, 2022.
(60) Provisional application No. 63/229,425, filed on Aug. 4, 2021.

(51) Int. Cl. A61K 38/20; A61K 38/17; A61K 39/395; A61P 35/00

(52) U.S. Cl. CPC A61K 38/2086; A61K 38/1793; A61K 39/3955; A61P 35/00

(57) ABSTRACT

The present disclosure provides methods of treating cancer by administering a heterodimeric protein comprising a first monomer comprising an IL15 protein-Fc domain fusion and a second monomer comprising an IL15Rα protein-Fc domain fusion and either increasing the number of NK cells at least 20-fold as compared to the number of NK cells prior to said administration or resulting in the accumulation of NK cells in the subject. The present disclosure also provides methods for inducing the proliferation of NK cells by administering a heterodimeric protein comprising a first monomer comprising an IL15 protein-Fc domain fusion and a second monomer comprising an IL15Rα protein-Fc domain fusion and either increasing the number of NK cells at least 20-fold as compared to the number of NK cells prior to said administration or resulting in the accumulation of NK cells in the subject.

### CROSS-REFERENCE TO RELATED APPLICATIONS

This application claims priority to and benefit of U.S. Provisional Application No. 63/229,425, filed on Aug. 4, 2021, the contents of which are hereby incorporated by reference in their entirety.

### TECHNICAL FIELD

The present disclosure pertains to the field of stimulating the immune response by expanding NK cells, for example in the treatment of cancer, using IL15-IL15R heterodimeric Fc-fusion proteins.

### SEQUENCE LISTING

The instant application contains a Sequence Listing which has been submitted electronically in XML format and is hereby incorporated by reference in its entirety. Said XML copy, created on Aug. 3, 2022, is named 000218-0049-WO1_SL.xml and is 64,067 bytes in size.

### BACKGROUND

Cancer is a leading cause of death worldwide with an estimated 14 million new cases and 8 million deaths, globally, in 2012 (Torre et al. Cancer Epidemiol Biomarkers Prev. 2016; 25(1):16-27). By 2018, this trend had risen with an increase to more than 18 million new cases and more than 9 million deaths (New global cancer data: GLOBOCAN 2018. https://www.uicc.org/news/new-global-cancer-data-globocan-2018). These trends suggest a growing crisis and a need for effective therapies for cancer treatment. Cancer immunotherapy (CIT) has evolved as a promising approach in oncology in recent years, and it broadly includes checkpoint inhibitors, adoptive cell transfer, targeted antibodies (T/NK-cell engagers), cancer vaccines, and cytokines.

Cytokines can boost immune cells by controlling proliferation, differentiation, and survival of leukocytes (Berraondo et al. Br J Cancer 2019; 120(1):6-15). Despite the known biology of cytokines and their role in the immune system and cancer biology, only a limited number of cytokines have been approved for cancer treatment in select indications that include IFNα (e.g., hairy cell leukemia and chronic myelogenous leukemia among others) and IL-2 (e.g., advanced melanoma and metastatic RCC). This is, in part, related to poor tolerability, a narrow therapeutic index, and the poor PK behavior of these cytokines (Berraondo et al. 2019, supra).

For example, recombinant IL-2, also known as aldesleukin (Proleukin®), has been in clinical use as a CIT agent for more than two decades. Despite its proven clinical benefit as an antitumor agent, Proleukin® can induce major toxicities such as capillary leak syndrome (CLS), and patients receiving Proleukin require extensive monitoring in an inpatient setting. IL-2 is a secreted cytokine that acts on cells, such as cluster of differentiation-4 positive (CD4⁺) regulatory T cells (Treg), endothelial cells, and activated T cells, that express IL-2Rα (CD25) together with CD122 and CD132 in a high-affinity trimeric receptor complex. IL-2 is also known to induce activation-induced cell death (AICD). The increase of Treg function and induction of AICD are two processes that are expected to diminish antitumor immunity over time.

Interleukin (IL)-15, like other common γ chain (CD132) cytokines such as IL-2, IL-4, IL-7, IL-9, and IL-21, plays an important role in regulating immune responses. In addition to the common γ chain, IL-15 and IL-2 also share the β subunit (CD122) in their heterotrimeric receptor complex and have overlapping biological effects. IL-15 and IL-2, however, have a unique α receptor subunit for downstream signaling. IL-15 and IL-2 are known to play an important role in cancer immunity and were shown to boost the immune system by inducing proliferation and activation of natural killer (NK) cells and cluster of differentiation-8 positive (CD8⁺) T cells.

IL-15 is presented in trans by monocytes and dendritic cells in the context of IL-15Rα (CD215) to other cells, such as NK cells and memory CD8⁺ T cells, that mainly express CD122 and CD132 (heterodimeric receptor complex of intermediate affinity). Thus, when IL-15/IL-15Rα binds to CD122 and CD132 on NK and T cells, it leads to an enhanced durable T cell response by inducing CD8⁺ T cell proliferation and maintenance of memory CD8⁺ T cells, as well as enhanced NK-cell proliferation and cytotoxicity. Importantly, the biological effect of IL-15/IL-15Rα is minimal on CD25-expressing Tregs and IL-15/IL-15Rα is thought to cause less vascular leakage than is associated with IL-2 and is not known to induce AICD.

Hence, IL-15 has potential advantages over IL-2 as a CIT agent. In the past decade, several IL-2 and IL-15-based therapeutics have been tested in various clinical trials aiming to achieve improved clinical benefit and reduced toxicities, such as recombinant human IL-15 (rhIL-15) and an engineered IL-15/IL-15Rα-Fc superagonist (ALT-803). However, the pharmacokinetic (PK) exposure, pharmacodynamics (PD) response, or acute toxicities have limited their clinical impact to date. For example, IV bolus administration of rhIL-15 or rhIL-15/rhIL-15Rα complex has resulted in low PK exposure due to high target-mediated drug disposition (TMDD) and rapid renal clearance (CL) (due to a small molecular size of around 60 kDa); and has required frequent dosing. Furthermore, IV bolus administration has been limited by acute toxicities, including CLS and hypotension. The PK and safety limitations associated with IV bolus administration led to the exploration of alternate routes of administrations, such as subcutaneous (SC) injection or continuous IV infusion to improve tolerability and PD effects. While some of these approaches improved PD response (i.e., expansion of NK and CD8⁺ T cells) and tolerability, SC administration of rhIL-15 and ALT-803 has been associated with frequent injection site reactions, and frequent dosing (SC) or continuous infusion over several days is required for each treatment cycle. The available clinical data for IL-15 pathway agonists has provided a rationale to develop IL-15 therapeutics with an optimized PK profile and improved therapeutic index.

Thus, there remains a need for a CIT agent, specifically for IL-15 pathway agonists.

### SUMMARY

In a first aspect, the present disclosure provides a method of treating a solid tumor in a human subject in need thereof, the method comprising administering to the subject a plurality of doses of a heterodimeric protein, wherein each does comprises a therapeutically effective amount of the heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.

In a second aspect, the present disclosure provides method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising an amino acid sequence encoded by a polynucleotide encoding the amino acid sequence set forth in SEQ ID NO: 9; and (ii) a second monomer comprising an amino acid sequence encoded by a polynucleotide encoding the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.

In a third aspect, the present disclosure provides method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein is sufficient to increase the number of NK cells at least 20-fold as compared to the number of NK cells prior to said administration.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In a fourth aspect, the present disclosure provides a method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.

In a fifth aspect, the present disclosure provides a method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising an amino acid sequence encoded by a polynucleotide encoding the amino acid sequence set forth in SEQ ID NO: 9 and (ii) a second monomer comprising an amino acid sequence encoded by a polynucleotide encoding the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.

In a sixth aspect, the present disclosure provides a method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein is sufficient to increase the number of NK cells at least 20-fold as compared to the number of NK cells prior to said administration.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, the subject is suffering from a solid tumor. In some embodiments, the number of NK cells increases at least 30-fold, 40-fold, 50-fold, 60-fold, 70-fold, 80-fold, 90-fold, 100-fold, 150-fold, 200-fold, 250-fold, or 300-fold relative to the number of NK cells prior to administration.

In some embodiments, the heterodimeric protein is administered at a dose of at least 0.04 mg/kg Q2W and the number of NK cells increases at least 20-fold after two doses. In some embodiments, the heterodimeric protein is administered at a dose of at least 0.06 mg/kg Q2W and the number of NK cells increases at least 50-fold after two doses. In some embodiments, the heterodimeric protein is administered at a dose of at least 0.06 mg/kg Q2W and the number of NK cells increases at least 100-fold after three doses.

In a seventh aspect, the present disclosure provides a method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein results in accumulation of NK cells in the subject.

In an eighth aspect, the present disclosure provides a method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising an amino acid sequence encoded by a polynucleotide encoding the amino acid sequence set forth in SEQ ID NO: 9; and (ii) a second monomer comprising an amino acid sequence encoded by a polynucleotide encoding the amino acid sequence set forth in SEQ ID NO: 10 or 16; wherein the administration of the plurality of doses of the heterodimeric protein results in accumulation of NK cells in the subject.

In a ninth aspect, the present disclosure provides method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein results in accumulation of NK cells in the subject.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In a tenth aspect, the present disclosure provides a method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein results in accumulation of NK cells in the subject.

In an eleventh aspect, the present disclosure provides a method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising an amino acid sequence encoded by a polynucleotide encoding the amino acid sequence set forth in SEQ ID NO: 9; and (ii) a second monomer comprising an amino acid sequence encoded by a polynucleotide encoding the amino acid sequence set forth in SEQ ID NO: 10 or 16; wherein the administration of the plurality of doses of the heterodimeric protein results in accumulation of NK cells in the subject.

In a twelfth aspect, the present disclosure provides a method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein results in accumulation of NK cells in the subject.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, the subject is suffering from a solid tumor. In some embodiments, the accumulation is for at least one cycle. In some embodiments, the accumulation is for at least two cycles, at least three cycles, or at least four cycles.

In any of the above methods, the NK cells may be CD16+ NK cells.

In some embodiments of any of the methods disclosed herein, the first monomer comprises the amino acid sequence set forth in SEQ ID NO: 57. In some embodiments of any of the methods disclosed herein, the second monomer comprises the amino acid sequence set forth in SEQ ID NO: 10. In some embodiments of any of the methods disclosed herein, the second monomer comprises the amino acid sequence set forth in SEQ ID NO: 58. In some embodiments of any of the methods disclosed herein, the second monomer comprises the amino acid sequence set forth in SEQ ID NO: 16. In some embodiments of any of the methods disclosed herein, the second monomer comprises the amino acid sequence set forth in SEQ ID NO: 59. In some embodiments, the first monomer comprises a C-terminal lysine. In some embodiments, the first monomer lacks a C-terminal lysine. In some embodiments, the second monomer comprises a C-terminal lysine. In some embodiments, the second monomer lacks a C-terminal lysine. In some embodiments of any of the methods disclosed herein, the heterodimeric protein is XENP24306. In some embodiments of any of the methods disclosed herein, the heterodimeric protein is XENP32803. In some embodiments of any of the methods disclosed herein, a combination of XENP24306 and XENP32803 are used.

In some embodiments, the first monomer lacks a C-terminal lysine. In some embodiments, the second monomer comprises a C-terminal lysine. In some embodiments, the second monomer lacks a C-terminal lysine. In some embodiments of any of the methods disclosed herein, a combination of XENP24306 with a C-terminal lysine and XENP24306 without a C-terminal lysine. In some embodiments of any of the methods disclosed herein, the heterodimeric protein is XENP32803 with a C-terminal lysine and XENP32803 without a C-terminal lysine. In some embodiments of any of the methods disclosed herein, a combination of (1) XENP24306 with a C-terminal lysine and XENP24306 without a C-terminal lysine; and (2) XENP32803 with a C-terminal lysine and XENP32803 without a C-terminal lysine are used.

In some embodiments of any of the methods disclosed herein, the XENP24306 protein represents between about 50-about 100%, about 70-about 95%, about 80-about 90%, or about 80-about 85% of the heterodimeric protein in the combination. In some embodiments of any of the methods disclosed herein, the XENP32803 protein represents between about 1-about 50%, about 5-about 30%, about 10-about 20%, or about 15-about 20% of the heterodimeric protein in the combination. In some embodiments of any of the methods disclosed herein, the XENP24306 protein represents about 85% of the heterodimeric protein in the combination, and the XENP32803 protein represents about 15% of the heterodimeric protein in the combination. In some embodiments of any of the methods disclosed herein, the XENP24306 protein represents about 84% of the heterodimeric protein in the combination, and the XENP32803 protein represents about 16% of the heterodimeric protein in the combination. In some embodiments of any of the methods disclosed herein, the XENP24306 protein represents about 83% of the heterodimeric protein in the combination, and the XENP32803 protein represents about 17% of the heterodimeric protein in the combination. In some embodiments of any of the methods disclosed herein, the XENP24306 protein represents about 82% of the heterodimeric protein in the combination, and the XENP32803 protein represents about 18% of the heterodimeric protein in the combination. In some embodiments of any of the methods disclosed herein, the XENP24306 protein represents about 81% of the heterodimeric protein in the combination, and the XENP32803 protein represents about 19% of the heterodimeric protein in the combination. In some embodiments of any of the methods disclosed herein, the XENP24306 protein represents about 80% of the heterodimeric protein in the combination, and the XENP32803 protein represents about 20% of the heterodimeric protein in the combination.

In some embodiments of any of the methods disclosed herein, a combination of two or more heterodimeric proteins is administered to the subject. In some embodiments, a combination of a first heterodimeric protein and a second heterodimeric protein is administered to the subject.

In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 16.

In some embodiments, said first and second heterodimeric proteins are administered simultaneously. In some embodiments, said first and second heterodimeric proteins are administered sequentially. In some embodiments, said first and second heterodimeric proteins are administered in the same composition. In some embodiments, the first and second heterodimeric proteins are administered in separate compositions.

In some embodiments, the solid tumor to be treated by any of the methods disclosed herein is locally advanced, recurrent or metastatic. In some embodiments, said solid tumor is selected from the group consisting of squamous cell cancer, cutaneous squamous cell cancer, small-cell lung cancer, non-small cell lung cancer, gastrointestinal cancer, gastric cancer, pancreatic cancer, glioblastoma, cervical cancer, ovarian cancer, liver cancer, bladder cancer, liposarcoma, soft-tissue sarcoma, urothelial carcinoma, ureter and renal pelvis, multiple myeloma, osteosarcoma, hepatoma, melanoma, stomach cancer, breast cancer, colon cancer, colorectal cancer, endometrial carcinoma, salivary gland carcinoma, renal cell carcinoma, esophageal cancer, prostate cancer, vulval cancer, thyroid cancer, hepatic carcinoma, Merkel cell carcinoma, germ cell cancer, micro-satellite instability-high cancer and head and neck squamous cell carcinoma. In some embodiments, said solid tumor is selected from melanoma, renal cell carcinoma, non-small cell lung cancer, head and neck squamous cell carcinoma, and triple negative breast cancer. In some embodiments, said solid tumor is selected from melanoma, renal cell carcinoma, and non-small cell lung cancer. In some embodiments, said solid tumor is selected from melanoma, non-small cell lung cancer, head and neck squamous cell carcinoma, and triple negative breast cancer.

In some embodiments, prior to administration of the plurality of doses of the heterodimeric protein, the subject has not been previously administered an agent to treat the solid tumor. In some embodiments, the subject is currently being administered a checkpoint inhibitor. In some embodiments, prior to administration of the plurality of doses of the heterodimeric protein, the subject has previously been administered a checkpoint inhibitor. In some embodiments, the checkpoint inhibitor targets PD-1. In some embodiments, the checkpoint inhibitor targets PD-L1. In some embodiments, the checkpoint inhibitor targets CTLA-4.

In some embodiments, the heterodimeric protein is administered at a dose of selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.09 mg/kg about 0.10 mg/kg, about 0.12 mg/kg, about 0.16 mg/kg, about 0.20 mg/kg, about 0.24 mg/kg and about 0.32 mg/kg body weight. In some embodiments, the heterodimeric protein is administered at a dose of selected from the group consisting of about 0.01 mg/kg, about 0.02 mg/kg, about 0.04 mg/kg, about 0.06 mg/kg, about 0.09 mg/kg, about 0.135 mg/kg, and about 0.2025 mg/kg body weight. In some embodiments, the heterodimeric protein is administered at a dose of selected from the group consisting of about 0.01 mg/kg, about 0.02 mg/kg, about 0.04 mg/kg, about 0.06 mg/kg, and about 0.09 mg/kg body weight. In some embodiments, the heterodimeric protein is administered at a dose of selected from the group consisting of about 0.01 mg/kg, about 0.02 mg/kg, about 0.04 mg/kg, about 0.06 mg/kg, about 0.09 mg/kg, and about 0.12 mg/kg body weight. In some embodiments, the heterodimeric protein is administered at a frequency selected from the group consisting of Q1W, Q2W, Q3W, Q4W, Q5W and QW6. In some embodiments, the heterodimeric protein is administered at a dose of selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.16 mg/kg, 0.20 mg/kg, 0.24 mg/kg and 0.32 mg/kg body weight. In some embodiments, the heterodimeric protein is administered at a dose of selected from the group consisting of 0.01 mg/kg, 0.02 mg/kg, 0.04 mg/kg, 0.06 mg/kg, 0.09 mg/kg, 0.135 mg/kg, and 0.2025 mg/kg body weight. In some embodiments, the heterodimeric protein is administered at a frequency selected from the group consisting of Q1W, Q2W, Q3W, Q4W, Q5W and Q6W.

In some embodiments, the combination of heterodimeric proteins (e.g. XENP24306+XENP32803) is administered at a dose of selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.09 mg/kg, about 0.10 mg/kg, about 0.12 mg/kg, about 0.16 mg/kg, about 0.20 mg/kg, about 0.24 mg/kg and about 0.32 mg/kg body weight. In some embodiments, the combination of heterodimeric proteins (e.g., XENP24306+XENP32803) is administered at a dose of selected from the group consisting of about 0.01 mg/kg, about 0.02 mg/kg, about 0.04 mg/kg, about 0.06 mg/kg, about 0.09 mg/kg, about 0.135 mg/kg, and about 0.2025 mg/kg body weight. In some embodiments, the combination of heterodimeric proteins (e.g., XENP24306+XENP32803) is administered at a dose of selected from the group consisting of about 0.01 mg/kg, about 0.02 mg/kg, about 0.04 mg/kg, about 0.06 mg/kg, and about 0.09 mg/kg body weight. In some embodiments, the combination of heterodimeric proteins (e.g., XENP24306+XENP32803) is administered at a dose of selected from the group consisting of about 0.01 mg/kg, about 0.02 mg/kg, about 0.04 mg/kg, about 0.06 mg/kg, about 0.09 mg/kg, and about 0.12 mg/kg body weight. In some embodiments, the combination of heterodimeric protein is administered at a frequency selected from the group consisting of Q1W, Q2W, Q3W, Q4W, Q5W and Q6W. In some embodiments, the combination of heterodimeric proteins (e.g., XENP24306+XENP32803) is administered at a dose of selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.16 mg/kg, 0.20 mg/kg, 0.24 mg/kg and 0.32 mg/kg body weight. In some embodiments, the combination of heterodimeric proteins (e.g., XENP24306+XENP32803) is administered at a dose of selected from the group consisting of 0.01 mg/kg, 0.02 mg/kg, 0.04 mg/kg, 0.06 mg/kg, 0.09 mg/kg, 0.135 mg/kg, and 0.2025 mg/kg body weight. In some embodiments, the combination of heterodimeric protein is administered at a frequency selected from the group consisting of Q1W, Q2W, Q3W, Q4W, Q5W and Q6W.

In some embodiments, the methods disclosed herein further comprise administering to the subject an agent targeting the PD-L1/PD-1 axis. In some embodiments, said agent targeting the PD-L1/PD-1 axis is an anti-PD-1 antibody. In some embodiments, the anti-PD-1 antibody is selected from nivolumab, pembrolizumab, pidilizumab, cemiplimab, spartalizumab, camrelizumab, sintilimab, tislelizumab, toripalimab, and AMP-514. In some embodiments, said agent targeting the PD-L1/PD-1 axis is an anti-PD-L1 antibody. In some embodiments, the anti-PD-L1 antibody is selected from avelumab (MSB0010718C), durvalumab, atezolizumab, BMS-936559, BMS-39886, envafolimab (KN035), and cosibelimab (CK-301).

These and other aspects will be readily apparent to the skilled artisan in light of the disclosure as a whole.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B show that a combination of XENP24306 (˜82%) and XENP32803 (˜18%) promotes dose-dependent proliferation of human NK cells (FIG. 1A) and CD8⁺ T cells (FIG. 1B) in human PBMCs. PBMC from 22 unique human donors were treated with indicated total concentrations of the combination of XENP24306 (˜82%) and XENP32803 (˜18%) for 4 days, and Ki67⁺ (marker of cell proliferation) frequency was determined by flow cytometry for CD3⁻ CD56+ NK cells (FIG. 1A) or CD3⁺CD8⁺ CD16⁻ T cells (FIG. 1B). Each point represents the average value of 22 donors and error bars represent SEM. Curve fits were generated using the least squares method. EC₅₀ values were determined by non-linear regression analysis using agonist versus response using a variable-slope (four-parameter) equation. [CD=cluster of differentiation; NK=natural killer; PBMC=peripheral blood mononuclear cell].

FIG. 2 shows a comparison of CD8⁺ terminal effector T cell proliferation induced by a combination of XENP24306 (˜82%) and XENP32803 (˜18%), recombinant wild-type IL-15 (rIL15) and wild-type IL-15/wild-type IL-15Rα heterodimer Fc fusion (XENP22853) in human PBMCs. [EC₅₀=half maximal effective concentration].

FIGS. 3A-3D show graphs representing CD8β⁺ T cells (FIG. 3A (males) and 3B (females)) and NK cells (FIG. 3C (males) and 3D (females)) absolute count in whole blood of cynomolgus monkeys treated with repeat doses of a combination of XENP24306 (˜82%) and XENP32803 (˜18%) and different doses (0; 0.03 mg/kg; 0.2 mg/kg and 0.6 mg/kg). Whole blood from cynomolgus monkeys was stained with antibodies to identify CD8⁺ T cells as CD45⁺ CD3⁺ CD8β⁺ CD4⁻ CD16⁻ and NK cells as CD45⁻ CD3⁻ CD16⁺. Each data point represents the mean of 3 to 5 cynomolgus monkeys per group; error bars denote SD.

FIG. 4 is a graph representing mean (±SD) heterodimeric protein (a combination of XENP24306 (˜82%) and XENP32803 (˜18%) serum concentration (ng/mL) versus time (days) profiles in cynomolgus monkeys (males and females combined) following heterodimeric protein Q2W intravenous dosing (doses of 0.03 mg/kg; 0.2 mg/kg and 0.6 mg/kg) for a total of 3 doses.

FIG. 5 is a graph representing the body weight loss in non-obese diabetic/severe combined immunodeficient gamma (NSG) mice engrafted with human PBMCs, wherein a combination of XENP24306 (˜82%) and XENP32803 (˜18%) was administered at various concentrations in the presence or absence of 3 mg/kg of XENP16432, which is an anti-PD1 bivalent antibody. Samples: (A) PBS; (B) 3.0 mg/kg XENP16432; (C) 0.3 mg/kg of a combination of XENP24306 (˜82%) and XENP32803 (˜18%); (D) 0.1 mg/kg of a combination of XENP24306 (˜82%) and XENP32803 (˜18%); (E) 0.03 mg/kg of a combination of XENP24306 (˜82%) and XENP32803 (˜18%); (F) 0.01 mg/kg of a combination of XENP24306 (˜82%) and XENP32803 (˜18%); (G) 0.3 mg/kg of a combination of XENP24306 (˜82%) and XENP32803 (˜18%)+3.0 mg/kg XENP16432; (H) 0.1 mg/kg of a combination of XENP24306 (˜82%) and XENP32803 (˜18%)+3.0 mg/kg XENP16432; (I) 0.03 mg/kg of a combination of XENP24306 (˜82%) and XENP32803 (˜18%)+3.0 mg/kg XENP16432; and (J) 0.01 mg/kg of a combination of XENP24306 (˜82%) and XENP32803 (˜18%)+3.0 mg/kg XENP16432.

FIG. 6 is a graph representing group medians of changes in tumor volume in non-obese diabetic/severe combined immunodeficient gamma (NSG) mice engrafted with human tumor cells (pp65-MCF7) and huPBMC as a source of human leukocytes, wherein a combination of XENP24306 (˜82%) and XENP32803 (˜18%) was administered at various concentrations in the presence or absence of 3 mg/kg of XENP16432. Samples: (A) PBS; (B) 3.0 mg/kg XENP16432; (C) 1.0 mg/kg of a combination of XENP24306 (˜82%) and XENP32803 (˜18%); (D) 0.3 mg/kg of a combination of XENP24306 (˜82%) and XENP32803 (˜18%); (E) 0.1 mg/kg of a combination of XENP24306 (˜82%) and XENP32803 (˜18%); (F) 1.0 mg/kg of a combination of XENP24306 (˜82%) and XENP32803 (˜18%)+3.0 mg/kg XENP16432; (G) 0.3 mg/kg of a combination of XENP24306 (˜82%) and XENP32803 (˜18%)+3.0 mg/kg XENP16432; and (H) 0.1 mg/kg of a combination of XENP24306 (˜82%) and XENP32803 (˜18%)+3.0 mg/kg XENP16432.

FIG. 7 is the monotherapy study schema for an IL15/IL15Rα heterodimeric protein (e.g., XENP24306, XENP32803, or a combination of XENP24306 (˜82%) and XENP32803 (˜18%), showing patients enrolled in two stages: a dose-escalation stage and an expansion stage and details on these two stages. DL=dose level; DLT=dose-limiting toxicity; MTD=maximum tolerated dose; PD=pharmacodynamic; Q2W=every 2 weeks; Q3W=every 3 weeks; Q4W=every 4 weeks; RCC=renal cell carcinoma; RED=recommended expansion dose. ^{a}PD effect is assessed by enumeration and Ki67 staining of peripheral blood NK cells and CD8⁺ T cells. ^{b} Safety threshold to change from n=1/dose level to 3+3+3 design is defined in Example 6. ^{c}Safety threshold to change from ≤100% dose increments to ≤50% dose increments is defined in Example 6. ^{d}If cumulative toxicities lead to unacceptable tolerability (e.g., frequent dose delays of the IL15/IL15Rα heterodimeric protein), the IL15/IL15Rα heterodimeric protein dosing frequency may be reduced.

FIG. 8 is the combination therapy study schema for an IL15/IL15Rα heterodimeric protein (e.g., XENP24306, XENP32803, or a combination of XENP24306 (˜82%) and XENP32803 (˜18%) in combination with atezolizumab (anti-PD-L1 antibody), showing patients enrolled in two stages: a dose-escalation stage and an expansion stage and details on these two stages. Bx=biopsy; CIT=cancer immunotherapy; cSCC=cutaneous squamous cell carcinoma; DL=dose level; DLT=dose-limiting toxicity; GC=gastric cancer; HNSCC=head and neck squamous cell carcinoma; MCC=Merkel cell carcinoma; MSI-H=microsatellite instability-high; MTD=maximum tolerated dose; NSCLC=non-small cell lung cancer; PD=pharmacodynamic; Q2W=every 2 weeks; Q3W=every 3 weeks; Q4W=every 4 weeks; RCC=renal cell carcinoma; RED=recommended expansion dose; SCLC=small cell lung carcinoma; TBD=to be determined; TNBC=triple-negative breast cancer; UCC-urothelial carcinoma. ^{a}Safety threshold to switch from ≤100% dose increase increments to ≤50% is defined in Example 6. ^{b}In the case that the initial monotherapy IL15/IL15Rα heterodimeric protein dose level of 0.01 mg/kg demonstrates PD activity, the IL15/IL15Rα heterodimeric protein starting dose will be no higher than 0.005 mg/kg in the initial combination therapy atezolizumab combination cohort. ^{c}If cumulative toxicities lead to unacceptable tolerability (e.g., frequent dose delays of IL15/IL15Rα heterodimeric protein), the IL15/IL15Rα heterodimeric protein/atezolizumab dosing frequency may be reduced. ^{d}PD effect that informs the initial IL15/IL15Rα heterodimeric protein dose level is defined in Example 6. ^{e}Patient must have received prior anti-PD-L1/PD-1 inhibitor as single agent or in combination and derived clinical benefit from the prior treatment. ^{f}Indications include melanoma, NSCLC, HNSCC, TNBC, UCC, RCC, SCLC, GC, MCC, cSCC, MSI-H cancers. ^{g}Will enroll patients with melanoma, RCC, UCC, NSCLC, HNSCC, and TNBC. ^{h}PD-L1 threshold may differ between indications and will be determined.

FIG. 9 provides the amino acid sequences for XENP24306 monomer 1 (SEQ ID NO: 9), XENP24306 monomer 2 (SEQ ID NO: 10), XENP32803 monomer 1 (SEQ ID NO: 9), and XENP32803 monomer 2 (SEQ ID NO: 16). In the monomer 1 sequences, the IL15 portion is underlined, the linker is offset with slashes and is bold and underlined, and the Fc portion follows the second slash and does not contain any formatting. In the monomer 2 sequences, the IL15Rα portion is underlined, the linker is offset with slashes and is bold and underlined, and the Fc portion follows the second slash and does not contain any formatting.

FIGS. 10A and 10B provide the amino acid sequences for the human IL-15 precursor protein (full-length human IL-15) (SEQ ID NO: 2), the mature or truncated human IL-15 protein (SEQ ID NO: 1), the full-length human IL-15Rα protein (SEQ ID NO: 3), the extracellular domain of the human IL-15Rα protein (SEQ ID NO: 54), the sushi domain of the human IL-15Rα protein (SEQ ID NO: 4), the full-length human IL-15Rβ protein (SEQ ID NO: 55) and the extracellular domain of the human IL-15Rβ protein (SEQ ID NO: 56).

FIGS. 11A to 11G provide the amino acid sequences for XENP2853 wild-type IL-15-Fc first monomer (SEQ ID NO: 11), XENP2822 protein (SEQ ID NO: 19 and SEQ ID NO: 20), XENP23504 protein (SEQ ID NO: 29 and SEQ ID NO: 30), XENP24045 protein (SEQ ID NO: 23 and SEQ ID NO: 24), XENP22821 protein (SEQ ID NO: 17 and SEQ ID NO: 18), XENP23343 protein (SEQ ID NO: 31 and SEQ ID NO: 32), XENP23557 protein (SEQ ID NO: 21 and SEQ ID NO: 22), XENP24113 protein (SEQ ID NO: 33 and SEQ ID NO: 34), XENP24051 protein (SEQ ID NO: 25 and SEQ ID NO: 26), XENP24341 protein (SEQ ID NO: 35 and SEQ ID NO: 36), XENP24052 protein (SEQ ID NO: 27 and SEQ ID NO: 28), and XENP24301 protein (SEQ ID NO: 37 and SEQ ID NO: 38).

FIGS. 12A and 12B provide a graph (A) and a table (B) showing the fold increase in natural killer (NK) cells (CD3-CD56+/CD16+) in the peripheral blood of each patient over time following treatment with 0.01 mg/kg, 0.02 mg/kg, 0.04 mg/kg, 0.06 mg/kg, or 0.09 mg/kg of the XENP combo (XENP24306 (˜82%) and XENP32803 (˜18%)). Patients receiving 0.04 mg/kg or higher of the XENP combo demonstrated more than a 20-fold increase (dashed line in A) in NK cells within the first 2 cycles of treatment, demonstrating that two doses of 0.04 mg/kg is sufficient to reach a 20-fold increase in expansion and accumulation of NK cells. C1D1=cycle 1, day 1; C1D4=cycle 1, day 4; C1D8=cycle 1, day 8; C2D1=cycle 2, day 1; C2D8=cycle 2, day 8; C3D1=cycle 3, day 1; C3D8=cycle 3, day 8; C4D1=cycle 4, day 1; and C4D8=cycle 4, day 8.

FIGS. 13A to 13C demonstrate that the XENP combo (XENP24306 (˜82%) and XENP32803 (˜18%)) mainly expands CD16+ natural killer (NK) cells. FIG. 13A shows the increase in absolute cells counts of total NK cells. Shaded area indicates the normal range of NK cells (95-640 cells/μL) in healthy individuals. FIG. 13B provides the flow cytometry gating for separating NK cells based on CD56 and CD16. FIG. 13C provides graphs depicting the absolute cell counts for the four different NK cell populations, demonstrating the accumulation of the CD16+ populations. C2D1=cycle 2, day 1; C3D1=cycle 3, day 1; and C4D1=cycle 4, day 1.

FIGS. 14A and 14B provide a graph (A) and a table (B) showing the fold increase in natural killer (NK) cells (CD3-CD56+/CD16+) in the peripheral blood of each patient over time following treatment with 0.01 mg/kg, 0.02 mg/kg, 0.04 mg/kg, 0.06 mg/kg, or 0.09 mg/kg of the XENP combo (XENP24306 (˜82%) and XENP32803 (˜18%)) in combination with 840 mg of atezolizumab. A patient receiving 0.04 mg/kg of the XENP combo and Atezolizumab demonstrated more than a 20-fold increase (dashed line in A) in NK cells within the first two cycles of treatment, demonstrating that two doses of 0.04 mg/kg of XENP combo when administered with atezolizumab is sufficient to reach a 20-fold increase in expansion and accumulation of NK cells. In panel A, * in legend indicates patient cross-over from Phase 1A into Phase 1B, and § indicates fold change in NK cell number was calculated based on C1D4 versus CID1 sample. C1D1=cycle 1, day 1; C1D4=cycle 1, day 4; C1D8=cycle 1, day 8; C2D1=cycle 2, day 1; C2D8=cycle 2, day 8; C3D1=cycle 3, day 1; C3D8=cycle 3, day 8; C4D1=cycle 4, day 1; and C4D8=cycle 4, day 8.

FIGS. 15A to 15C demonstrate that the XENP combo (XENP24306 (˜82%) and XENP32803 (˜18%)) when administered with 840 mg of atezolizumab mainly expands CD16+ natural killer (NK) cells. FIG. 15A shows the increase in absolute cells counts of total NK cells. Shaded area indicates the normal range of NK cells (95-640 cells/μL) in healthy individuals. Subject 15007 (orange pentagon) crossed over from Phase 1A into Phase 1B. FIG. 15B provides the flow cytometry gating for separating NK cells based on CD56 and CD16. FIG. 15C provides graphs depicting the absolute cell counts for the four different NK cell populations, demonstrating the accumulation of the CD16+ populations. C2D1=cycle 2, day 1; C3D1=cycle 3, day 1; and C4D1=cycle 4, day 1.

### DETAILED DESCRIPTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Fourth edition, Cold Spring Harbor Laboratory Press, 2012; Ausubel et al., CURRENT PROTOCOLS IN MOLECULAR BIOLOGY, John Wiley & Sons, New York, 1987-2010 and periodic updates.

The term “herein” means the entire application.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the multiple dependent claims.

Any publications, patents and published patent applications referred to in this application are specifically incorporated by reference herein. In case of conflict, the present specification, including its specific definitions, will control.

Throughout this specification, the word “comprise” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be typically within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

The term “ablation,” as used herein, refers to a decrease or removal of activity. Thus, for example, “ablating FcγR binding” means that the Fc region amino acid variant has less than 50% starting binding as compared to an Fc region not containing the specific variant, with less than 70%, less than 80%, less than 90%, less than 95% or less than 98% loss of activity being preferred, and in general, with the activity being below the level of detectable binding in a BIACORE® assay (Pharmacia Biosensor AB, Uppsala, Sweden and Piscataway, N.J.). Unless otherwise noted, the Fc domains described herein retain binding to the FcRn receptor.

As used herein, the term “accumulation” refers to the increase in number of cells (e.g., NK cells or CD16+ NK cells) from one treatment cycle to the next. The number of cells (e.g., NK cells or CD16+ NK cells) is measured by the area under the curve (AUC) of the number of cells for each treatment cycle. When the number of cells in a second cycle is greater than the number of cells in a first (earlier) cycle, there has been accumulation for one cycle. When the number of cells in a third cycle is greater than the number of cells in a second (earlier than the third) cycle, and the number of cells in the second cycle is greater than the number of cells in a first (earlier than the second)) cycle, there has been accumulation for two cycles.

“Administering” or “administration of” a substance, a compound or an agent to a subject refers to the contact of that substance, compound or agent to the subject or a cell, tissue, organ or bodily fluid of the subject. Such administration can be carried out using one of a variety of methods known to those skilled in the art. For example, a compound or an agent can be administered intravenously or subcutaneously. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a subject to self-administer a drug, or to have the drug administered by another and/or who provides a subject with a prescription for a drug is administering the drug to the subject.

As used herein, the term “affinity” of a molecule refers to the strength of interaction between the molecule and a binding partner, such as α receptor, a ligand or an antigen. A molecule's affinity for its binding partner is typically expressed as the binding affinity equilibrium dissociation constant (K_{D}) of a particular interaction, wherein the lower the K_{D}, the higher the affinity. A K_{D} binding affinity constant can be measured by surface plasmon resonance, for example using the BIACORE® system (Pharmacia Biosensor AB, Uppsala, Sweden and Piscataway, N.J.) See also, Jonsson et al., Ann. Biol. Clin. 51:19 26 (1993); Jonsson et al., Biotechniques 11:620 627 (1991); Jonsson et al., J. Mol. Recognit. 8:125 131 (1995); Johnsson et al., Anal. Biochem. 198:268 277 (1991); Hearty S et al., Methods Mol Biol. 907:411-42 (2012), each incorporated herein by reference. The K_{D} may also be measured using a KinExA® system (Sapidyne Instruments, Hanover, Germany and Boise, ID). In some embodiments, the IL-15 variant of the heterodimeric protein described herein has reduced binding affinity towards IL-2/IL-15βγ receptor, compared with wild-type IL-15. In some embodiments, the first and/or the second Fc variant of the heterodimeric protein described herein has reduced affinity towards human, cynomolgus monkey, and mouse Fcγ receptors. In some embodiments, the first and/or the second Fc variant of the heterodimeric protein described herein does not bind to human, cynomolgus monkey, and mouse Fcγ receptors.

The terms “amino acid” and “amino acid identity,” as used herein, refer to one of the 20 naturally occurring amino acids that are coded for by DNA and RNA.

The term “amino acid substitution” or “substitution,” as used herein, refers to the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not considered an amino acid substitution.

The terms “amino acid insertion,” “amino acid addition” or “addition” or “insertion,” as used herein, refer to the addition of an amino acid sequence at a particular position in a parent polypeptide sequence. For example, −233E, _233E or 233E designates an insertion of glutamic acid after position 233 and before position 234. Additionally, −233ADE, _233ADE or 233ADE designates an insertion of AlaAspGlu after position 233 and before position 234.

The term “amino acid deletion” or “deletion,” as used herein, refers to the removal of an amino acid sequence at a particular position in a parent polypeptide sequence. For example, E233- or E233 #, E233( ), E233_ or E233 del designates a deletion of glutamic acid at position 233. Additionally, EDA233−, EDA233_ or EDA233 # designates a deletion of the sequence GluAspAla that begins at position 233.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, etc.) that specifically bind to a given antigen. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. There are two types of light chain: λ and κ. In humans, λ and κ light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety). The methods, uses, and compositions-for-use disclosed herein utilize IgG antibodies

The term “atezolizumab,” as used herein, refers to anti-PD-L1 antagonist antibody having the International Nonproprietary Names for Pharmaceutical Substances (INN) List 112 (WHO Drug Information, Vol. 28, No. 4, 2014, p. 488), or the CAS Registry Number 1380723-44-3.

As used herein, the term “CD16+ NK cells” refers to NK cells expressing CD16 at an intermediate (CD16_{intermediate}) or a high (CD16_{high}) level. The subpopulation of “CD16_{low}” includes very low and negative expression of CD16 and can also be identified as “CD16− NK cells.”

As used herein, the term “checkpoint inhibitor” refers to a compound which targets and blocks checkpoint proteins. A checkpoint inhibitor interferes with the interaction between a checkpoint protein and its partner protein. Examples of checkpoint inhibitors include, but are not limited, to agents that target the PD-1/PD-L1 axis and agents that target CTLA-4.

As used herein, the term “cycle” refers to each administration event in a series of regularly repeated administration steps. For example, if a therapeutic agent (e.g. a heterodimeric protein of the present disclosure) is administered once every two weeks (Q2W), the first cycle begins on day 1 and ends on day 14, the second cycle begins on day 15 and ends on day 28, the third cycle begins on day 29 and ends on day 42, and so on. Measurements may be taken, and combination therapies may be administered, mid-cycle. Mid-cycle events can be defined by the cycle and the day in the cycle in which they occur, e.g., a measurement taken one week into a two-week cycle might be numbered Cycle 1, Day 8 (or C1D8). For combination therapies, a cycle will be defined by the period in which it takes the administration pattern to repeat. For example, if a first therapeutic agent is administered Q2W and a second therapeutic agent is administered Q1W, then the cycle is a two-week cycle. In such a case, if both agents are administered on C1D1, then the second dose of the second agent would be administered on CID8, and the second dose of the first agent combined with the third dose of the second agent would be administered one week later to start the second cycle (i.e. C2D1). If a first therapeutic agent is administered Q1W and a second therapeutic agent is administered every three days (Q3D), then it will take three weeks for the administration pattern to repeat, so the cycle would be a three-week cycle and would include 3 administrations of the first agent and seven administrations of the second agent.

As used herein, the term “effector function” refers to a biochemical event that results from the interaction of an antibody Fc region with an Fc receptor or another effector molecule (e.g., Fc receptor-Like (FcRL) molecules, complement component C1q, and Tripartite motif-containing protein 21 (TRIM21)). Effector functions include, but are not limited to, antibody dependent cell-mediated cytotoxicity (ADCC), antibody dependent cell-mediated phagocytosis (ADCP) and complement-dependent cellular cytotoxicity (CDC). The term “ADCC” or “antibody dependent cell-mediated cytotoxicity,” as used herein, refers to the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the present disclosure ablate ADCC activity entirely. The term “ADCP” or “antibody dependent cell-mediated phagocytosis,” as used herein, refers to the cell-mediated reaction wherein nonspecific cytotoxic cells that express FcγRs recognize bound antibody on a target cell and subsequently cause phagocytosis of the target cell. The term “CDC” or “complement-dependent cellular cytotoxicity,” as used herein, refers to an effector function which leads to the activation of the classical complement pathway, which is triggered by the binding of an antibody to an antigen on the target cell, which activates a series of cascades containing complement-related protein groups in blood.

As used herein, the terms “Fc,” “Fc region” or “Fc domain” are used interchangeably herein and refer to the polypeptide comprising the constant region of an antibody excluding, in some instances, the first constant region immunoglobulin domain (e.g., CH1) or a portion thereof, and in some cases, part of the hinge. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, an Fc refers to a truncated CH1 domain, and CH2 and CH3 of an immunoglobulin. Although the boundaries of the Fc region may vary, the human IgG heavy chain Fc region is usually defined to include residues E216 or C226 or P230 to its carboxyl-terminus, wherein the numbering is according to the EU numbering. However, the C-terminal lysine (Lys447) of the Fc region may or may not be present. In some embodiments, as is more fully described herein, amino acid modifications are made to the Fc region, for example to alter binding to one or more FcγR receptors or to the FcRn receptor. In some embodiments, the Fc domain is derived from a human IgG1 heavy chain Fc domain. In some embodiments, the Fc domain is derived from a human IgG2 heavy chain Fc domain. The “EU format as set forth in Edelman” or “EU numbering” or “EU index” refers to the residue numbering of the human Fc domain as described in Edelman G M et al. (Proc. Natl. Acad. USA (1969), 63, 78-85, hereby entirely incorporated by reference).

As used herein, the terms “Fc fusion protein” and “immunoadhesin” are used interchangeably and refer to a protein comprising an Fc region, generally linked (optionally through a linker moiety, as described herein) to a different protein, such as to IL-15 and/or IL-15R, as described herein. In some instances, two Fc fusion proteins can form a homodimeric Fc fusion protein or a heterodimeric Fc fusion protein with the latter being preferred.

As used herein, the term “Fc variant” or “variant Fc” refers to a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. For all positions discussed in the present invention that relate to antibodies, unless otherwise noted, amino acid position numbering is according to the EU index. The modification can be an addition, deletion, or substitution. Substitutions can include naturally occurring amino acids and, in some cases, synthetic amino acids. Examples include, but are not limited to, U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all of them entirely incorporated by reference. In some embodiments, the substitutions comprise only naturally occurring amino acids. In some embodiments, the substitutions do not comprise any synthetic amino acids.

The terms “Fc gamma receptor,” “FcγR” and “FcgammaR,” as used herein, are used interchangeably and refer to any member of the family of proteins that bind the IgG antibody Fc region and is encoded by an FcγR gene. An FcγR may be from any organism. In some embodiments, the FcγR is a human FcγR. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes.

The term “FcRn” or “neonatal Fc Receptor,” as used herein, refers to a protein that binds the IgG antibody Fc region and is encoded at least in part by an FcRn gene. The FcRn may be from any organism. In some embodiments, the FcRn is a human FcRn. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life. In general, unless otherwise noted, the Fc monomers disclosed herein retain binding to the FcRn receptor (and, as noted below, can include amino acid variants to increase binding to the FcRn receptor).

The term “modification,” as used herein, refers to an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always referring to an amino acid coded for by DNA, e.g., the 20 amino acids that have codons in DNA and RNA.

The terms “nucleic acid,” “polynucleotide” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T. In some embodiments, the polynucleotide comprises only natural nucleotides. In some embodiments, the polynucleotide does not comprise any analogues of a natural nucleotide.

The term “non-naturally occurring modification,” as used herein, refers to an amino acid modification that is not isotypic. For example, because none of the IgGs comprise a serine at position 434, the substitution 434S in IgG1, IgG2, IgG3 or IgG4 (or hybrids thereof) is considered a non-naturally occurring modification.

The terms “patient,” “subject” and “individual” are used interchangeably herein and refer to either a human or a non-human animal in need to treatment. These terms include mammals, such as humans, and primates (e.g., monkey). In some embodiments, the subject is a human. In some embodiments, the subject is in need of treatment of cancer. The terms “treating” and “treatment,” as used herein, refer to reduction in severity and/or frequency of symptoms, elimination of symptoms and/or underlying cause, prevention of the occurrence of symptoms and/or their underlying cause, and improvement or remediation of damage.

As used herein, “percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference.

As used herein, the terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids. In some embodiments, the polypeptide only comprises naturally-occurring amino acids. In some embodiments, the polypeptide does not comprise any chemical analogues or modified derivatives of a corresponding naturally-occurring amino acids. Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are known in the art.

The term “position,” as used herein, refers to a location in the sequence of a protein. Positions may be numbered sequentially, or according to an established format, for example the EU index for antibody numbering. A position may be defined relative to a reference sequence. In such cases, the reference sequence is provided for comparison purposes, and the heterodimeric protein of the disclosure (or a portion thereof) may comprise additional amino acid alterations (e.g., substitutions, insertions, and deletions) relative to the reference sequence. In some embodiments, the heterodimeric protein of the disclosure (or a portion thereof) does not comprise any additional amino acid alterations relative to the reference sequence.

The term “residue,” as used herein, refers to a position in a protein and its associated amino acid identity. For example, Asparagine 297 (also referred to as Asn297 or N297) is a residue at position 297 in a specific protein.

As used herein, the term “therapeutically effective amount” and “effective amount” are used interchangeably herein and refer to that amount of the therapeutic agent being administered, as a single agent or in combination with one or more additional agents, which will relieve to some extent one or more of the symptoms of the condition being treated. In some embodiments, the therapeutically effective amount is an amount sufficient to effect the beneficial or desired clinical results. With respect to the treatment of cancer, a therapeutically effective amount refers to that amount which has at least one of the following effects: palliate, ameliorate, stabilize, reverse, prevent, slow or delay the progression of (and/or symptoms associated with) of cancer. The effective amounts that may be used in the present disclosure varies depending upon the manner of administration, the age, body weight, and general health of the subject. The appropriate amount and dosage regimen can be determined using routine skill in the art.

The terms “wild type” or “WT” are used interchangeably herein and refer to an amino acid sequence or a nucleotide sequence that is found in nature, including allelic variations. A WT protein has an amino acid sequence or is encoded by a nucleotide sequence that has not been intentionally modified.

### General

The present disclosure relates to methods of treating a solid tumor in a subject in need thereof, the method comprising administering to the subject a plurality of doses of a heterodimeric protein, wherein each dose comprises a therapeutically effective amount of a heterodimeric Fc fusion protein (or a combination of heterodimeric Fc fusion proteins) that includes IL-15 and IL-15 receptor alpha (IL-15Rα) protein domains; wherein the administration of the plurality of doses of the heterodimeric protein (1) is sufficient to increase the number of NK cells at least 20-fold as compared to the number of NK cells prior to said administration or (2) results in accumulation of NK cells in the subject. The present disclosure relates to methods for inducing the proliferation of NK cells in a subject, the method comprising administering to the subject a plurality of doses of a heterodimeric protein, wherein each dose comprises an effective amount of a heterodimeric Fc fusion protein (or a combination of heterodimeric Fc fusion proteins) that includes IL-15 and IL-15 receptor alpha (IL-15Rα) protein domains; wherein the administration of the plurality of doses of the heterodimeric protein (1) is sufficient to increase the number of NK cells at least 20-fold as compared to the number of NK cells prior to said administration or (2) results in accumulation of NK cells in the subject. The Fc domains can be derived from IgG Fc domains, e.g., IgG1, IgG2, IgG3 or IgG4 Fc domains.

### IL15-IL15Rα Heterodimeric Fc-Fusion Proteins

Any of the IL15-IL15Rα heterodimeric Fc-fusion proteins disclosed in US2018/0118805, the entire disclosure of which is incorporated by reference herein, or a combination thereof, may be used in the methods disclosed herein. These include, inter alia, the Fc variants such as steric variants (e.g., “knob and holes,” “skew,” “electrostatic steering,” “charged pairs” variants), pI variants, isotypic variants, FcγR variants, and ablation variants (e.g., “FcγR ablation variants” or “Fc knock out (FcKO or KO)” variants) as well as the various IL-15 and IL15Rα proteins disclosed therein.

Thus, in some embodiments, the heterodimeric protein useful in the methods disclosed herein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains, respectively, comprise a set of amino acid substitutions selected from the group consisting of S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L; K370S:S364K/E357Q; S267K/S364K/E357Q:S267K/L368D/K370S; L368D/K370S:S364K/E357Q; S364K:L368D/K370S; S364K:L368E/K370S; D401K:T411E/K360E/Q362E; S364K/E357L:L368D/K370S; and S364K/E357Q:K370S, according to EU numbering.

In some embodiments, said first and said second Fc domains, respectively, comprise the S267K/L368D/K370S:S267K/S364K/E357Q set of amino acid substitutions, according to EU numbering. In some embodiments, said first and said second Fc domains, respectively, comprise the S364K/E357Q:L368D/K370S set of amino acid substitutions, according to EU numbering. In some embodiments, said first and said second Fc domains, respectively, comprise the L368D/K370S:S364K set of amino acid substitutions, according to EU numbering. In some embodiments, said first and said second Fc domains, respectively, comprise the L368E/K370S:S364K set of amino acid substitutions, according to EU numbering. In some embodiments, said first and said second Fc domains, respectively, comprise the T411E/K360E/Q362E:D401K set of amino acid substitutions, according to EU numbering. In some embodiments, said first and said second Fc domains, respectively, comprise the L368D/K370S:S364K/E357L set of amino acid substitutions, according to EU numbering. In some embodiments, said first and said second Fc domains, respectively, comprise the K370S:S364K/E357Q set of amino acid substitutions, according to EU numbering. In some embodiments, said first and said second Fc domains, respectively, comprise the S267K/S364K/E357Q:S267K/L368D/K370S set of amino acid substitutions, according to EU numbering. In some embodiments, said first and said second Fc domains, respectively, comprise the L368D/K370S:S364K/E357Q set of amino acid substitutions, according to EU numbering. In some embodiments, said first and said second Fc domains, respectively, comprise the S364K:L368D/K370S set of amino acid substitutions, according to EU numbering. In some embodiments, said first and said second Fc domains, respectively, comprise the S364K:L368E/K370S set of amino acid substitutions, according to EU numbering. In some embodiments, said first and said second Fc domains, respectively, comprise the D401K:T411E/K360E/Q362E set of amino acid substitutions, according to EU numbering. In some embodiments, said first and said second Fc domains, respectively, comprise the S364K/E357L:L368D/K370S set of amino acid substitutions, according to EU numbering. In some embodiments, said first and said second Fc domains, respectively, comprise the S364K/E357Q:K370S set of amino acid substitutions, according to EU numbering.

In some embodiments, each of said first and/or second Fc domains independently further comprises amino acid substitutions selected from the group consisting of Q295E, N384D, Q418E and N421D, or a combination thereof according to EU numbering. In some embodiments, the first Fc domain further comprises amino acid substitutions selected from the group consisting of Q295E, N384D, Q418E and N421D, or a combination thereof, according to EU numbering. In some embodiments, the second Fc domain further comprises amino acid substitutions selected from the group consisting of Q295E, N384D, Q418E and N421D, or a combination thereof, according to EU numbering. In some embodiments, each of said first and second Fc domains further comprises amino acid substitutions selected from the group consisting of Q295E, N384D, Q418E and N421D, or a combination thereof, according to EU numbering. In some embodiments, the first Fc domain further comprises amino acid substitutions Q295E, N384D, Q418E and N421D, according to EU numbering. In some embodiments, the second Fc domain further comprises amino acid substitutions Q295E, N384D, Q418E and N421D, according to EU numbering. In some embodiments, each of said first and second Fc domains further comprises amino acid substitutions Q295E, N384D, Q418E and N421D, according to EU numbering.

In some embodiments, the first Fc domain does not comprise a free Cysteine at position 220. In some embodiments, the first Fc domain comprises the amino acid substitution C220S, according to EU numbering. In some embodiments, the second Fc domain does not comprise a free Cysteine at position 220. In some embodiments, the second Fc domain comprises the amino acid substitution C220S, according to EU numbering. In some embodiments, the first and second Fc domains do not comprise a free Cysteine at position 220. In some embodiments, the first and second Fc domains both comprise the amino acid substitution C220S, according to EU numbering.

In some embodiments, the first Fc domain further comprises any one of amino acid substitutions selected from the group consisting of E233P, L234V, L2³5A, G236 del, G236R, S239K, S267K, A327G, and L328R or a combination thereof, according to EU numbering. In some embodiments, the first Fc domain further comprises amino acid substitutions E233P, L234V, L235A, G236 del, and S267K, according to EU numbering. In some embodiments, the second Fc domain further comprises any one of amino acid substitutions selected from the group consisting of E233P, L234V, L235A, G236 del, G236R, S239K, S267K, A327G, and L328R, or a combination thereof, according to EU numbering. In some embodiments, the second Fc domain further comprises amino acid substitutions E233P, L234V, L235A, G236 del, and S267K, according to EU numbering. In some embodiments, the first and second Fc domains each comprise amino acid substitutions E233P, L234V, L235A, G236 del, and S267K, according to EU numbering.

The position of the various Fc domain substitutions is in reference to the corresponding position in the wild-type IgG1 Fc domain (SEQ ID NO: 12). The amino acid sequence of the wild-type IgG1 Fc domain (SEQ ID NO: 12) is an exemplary sequence provided for comparison purposes, and the Fc domain of the heterodimeric protein may comprise additional amino acid alterations (e.g., substitutions, insertions, and deletions) relative to the wild-type IgG1 Fc domain (SEQ ID NO: 12). For example, the Fc domain of the heterodimeric protein may be derived from a different wild-type human IgG1 allele. In some embodiments, the Fc domain of the heterodimeric protein does not comprise any additional amino acid alterations relative to the wild-type IgG1 Fc domain (SEQ ID NO: 12). The skilled artisan would be able to determine the corresponding substitutions in an Fc domain derived from an IgG2, an IgG3 or an IgG4 Fc domain. For example, the skilled artisan would recognize that residues E233, L234, L235 and G236 are present in Fc domains derived from IgG1 or IgG3 Fc domains. In some embodiments, the position of the various Fc domain substitutions is in reference to the corresponding position in the wild-type IgG3 Fc domain (SEQ ID NO: 14). The amino acid sequence of the wild-type IgG3 Fc domain (SEQ ID NO: 14) is an exemplary sequence provided for comparison purposes, and the Fc domain of the heterodimeric protein may comprise additional amino acid alterations (e.g., substitutions, insertions, and deletions) relative to the wild-type IgG3 Fc domain (SEQ ID NO: 14). For example, the Fc domain of the heterodimeric protein may be derived from a different wild-type human IgG3 allele. In some embodiments, the Fc domain of the heterodimeric protein does not comprise any additional amino acid alterations relative to the wild-type IgG3 Fc domain (SEQ ID NO: 14).

In some embodiments, each of said first and/or second Fc domains independently further comprises amino acid substitutions selected from the group consisting of G236R/L328R; E233P/L234V/L235A/G236 del/S239K; E233P/L234V/L235A/G236 del/S267K; E233P/L234V/L235A/G236 del/S239K/A327G; E233P/L234V/L235A/G236 del/S267K/A327G; and E233P/L234V/L235A/G236 del, according to EU numbering and wherein the Fc domains are derived from IgG1 or IgG3 Fc domains. In some embodiments, said first second Fc domain further comprises amino acid substitutions selected from the group consisting of G236R/L328R; E233P/L234V/L235A/G236 del/S239K; E233P/L234V/L235A/G236 del/S267K; E233P/L234V/L235A/G236 del/S239K/A327G; E233P/L234V/L235A/G236 del/S267K/A327G; and E233P/L234V/L235A/G236 del, according to EU numbering and wherein the Fc domains are derived from IgG1 or IgG3 Fc domains. In some embodiments, said second Fc domain further comprises amino acid substitutions selected from the group consisting of G236R/L328R; E233P/L234V/L235A/G236 del/S239K; E233P/L234V/L235A/G236 del/S267K; E233P/L234V/L235A/G236 del/S239K/A327G; E233P/L234V/L235A/G236 del/S267K/A327G; and E233P/L234V/L235A/G236 del, according to EU numbering and wherein the Fc domains are derived from IgG1 or IgG3 Fc domains. In some embodiments, said first and second Fc domains further comprise amino acid substitutions selected from the group consisting of G236R/L328R; E233P/L234V/L235A/G236 del/S239K; E233P/L234V/L235A/G236 del/S267K; E233P/L234V/L235A/G236 del/S239K/A327G; E233P/L234V/L235A/G236 del/S267K/A327G; and E233P/L234V/L235A/G236 del, according to EU numbering and wherein the Fc domains are derived from IgG1 or IgG3 Fc domains.

The skilled artisan would also recognize that the corresponding residues in a Fc domain derived IgG2 Fc domain are P233, V234, and A235 and that an Fc domain derived from IgG2 lacks a residue corresponding to residue G236. Accordingly, the skilled artisan would recognize that reference to E233P, L234V, L235A, and G236 del herein is a reference to P233, V234, A235 and −236 if the Fc domain is derived from an IgG2 Fc domain (i.e., the PVA-sequence present in wild type IgG2). In some embodiments, the position of the various Fc domain substitutions is in reference to the corresponding position in the wild-type IgG2 Fc domain (SEQ ID NO: 13). The amino acid sequence of the wild-type IgG2 Fc domain (SEQ ID NO: 13) is an exemplary sequence provided for comparison purposes, and the Fc portion of the heterodimeric protein may comprise additional amino acid alterations (e.g., substitutions, insertions, and deletions) relative to the wild-type IgG2 Fc domain (SEQ ID NO: 13). For example, the Fc domain of the heterodimeric protein may be derived from a different wild-type human IgG2 allele. In some embodiments, the Fc domain of the heterodimeric protein does not comprise any additional amino acid alterations relative to the wild-type IgG2 Fc domain (SEQ ID NO: 13).

In some embodiments, each of said first and/or second Fc domains independently further comprises amino acid substitutions selected from the group consisting of L328R; S239K; S267K; S239K/A327G; and S267K/A327G, according to EU numbering and wherein the Fc domains are derived from IgG2 Fc domain. In some embodiments, said first Fc domain further comprises amino acid substitutions selected from the group consisting of L328R; S239K; S267K; S239K/A327G; and S267K/A327G, according to EU numbering and wherein the Fc domains are derived from IgG2 Fc domain. In some embodiments, said second Fc domain further comprises amino acid substitutions selected from the group consisting of L328R; S239K; S267K; S239K/A327G; and S267K/A327G, according to EU numbering and wherein the Fc domains are derived from IgG2 Fc domain. In some embodiments, said first and second Fc domains further comprise amino acid substitutions selected from the group consisting of L328R; S239K; S267K; S239K/A327G; and S267K/A327G, according to EU numbering and wherein the Fc domains are derived from IgG2 Fc domain.

The skilled artisan would also recognize that in a Fc domain derived from an IgG4, residue 234 is a phenylalanine. Accordingly, the skilled artisan would recognize that reference to L234 herein (e.g., L234V) is a reference to F234 (e.g., F234V) if the Fc domain is derived from an IgG4 Fc domain. In some embodiments, the position of the various Fc domain substitutions is in reference to the corresponding position in the wild-type IgG4 Fc domain (SEQ ID NO: 15). The amino acid sequence of the wild-type IgG4 Fc domain (SEQ ID NO: 15) is an exemplary sequence provided for comparison purposes, and the Fc domain of the heterodimeric protein may comprise additional amino acid alterations (e.g., substitutions, insertions, and deletions) relative to the wild-type IgG4 Fc domain (SEQ ID NO: 15). For example, the Fc domain of the heterodimeric protein may be derived from a different wild-type human IgG4 allele. In some embodiments, the Fc domain of the heterodimeric protein does not comprise any additional amino acid alterations relative to the wild-type IgG4 Fc domain (SEQ ID NO: 15).

In some embodiments, each of said first and/or second Fc domains independently further comprises amino acid substitutions selected from the group consisting of G236R/L328R; E233P/F234V/L235A/G236 del/S239K; E233P/F234V/L235A/G236 del/S267K; E233P/F234V/L235A/G236 del/S239K/A327G; E233P/F234V/L235A/G236 del/S267K/A327G; and E233P/F234V/L235A/G236 del, according to EU numbering and wherein the Fc domains are derived from IgG4 Fc domain. In some embodiments, first Fc domain further comprises amino acid substitutions selected from the group consisting of G236R/L328R; E233P/F234V/L235A/G236 del/S239K; E233P/F234V/L235A/G236 del/S267K; E233P/F234V/L235A/G236 del/S239K/A327G; E233P/F234V/L235A/G236 del/S267K/A327G; and E233P/F234V/L235A/G236 del, according to EU numbering and wherein the Fc domains are derived from IgG4 Fc domain. In some embodiments, said second Fc domain further comprises amino acid substitutions selected from the group consisting of G236R/L328R; E233P/F234V/L235A/G236 del/S239K; E233P/F234V/L235A/G236 del/S267K; E233P/F234V/L235A/G236 del/S239K/A327G; E233P/F234V/L235A/G236 del/S267K/A327G; and E233P/F234V/L235A/G236 del, according to EU numbering and wherein the Fc domains are derived from IgG4 Fc domain. In some embodiments, said first and second Fc domains further comprise amino acid substitutions selected from the group consisting of G236R/L328R; E233P/F234V/L235A/G236 del/S239K; E233P/F234V/L235A/G236 del/S267K; E233P/F234V/L235A/G236 del/S239K/A327G; E233P/F234V/L235A/G236 del/S267K/A327G; and E233P/F234V/L235A/G236 del, according to EU numbering and wherein the Fc domains are derived from IgG4 Fc domain.

In some embodiments, the first Fc domain further comprises the amino acid substitution M428L or N434S, according to EU numbering. In some embodiments, the first Fc domain further comprises the amino acid substitution M428L, according to EU numbering. In some embodiments, the first Fc domain further comprises the amino acid substitution N434S, according to EU numbering. In some embodiments, the second Fc domain further comprises the amino acid substitution M428L or N434S, according to EU numbering. In some embodiments, the second Fc domain further comprises the amino acid substitution M428L, according to EU numbering. In some embodiments, the second Fc domain further comprises the amino acid substitution N434S, according to EU numbering. In some embodiments, the first Fc domain further comprises amino acid substitutions M428L and N434S, according to EU numbering. In some embodiments, the second Fc domain further comprises amino acid substitutions M428L and N434S, according to EU numbering. In some embodiments, the first and second Fc domains each further comprise amino acid substitutions M428L and N434S, according to EU numbering.

In some embodiments, said first and/or second Fc domain further comprises amino acid substitution K246T, according to EU numbering. In some embodiments, the first Fc domain further comprises amino acid substitution K246T, according to EU numbering. In some embodiments, the second Fc domain further comprises amino acid substitution K246T, according to EU numbering. When the K246T substitution appears in the second Fc domain, it may also be called a K100T mutation based on the amino acid numbering of the second monomer (see, e.g., SEQ ID NO: 10 and 16). In some embodiments, the first and second Fc domains further comprise amino acid substitution K246T, according to EU numbering.

In some embodiments, the first Fc domain comprises amino acid substitutions L368D and K370S; the second Fc domain comprises amino acid substitutions S364K and E357Q; and each of said first and second Fc domains further comprises amino acid substitutions C220S, E233P, L234V, L235A, G236 del, S267K, M428L and N434S; wherein, according to EU numbering. In some embodiments, the first Fc domain comprises amino acid substitutions S364K and E357Q; the second Fc domain comprises amino acid substitutions L368D and K370S; and each of said first and second Fc domains further comprises amino acid substitutions C220S, E233P, L234V, L235A, G236 del, S267K, M428L and N434S, according to EU numbering.

In some embodiments, the first Fc domain comprises amino acid substitutions L368D and K370S; the second Fc domain comprises amino acid substitutions K246T, S364K, and E357Q; and each of said first and second Fc domains further comprises amino acid substitutions C220S, E233P, L234V, L235A, G236 del, S267K, M428L and N434S, according to EU numbering. In some embodiments, the first Fc domain comprises amino acid substitutions S364K and E357Q; the second Fc domain comprises amino acid substitutions K246T, L368D and K370S; and each of said first and second Fc domains further comprises amino acid substitutions C220S, E233P, L234V, L235A, G236 del, S267K, M428L and N434S, according to EU numbering.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, any one of the amino acid substitutions of the Fe variant domains described herein are on one of the monomers or on both monomers (e.g., on the first Fc domain; on the second Fc domain or on both Fc domains).

In some embodiments, the Fc domain of the first monomer is derived from IgG1, IgG2, IgG3, or IgG4. In some embodiments, the Fc domain of the first monomer is derived from IgG1. In some embodiments, the Fc domain of the first monomer is derived from IgG2. In some embodiments, the Fc domain of the first monomer is derived from IgG3. In some embodiments, the Fc domain of the first monomer is derived from IgG4. In some embodiments, the Fc domain of the second monomer is derived from IgG1, IgG2, IgG3, or IgG4. In some embodiments, the Fc domain of the second monomer is derived from IgG1. In some embodiments, the Fc domain of the second monomer is derived from IgG2. In some embodiments, the Fc domain of the second monomer is derived from IgG3. In some embodiments, the Fc domain of the second monomer is derived from IgG4.

As used herein, “IL-15,” “IL15” or “Interleukin 15” may be used interchangeably and refer to a four-α-helix protein belonging to a family of cytokines. IL-15 signals through α receptor complex composed of the IL-2/IL-15 receptor β (IL-15Rβ) (CD122) subunit. In some embodiments, the IL-15 protein comprises the polypeptide sequence set forth in SEQ ID NO:2 (full-length human IL-15). In some embodiments, the IL-15 protein comprises the polypeptide sequence set forth in SEQ ID NO:1 (truncated or mature human IL-15). In some embodiments, the IL-15 protein comprises a polypeptide sequence selected from the group consisting of SEQ ID NO:1 and SEQ ID NO:2.

In some embodiments, the IL-15 protein of the first monomer is an IL-15 protein variant having a different amino acid sequence than wild type IL-15 protein (SEQ ID NO: 1). In some embodiments, the IL-15 variant is engineered to have reduced binding affinity (compared with wild-type IL-15) towards IL-2/IL-15βγ receptor complex with the goal of improving tolerability and extending pharmacokinetics by reducing acute toxicity, and ultimately promote antitumor immunity through IL-15 mediated signaling on CD8⁺ T cells and NK cells. In certain embodiments, the sequence of the IL-15 protein variant of the first monomer has at least one (i.e., 1, 2, 3, 4, 5, 6, 7, 8, 9, 10, or more) amino acid substitutions compared to the wild-type IL-15 sequence protein (SEQ ID NO: 1). In some embodiments, the amino acid substitution may include one or more of an amino acid substitution or deletion in the domain of IL-15 that interacts with IL-15R and/or IL-2/IL-15βγ receptor complex. In some embodiments, the amino acid substitution may include one or more of an amino acid substitution or deletion in the domain of IL-15 protein which causes a decreased binding affinity, compared with the affinity of a wild-type IL-15, towards IL-2/IL-15βγ receptor complex. In some embodiments, the IL-15 protein comprises one or more amino acid substitutions selected from the group consisting of N1D, N4D, D8N, D30N, D61N, E64Q, N65D and Q108E. In some embodiments, said IL15 protein comprises one or more amino acid substitutions selected from the group consisting of E87C, V49C, L52C, E89C, Q48C, E53C, C42S and L45C. The amino acid substitutions for the IL-15 protein disclosed herein are relative to wild-type IL-15 (mature form; SEQ ID NO: 1). The amino acid sequence of wild-type IL-15 (mature form; SEQ ID NO: 1) is an exemplary sequence provided for comparison purposes, and the IL-15 protein of the heterodimeric protein may comprise additional amino acid alterations (e.g., substitutions, insertions, and deletions) relative to wild-type IL-15. For example, the IL-15 protein of the heterodimeric protein may be derived from a different wild-type human IL-15 allele. In some embodiments, the IL-15 protein of the heterodimeric protein does not comprise any additional amino acid alterations relative to wild-type IL-15. In some embodiments, the IL-15 protein variant present in the first monomer comprises the amino acid sequence set forth in SEQ ID NO:5 (XENP24306/XENP32803).

In some embodiments, the IL-15 protein comprises amino acid substitutions D30N, E64Q and N65D. In some embodiments, the IL-15 protein comprises the following amino acid substitutions: N4D and N65D. In some embodiments, the IL-15 protein comprises the following amino acid substitutions: D30N and N65D. In some embodiments, the IL-15 protein present in the first monomer comprises an N65D amino acid substitution and one or more amino acid substitutions selected from the group consisting of N4D, D30N, E64Q. In some embodiments, the IL-15 protein present in the first monomer comprises an N65D amino acid substitution and one or more amino acid substitutions selected from the group consisting of N4D, D30N, E64Q. In some embodiments, the IL-15 protein present in the first monomer comprises an N65D amino acid substitution and consists of the amino acid substitutions N4D, D30N, E64Q. The amino acid substitutions for the IL-15 protein disclosed herein are relative to wild-type IL-15 (SEQ ID NO: 1). The amino acid sequence of wild-type IL-15 (SEQ ID NO: 1) is an exemplary sequence provided for comparison purposes, and the IL-15 protein of the heterodimeric protein may comprise additional amino acid alterations (e.g., substitutions, insertions, and deletions) relative to wild-type IL-15. For example, the IL-15 protein of the heterodimeric protein may be derived from a different wild-type human IL-15 allele. In some embodiments, the IL-15 protein of the heterodimeric protein does not comprise any additional amino acid alterations relative to wild-type IL-15.

IL-15Rα protein is a transmembrane protein with very high affinity for IL-15 that facilitates IL-15 trafficking from the endoplasmic reticulum (ER) through the cytoplasm and presentation of IL-15/IL-15Rα complexes on the cell surface. As used herein, the term “sushi domain of IL-15Rα” refers to the truncated extracellular region of IL-15Rα or recombinant human IL-15 receptor α. In some embodiments, the IL-15Rα protein comprises a polypeptide sequence of SEQ ID NO:3 (full-length human IL-15Rα). In some embodiments, the IL-15Rα protein comprises a polypeptide sequence of SEQ TD NO: 4 (sushi domain of human IL-15Rα).

In some embodiments, said IL15Rα protein comprises one or more amino acid alterations selected from the group consisting of DPC or DCA insertions after residue 65 (65DPC or D96/P97/C98, 65DCA or D96/C97/A98), S40C, K34C, G38C, L42C and A37C. The numbering of these amino acid substitutions for the IL-15Rα protein is relative to the sushi domain of human IL-15Rα (SEQ ID NO: 4). The amino acid sequence of the sushi domain of human IL-15Rα (SEQ ID NO: 4) is an exemplary sequence provided for comparison purposes, and the IL-15Rα protein of the heterodimeric protein may comprise additional amino acid alterations (e.g., substitutions, insertions, and deletions) relative to the sushi domain of human IL-15Rα (SEQ ID NO: 4). For example, the IL-15Rα protein of the heterodimeric protein may be derived from a different wild-type human IL-15Rα allele. In some embodiments, the IL-15Rα protein of the heterodimeric protein does not comprise any additional amino acid alterations relative to the sushi domain of human IL-15Rα (SEQ ID NO: 4).

In some embodiments, IL15 protein and the IL15Rα protein comprise a set of amino acid substitutions or additions selected from the group consisting of E87C: 65DPC (DPC insertions after residue 65 or D96/P97/C98); E87C: 65DCA (DCA insertions after residue 65 or D96/C97/A98); V49C:S40C; L52C:S40C; E89C:K34C; Q48C:G38C; E53C:L42C; C42S:A37C; and L45C:A37C, respectively. The numbering of these amino acid substitutions for the IL-15Rα protein is relative to the sushi domain of human IL-15Rα (SEQ ID NO: 4). The amino acid sequence of the sushi domain of human IL-15Rα (SEQ ID NO: 4) is an exemplary sequence provided for comparison purposes, and the IL-15Rα protein of the heterodimeric protein may comprise additional amino acid alterations (e.g., substitutions, insertions, and deletions) relative to the sushi domain of human IL-15Rα (SEQ ID NO: 4). For example, the IL-15Rα of the heterodimeric protein may be derived from a different wild-type human IL-15Rα allele. In some embodiments, the IL-15Rα protein of the heterodimeric protein does not comprise any additional amino acid alterations relative to the sushi domain of human IL-15Rα (SEQ ID NO: 4).

In some embodiments, the IL-15Rα protein comprises the amino acid sequence of SEQ ID NO:3 (full-length human IL-15Rα). In some embodiments, the IL-15Rα protein comprises the amino acid sequence SEQ ID NO: 4 (sushi domain of human IL-15Rα). In some embodiments, the IL-15 protein comprises amino acid substitutions D30N, E64Q and N65D; and the IL-15Rα protein comprises SEQ ID NO: 4 (sushi domain of human IL-15Rα).

The heterodimeric protein of the disclosure is an IL-15/IL-15Rα-Fc heterodimeric fusion protein. The N-terminus of one side of the heterodimeric Fc domain is covalently attached to the C-terminus of IL-15 protein, while the other side is covalently attached to the sushi domain (truncated extracellular region) of IL-15Rα. In some embodiments, the IL-15 protein and IL-15Rα (sushi domain) may have a variable length linker between the C-terminus of IL-15 and IL-15Rα and the N-terminus of each of the Fc regions. In some embodiments, the IL-15 protein is covalently attached to the N-terminus of the first Fc domain via a first linker. In some embodiments, the IL-15Rα protein is covalently attached to the N-terminus of the second Fc domain using a second linker. The term “linker,” as used herein, refers to a polypeptide sequence that joins two or more domains. The characteristics of linkers and their suitability for particular purposes are known in the art. See, e.g., Chen et al. Adv Drug Deliv Rev. October 15; 65(10): 1357-1369 (2013) (disclosing various types of linkers, their properties, and associated linker designing tools and databases), which is incorporated herein by reference. In some embodiments, the linker is flexible, rigid, or in vivo cleavable. In some embodiments, the linker is flexible. Flexible linkers typically comprise small non-polar amino acids (e.g., Gly) or polar amino acids (e.g., Ser or Thr). Examples of flexible linkers that can be used in the present disclosure are sequences consisting primarily of stretches of Gly and Ser residues (“GS” linker). In some embodiments, flexible linkers comprise repeats of 4 Gly and Ser residues. In some embodiments, the flexible linker comprises 1-5 repeats of five Gly and Ser residues. Non-limiting examples of flexible linker include (Gly-Gly-Gly-Gly-Ser)n (SEQ ID NO: 39), (Ser-Ser-Ser-Ser-Gly)n (SEQ ID NO: 40), (Gly-Ser-Ser-Gly-Gly)n (SEQ ID NO: 41), and (Gly-Gly-Ser-Gly-Gly)n (SEQ ID NO: 42), where n may be any integer between 1 and 5. In some embodiments, the linker is between 5 and 25 amino acid residues long. In some embodiments, the flexible linker comprises 5, 10, 15, 20, or 25 residues. Other suitable linkers may be selected from the group consisting of AS (SEQ ID NO: 43), AST (SEQ ID NO: 44), TVAAPS (SEQ ID NO: 45), TVA (SEQ ID NO: 46), ASTSGPS (SEQ ID NO: 47), KESGSVSSEQLAQFRSLD (SEQ ID NO: 48), EGKSSGSGSESKST (SEQ ID NO: 49), (Gly)6 (SEQ ID NO: 50), (Gly)8 (SEQ ID NO: 51), and GSAGSAAGSGEF (SEQ ID NO: 52). In general, a flexible linker provides good flexibility and solubility and may serve as a passive linker to keep a distance between functional domains. The length of the flexible linkers can be adjusted to allow for proper folding or to achieve optimal biological activity of the fusion proteins. In some embodiments, the linker comprises the sequence (Gly-Gly-Gly-Gly-Ser; SEQ ID NO: 53). In some embodiments, the first and second linker comprise different sequences. In some embodiments, the first and second linker comprise the same sequence. In some embodiments, the first and second linker comprise the sequence set forth in SEQ ID NO: 53. In some embodiments, the first and second linker consists of the sequence set forth in SEQ ID NO: 53.

In some embodiments, the heterodimeric protein useful in the methods disclosed herein comprises (i) a first monomer comprising IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein each of said first and second Fc domains independently comprises amino acid substitutions E233P, L234V, L235A, G236 del, and S267K, according to EU numbering; and wherein said IL-15 protein comprises an N65D amino acid substitution and one or more amino acid substitutions selected from the group consisting of N4D, D30N, E64Q. The position of the various Fc domain substitutions is in reference to the corresponding position in the wild-type IgG1 Fc domain (SEQ ID NO: 12). The amino acid sequence of the wild-type IgG1 Fc domain (SEQ ID NO: 12) is an exemplary sequence provided for comparison purposes, and the IL-15Rα protein of the heterodimeric protein may comprise additional amino acid alterations (e.g., substitutions, insertions, and deletions) relative to the wild-type IgG1 Fc domain (SEQ ID NO: 12). For example, the Fc domain of the heterodimeric protein may be derived from a different wild-type human IgG1 allele. In some embodiments, the Fc domain of the heterodimeric protein does not comprise any additional amino acid alterations relative to the wild-type IgG1 Fc domain (SEQ ID NO: 12). The amino acid substitutions for the IL-15 protein disclosed herein are relative to wild-type IL-15 (mature form; SEQ ID NO: 1). The amino acid sequence of wild-type IL-15 (mature form; SEQ ID NO: 1) is an exemplary sequence provided for comparison purposes, and the IL-15 protein of the heterodimeric protein may comprise additional amino acid alterations (e.g., substitutions, insertions, and deletions) relative to wild-type IL-15. For example, the IL-15 protein of the heterodimeric protein may be derived from a different wild-type human IL-15 allele. In some embodiments, the IL-15 protein of the heterodimeric protein does not comprise any additional amino acid alterations relative to wild-type IL-15.

The skilled artisan would be able to determine the corresponding substitutions in an Fc domain derived from an IgG2, an IgG3 or an IgG4 Fc domain. For example, the skilled artisan would recognize that residues E233, L234, L235, G236 and A327 are present in Fc domains derived from IgG1 or IgG3 Fc domains. In some embodiments, the position of the various Fc domain substitutions is in reference to the corresponding position in the wild-type IgG3 Fc domain (SEQ ID NO: 14). The amino acid sequence of the wild-type IgG3 Fc domain (SEQ ID NO: 14) is an exemplary sequence provided for comparison purposes, and the IL-15Rα protein of the heterodimeric protein may comprise additional amino acid alterations (e.g., substitutions, insertions, and deletions) relative to the wild-type IgG3 Fc domain (SEQ ID NO: 14). For example, the Fc domain of the heterodimeric protein may be derived from a different wild-type human IgG3 allele. In some embodiments, the Fc domain of the heterodimeric protein does not comprise any additional amino acid alterations relative to the wild-type IgG3 Fc domain (SEQ ID NO: 14). The skilled artisan would recognize, therefore, that each of said first and second Fc domains independently comprises amino acid substitutions E233P, L234V, L235A, G236 del, and S267K, according to EU numbering, when the Fc domains are derived from an IgG1 or an IgG3 Fc domain.

The skilled artisan would also recognize that the corresponding residues in a Fc domain derived IgG2 Fc domain are P233, V234, A235 and G327 and that an Fc domain derived from IgG2 lacks a residue corresponding to residue G236. Accordingly, the skilled artisan would recognize that reference to E233P, L234V, L235A G236 del and A327G herein is a reference to P233, V234, A235, −236 and no substitution in residue 327, if the Fc domain is derived from an IgG2 Fc domain (i.e., the PVA-sequence present in wild type IgG2). In some embodiments, the position of the various Fc domain substitutions is in reference to the corresponding position in the wild-type IgG2 Fc domain (SEQ ID NO: 13). The amino acid sequence of the wild-type IgG2 Fc domain (SEQ ID NO: 13) is an exemplary sequence provided for comparison purposes, and the IL-15Rα protein of the heterodimeric protein may comprise additional amino acid alterations (e.g., substitutions, insertions, and deletions) relative to the wild-type IgG2 Fc domain (SEQ ID NO: 13). For example, the Fc domain of the heterodimeric protein may be derived from a different wild-type human IgG2 allele. In some embodiments, the Fc domain of the heterodimeric protein does not comprise any additional amino acid alterations relative to the wild-type IgG2 Fc domain (SEQ ID NO: 13). The skilled artisan would recognize, therefore, that each of said first and second Fc domains independently comprises the amino acid substitution S267K, according to EU numbering, when the Fc domains are derived from an IgG2 Fc domain.

The skilled artisan would also recognize that in a Fc domain derived from an IgG4 residue 234 is a phenylalanine and residue 327 is a glycine. Accordingly, the skilled artisan would recognize that reference to L234 herein (e.g., L234V) and A327 (e.g., A327G) is a reference to F234 (e.g., F234V) and no substitution in residue 327, respectively, if the Fc domain is derived from an IgG4 Fc domain. In some embodiments, the position of the various Fc domain substitutions is in reference to the corresponding position in the wild-type IgG4 Fc domain (SEQ ID NO: 15). The amino acid sequence of the wild-type IgG4 Fc domain (SEQ ID NO: 15) is an exemplary sequence provided for comparison purposes, and the IL-15Rα protein of the heterodimeric protein may comprise additional amino acid alterations (e.g., substitutions, insertions, and deletions) relative to the wild-type IgG4 Fc domain (SEQ ID NO: 15). For example, the Fc domain of the heterodimeric protein may be derived from a different wild-type human IgG4 allele. In some embodiments, the Fc domain of the heterodimeric protein does not comprise any additional amino acid alterations relative to the wild-type IgG4 Fc domain (SEQ ID NO: 15). The skilled artisan would recognize, therefore, that each of said first and second Fc domains independently comprises amino acid substitutions E233P, F234V, L235A, G236 del, and S267K, according to EU numbering, when the Fc domains are derived from an IgG4 Fc domain.

In some embodiments, the first Fc domain and/or the second Fc domain are independently engineered to further prolong systemic exposure and increase half-life through enhanced FcRn binding at a lower pH (6.0). In some embodiments, additional engineering on the Fc region makes the heterodimeric protein of the disclosure effectorless (i.e., abolish the binding to Fcγ receptors) and eliminates antibody-mediated CL of T cells and NK cells. In some embodiments, the first and/or second Fc domain are independently engineered to encourage heterodimerization formation over homodimerization formation. In some embodiments, the first and/or second Fc domain are independently engineered to have improved PK. In some embodiments, the first and/or second Fc domain are independently engineered to allow purification of homodimers away from heterodimers by increasing the pI difference between the two monomers. In certain embodiments, the Fc variant domain may further comprise a molecule or sequence that lacks one or more native Fc amino acid residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a neonatal receptor, (7) antibody-dependent cell-mediated cytotoxicity (ADCC), or (8) antibody dependent cellular phagocytosis (ADCP). Fc variants are described in further detail hereinafter.

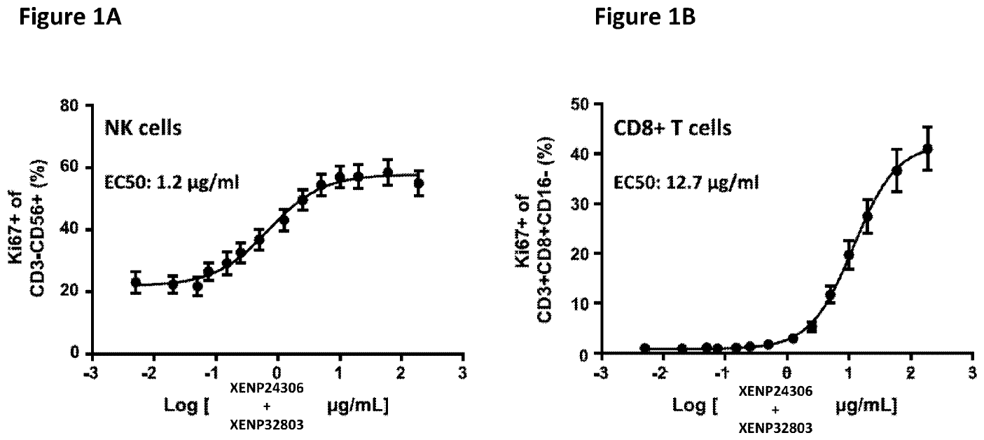

In some embodiments, the first or second Fc domain of the present disclosure may comprise “skew” variants (e.g., a set of amino acid substitutions as shown in FIGS. 1A-1C of U.S. Pat. No. 10,259,887; all of which are herein incorporated by reference in its entirety). Skew variants encourage heterodimerization formation over homodimerization formation. In some embodiments, the skew variants are selected from the group consisting of S364K/E357Q (on the first Fc domain): L368D/K370S (on the second Fc domain); L368D/K370S:S364K; L368E/K370S:S364K; T411E/K360E/Q362E:D401K; L368D/K370S: S364K/E357L, K370S: S364K/E357Q, T366S/L368A/Y407V: T366W and T366S/L368A/Y407V/Y349C: T366W/S354C, according to EU numbering. In some embodiments, said first Fc domain further comprises amino acid substitutions L368D and K370S and said second Fc domain further comprises amino acid substitutions S364K and E357Q, according to EU numbering. In some embodiments, said first Fc domain further comprises amino acid substitutions S364K and E357Q and said second Fc domain further comprises amino acid substitutions L368D and K370S, according to EU numbering.

In some embodiments, the first Fc domain further comprises amino acid substitutions selected from the group consisting of Q295E, N384D, Q418E and N421D, or a combination thereof, according to EU numbering. In some embodiments, the second Fc domain further comprises any one of amino acid substitutions selected from the group consisting of Q295E, N384D, Q418E and N421D, or a combination thereof, according to EU numbering. In some embodiments, said first Fc domain further comprises amino acid substitutions Q295E, N384D, Q418E and N421D, according to EU numbering. In some embodiments, said second Fc domain further comprises amino acid substitutions Q295E, N384D, Q418E and N421D, according to EU numbering. In some embodiments, said first and second Fc domains further comprise amino acid substitutions Q295E, N384D, Q418E and N421D, according to EU numbering.

In some embodiments, the first Fc domain does not comprise a free Cysteine at position 220. In some embodiments, the first Fc domain comprises the amino acid substitution C220S, according to EU numbering. In some embodiments, the second Fc domain does not comprise a free Cysteine at position 220. In some embodiments, the second Fc domain comprises the amino acid substitution C220S, according to EU numbering. In some embodiments, the first and second Fc domains do not comprise a free Cysteine at position 220. In some embodiments, the first and second Fc domains comprise the amino acid substitution C220S, according to EU numbering.

In some embodiments, the first or the second Fc domain of the present disclosure may include amino acid substitutions for improved PK (Xtend substitutions). In some embodiments, the first and/or second Fc domains of the present disclosure independently comprise amino acid substitutions M428L and/or N434S, according to EU numbering. In some embodiments, the first Fc domain comprises the amino acid substitution M428L or N434S. In some embodiments, the first Fc domain comprises amino acid substitutions M428L and N434S. In some embodiments, the first Fc domain comprises the amino acid substitution M428L. In some embodiments, the first Fc domain comprises the amino acid substitution N434S. In some embodiments, the second Fc domain comprises the amino acid substitution M428L or N434S. In some embodiments, the second Fc domain comprises amino acid substitutions M428L and N434S. In some embodiments, the second Fc domain comprises the amino acid substitution M428L. In some embodiments, the second Fc domain comprises the amino acid substitution N434S.

In some embodiments, said first and/or second Fc domain further comprises amino acid substitution K246T, according to EU numbering. In some embodiments, the first Fc domain further comprises amino acid substitution K246T, according to EU numbering. In some embodiments, the second Fc domain further comprises amino acid substitution K246T, according to EU numbering. When the K246T substitution appears in the second Fc domain, it may also be referred to as a K100T mutation based on the amino acid numbering of the second monomer (see, e.g., SEQ ID NO: 10 and 16). In some embodiments, the first and second Fc domains further comprise amino acid substitution K246T, according to EU numbering.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, any one of the amino acid substitutions of the Fc variant domains described herein are on one of the monomers or on both monomers (e.g., on the first Fc domain; on the second Fc domain or on both Fc domains).

In some embodiments, the Fc domain of the first monomer is derived from IgG1, IgG2, IgG3, or IgG4. In some embodiments, the Fc domain of the first monomer is derived from IgG1. In some embodiments, the Fc domain of the first monomer is derived from IgG2. In some embodiments, the Fc domain of the first monomer is derived from IgG3. In some embodiments, the Fc domain of the first monomer is derived from IgG4. In some embodiments, the Fc domain of the second monomer is derived from IgG1, IgG2, IgG3, or IgG4. In some embodiments, the Fc domain of the second monomer is derived from IgG1. In some embodiments, the Fc domain of the second monomer is derived from IgG2. In some embodiments, the Fc domain of the second monomer is derived from IgG3. In some embodiments, the Fc domain of the second monomer is derived from IgG4.

In some embodiments, said first Fc domain comprises the following amino acid substitutions: C220S, E233P, L234V, L235A, G236 del, S267K, L368D, K370S, M428L and N434S, according to EU numbering. In some embodiments, said second Fc domain comprises the following amino acid substitutions, according to EU numbering: C220S, E233P, L234V, L235A, G236 del, S267K, S364K, E357Q, M428L and N434S. In some embodiments, said second Fc domain comprises the following amino acid substitutions: C220S, E233P, L234V, L235A, G236 del, S267K, L368D, K370S, M428L and N434S, according to EU numbering. In some embodiments, said first Fc domain comprises the following amino acid substitutions: C220S, E233P, L234V, L235A, G236 del, S267K, S364K, E357Q, M428L and N434S, according to EU numbering. In some embodiments, the first Fc domain does not comprise any additional amino acid alterations compared to a wild-type IgG Fc domain. In some embodiments, the first Fc domain does not comprise any additional amino acid alterations compared to a wild-type IgG1 Fc domain. In some embodiments, the first Fc domain does not comprise any additional amino acid alterations compared to SEQ ID NO: 12. In some embodiments, the second Fc domain does not comprise any additional amino acid alterations compared to a wild-type IgG Fc domain. In some embodiments, the second Fc domain does not comprise any additional amino acid alterations compared to a wild-type IgG1 Fc domain. In some embodiments, the second Fc domain does not comprise any additional amino acid alterations compared to SEQ ID NO: 12.

In some embodiments, each of said first and second Fc domains independently comprises an additional set of amino acid substitutions selected from the group consisting of G236R, S239K, L328R, and A327G, according to EU numbering.

In some embodiments, the Fc domain of the first monomer is derived from IgG1, IgG2, IgG3, or IgG4. In some embodiments, the Fc domain of the first monomer is derived from IgG1. In some embodiments, the Fc domain of the first monomer is derived from IgG2. In some embodiments, the Fc domain of the first monomer is derived from IgG3. In some embodiments, the Fc domain of the first monomer is derived from IgG4. In some embodiments, the Fc domain of the second monomer is derived from IgG1, IgG2, IgG3, or IgG4. In some embodiments, the Fc domain of the second monomer is derived from IgG1. In some embodiments, the Fc domain of the second monomer is derived from IgG2. In some embodiments, the Fc domain of the second monomer is derived from IgG3. In some embodiments, the Fc domain of the second monomer is derived from IgG4.

In some embodiments, the heterodimeric protein comprises (i) a first monomer comprising IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236 del, S267K, Q295E, L368D, K370S, N384D, Q418E, N421D, M428L, and N434S, and wherein the second Fc domain comprises amino acid substitutions C220S, E233P, L234V, L235A, G236 del, S267K, E357Q, S364K, M428L, and N434S, according to EU numbering; and wherein said IL-15 protein comprises amino acid substitutions D30N, E64Q and N65D, compared to a wild-type IL-15 protein (SEQ ID NO:1).

In some embodiments, the heterodimeric protein comprises (i) a first monomer comprising IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236 del, S267K, Q295E, E357Q, S364K, N384D, Q418E, N421D, M428L, and N434S; and wherein the second Fc domain comprises amino acid substitutions C220S, E233P, L234V, L235A, G236 del, S267K, L368D, K370S, M428L, and N434S, according to EU numbering; and wherein said IL-15 protein comprises amino acid substitutions D30N, E64Q and N65D, compared to a wild-type IL-15 protein (SEQ ID NO:1).

In some embodiments, the heterodimeric protein comprises (i) a first monomer comprising IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236 del, S267K, Q295E, L368D, K370S, N384D, Q418E, N421D, M428L, and N434S, and wherein the second Fc domain comprises amino acid substitutions C220S, E233P, L234V, L235A, G236 del, K246T, S267K, E357Q, S364K, M428L, and N434S, according to EU numbering; and wherein said IL-15 protein comprises amino acid substitutions D30N, E64Q and N65D, compared to a wild-type IL-15 protein (SEQ ID NO:1).

In some embodiments, the heterodimeric protein comprises (i) a first monomer comprising IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236 del, S267K, Q295E, E357Q, S364K, N384D, Q418E, N421D, M428L, and N434S; and wherein the second Fc domain comprises amino acid substitutions C220S, E233P, L234V, L235A, G236 del, K246T, S267K, L368D, K370S, M428L, and N434S, according to EU numbering; and wherein said IL-15 protein comprises amino acid substitutions D30N, E64Q and N65D, compared to a wild-type IL-15 protein (SEQ ID NO:1).

It is known in the art that during the processing and expression of Fc-containing proteins that the C-terminal lysine may be cleaved (also known in the art as C-terminal lysine clipping). Accordingly, for each sequence disclosed herein that contains a C-terminal lysine, the corresponding sequence without the C-terminal lysine (i.e., the C-terminal lysine cleavage product) is also contemplated. In some embodiments, the first monomer comprises a C-terminal lysine. In some embodiments, the first monomer lacks a C-terminal lysine. In some embodiments, the second monomer comprises a C-terminal lysine. In some embodiments, the second monomer lacks a C-terminal lysine.

It is also known in the art that the C-terminal cleavage process is imprecise and that additional C-terminal residues are cleaved. Accordingly, for each sequence disclosed herein that contains a C-terminal lysine, the corresponding sequence without the two C-terminal residues is also contemplated. In some embodiments, for each sequence disclosed herein that contains a C-terminal lysine, the corresponding sequence without the three C-terminal residues is also contemplated. In some embodiments, for each sequence disclosed herein that contains a C-terminal lysine, the corresponding sequence without the four C-terminal residues is also contemplated. In some embodiments, for each sequence disclosed herein that contains a C-terminal lysine, the corresponding sequence without the five C-terminal residues is also contemplated. In some embodiments, for each sequence disclosed herein that contains a C-terminal lysine, the corresponding sequence without the six C-terminal residues is also contemplated. In some embodiments, for each sequence disclosed herein that contains a C-terminal lysine, the corresponding sequence without the seven C-terminal residues is also contemplated. In some embodiments, for each sequence disclosed herein that contains a C-terminal lysine, the corresponding sequence without the eight C-terminal residues is also contemplated. In some embodiments, for each sequence disclosed herein that contains a C-terminal lysine, the corresponding sequence without the nine C-terminal residues is also contemplated. In some embodiments, for each sequence disclosed herein that contains a C-terminal lysine, the corresponding sequence without the ten C-terminal residues is also contemplated. In some embodiments, for each sequence disclosed herein that contains a C-terminal lysine, the corresponding sequence without the eleven C-terminal residues is also contemplated. In some embodiments, for each sequence disclosed herein that contains a C-terminal lysine, the corresponding sequence without the twelve C-terminal residues is also contemplated. In some embodiments, for each sequence disclosed herein that contains a C-terminal lysine, the corresponding sequence without the thirteen C-terminal residues is also contemplated. In some embodiments, for each sequence disclosed herein that contains a C-terminal lysine, the corresponding sequence without the fourteen C-terminal residues is also contemplated. In some embodiments, for each sequence disclosed herein that contains a C-terminal lysine, the corresponding sequence without the fifteen C-terminal residues is also contemplated.

In some embodiments, the first monomer comprises the amino acid sequence set forth in SEQ ID NO: 9. In some embodiments, the first monomer comprises the amino acid sequence set forth in SEQ ID NO: 57. In some embodiments, the second monomer comprises the amino acid sequence set forth in SEQ ID NO: 10. In some embodiments, the second monomer comprises the amino acid sequence set forth in SEQ ID NO: 58. In some embodiments, the second monomer comprises the amino acid sequence set forth in SEQ ID NO: 16. In some embodiments, the second monomer comprises the amino acid sequence set forth in SEQ ID NO: 59. In some embodiments, the first monomer comprises the amino acid sequence set forth in SEQ ID NO: 9, and the second monomer comprises the amino acid sequence set forth in SEQ ID NO: 10. In some embodiments, the first monomer comprises the amino acid sequence set forth in SEQ ID NO: 57, and the second monomer comprises the amino acid sequence set forth in SEQ ID NO: 10. In some embodiments, the first monomer comprises the amino acid sequence set forth in SEQ ID NO: 9, and the second monomer comprises the amino acid sequence set forth in SEQ ID NO: 58. In some embodiments, the first monomer comprises the amino acid sequence set forth in SEQ ID NO: 57, and the second monomer comprises the amino acid sequence set forth in SEQ ID NO: 58. In some embodiments, the first monomer comprises the amino acid sequence set forth in SEQ ID NO: 9, and the second monomer comprises the amino acid sequence set forth in SEQ ID NO: 16. In some embodiments, the first monomer comprises the amino acid sequence set forth in SEQ ID NO: 57, and the second monomer comprises the amino acid sequence set forth in SEQ ID NO: 16. In some embodiments, the first monomer comprises the amino acid sequence set forth in SEQ ID NO: 9, and the second monomer comprises the amino acid sequence set forth in SEQ ID NO: 59. In some embodiments, the first monomer comprises the amino acid sequence set forth in SEQ ID NO: 57, and the second monomer comprises the amino acid sequence set forth in SEQ ID NO: 59.

In some embodiments, the first monomer comprises (1) IL-15 and (2) a first Fc domain that comprises the sequence set forth in SEQ ID NO: 6. In some embodiments, the second monomer comprises (1) IL-15Rα and (2) a second Fc domain that comprises the sequence set forth in SEQ ID NO: 7.

In some embodiments, the amino acid substitutions present in the heterodimeric protein are disclosed in U.S. Patent Publication US 2018/0118805 and are incorporated herein by reference in its entirety.

The sequences referenced herein are provided in Table 1, infra.

TABLE 1
Compilation of amino acid sequences described in the present disclosure.

| | | |
|---|---|---|
| SEQ ID | Wild-type | NWVNVISDLKKIEDLIQSMHIDATLYTESDVHPS |
| NO: 1 | mature or | CKVTAMKCFLLELQVISLESGDASIHDTVENLIIL |
| | truncated IL-15 | ANNSLSSNGNVTESGCKECEELEEKNIKEFLQSF |
| | protein | VHIVQMFINTS |
| SEQ ID | Wild-type full- | MRISKPHLRSISIQCYLCLLLNSHFLTEAGIHVFIL |
| NO: 2 | length IL-15 | GCFSAGLPKTEANWVNVISDLKKIEDLIQSMHID |
| | protein | ATLYTESDVHPSCKVTAMKCFLLELQVISLESGD |
| | | ASIHDTVENLIILANNSLSSNGNVTESGCKECEEL |
| | | EEKNIKEFLQSFVHIVQMFINTS |
| SEQ ID | Wild-type full- | MAPRRARGCRTLGLPALLLLLLLRPPATRGITCPP |
| NO: 3 | length IL-15Rα | PMSVEHADIWVKSYSLYSRERYICNSGFKRKAG |
| | protein | TSSLTECVLNKATNVAHWTTPSLKCIRDPALVHQ |
| | | RPAPPSTVTTAGVTPQPESLSPSGKEPAASSPSSN |
| | | NTAATTAAIVPGSQLMPSKSPSTGTTEISSHESSH |
| | | GTPSQTTAKNWELTASASHQPPGVYPQGHSDTT |
| | | VAISTSTVLLCGLSAVSLLACYLKSRQTPPLASVE |
| | | MEAMEALPVTWGTSSRDEDLENCSHHL |
| SEQ ID | Sushi domain of | ITCPPPMSVEHADIWVKSYSLYSRERYICNSGFKR |
| NO: 4 | IL-15Rα protein | KAGTSSLTECVLNKATNVAHWTTPSLKCIR |
| SEQ ID | XENP24306 or | NWVNVISDLKKIEDLIQSMHIDATLYTESNVHPS |
| NO: 5 | XENP32803 IL-15 | CKVTAMKCFLLELQVISLESGDASIHDTVQDLIIL |
| | protein | ANNSLSSNGNVTESGCKECEELEEKNIKEFLQSF |
| | variant | VHIVQMFINTS |
| SEQ ID | XENP24306 or | EPKSSDKTHTCPPCPAPPVAGPSVFLFPPKPKDTL |
| NO: 6 | XENP32803 | MISRTPEVTCVVVDVKHEDPEVKFNWYVDGVE |
| | First IgG1 Fc | VHNAKTKPREEEYNSTYRVVSVLTVLHQDWLN |
| | domain (IL-15 | GKEYKCKVSNKALPAPIEKTISKAKGQPREPQVY |
| | monomer) | TLPPSREEMTKNQVSLTCDVSGFYPSDIAVEWES |
| | | DGQPENNYKTTPPVLDSDGSFFLYSKLTVDKSR |
| | | WEQGDVFSCSVLHEALHSHYTQKSLSLSPGK |
| SEQ ID | XENP24306 | EPKSSDKTHTCPPCPAPPVAGPSVFLFPPKPKDTL |
| NO: 7 | Second IgG1 Fc | MISRTPEVTCVVVDVKHEDPEVKFNWYVDGVE |
| | domain (IL- | VHNAKTKPREEQYNSTYRVVSVLTVLHQDWLN |
| | 15Rα monomer) | GKEYKCKVSNKALPAPIEKTISKAKGQPREPQVY |
| | | TLPPSREQMTKNQVKLTCLVKGFYPSDIAVEWES |
| | | NGQPENNYKTTPPVLDSDGSFFLYSKLTVDKSR |
| | | WQQGNVFSCSVLHEALHSHYTQKSLSLSPGK |
| SEQ ID | XENP32803 | EPKSSDKTHTCPPCPAPPVAGPSVFLFPPTPKDTL |
| NO: 8 | Second IgG1 Fc | MISRTPEVTCVVVDVKHEDPEVKFNWYVDGVE |
| | domain (IL- | VHNAKTKPREEQYNSTYRVVSVLTVLHQDWLN |
| | 15Rα monomer) | GKEYKCKVSNKALPAPIEKTISKAKGQPREPQVY |
| | | TLPPSREQMTKNQVKLTCLVKGFYPSDIAVEWES |
| | | NGQPENNYKTTPPVLDSDGSFFLYSKLTVDKSR |
| | | WQQGNVFSCSVLHEALHSHYTQKSLSLSPGK |
| SEQ ID | XENP24306 or | NWVNVISDLKKIEDLIQSMHIDATLYTESNVHPS |
| NO: 9 | XENP32803 | CKVTAMKCFLLELQVISLESGDASIHDTVQDLIIL |
| | First monomer | ANNSLSSNGNVTESGCKECEELEEKNIKEFLQSF |
| | (IL-15-first Fc | VHIVQMFINTSGGGGSEPKSSDKTHTCPPCPAPPV |
| | domain | AGPSVFLFPPKPKDTLMISRTPEVTCVVVDVKHE |
| | monomer) | DPEVKFNWYVDGVEVHNAKTKPREEEYNSTYR |
| | | VVSVLTVLHQDWLNGKEYKCKVSNKALPAPIEK |
| | | TISKAKGQPREPQVYTLPPSREEMTKNQVSLTCD |
| | | VSGFYPSDIAVEWESDGQPENNYKTTPPVLDSDG |
| | | SFFLYSKLTVDKSRWEQGDVFSCSVLHEALHSH |
| | | YTQKSLSLSPG |
| SEQ ID | XENP24306 or | NWVNVISDLKKIEDLIQSMHIDATLYTESNVHPS |
| NO: 57 | XENP32803 | CKVTAMKCFLLELQVISLESGDASIHDTVQDLIIL |
| | First monomer | ANNSLSSNGNVTESGCKECEELEEKNIKEFLQSF |
| | (IL-15-first Fc | VHIVQMFINTSGGGGSEPKSSDKTHTCPPCPAPPV |
| | domain | AGPSVFLFPPKPKDTLMISRTPEVTCVVVDVKHE |
| | monomer) | DPEVKFNWYVDGVEVHNAKTKPREEEYNSTYR |
| | | VVSVLTVLHQDWLNGKEYKCKVSNKALPAPIEK |
| | | TISKAKGQPREPQVYTLPPSREEMTKNQVSLTCD |
| | | VSGFYPSDIAVEWESDGQPENNYKTTPPVLDSDG |
| | | SFFLYSKLTVDKSRWEQGDVFSCSVLHEALHSH |
| | | YTQKSLSLSPGK |
| SEQ ID | XENP24306 | ITCPPPMSVEHADIWVKSYSLYSRERYICNSGFKR |
| NO: 10 | Second monomer | KAGTSSLTECVLNKATNVAHWTTPSLKCIRGGG |
| | (IL-15Rα-second | GSEPKSSDKTHTCPPCPAPPVAGPSVFLFPPKPKD |
| | Fc domain | TLMISRTPEVTCVVVDVKHEDPEVKFNWYVDGV |
| | monomer) | EVHNAKTKPREEQYNSTYRVVSVLTVLHQDWL |
| | | NGKEYKCKVSNKALPAPIEKTISKAKGQPREPQV |
| | | YTLPPSREQMTKNQVKLTCLVKGFYPSDIAVEW |
| | | ESNGQPENNYKTTPPVLDSDGSFFLYSKLTVDKS |
| | | RWQQGNVFSCSVLHEALHSHYTQKSLSLSPG |
| SEQ ID | XENP24306 | ITCPPPMSVEHADIWVKSYSLYSRERYICNSGFKR |
| NO: 58 | Second monomer | KAGTSSLTECVLNKATNVAHWTTPSLKCIRGGG |
| | (IL-15Rα-second | GSEPKSSDKTHTCPPCPAPPVAGPSVFLFPPKPKD |
| | Fc domain | TLMISRTPEVTCVVVDVKHEDPEVKFNWYVDGV |
| | monomer) | EVHNAKTKPREEQYNSTYRVVSVLTVLHQDWL |
| | | NGKEYKCKVSNKALPAPIEKTISKAKGQPREPQV |
| | | YTLPPSREQMTKNQVKLTCLVKGFYPSDIAVEW |
| | | ESNGQPENNYKTTPPVLDSDGSFFLYSKLTVDKS |
| | | RWQQGNVFSCSVLHEALHSHYTQKSLSLSPGK |
| SEQ ID | XENP22853 | NWVNVISDLKKIEDLIQSMHIDATLYTESDVHPS |
| NO: 11 | Wild-type first | CKVTAMKCFLLELQVISLESGDASIHDTVENLIIL |
| | monomer (IL-15- | ANNSLSSNGNVTESGCKECEELEEKNIKEFLQSF |
| | first Fc domain | VHIVQMFINTSGGGGSEPKSSDKTHTCPPCPAPPV |
| | monomer) | AGPSVFLFPPKPKDTLMISRTPEVTCVVVDVKHE |
| | | DPEVKFNWYVDGVEVHNAKTKPREEEYNSTYR |
| | | VVSVLTVLHQDWLNGKEYKCKVSNKALPAPIEK |
| | | TISKAKGQPREPQVYTLPPSREEMTKNQVSLTCD |
| | | VSGFYPSDIAVEWESDGQPENNYKTTPPVLDSDG |
| | | SFFLYSKLTVDKSRWEQGDVFSCSVLHEALHSH |
| | | YTQKSLSLSPGK |
| SEQ ID | Unmodified Fc | EPKSCDKTHTCPPCPAPELLGGPSVFLFPPKPKDT |
| NO: 12 | IgG1 domain | LMISRTPEVTCVVVDVSHEDPEVKFNWYVDGVE |
| | (allele 3; | VHNAKTKPREEQYNSTYRVVSVLTVLHQDWLN |
| | Y14737) | GKEYKCKVSNKALPAPIEKTISKAKGQPREPQVY |
| | | TLPPSREEMTKNQVSLTCLVKGFYPSDIAVEWES |
| | | NGQPENNYKTTPPVLDSDGSFFLYSKLTVDKSR |
| | | WQQGNVFSCSVMHEALHNHYTQKSLSLSPGK |
| SEQ ID | Unmodified Fc | ERKCCVECPPCPAPPVAGPSVFLFPPKPKDTLMIS |
| NO: 13 | IgG2 domain | RTPEVTCVVVDVSHEDPEVQFNWYVDGVEVHN |
| | (allele 1; | AKTKPREEQFNSTFRVVSVLTVVHQDWLNGKEY |
| | J00230/AH005273) | KCKVSNKGLPAPIEKTISKTKGQPREPQVYTLPPS |
| | | REEMTKNQVSLTCLVKGFYPSDIAVEWESNGQP |
| | | ENNYKTTPPMLDSDGSFFLYSKLTVDKSRWQQG |
| | | NVFSCSVMHEALHNHYTQKSLSLSPGK |
| SEQ ID | Unmodified Fc | EPKSCDTPPPCPRCPAPELLGGPSVFLFPPKPKDT |
| NO: 14 | IgG3 domain | LMISRTPEVTCVVVDVSHEDPEVQFKWYVDGVE |
| | (allele 8; | VHNAKTKPREEQYNSTFRVVSVLTVLHQDWLN |
| | AJ390241/X03604) | GKEYKCKVSNKALPAPIEKTISKTKQPREPQVYT |
| | | LPPSREEMTKNQVSLTCLVKGFYPSDIAVEWESN |
| | | GQPENNYNTTPPMLDSDGSFFLYSKLTVDKSRW |
| | | QQGNIFSCSVMHEALHNRFTQKSLSLSPGK |
| SEQ ID | Unmodified Fc | ESKYGPPCPSCPAPEFLGGPSVFLFPPKPKDTLMI |
| NO: 15 | IgG4 domain | SRTPEVTCVVVDVSQEDPEVQFNWYVDGVEVH |
| | (allele 1; | NAKTKPREEQFNSTYRVVSVLTVLHQDWLNGKE |
| | K01316/AH005273) | YKCKVSNKGLPSSIEKTISKAKGQPREPQVYTLPP |
| | | SQEEMTKNQVSLTCLVKGFYPSDIAVEWESNGQ |
| | | PENNYKTTPPVLDSDGSFFLYSRLTVDKSRWQEG |
| | | NVFSCSVMHEALHNHYTQKSLSLSLGK |
| SEQ ID | XENP32803 | ITCPPPMSVEHADIWVKSYSLYSRERYICNSGFKR |
| NO: 16 | Second monomer | KAGTSSLTECVLNKATNVAHWTTPSLKCIRGGG |
| | (IL-15Rα | GSEPKSSDKTHTCPPCPAPPVAGPSVFLFPPTPKD |
| | monomer) | TLMISRTPEVTCVVVDVKHEDPEVKFNWYVDGV |
| | | EVHNAKTKPREEQYNSTYRVVSVLTVLHQDWL |
| | | NGKEYKCKVSNKALPAPIEKTISKAKGQPREPQV |
| | | YTLPPSREQMTKNQVKLTCLVKGFYPSDIAVEW |
| | | ESNGQPENNYKTTPPVLDSDGSFFLYSKLTVDKS |
| | | RWQQGNVFSCSVLHEALHSHYTQKSLSLSPG |
| SEQ ID | XENP32803 | ITCPPPMSVEHADIWVKSYSLYSRERYICNSGFKR |
| NO: 59 | Second monomer | KAGTSSLTECVLNKATNVAHWTTPSLKCIRGGG |
| | (IL-15Rα | GSEPKSSDKTHTCPPCPAPPVAGPSVFLFPPTPKD |
| | monomer) | TLMISRTPEVTCVVVDVKHEDPEVKFNWYVDGV |
| | | EVHNAKTKPREEQYNSTYRVVSVLTVLHQDWL |
| | | NGKEYKCKVSNKALPAPIEKTISKAKGQPREPQV |
| | | YTLPPSREQMTKNQVKLTCLVKGFYPSDIAVEW |
| | | ESNGQPENNYKTTPPVLDSDGSFFLYSKLTVDKS |
| | | RWQQGNVFSCSVLHEALHSHYTQKSLSLSPGK |

In some embodiments, the heterodimeric protein of the disclosure is selected from the group consisting of XENP20818, XENP20819, XENP21471, XENP21472, XENP21473, XENP21474, XENP21475, XENP21476, XENP21477, XENP21988, XENP21989, XENP21990, XENP21991, XENP21992, XENP22013, XENP22014, XENP22015, XENP22017, XENP22815, XENP22816, XENP22817, XENP22818, XENP22819, XENP22820, XENP22821, XENP22822, XENP22823, XENP22824, XENP22825, XENP22826, XENP22827, XENP22828, XENP22829, XENP22830, XENP22831, XENP22832, XENP22833, XENP22834, XENP23343, XENP23472, XENP23504, XENP23554, XENP23555, XENP23557, XENP23559, XENP23560, XENP23561, XENP24017, XENP24018, XENP24019, XENP24020, XENP24043, XENP24044, XENP24046, XENP24051, XENP24052, XENP24113, XENP24301, XENP24306, XENP24341, and XENP32803 heterodimeric proteins, the sequences of which are disclosed in FIGS. 104A-104AY of U.S. Pat. No. 10,501,543 and are incorporated by reference herein.

In some embodiments, the heterodimeric protein of the disclosure is selected from the group consisting of XENP22822, XENP23504, XENP24045, XENP24306, XENP22821, XENP23343, XENP23557, XENP24113, XENP24051, XENP24341, XENP24052, XENP24301, and XENP32803 heterodimeric proteins, which are described in Table 2 below. The sequences of XENP22822, XENP23504, XENP24045, XENP24306, XENP22821, XENP23343, XENP23557, XENP24113, XENP24051, XENP24341, XENP24052, and XENP24301 are also provided in US 2018/0118805 and are incorporated by reference herein. In some embodiments, the heterodimeric protein of the disclosure is XENP24306. In some embodiments, the heterodimeric protein of the disclosure is XENP32803. In some embodiments, a combination of two or more (e.g., 2, 3, 4, 5, etc.) heterodimeric proteins of the disclosure are used in the methods disclosed herein. In some embodiments, a combination of two heterodimeric proteins of the disclosure is used in the methods disclosed herein. In some embodiments, a combination of XENP24306 and XENP32803 is used in the methods disclosed herein.

In some embodiments, the XENP24306 protein represents about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 75%, about 70%, about 65%, about 60%, about 55%, about 50%, about 45%, about 40%, about 35%, about 30%, about 25%, about 20%, about 15%, about 10%, or about 5% of the heterodimeric protein in the combination. In some embodiments, the XENP24306 protein represents about 85% of the heterodimeric protein in the combination. In some embodiments, the XENP24306 protein represents about 84% of the heterodimeric protein in the combination. In some embodiments, the XENP24306 protein represents about 83% of the heterodimeric protein in the combination. In some embodiments, the XENP24306 protein represents about 82% of the heterodimeric protein in the combination. In some embodiments, the XENP24306 protein represents about 81% of the heterodimeric protein in the combination. In some embodiments, the XENP24306 protein represents about 80% of the heterodimeric protein in the combination.

In some embodiments, the XENP32803 protein represents about 95%, about 90%, about 85%, about 80%, about 75%, about 70%, about 75%, about 70%, about 65%, about 55%, about 50%, about 45%, about 40%, about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% of the heterodimeric protein in the combination. In some embodiments, the XENP32803 protein represents about 15% of the heterodimeric protein in the combination. In some embodiments, the XENP32803 protein represents about 16% of the heterodimeric protein in the combination. In some embodiments, the XENP32803 protein represents about 17% of the heterodimeric protein in the combination. In some embodiments, the XENP32803 protein represents about 18% of the heterodimeric protein in the combination. In some embodiments, the XENP32803 protein represents about 19% of the heterodimeric protein in the combination. In some embodiments, the XENP32803 protein represents about 20% of the heterodimeric protein in the combination.

In some embodiments, the XENP24306 protein represents between about 50-100%, about 70-95%, about 80-90%, or about 80-85% of the heterodimeric protein in the combination. In some embodiments of any of the methods disclosed herein, the XENP32803 protein represents between about 1-50%, about 5-30%, about 10-20%, or about 15-20% of the heterodimeric protein in the combination. In some embodiments, the XENP24306 protein represents about 85% of the heterodimeric protein in the combination, and the XENP32803 protein represents about 15% of the heterodimeric protein in the combination. In some embodiments, the XENP24306 protein represents about 84% of the heterodimeric protein in the combination, and the XENP32803 protein represents about 16% of the heterodimeric protein in the combination. In some embodiments, the XENP24306 protein represents about 83% of the heterodimeric protein in the combination, and the XENP32803 protein represents about 17% of the heterodimeric protein in the combination. In some embodiments, the XENP24306 protein represents about 82% of the heterodimeric protein in the combination, and the XENP32803 protein represents about 18% of the heterodimeric protein in the combination. In some embodiments, the XENP24306 protein represents about 81% of the heterodimeric protein in the combination, and the XENP32803 protein represents about 19% of the heterodimeric protein in the combination. In some embodiments, the XENP24306 protein represents about 80% of the heterodimeric protein in the combination, and the XENP32803 protein represents about 20% of the heterodimeric protein in the combination.

In some embodiments, the XENP24306 protein represents 99%, 98%, 97%, 96%, 95%, 94%, 93%, 92%, 91%, 90%, 89%, 88%, 87%, 86%, 85%, 84%, 83%, 82%, 81%, 80%, 75%0, 70%, 65%, 60%, 55%, 50%, 45%, 40%, 35%, 30%, 25%0, 20%, 15%, 10%, or 5% of the heterodimeric protein in the combination. In some embodiments, the XENP24306 protein represents 85% of the heterodimeric protein in the combination. In some embodiments, the XENP24306 protein represents 84% of the heterodimeric protein in the combination. In some embodiments, the XENP24306 protein represents 83% of the heterodimeric protein in the combination. In some embodiments, the XENP24306 protein represents 82% of the heterodimeric protein in the combination. In some embodiments, the XENP24306 protein represents 81% of the heterodimeric protein in the combination. In some embodiments, the XENP24306 protein represents 80% of the heterodimeric protein in the combination.

In some embodiments, the XENP32803 protein represents 95%, 90%, 85%, 80%, 75%, 70%, 75%, 70%, 65%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% of the heterodimeric protein in the combination. In some embodiments, the XENP32803 protein represents 15% of the heterodimeric protein in the combination. In some embodiments, the XENP32803 protein represents 16% of the heterodimeric protein in the combination. In some embodiments, the XENP32803 protein represents 17% of the heterodimeric protein in the combination. In some embodiments, the XENP32803 protein represents 18% of the heterodimeric protein in the combination. In some embodiments, the XENP32803 protein represents 19% of the heterodimeric protein in the combination. In some embodiments, the XENP32803 protein represents 20% of the heterodimeric protein in the combination.

In some embodiments, the XENP24306 protein represents between 50-100%, 70-95%, 80-90%, or 80-85% of the heterodimeric protein in the combination. In some embodiments of any of the methods disclosed herein, the XENP32803 protein represents between 1-50%, 5-30%, 10-20%, or 15-20% of the heterodimeric protein in the combination. In some embodiments, the XENP24306 protein represents 85% of the heterodimeric protein of the heterodimeric protein in the combination, and the XENP32803 protein represents 15% of the heterodimeric protein in the combination. In some embodiments, the XENP24306 protein represents 84% of the heterodimeric protein in the combination, and the XENP32803 protein represents 16% of the heterodimeric protein in the combination. In some embodiments, the XENP24306 protein represents 83% of the heterodimeric protein in the combination, and the XENP32803 protein represents 17% of the heterodimeric protein in the combination. In some embodiments, the XENP24306 protein represents 82% of the heterodimeric protein in the combination, and the XENP32803 protein represents 18% of the heterodimeric protein in the combination. In some embodiments, the XENP24306 protein represents 81% of the heterodimeric protein in the combination, and the XENP32803 protein represents 19% of the heterodimeric protein in the combination. In some embodiments, the XENP24306 protein represents 80% of the heterodimeric protein in the combination, and the XENP32803 protein represents 20% of the heterodimeric protein in the combination.

TABLE 2

| | |
|---|---|
| XENP22821 | Monomer 1 (IL-15 (N65D)-first Fc domain). SEQ ID NO: 17 |
| | NWVNVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSGGGGSEPKSSDKTHTCPPCPAP |
| | PVAGPSVFLFPPKPKDTLMISRTPEVTCVVVDVKHEDPEVKFNW |
| | YVDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLYS |
| | KLTVDKSRWEQGDVFSCSVMHEALHNHYTQKSLSLSPGK |
| | Monomer 2 (IL-15Rα-second Fc domain). SEQ ID NO: 18 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPPV |
| | AGPSVFLFPPKPKDTLMISRTPEVTCVVVDVKHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSREQMTKNQVKL |
| | TCLVKGFYPSDIAVEWESNGQPENNYKTTPPVLDSDGSFFLYSK |
| | LTVDKSRWQQGNVFSCSVMHEALHNHYTQKSLSLSPGK |
| XENP22822 | Monomer 1 (IL-15 (Q108E)-first Fc domain). SEQ ID NO: 19 |
| | NWVNVISDLKKIEDLIQSMHIDATLYTESDVHPSCKVTAMKCFL |
| | LELQVISLESGDASIHDTVENLIILANNSLSSNGNVTESGCKECEE |
| | LEEKNIKEFLQSFVHIVEMFINTSGGGGSEPKSSDKTHTCPPCPAP |
| | PVAGPSVFLFPPKPKDTLMISRTPEVTCVVVDVKHEDPEVKFNW |
| | YVDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLYS |
| | KLTVDKSRWEQGDVFSCSVMHEALHNHYTQKSLSLSPGK |
| | Monomer 2 (IL-15Rα-second Fc domain). SEQ ID NO: 20 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPPV |
| | AGPSVFLFPPKPKDTLMISRTPEVTCVVVDVKHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSREQMTKNQVKL |
| | TCLVKGFYPSDIAVEWESNGQPENNYKTTPPVLDSDGSFFLYSK |
| | LTVDKSRWQQGNVFSCSVMHEALHNHYTQKSLSLSPGK |
| XENP23557 | Monomer 1 (IL-15 (N4D/N65D)-first Fc domain). SEQ ID NO: 21 |
| | NWVDVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSGGGGSEPKSSDKTHTCPPCPAP |
| | PVAGPSVFLFPPKPKDTLMISRTPEVTCVVVDVKHEDPEVKFNW |
| | YVDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLYS |
| | KLTVDKSRWEQGDVFSCSVMHEALHNHYTQKSLSLSPGK |
| | Monomer 2 (IL-15Rα-second Fc domain), SEQ ID NO: 22 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAP |
| | PVAGPSVFLFPPKPKDTLMISRTPEVTCVVVDVKHEDPEVKFNW |
| | YVDGVEVHNAKTKPREEQYNSTYRVVSVLTVLHQDWLNGKEY |
| | KCKVSNKALPAPIEKTISKAKGQPREPQVYTLPPSREQMTKNQV |
| | KLTCLVKGFYPSDIAVEWESNGQPENNYKTTPPVLDSDGSFFLY |
| | SKLTVDKSRWQQGNVFSCSVMHEALHNHYTQKSLSLSPGK |
| XENP24045 | Monomer 1 (IL-15 (D30N/E64Q/N65D)-first Fc domain). SEQ ID NO: 23 |
| | NWVNVISDLKKIEDLIQSMHIDATLYTESNVHPSCKVTAMKCFL |
| | LELQVISLESGDASIHDTVQDLIILANNSLSSNGNVTESGCKECEE |
| | LEEKNIKEFLQSFVHIVQMFINTSGGGGSEPKSSDKTHTCPPCPAP |
| | PVAGPSVFLFPPKPKDTLMISRTPEVTCVVVDVKHEDPEVKFNW |
| | YVDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLYS |
| | KLTVDKSRWEQGDVFSCSVMHEALHNHYTQKSLSLSPGK |
| | Monomer 2 (IL-15Rα-second Fc domain). SEQ ID NO: 24 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAP |
| | PVAGPSVFLFPPKPKDTLMISRTPEVTCVVVDVKHEDPEVKFNW |
| | YVDGVEVHNAKTKPREEQYNSTYRVVSVLTVLHQDWLNGKEY |
| | KCKVSNKALPAPIEKTISKAKGQPREPQVYTLPPSREQMTKNQV |
| | KLTCLVKGFYPSDIAVEWESNGQPENNYKTTPPVLDSDGSFFLY |
| | SKLTVDKSRWQQGNVFSCSVMHEALHNHYTQKSLSLSPGK |
| XENP24051 | Monomer 1 (IL-15 (N1D/N65D)-first Fc domain), SEQ ID NO: 25 |
| | DWVNVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSEPKSSDKTHTCPPCPAPPVAGP |
| | SVFLFPPKPKDTLMISRTPEVTCVVVDVKHEDPEVKFNWYVDG |
| | VEVHNAKTKPREEEYNSTYRVVSVLTVLHQDWLNGKEYKCKV |
| | SNKALPAPIEKTISKAKGQPREPQVYTLPPSREEMTKNQVSLTCD |
| | VSGFYPSDIAVEWESDGQPENNYKTTPPVLDSDGSFFLYSKLTV |
| | DKSRWEQGDVFSCSVMHEALHNHYTQKSLSLSPGK |
| | Monomer 2 (IL-15Rα-second Fc domain). SEQ ID NO: 26 |
| | ITCPPPMSVEHADIWVKSYSLYSRERYICNSGFKRKAGTSSLTEC |
| | VLNKATNVAHWTTPSLKCIREPKSSDKTHTCPPCPAPPVAGPSV |
| | FLFPPKPKDTLMISRTPEVTCVVVDVKHEDPEVKFNWYVDGVE |
| | VHNAKTKPREEQYNSTYRVVSVLTVLHQDWLNGKEYKCKVSN |
| | KALPAPIEKTISKAKGQPREPQVYTLPPSREQMTKNQVKLTCLV |
| | KGFYPSDIAVEWESNGQPENNYKTTPPVLDSDGSFFLYSKLTVD |
| | KSRWQQGNVFSCSVMHEALHNHYTQKSLSLSPGK |
| XENP24052 | Monomer 1 (IL-15 (N4D/N65D)-first Fc domain). SEQ ID NO: 27 |
| | NWVDVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSEPKSSDKTHTCPPCPAPPVAGP |
| | SVFLFPPKPKDTLMISRTPEVTCVVVDVKHEDPEVKFNWYVDG |
| | VEVHNAKTKPREEEYNSTYRVVSVLTVLHQDWLNGKEYKCKV |
| | SNKALPAPIEKTISKAKGQPREPQVYTLPPSREEMTKNQVSLTCD |
| | VSGFYPSDIAVEWESDGQPENNYKTTPPVLDSDGSFFLYSKLTV |
| | DKSRWEQGDVFSCSVMHEALHNHYTQKSLSLSPGK |
| | Monomer 2 (IL-15Rα-second Fc domain). SEQ ID NO: 28 |
| | ITCPPPMSVEHADIWVKSYSLYSRERYICNSGFKRKAGTSSLTEC |
| | VLNKATNVAHWTTPSLKCIREPKSSDKTHTCPPCPAPPVAGPSV |
| | FLFPPKPKDTLMISRTPEVTCVVVDVKHEDPEVKFNWYVDGVE |
| | VHNAKTKPREEQYNSTYRVVSVLTVLHQDWLNGKEYKCKVSN |
| | KALPAPIEKTISKAKGQPREPQVYTLPPSREQMTKNQVKLTCLV |
| | KGFYPSDIAVEWESNGQPENNYKTTPPVLDSDGSFFLYSKLTVD |
| | KSRWQQGNVFSCSVMHEALHNHYTQKSLSLSPGK |
| XENP23504 | Monomer 1 (IL-15 (Q108E)-first Fc domain). SEQ ID NO: 29 |
| | NWVNVISDLKKIEDLIQSMHIDATLYTESDVHPSCKVTAMKCFL |
| | LELQVISLESGDASIHDTVENLIILANNSLSSNGNVTESGCKECEE |
| | LEEKNIKEFLQSFVHIVEMFINTSGGGGSEPKSSDKTHTCPPCPAP |
| | PVAGPSVFLFPPKPKDTLMISRTPEVTCVVVDVKHEDPEVKFNW |
| | YVDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLYS |
| | KLTVDKSRWEQGDVFSCSVLHEALHSHYTQKSLSLSPGK |
| | Monomer 2 (IL-15Rα-second Fc domain). SEQ ID NO: 30 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPPV |
| | AGPSVFLFPPKPKDTLMISRTPEVTCVVVDVKHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSREQMTKNQVKL |
| | TCL VKGFYPSDIAVEWESNGQPENNYKTTPPVLDSDGSFFLYSK |
| | LTVDKSRWQQGNVFSCSVLHEALHSHYTQKSLSLSPGK |
| XENP23343 | Monomer 1 (IL-15 (N65D)-first Fc domain). SEQ ID NO: 31 |
| | NWVNVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSGGGGSEPKSSDKTHTCPPCPAP |
| | PVAGPSVFLFPPKPKDTLMISRTPEVTCVVVDVKHEDPEVKFNW |
| | YVDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLYS |
| | KLTVDKSRWEQGDVFSCSVLHEALHSHYTQKSLSLSPGK |
| | Monomer 2 (IL-15Rα-second Fc domain). SEQ ID NO: 32 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPPV |
| | AGPSVFLFPPKPKDTLMISRTPEVTCVVVDVKHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSREQMTKNQVKL |
| | TCLVKGFYPSDIAVEWESNGQPENNYKTTPPVLDSDGSFFLYSK |
| | LTVDKSRWQQGNVFSCSVLHEALHSHYTQKSLSLSPGK |
| XENP24113 | Monomer 1 (IL-15 (N4D/N65D)-first Fc domain). SEQ ID NO: 33 |
| | NWVDVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSGGGGSEPKSSDKTHTCPPCPAP |
| | PVAGPSVFLFPPKPKDTLMISRTPEVTCVVVDVKHEDPEVKFNW |
| | YVDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLYS |
| | KLTVDKSRWEQGDVFSCSVLHEALHSHYTQKSLSLSPGK |
| | Monomer 2 (IL-15Rα-second Fc domain). SEQ ID NO: 34 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPPV |
| | AGPSVFLFPPKPKDTLMISRTPEVTCVVVDVKHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSREQMTKNQVKL |
| | TCLVKGFYPSDIAVEWESNGQPENNYKTTPPVLDSDGSFFLYSK |
| | LTVDKSRWQQGNVFSCSVLHEALHSHYTQKSLSLSPGK |
| XENP24341 | Monomer 1 (IL-15 (N1D/N65D)-first Fc domain). SEQ ID NO: 35 |
| | DWVNVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSEPKSSDKTHTCPPCPAPPVAGP |
| | SVFLFPPKPKDTLMISRTPEVTCVVVDVKHEDPEVKFNWYVDG |
| | VEVHNAKTKPREEEYNSTYRVVSVLTVLHQDWLNGKEYKCKV |
| | SNKALPAPIEKTISKAKGQPREPQVYTLPPSREEMTKNQVSLTCD |
| | VSGFYPSDIAVEWESDGQPENNYKTTPPVLDSDGSFFLYSKLTV |
| | DKSRWEQGDVFSCSVLHEALHSHYTQKSLSLSPGK |
| | Monomer 2 (IL-15Rα-second Fc domain). SEQ ID NO: 36 |
| | ITCPPPMSVEHADIWVKSYSLYSRERYICNSGFKRKAGTSSLTEC |
| | VLNKATNVAHWTTPSLKCIREPKSSDKTHTCPPCPAPPVAGPSV |
| | FLFPPKPKDTLMISRTPEVTCVVVDVKHEDPEVKFNWYVDGVE |
| | VHNAKTKPREEQYNSTYRVVSVLTVLHQDWLNGKEYKCKVSN |
| | KALPAPIEKTISKAKGQPREPQVYTLPPSREQMTKNQVKLTCLV |
| | KGFYPSDIAVEWESNGQPENNYKTTPPVLDSDGSFFLYSKLTVD |
| | KSRWQQGNVFSCSVLHEALHSHYTQKSLSLSPGK |
| XENP24301 | Monomer 1 (IL-15 (N4D/N65D)-first Fc domain), SEQ ID NO: 37 |
| | NWVDVISDLKKIEDLIQSMHIDATLYTESDVHPSCKVTAMKCFL |
| | LELQVISLESGDASIHDTVEDLIILANNSLSSNGNVTESGCKECEE |
| | LEEKNIKEFLQSFVHIVQMFINTSEPKSSDKTHTCPPCPAPPVAGP |
| | SVFLFPPKPKDTLMISRTPEVTCVVVDVKHEDPEVKFNWYVDG |
| | VEVHNAKTKPREEEYNSTYRVVSVLTVLHQDWLNGKEYKCKV |
| | SNKALPAPIEKTISKAKGQPREPQVYTLPPSREEMTKNQVSLTCD |
| | VSGFYPSDIA VEWESDGQPENNYKTTPPVLDSDGSFFLYSKLTV |
| | DKSRWEQGDVFSCSVLHEALHSHYTQKSLSLSPGK |
| | Monomer 2 (IL-15Rα-second Fc domain). SEQ ID NO: 38 |
| | ITCPPPMSVEHADIWVKSYSLYSRERYICNSGFKRKAGTSSLTEC |
| | VLNKATNVAHWTTPSLKCIREPKSSDKTHTCPPCPAPPVAGPSV |
| | FLFPPKPKDTLMISRTPEVTCVVVDVKHEDPEVKFNWYVDGVE |
| | VHNAKTKPREEQYNSTYRVVSVLTVLHQDWLNGKEYKCKVSN |
| | KALPAPIEKTISKAKGQPREPQVYTLPPSREQMTKNQVKLTCLV |
| | KGFYPSDIAVEWESNGQPENNYKTTPPVLDSDGSFFLYSKLTVD |
| | KSRWQQGNVFSCSVLHEALHSHYTQKSLSLSPGK |

Methods of Treatment with IL15-IL15Rα Heterodimeric Fc-Fusion Proteins

In one aspect, the present disclosure provides methods of treating a solid tumor in a subject in need thereof, the method comprising administering to the subject a plurality of doses of a heterodimeric protein, wherein each dose comprises a therapeutically effective amount of any of the heterodimeric proteins disclosed herein or any combinations thereof. In some embodiments, the administration of the plurality of doses of the heterodimeric protein is sufficient to increase the number of NK cells at least 20-fold as compared to the number of NK cells prior to said administration. In some embodiments, the administration of the plurality of doses of the heterodimeric protein results in accumulation of NK cells in the subject.

In another aspect, the present disclosure provides a plurality of doses of any of the heterodimeric protein disclosed herein or any combinations thereof, for use in the treatment of a solid tumor in a subject in need thereof. In some embodiments, the administration of the plurality of doses of the heterodimeric protein is sufficient to increase the number of NK cells at least 20-fold as compared to the number of NK cells prior to said administration. In some embodiments, the administration of the plurality of doses of the heterodimeric protein results in accumulation of NK cells in the subject.

In another aspect, the present disclosure provides the use of a therapeutically effective amount of any of the heterodimeric proteins as disclosed herein or any combinations thereof, in the manufacture of a plurality of doses of a medicament for the treatment of a solid tumor in a subject in need thereof. In some embodiments, the administration of the plurality of doses of the heterodimeric protein is sufficient to increase the number of NK cells at least 20-fold as compared to the number of NK cells prior to said administration. In some embodiments, the administration of the plurality of doses of the heterodimeric protein results in accumulation of NK cells in the subject.

In another aspect, the present disclosure provides methods for inducing the proliferation of NK cells in a subject, the method comprising administering to the subject a plurality of doses of a heterodimeric protein, wherein each dose comprises an effective amount of any of the heterodimeric proteins disclosed herein or any combinations thereof. In some embodiments, the administration of the plurality of doses of the heterodimeric protein is sufficient to increase the number of NK cells at least 20-fold as compared to the number of NK cells prior to said administration. In some embodiments, the administration of the plurality of doses of the heterodimeric protein results in accumulation of NK cells in the subject.

In another aspect, the present disclosure provides methods for inducing the proliferation of NK cells in a subject, the method comprising administering to the subject a plurality of doses of a heterodimeric protein, wherein each dose comprises an effective amount of any of the heterodimeric proteins disclosed herein or any combinations thereof, and wherein the proliferative response of NK cells is stronger than the proliferative response of CD8⁺ effector memory T cells upon the administration of an effective amount of any of the heterodimeric proteins disclosed herein or any combinations thereof. In some embodiments, the administration of the plurality of doses of the heterodimeric protein is sufficient to increase the number of NK cells at least 20-fold as compared to the number of NK cells prior to said administration. In some embodiments, the administration of the plurality of doses of the heterodimeric protein results in accumulation of NK cells in the subject.

In some embodiments, the number of NK cells increases at least 30-fold, 40-fold, 50-fold, 60-fold, 70-fold, 80-fold, 90-fold, 100-fold, 150-fold, 200-fold, 250-fold, or 300-fold relative to the number of NK cells prior to administration. In some embodiments, the number of NK cells increases at least 30-fold relative to the number of NK cells prior to administration. In some embodiments, the number of NK cells increases at least 40-fold relative to the number of NK cells prior to administration. In some embodiments, the number of NK cells increases at least 50-fold relative to the number of NK cells prior to administration. In some embodiments, the number of NK cells increases at least 60-fold relative to the number of NK cells prior to administration. In some embodiments, the number of NK cells increases at least 70-fold relative to the number of NK cells prior to administration. In some embodiments, the number of NK cells increases at least 80-fold relative to the number of NK cells prior to administration. In some embodiments, the number of NK cells increases at least 90-fold relative to the number of NK cells prior to administration. In some embodiments, the number of NK cells increases at least 100-fold relative to the number of NK cells prior to administration. In some embodiments, the number of NK cells increases at least 110-fold relative to the number of NK cells prior to administration. In some embodiments, the number of NK cells increases at least 120-fold relative to the number of NK cells prior to administration. In some embodiments, the number of NK cells increases at least 130-fold relative to the number of NK cells prior to administration. In some embodiments, the number of NK cells increases at least 140-fold relative to the number of NK cells prior to administration. In some embodiments, the number of NK cells increases at least 150-fold relative to the number of NK cells prior to administration. In some embodiments, the number of NK cells increases at least 160-fold relative to the number of NK cells prior to administration. In some embodiments, the number of NK cells increases at least 170-fold relative to the number of NK cells prior to administration. In some embodiments, the number of NK cells increases at least 180-fold relative to the number of NK cells prior to administration. In some embodiments, the number of NK cells increases at least 190-fold relative to the number of NK cells prior to administration. In some embodiments, the number of NK cells increases at least 200-fold relative to the number of NK cells prior to administration. In some embodiments, the number of NK cells increases at least 210-fold relative to the number of NK cells prior to administration. In some embodiments, the number of NK cells increases at least 220-fold relative to the number of NK cells prior to administration. In some embodiments, the number of NK cells increases at least 230-fold relative to the number of NK cells prior to administration. In some embodiments, the number of NK cells increases at least 240-fold relative to the number of NK cells prior to administration. In some embodiments, the number of NK cells increases at least 250-fold relative to the number of NK cells prior to administration. In some embodiments, the number of NK cells increases at least 260-fold relative to the number of NK cells prior to administration. In some embodiments, the number of NK cells increases at least 270-fold relative to the number of NK cells prior to administration. In some embodiments, the number of NK cells increases at least 280-fold relative to the number of NK cells prior to administration. In some embodiments, the number of NK cells increases at least 290-fold relative to the number of NK cells prior to administration. In some embodiments, the number of NK cells increases at least 300-fold relative to the number of NK cells prior to administration.

In some embodiments, the accumulation is for at least one cycle. In some embodiments, the accumulation is for at least two cycles. In some embodiments, the accumulation is for at least three cycles. In some embodiments, the accumulation is for at least four cycles. In some embodiments, the accumulation is for at least five cycles. In some embodiments, the accumulation is for at least six cycles. In some embodiments, the accumulation is for at least seven cycles. In some embodiments, the accumulation is for at least eight cycles. In some embodiments, the accumulation is for at least nine cycles. In some embodiments, the accumulation is for at least ten cycles. In some embodiments, the accumulation is for at least eleven cycles. In some embodiments, the accumulation is for at least twelve cycles. In some embodiments, the accumulation is for at least thirteen cycles. In some embodiments, the accumulation is for at least fourteen cycles. In some embodiments, the accumulation is for at least fifteen cycles. In some embodiments, the accumulation is for at least sixteen cycles. In some embodiments, the accumulation is for at least seventeen cycles. In some embodiments, the accumulation is for at least eighteen cycles. In some embodiments, the accumulation is for at least nineteen cycles. In some embodiments, the accumulation is for at least twenty cycles. In some embodiments, the accumulation is for at least twenty-one cycles. In some embodiments, the accumulation is for at least twenty-two cycles. In some embodiments, the accumulation is for at least twenty-three cycles. In some embodiments, the accumulation is for at least twenty-four cycles. In some embodiments, the accumulation is for at least twenty-five cycles. In some embodiments, the accumulation is for at least twenty-six cycles.

In some embodiments, the accumulation is for no more than one cycle. In some embodiments, the accumulation is for no more than two cycles. In some embodiments, the accumulation is for no more than three cycles. In some embodiments, the accumulation is for no more than four cycles. In some embodiments, the accumulation is for no more than five cycles. In some embodiments, the accumulation is for no more than six cycles. In some embodiments, the accumulation is for no more than seven cycles. In some embodiments, the accumulation is for no more than eight cycles. In some embodiments, the accumulation is for no more than nine cycles. In some embodiments, the accumulation is for no more than ten cycles. In some embodiments, the accumulation is for no more than eleven cycles. In some embodiments, the accumulation is for no more than twelve cycles. In some embodiments, the accumulation is for no more than thirteen cycles. In some embodiments, the accumulation is for no more than fourteen cycles. In some embodiments, the accumulation is for no more than fifteen cycles. In some embodiments, the accumulation is for no more than sixteen cycles. In some embodiments, the accumulation is for no more than seventeen cycles. In some embodiments, the accumulation is for no more than eighteen cycles. In some embodiments, the accumulation is for no more than nineteen cycles. In some embodiments, the accumulation is for no more than twenty cycles. In some embodiments, the accumulation is for no more than twenty-one cycles. In some embodiments, the accumulation is for no more than twenty-two cycles. In some embodiments, the accumulation is for no more than twenty-three cycles. In some embodiments, the accumulation is for no more than twenty-four cycles. In some embodiments, the accumulation is for no more than twenty-five cycles. In some embodiments, the accumulation is for no more than twenty-six cycles.

In some embodiments, the plurality of doses comprises at least two doses. In some embodiments, the plurality of doses comprises at least three doses. In some embodiments, the plurality of doses comprises at least four doses. In some embodiments, the plurality of doses comprises at least five doses. In some embodiments, the plurality of doses comprises at least six doses. In some embodiments, the plurality of doses comprises at least seven doses. In some embodiments, the plurality of doses comprises at least eight doses. In some embodiments, the plurality of doses comprises at least nine doses. In some embodiments, the plurality of doses comprises at least ten doses. In some embodiments, the plurality of doses comprises at least eleven doses. In some embodiments, the plurality of doses comprises at least twelve doses. In some embodiments, the plurality of doses comprises at least thirteen doses. In some embodiments, the plurality of doses comprises at least fourteen doses. In some embodiments, the plurality of doses comprises at least fifteen doses. In some embodiments, the plurality of doses comprises at least sixteen doses. In some embodiments, the plurality of doses comprises at least seventeen doses. In some embodiments, the plurality of doses comprises at least eighteen doses. In some embodiments, the plurality of doses comprises at least nineteen doses. In some embodiments, the plurality of doses comprises at least twenty doses. In some embodiments, the plurality of doses comprises at least twenty-one doses. In some embodiments, the plurality of doses comprises at least twenty-two doses. In some embodiments, the plurality of doses comprises at least twenty-three doses. In some embodiments, the plurality of doses comprises at least twenty-four doses. In some embodiments, the plurality of doses comprises at least twenty-five doses. In some embodiments, the plurality of doses comprises at least twenty-six doses.

In some embodiments, the plurality of doses comprises no more than two doses. In some embodiments, the plurality of doses comprises no more than three doses. In some embodiments, the plurality of doses comprises no more than four doses. In some embodiments, the plurality of doses comprises no more than five doses. In some embodiments, the plurality of doses comprises no more than six doses. In some embodiments, the plurality of doses comprises no more than seven doses. In some embodiments, the plurality of doses comprises no more than eight doses. In some embodiments, the plurality of doses comprises no more than nine doses. In some embodiments, the plurality of doses comprises no more than ten doses. In some embodiments, the plurality of doses comprises no more than eleven doses. In some embodiments, the plurality of doses comprises no more than twelve doses. In some embodiments, the plurality of doses comprises no more than thirteen doses. In some embodiments, the plurality of doses comprises no more than fourteen doses. In some embodiments, the plurality of doses comprises no more than fifteen doses. In some embodiments, the plurality of doses comprises no more than sixteen doses. In some embodiments, the plurality of doses comprises no more than seventeen doses. In some embodiments, the plurality of doses comprises no more than eighteen doses. In some embodiments, the plurality of doses comprises no more than nineteen doses. In some embodiments, the plurality of doses comprises no more than twenty doses. In some embodiments, the plurality of doses comprises no more than twenty-one doses. In some embodiments, the plurality of doses comprises no more than twenty-two doses. In some embodiments, the plurality of doses comprises no more than twenty-three doses. In some embodiments, the plurality of doses comprises no more than twenty-four doses. In some embodiments, the plurality of doses comprises no more than twenty-five doses. In some embodiments, the plurality of doses comprises no more than twenty-six doses. In some embodiments, the plurality of doses comprises no more than fifty-two doses. In some embodiments, the plurality of doses comprises no more than seventy-eight doses. In some embodiments, the plurality of doses comprises no more than one hundred four doses. In some embodiments, the plurality of doses comprises no more than one hundred thirty doses.

In some embodiments, the number of NK cells increases at least 20-fold relative to the number of NK cells prior to administration after two doses. In some embodiments, the number of NK cells increases at least 20-fold relative to the number of NK cells prior to administration after three doses. In some embodiments, the number of NK cells increases at least 20-fold relative to the number of NK cells prior to administration after four doses. In some embodiments, the number of NK cells increases at least 20-fold relative to the number of NK cells prior to administration after five doses. In some embodiments, the number of NK cells increases at least 20-fold relative to the number of NK cells prior to administration after six doses. In some embodiments, the number of NK cells increases at least 20-fold relative to the number of NK cells prior to administration after seven doses. In some embodiments, the number of NK cells increases at least 20-fold relative to the number of NK cells prior to administration after eight doses. In some embodiments, the number of NK cells increases at least 20-fold relative to the number of NK cells prior to administration after nine doses. In some embodiments, the number of NK cells increases at least 20-fold relative to the number of NK cells prior to administration after ten doses.

In some embodiments, the number of NK cells increases at least 50-fold relative to the number of NK cells prior to administration after two doses. In some embodiments, the number of NK cells increases at least 50-fold relative to the number of NK cells prior to administration after three doses. In some embodiments, the number of NK cells increases at least 50-fold relative to the number of NK cells prior to administration after four doses. In some embodiments, the number of NK cells increases at least 50-fold relative to the number of NK cells prior to administration after five doses. In some embodiments, the number of NK cells increases at least 50-fold relative to the number of NK cells prior to administration after six doses. In some embodiments, the number of NK cells increases at least 50-fold relative to the number of NK cells prior to administration after seven doses. In some embodiments, the number of NK cells increases at least 50-fold relative to the number of NK cells prior to administration after eight doses. In some embodiments, the number of NK cells increases at least 50-fold relative to the number of NK cells prior to administration after nine doses. In some embodiments, the number of NK cells increases at least 50-fold relative to the number of NK cells prior to administration after ten doses.

In some embodiments, the number of NK cells increases at least 100-fold relative to the number of NK cells prior to administration after two doses. In some embodiments, the number of NK cells increases at least 100-fold relative to the number of NK cells prior to administration after three doses. In some embodiments, the number of NK cells increases at least 100-fold relative to the number of NK cells prior to administration after four doses. In some embodiments, the number of NK cells increases at least 100-fold relative to the number of NK cells prior to administration after five doses. In some embodiments, the number of NK cells increases at least 100-fold relative to the number of NK cells prior to administration after six doses. In some embodiments, the number of NK cells increases at least 100-fold relative to the number of NK cells prior to administration after seven doses. In some embodiments, the number of NK cells increases at least 100-fold relative to the number of NK cells prior to administration after eight doses. In some embodiments, the number of NK cells increases at least 100-fold relative to the number of NK cells prior to administration after nine doses. In some embodiments, the number of NK cells increases at least 100-fold relative to the number of NK cells prior to administration after ten doses.

In some embodiments, the number of NK cells increases at least 20-fold relative to the number of NK cells prior to administration after no more than no more than two doses. In some embodiments, the number of NK cells increases at least 20-fold relative to the number of NK cells prior to administration after no more than no more than three doses. In some embodiments, the number of NK cells increases at least 20-fold relative to the number of NK cells prior to administration after no more than four doses. In some embodiments, the number of NK cells increases at least 20-fold relative to the number of NK cells prior to administration after no more than five doses. In some embodiments, the number of NK cells increases at least 20-fold relative to the number of NK cells prior to administration after no more than six doses. In some embodiments, the number of NK cells increases at least 20-fold relative to the number of NK cells prior to administration after no more than seven doses. In some embodiments, the number of NK cells increases at least 20-fold relative to the number of NK cells prior to administration after no more than eight doses. In some embodiments, the number of NK cells increases at least 20-fold relative to the number of NK cells prior to administration after no more than nine doses. In some embodiments, the number of NK cells increases at least 20-fold relative to the number of NK cells prior to administration after no more than ten doses.

In some embodiments, the number of NK cells increases at least 50-fold relative to the number of NK cells prior to administration after no more than two doses. In some embodiments, the number of NK cells increases at least 50-fold relative to the number of NK cells prior to administration after no more than three doses. In some embodiments, the number of NK cells increases at least 50-fold relative to the number of NK cells prior to administration after no more than four doses. In some embodiments, the number of NK cells increases at least 50-fold relative to the number of NK cells prior to administration after no more than five doses. In some embodiments, the number of NK cells increases at least 50-fold relative to the number of NK cells prior to administration after no more than six doses. In some embodiments, the number of NK cells increases at least 50-fold relative to the number of NK cells prior to administration after no more than seven doses. In some embodiments, the number of NK cells increases at least 50-fold relative to the number of NK cells prior to administration after no more than eight doses. In some embodiments, the number of NK cells increases at least 50-fold relative to the number of NK cells prior to administration after no more than nine doses. In some embodiments, the number of NK cells increases at least 50-fold relative to the number of NK cells prior to administration after no more than ten doses.

In some embodiments, the number of NK cells increases at least 100-fold relative to the number of NK cells prior to administration after no more than two doses. In some embodiments, the number of NK cells increases at least 100-fold relative to the number of NK cells prior to administration after no more than three doses. In some embodiments, the number of NK cells increases at least 100-fold relative to the number of NK cells prior to administration after no more than four doses. In some embodiments, the number of NK cells increases at least 100-fold relative to the number of NK cells prior to administration after no more than five doses. In some embodiments, the number of NK cells increases at least 100-fold relative to the number of NK cells prior to administration after no more than six doses. In some embodiments, the number of NK cells increases at least 100-fold relative to the number of NK cells prior to administration after no more than seven doses. In some embodiments, the number of NK cells increases at least 100-fold relative to the number of NK cells prior to administration after no more than eight doses. In some embodiments, the number of NK cells increases at least 100-fold relative to the number of NK cells prior to administration after no more than nine doses. In some embodiments, the number of NK cells increases at least 100-fold relative to the number of NK cells prior to administration after no more than ten doses.

In some embodiments, the effective amount of the heterodimeric protein of the disclosure is an amount sufficient to increase the number of NK cells at least 20-fold relative to the number of NK cells prior to administration after two doses. In some embodiments, the effective amount of the heterodimeric protein of the disclosure is an amount sufficient to increase the number of NY cells at least 20-fold relative to the number of NK cells prior to administration after three doses. In some embodiments, the effective amount of the heterodimeric protein of the disclosure is an amount sufficient to increase the number of NK cells at least 20-fold relative to the number of NK cells prior to administration after four doses. In some embodiments, the effective amount of the heterodimeric protein of the disclosure is an amount sufficient to increase the number of NK cells at least 20-fold relative to the number of NK cells prior to administration after five doses. In some embodiments, the effective amount of the heterodimeric protein of the disclosure is an amount sufficient to increase the number of NK cells at least 20-fold relative to the number of NK cells prior to administration after six doses. In some embodiments, the effective amount of the heterodimeric protein of the disclosure is an amount sufficient to increase the number of NK cells at least 20-fold relative to the number of NK cells prior to administration after seven doses. In some embodiments, the effective amount of the heterodimeric protein of the disclosure is an amount sufficient to increase the number of NK cells at least 20-fold relative to the number of NK cells prior to administration after eight doses. In some embodiments, the effective amount of the heterodimeric protein of the disclosure is an amount sufficient to increase the number of NY cells at least 20-fold relative to the number of NK cells prior to administration after nine doses. In some embodiments, the effective amount of the heterodimeric protein of the disclosure is an amount sufficient to increase the number of NK cells at least 20-fold relative to the number of NK cells prior to administration after ten doses.

In some embodiments, the effective amount of the heterodimeric protein of the disclosure is an amount sufficient to increase the number of NK cells at least 50-fold relative to the number of NK cells prior to administration after two doses. In some embodiments, the effective amount of the heterodimeric protein of the disclosure is an amount sufficient to increase the number of NK cells at least 50-fold relative to the number of NK cells prior to administration after three doses. In some embodiments, the effective amount of the heterodimeric protein of the disclosure is an amount sufficient to increase the number of NK cells at least 50-fold relative to the number of NK cells prior to administration after four doses. In some embodiments, the effective amount of the heterodimeric protein of the disclosure is an amount sufficient to increase the number of NK cells at least 50-fold relative to the number of NK cells prior to administration after five doses. In some embodiments, the effective amount of the heterodimeric protein of the disclosure is an amount sufficient to increase the number of NK cells at least 50-fold relative to the number of NK cells prior to administration after six doses. In some embodiments, the effective amount of the heterodimeric protein of the disclosure is an amount sufficient to increase the number of NK cells at least 50-fold relative to the number of NK cells prior to administration after seven doses. In some embodiments, the effective amount of the heterodimeric protein of the disclosure is an amount sufficient to increase the number of NK cells at least 50-fold relative to the number of NK cells prior to administration after eight doses. In some embodiments, the effective amount of the heterodimeric protein of the disclosure is an amount sufficient to increase the number of NK cells at least 50-fold relative to the number of NK cells prior to administration after nine doses. In some embodiments, the effective amount of the heterodimeric protein of the disclosure is an amount sufficient to increase the number of NK cells at least 50-fold relative to the number of NK cells prior to administration after ten doses.

In some embodiments, the effective amount of the heterodimeric protein of the disclosure is an amount sufficient to increase the number of NK cells at least 100-fold relative to the number of NK cells prior to administration after two doses. In some embodiments, the effective amount of the heterodimeric protein of the disclosure is an amount sufficient to increase the number of NK cells at least 100-fold relative to the number of NK cells prior to administration after three doses. In some embodiments, the effective amount of the heterodimeric protein of the disclosure is an amount sufficient to increase the number of NK cells at least 100-fold relative to the number of NK cells prior to administration after four doses. In some embodiments, the effective amount of the heterodimeric protein of the disclosure is an amount sufficient to increase the number of NK cells at least 100-fold relative to the number of NK cells prior to administration after five doses. In some embodiments, the effective amount of the heterodimeric protein of the disclosure is an amount sufficient to increase the number of NK cells at least 100-fold relative to the number of NK cells prior to administration after six doses. In some embodiments, the effective amount of the heterodimeric protein of the disclosure is an amount sufficient to increase the number of NK cells at least 100-fold relative to the number of NK cells prior to administration after seven doses. In some embodiments, the effective amount of the heterodimeric protein of the disclosure is an amount sufficient to increase the number of NK cells at least 100-fold relative to the number of NK cells prior to administration after eight doses. In some embodiments, the effective amount of the heterodimeric protein of the disclosure is an amount sufficient to increase the number of NK cells at least 100-fold relative to the number of NK cells prior to administration after nine doses. In some embodiments, the effective amount of the heterodimeric protein of the disclosure is an amount sufficient to increase the number of NK cells at least 100-fold relative to the number of NK cells prior to administration after ten doses.

In some embodiments, the effective amount of the heterodimeric protein of the disclosure is an amount sufficient to increase the number of NK cells at least 20-fold relative to the number of NK cells prior to administration after no more than no more than two doses. In some embodiments, the effective amount of the heterodimeric protein of the disclosure is an amount sufficient to increase the number of NK cells at least 20-fold relative to the number of NK cells prior to administration after no more than no more than three doses. In some embodiments, the effective amount of the heterodimeric protein of the disclosure is an amount sufficient to increase the number of NK cells at least 20-fold relative to the number of NK cells prior to administration after no more than four doses. In some embodiments, the effective amount of the heterodimeric protein of the disclosure is an amount sufficient to increase the number of NK cells at least 20-fold relative to the number of NK cells prior to administration after no more than five doses. In some embodiments, the effective amount of the heterodimeric protein of the disclosure is an amount sufficient to increase the number of NK cells at least 20-fold relative to the number of NK cells prior to administration after no more than six doses. In some embodiments, the effective amount of the heterodimeric protein of the disclosure is an amount sufficient to increase the number of NK cells at least 20-fold relative to the number of NK cells prior to administration after no more than seven doses. In some embodiments, the effective amount of the heterodimeric protein of the disclosure is an amount sufficient to increase the number of NK cells at least 20-fold relative to the number of NK cells prior to administration after no more than eight doses. In some embodiments, the effective amount of the heterodimeric protein of the disclosure is an amount sufficient to increase the number of NK cells at least 20-fold relative to the number of NK cells prior to administration after no more than nine doses. In some embodiments, the effective amount of the heterodimeric protein of the disclosure is an amount sufficient to increase the number of NK cells at least 20-fold relative to the number of NK cells prior to administration after no more than ten doses.

In some embodiments, the effective amount of the heterodimeric protein of the disclosure is an amount sufficient to increase the number of NK cells at least 50-fold relative to the number of NK cells prior to administration after no more than two doses. In some embodiments, the effective amount of the heterodimeric protein of the disclosure is an amount sufficient to increase the number of NK cells at least 50-fold relative to the number of NK cells prior to administration after no more than three doses. In some embodiments, the effective amount of the heterodimeric protein of the disclosure is an amount sufficient to increase the number of NK cells at least 50-fold relative to the number of NK cells prior to administration after no more than four doses. In some embodiments, the effective amount of the heterodimeric protein of the disclosure is an amount sufficient to increase the number of NK cells at least 50-fold relative to the number of NK cells prior to administration after no more than five doses. In some embodiments, the effective amount of the heterodimeric protein of the disclosure is an amount sufficient to increase the number of NK cells at least 50-fold relative to the number of NK cells prior to administration after no more than six doses. In some embodiments, the effective amount of the heterodimeric protein of the disclosure is an amount sufficient to increase the number of NK cells at least 50-fold relative to the number of NK cells prior to administration after no more than seven doses. In some embodiments, the effective amount of the heterodimeric protein of the disclosure is an amount sufficient to increase the number of NK cells at least 50-fold relative to the number of NNK cells prior to administration after no more than eight doses. In some embodiments, the effective amount of the heterodimeric protein of the disclosure is an amount sufficient to increase the number of NK cells at least 50-fold relative to the number of NK cells prior to administration after no more than nine doses. In some embodiments, the effective amount of the heterodimeric protein of the disclosure is an amount sufficient to increase the number of NK cells at least 50-fold relative to the number of NK cells prior to administration after no more than ten doses.

In some embodiments, the effective amount of the heterodimeric protein of the disclosure is an amount sufficient to increase the number of NK cells at least 100-fold relative to the number of NK cells prior to administration after no more than two doses. In some embodiments, the effective amount of the heterodimeric protein of the disclosure is an amount sufficient to increase the number of NK cells at least 100-fold relative to the number of NK cells prior to administration after no more than three doses. In some embodiments, the effective amount of the heterodimeric protein of the disclosure is an amount sufficient to increase the number of NK cells at least 100-fold relative to the number of NK cells prior to administration after no more than four doses. In some embodiments, the effective amount of the heterodimeric protein of the disclosure is an amount sufficient to increase the number of NK cells at least 100-fold relative to the number of NK cells prior to administration after no more than five doses. In some embodiments, the effective amount of the heterodimeric protein of the disclosure is an amount sufficient to increase the number of NK cells at least 100-fold relative to the number of NK cells prior to administration after no more than six doses. In some embodiments, the effective amount of the heterodimeric protein of the disclosure is an amount sufficient to increase the number of NK cells at least 100-fold relative to the number of NK cells prior to administration after no more than seven doses. In some embodiments, the effective amount of the heterodimeric protein of the disclosure is an amount sufficient to increase the number of NK cells at least 100-fold relative to the number of NK cells prior to administration after no more than eight doses. In some embodiments, the effective amount of the heterodimeric protein of the disclosure is an amount sufficient to increase the number of NK cells at least 100-fold relative to the number of NK cells prior to administration after no more than nine doses. In some embodiments, the effective amount of the heterodimeric protein of the disclosure is an amount sufficient to increase the number of NK cells at least 100-fold relative to the number of NK cells prior to administration after no more than ten doses.

In some embodiments, the NK cells are CD16+ NK cells. In some embodiments, the NK cells are CD16+/CD56+ NK cells. In some embodiments, the NK cells are CD16+/CD56− NK cells. In some embodiments, the NK cells are CD16+/CD56ₗₒNK cells. In some embodiments, the NK cells are CD16+/CD56_{intermediate} NK cells. In some embodiments, the NK cells are CD16+/CD56ₕᵢ NK cells. In some embodiments, the NK cells are CD3−/CD16+/CD56+ NK cells. In some embodiments, the NK cells are CD3−/CD16+/CD56− NK cells. In some embodiments, the NK cells are CD3−/CD16+/CD56ₗₒ NK cells. In some embodiments, the NK cells are CD3−/CD16+/CD56_{intermediate} NK cells. In some embodiments, the NK cells are CD3−/CD16+/CD56ₕᵢ NK cells.

In some embodiments, the first monomer comprises an amino acid sequence encoded by a polynucleotide that encodes the amino acid sequence set forth in SEQ ID NO: 9. In some embodiments, the first monomer comprises an amino acid sequence encoded by a polynucleotide that encodes the amino acid sequence set forth in SEQ ID NO: 57. In some embodiments, the second monomer comprises an amino acid sequence encoded by a polynucleotide that encodes the amino acid sequence set forth in SEQ ID NO: 10. In some embodiments, the second monomer comprises an amino acid sequence encoded by a polynucleotide that encodes the amino acid sequence set forth in SEQ ID NO: 58. In some embodiments, the second monomer comprises an amino acid sequence encoded by a polynucleotide that encodes the amino acid sequence set forth in SEQ ID NO: 16. In some embodiments, the second monomer comprises an amino acid sequence encoded by a polynucleotide that encodes the amino acid sequence set forth in SEQ ID NO: 59.

In some embodiments, the first monomer comprises the amino acid sequence set forth in SEQ ID NO: 9. In some embodiments, the first monomer comprises the amino acid sequence set forth in SEQ ID NO: 57. In some embodiments, the second monomer comprises the amino acid sequence set forth in SEQ ID NO: 10. In some embodiments, the second monomer comprises the amino acid sequence set forth in SEQ ID NO: 58. In some embodiments, the second monomer comprises the amino acid sequence set forth in SEQ ID NO: 16. In some embodiments, the second monomer comprises the amino acid sequence set forth in SEQ ID NO: 59. In some embodiments, the heterodimeric protein is XENP24306, XENP32803, or a combination thereof. In some embodiments, the heterodimeric protein is XENP24306 with a C-terminal lysine, XENP24306 without a C-terminal lysine, XENP32803 with a C-terminal lysine, XENP32803 without a C-terminal lysine, or any combination thereof.

In some embodiments, the first monomer comprises an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1. In some embodiments, the second monomer comprises an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1. In some embodiments, the first Fc domain comprises amino acid substitutions L368D and K370S; the second Fc domain comprises amino acid substitutions S364K and E357Q; and each of said first and second Fc domains further comprises amino acid substitutions C220S, E233P, L234V, L235A, G236 del, S267K, M428L and N434S, according to EU numbering. In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, the first monomer lacks a C-terminal lysine. In some embodiments, the second monomer comprises a C-terminal lysine. In some embodiments, the second monomer lacks a C-terminal lysine. In some embodiments of any of the methods disclosed herein, a combination of XENP24306 with a C-terminal lysine and XENP24306 without a C-terminal lysine. In some embodiments of any of the methods disclosed herein, the heterodimeric protein is XENP32803 with a C-terminal lysine and XENP32803 without a C-terminal lysine. In some embodiments of any of the methods disclosed herein, a combination of (1) XENP24306 with a C-terminal lysine and XENP24306 without a C-terminal lysine; and (2) XENP32803 with a C-terminal lysine and XENP32803 without a C-terminal lysine are used. In some embodiments of any of the methods disclosed herein, a combination of (1) XENP24306 with a C-terminal lysine; and (2) XENP32803 with a C-terminal lysine are used. In some embodiments of any of the methods disclosed herein, a combination of (1) XENP24306 with a C-terminal lysine; and (2) XENP32803 without a C-terminal lysine are used. In some embodiments of any of the methods disclosed herein, a combination of (1) XENP24306 with a C-terminal lysine; and (2) XENP32803 with a C-terminal lysine and XENP32803 without a C-terminal lysine are used. In some embodiments of any of the methods disclosed herein, a combination of (1) XENP24306 without a C-terminal lysine; and (2) XENP32803 with a C-terminal lysine are used. In some embodiments of any of the methods disclosed herein, a combination of (1) XENP24306 without a C-terminal lysine; and (2) XENP32803 without a C-terminal lysine are used. In some embodiments of any of the methods disclosed herein, a combination of (1) XENP24306 without a C-terminal lysine; and (2) XENP32803 with a C-terminal lysine and XENP32803 without a C-terminal lysine are used.

In some embodiments, the first monomer comprises the amino acid sequence set forth in SEQ ID NO: 9, and the second monomer comprises the amino acid sequence set forth in SEQ ID NO: 10. In some embodiments, the first monomer comprises the amino acid sequence set forth in SEQ ID NO: 57, and the second monomer comprises the amino acid sequence set forth in SEQ ID NO: 10. In some embodiments, the first monomer comprises the amino acid sequence set forth in SEQ ID NO: 9, and the second monomer comprises the amino acid sequence set forth in SEQ ID NO: 58. In some embodiments, the first monomer comprises the amino acid sequence set forth in SEQ ID NO: 57, and the second monomer comprises the amino acid sequence set forth in SEQ ID NO: 58. In some embodiments, the first monomer comprises the amino acid sequence set forth in SEQ ID NO: 9, and the second monomer comprises the amino acid sequence set forth in SEQ ID NO: 16. In some embodiments, the first monomer comprises the amino acid sequence set forth in SEQ ID NO: 57, and the second monomer comprises the amino acid sequence set forth in SEQ ID NO: 16. In some embodiments, the first monomer comprises the amino acid sequence set forth in SEQ ID NO: 9, and the second monomer comprises the amino acid sequence set forth in SEQ ID NO: 59. In some embodiments, the first monomer comprises the amino acid sequence set forth in SEQ ID NO: 57, and the second monomer comprises the amino acid sequence set forth in SEQ ID NO: 59.

In some embodiments, a combination of two or more (e.g., 2, 3, 4, 5, 6, etc.) heterodimeric proteins are used in the methods described herein. In some embodiments, a combination of a first heterodimeric protein and a second heterodimeric protein is administered to the subject.

In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 9, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 59. In some embodiments, the first heterodimeric protein comprises a first monomer comprising the amino acid sequence set forth in SEQ ID NO: 9, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 59.

In some embodiments, the first heterodimeric protein represents about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 75%, about 70%, about 65%, about 60%, about 55%, about 50%, about 45%, about 40%, about 35%, about 30%, about 25%, about 20%, about 15%, about 10%, or about 5% of the heterodimeric protein in the combination. In some embodiments, the first heterodimeric protein represents about 85% of the heterodimeric protein in the combination. In some embodiments, the first heterodimeric protein represents about 84% of the heterodimeric protein in the combination. In some embodiments, the first heterodimeric protein represents about 83% of the heterodimeric protein in the combination. In some embodiments, the first heterodimeric protein represents about 82% of the heterodimeric protein in the combination. In some embodiments, the first heterodimeric protein represents about 81% of the heterodimeric protein in the combination. In some embodiments, the first heterodimeric protein represents about 80% of the heterodimeric protein in the combination.

In some embodiments, the second heterodimeric protein represents about 95%, about 90%, about 85%, about 80%, about 75%, about 70%, about 75%, about 70%, about 65%, about 55%, about 50%, about 45%, about 40%, about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% of the combination. In some embodiments, the second heterodimeric protein represents about 15% of the heterodimeric protein in the combination. In some embodiments, the second heterodimeric protein represents about 16% of the heterodimeric protein in the combination. In some embodiments, the second heterodimeric protein represents about 17% of the heterodimeric protein in the combination. In some embodiments, the second heterodimeric protein represents about 18% of the heterodimeric protein in the combination. In some embodiments, the second heterodimeric protein represents about 19% of the heterodimeric protein in the combination. In some embodiments, the second heterodimeric protein represents about 20% of the heterodimeric protein in the combination.

In some embodiments, the first heterodimeric protein represents between about 50-about 100%, about 70-about 95%, about 80-about 90%, or about 80-about 85% of the heterodimeric protein in the combination. In some embodiments of any of the methods disclosed herein, the second heterodimeric protein represents between about 1-about 50%, about 5-about 30%, about 10-about 20%, or about 15-about 20% of the heterodimeric protein in the combination. In some embodiments, the first heterodimeric protein represents about 85% of the heterodimeric protein in the combination, and the second heterodimeric protein represents about 15% of the heterodimeric protein in the combination. In some embodiments, the first heterodimeric protein represents about 84% of the heterodimeric protein in the combination, and the second heterodimeric protein represents about 16% of the heterodimeric protein in the combination. In some embodiments, the first heterodimeric protein represents about 83% of the heterodimeric protein in the combination, and the second heterodimeric protein represents about 17% of the heterodimeric protein in the combination. In some embodiments, the first heterodimeric protein represents about 82% of the heterodimeric protein in the combination, and the second heterodimeric protein represents about 18% of the heterodimeric protein in the combination. In some embodiments, the first heterodimeric protein represents about 81% of the heterodimeric protein in the combination, and the second heterodimeric protein represents about 19% of the heterodimeric protein in the combination. In some embodiments, the first heterodimeric protein represents about 80% of the heterodimeric protein in the combination, and the second heterodimeric protein represents about 20% of the heterodimeric protein in the combination.

In some embodiments, the first heterodimeric protein represents 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, 25%, 20%, 15%, 10%, or 5% of the heterodimeric protein in the combination. In some embodiments, the first heterodimeric protein represents 85% of the heterodimeric protein in the combination. In some embodiments, the first heterodimeric protein represents 84% of the heterodimeric protein in the combination. In some embodiments, the first heterodimeric protein represents 83% of the heterodimeric protein in the combination. In some embodiments, the first heterodimeric protein represents 82% of the heterodimeric protein in the combination. In some embodiments, the first heterodimeric protein represents 81% of the heterodimeric protein in the combination. In some embodiments, the first heterodimeric protein represents 80% of the heterodimeric protein in the combination.

In some embodiments, the second heterodimeric protein represents 95%, 90%, 85%, 80%, 75%, 70%, 75%, 70%, 65%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% of the combination. In some embodiments, the second heterodimeric protein represents 15% of the heterodimeric protein in the combination. In some embodiments, the second heterodimeric protein represents 16% of the heterodimeric protein in the combination. In some embodiments, the second heterodimeric protein represents 17% of the heterodimeric protein in the combination. In some embodiments, the second heterodimeric protein represents 18% of the heterodimeric protein in the combination. In some embodiments, the second heterodimeric protein represents 19% of the heterodimeric protein in the combination. In some embodiments, the second heterodimeric protein represents 20% of the heterodimeric protein in the combination.

In some embodiments, the first heterodimeric protein represents between 50-100%, 70-95%, 80-90%, or 80-85% of the heterodimeric protein in the combination. In some embodiments of any of the methods disclosed herein, the second heterodimeric protein represents between 1-50%, 5-30%, 10-20%, or 15-20% of the heterodimeric protein in the combination. In some embodiments, the first heterodimeric protein represents 85% of the heterodimeric protein in the combination, and the second heterodimeric protein represents 15% of the heterodimeric protein in the combination. In some embodiments, the first heterodimeric protein represents 84% of the heterodimeric protein in the combination, and the second heterodimeric protein represents 16% of the heterodimeric protein in the combination. In some embodiments, the first heterodimeric protein represents 83% of the heterodimeric protein in the combination, and the second heterodimeric protein represents 17% of the heterodimeric protein in the combination. In some embodiments, the first heterodimeric protein represents 82% of the heterodimeric protein in the combination, and the second heterodimeric protein represents 18% of the heterodimeric protein in the combination. In some embodiments, the first heterodimeric protein represents 81% of the heterodimeric protein in the combination, and the second heterodimeric protein represents 19% of the heterodimeric protein in the combination. In some embodiments, the first heterodimeric protein represents 80% of the heterodimeric protein in the combination, and the second heterodimeric protein represents 20% of the heterodimeric protein in the combination.

In some embodiments, said first and second heterodimeric proteins are administered simultaneously. In some embodiments, said first and second heterodimeric proteins are administered sequentially. In some embodiments, the first heterodimeric protein is administered before the second heterodimeric protein. In some embodiments, the second heterodimeric protein is administered before the first heterodimeric protein. In some embodiments, said first and second heterodimeric proteins are administered in the same composition. In some embodiments, the first and second heterodimeric proteins are administered in separate compositions.

A solid tumor refers to an abnormal mass of tissue that usually does not contain cysts or liquid areas. Different types of solid tumors are named for the type of cells that form them. Examples of solid tumors to be treated by the methods and uses disclosed herein include, but are not limited, carcinomas, lymphomas, blastomas and sarcomas. More particular examples of such tumors include squamous cell cancer, cutaneous squamous cell carcinoma (cSCC), small-cell lung carcinoma (SCLC), non-small cell lung cancer (NSCLC), gastrointestinal cancer, gastric cancer (GC), pancreatic cancer, glioblastoma, cervical cancer, ovarian cancer, liver cancer, bladder cancer, liposarcoma, soft-tissue sarcoma, urothelial carcinoma (UCC), ureter and renal pelvis, multiple myeloma, osteosarcoma, hepatoma, melanoma, stomach cancer, breast cancer, colon cancer, colorectal cancer, endometrial carcinoma, salivary gland carcinoma, renal cell carcinoma (RCC), liver cancer, esophageal cancer, prostate cancer, vulval cancer, thyroid cancer, hepatic carcinoma, Merkel cell carcinoma (MCC), germ cell cancer, micro-satellite instability-high (MSI-H) cancer and head and neck cancer. In some embodiments, the solid tumor is a locally advanced, recurrent, or metastatic incurable solid tumor. In some embodiments, the solid tumor is selected from the group consisting of melanoma, NSCLC, head and neck squamous cell carcinoma (HNSCC), triple-negative breast cancer (TNBC), UCC, RCC, SCLC, GC, MCC, cSCC and MSI-H cancers. In some embodiments, the solid tumor is selected from melanoma, RCC, NSCLC, HNSCC and TNBC. In some embodiments, the solid tumor is melanoma. In some embodiments, the solid tumor is RCC. In some embodiments, the solid tumor is selected from melanoma, RCC and NSCLC. In some embodiments, the solid tumor is selected from melanoma, NSCLC, HNSCC and TNBC. In some embodiments, the solid tumor is NSCLC. In some embodiments, the solid tumor is HNSCC. In some embodiments, the solid tumor is TNBC. In some embodiments, the solid tumor is a solid tumor for which standard therapy does not exist, has proven to be ineffective or intolerable, or is considered inappropriate, or for whom a clinical trial of an investigational agent is a recognized standard of care.

The methods and uses herein described include administering to the subject a therapeutically effective amount of any of the heterodimeric proteins described herein, or a combination thereof, or a composition described herein to produce such effect. Identifying a subject in need of such treatment can be in the judgment of a subject or a health care professional and can be subjective (e.g., opinion) or objective (e.g., measurable by a test or diagnostic method). Such treatment will be suitably administered to subjects suffering from, having, susceptible to, or at risk for cancer.

The heterodimeric protein (or combination thereof) may be administered parenterally. In some embodiments, the parenteral administration is intravenous. In some embodiments, the heterodimeric protein is administered intravenously. In some embodiments, the heterodimeric protein is administered as a composition comprising a pharmaceutically acceptable buffer. Suitable carriers and their formulations are described, for example, in Remington's Pharmaceutical Sciences by E. W. Martin. In some embodiments, the heterodimeric protein is provided in a dosage form that is suitable for parenteral (e.g. intravenous) administration.

Compositions comprising the heterodimeric protein may be provided in unit dosage forms (e.g., in single-dose ampoules, syringes or bags). In some embodiments, the heterodimeric protein is provided in vials containing several doses. A suitable preservative may be added to the composition (see below). The composition may be in the form of a solution, a suspension, an emulsion, an infusion device, or a delivery device for implantation, or it may be presented as a dry powder to be reconstituted with water or another suitable vehicle before use. Apart from the heterodimeric proteins disclosed herein, the composition may include suitable acceptable carriers and/or excipients. In some embodiments, the composition is suitable for parenteral administration. The heterodimeric protein(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

The pharmaceutical compositions comprising the heterodimeric protein may be in a form suitable for sterile injection. To prepare such a composition, the protein is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

The amount of the heterodimeric protein of the disclosure to be administered varies depending upon the manner of administration, the age and body weight of the patient, and the clinical symptoms of the cancer to be treated. In some embodiments, the subject is a human subject. Human dosage amounts can initially be determined by extrapolating from the amount of protein used in mice or non-human primates. In certain embodiments, the dosage may vary from between about 0.0001 mg protein/kg body weight to about 5 mg compound/kg body weight; or from about 0.001 mg/kg body weight to about 4 mg/kg body weight or from about 0.005 mg/kg body weight to about 1 mg/kg body weight or from about 0.005 mg/kg body weight to about 0.3 mg/kg body weight or from about 0.005 mg/kg body weight to about 0.2 mg/kg body weight or from about 0.005 mg/kg body weight to about 0.02 mg/kg body weight. In some embodiments, this dose may be about 0.0001, about 0.00025, about 0.0003, about 0.0005, about 0.001, about 0.003, about 0.005, about 0.008, about 0.01, about 0.015, about 0.02, about 0.03, about 0.04, about 0.05, about 0.06, about 0.07, about 0.08, about 0.09, about 0.1, about 0.12, about 0.135, about 0.15, about 0.16, about 0.2, about 0.2025, about 0.24, about 0.25, about 0.3, about 0.32, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.15, about 1.2, about 1.25, about 1.3, about 1.35, about 1.4, about 1.45, about 1.5, about 1.6, about 1.7, about 1.8, about 1.9, about 2, about 2.5, about 3, about 3.5, about 4, about 4.5, or about 5 mg/kg body weight. In some embodiments, the dose is about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight. In some embodiments, the dosage is about 0.0025 mg/kg body weight. In some embodiments, the dosage is about 0.01 mg/kg body weight. In some embodiments, the dosage is about 0.015 mg/kg body weight. In some embodiments, the dosage is about 0.02 mg/kg body weight. In some embodiments, the dosage is about 0.03 mg/kg body weight. In some embodiments, the dosage is about 0.04 mg/kg body weight. In some embodiments, the dosage is about 0.06 mg/kg body weight. In some embodiments, the dosage is about 0.08 mg/kg body weight. In some embodiments, the dosage is about 0.09 mg/kg body weight. In some embodiments, the dosage is about 0.12 mg/kg body weight. In some embodiments, the dosage is about 0.135 mg/kg body weight. In some embodiments, the dosage is about 0.16 mg/kg body weight. In some embodiments, the dosage is about 0.2025 mg/kg body weight. In some embodiments, the dosage is about 0.24 mg/kg body weight. In some embodiments, the dosage is about 0.32 mg/kg body weight. In some embodiments, the heterodimeric protein of the disclosure is administered by IV infusion according to these dosages.

In certain embodiments, the dosage may vary from between 0.0001 mg protein/kg body weight to 5 mg compound/kg body weight; or from 0.001 mg/kg body weight to 4 mg/kg body weight or from 0.005 mg/kg body weight to 1 mg/kg body weight or from 0.005 mg/kg body weight to 0.3 mg/kg body weight or from 0.005 mg/kg body weight to 0.2 mg/kg body weight or from 0.005 mg/kg body weight to 0.02 mg/kg body weight. In some embodiments, this dose may be 0.0001, 0.0003, 0.0005, 0.001, 0.003, 0.005, 0.008, 0.01, 0.015, 0.02, 0.03, 0.05, 0.08, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.15, 1.2, 1.25, 1.3, 1.35, 1.4, 1.45, 1.5, 1.6, 1.7, 1.8, 1.9, 2, 2.5, 3, 3.5, 4, 4.5, or 5 mg/kg body weight. In some embodiments, the dose is selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.09 mg/kg, 0.10 mg/kg, 0.12 mg/kg, 0.135 mg/kg, 0.16 mg/kg, 0.20 mg/kg, 0.2025 mg/kg, 0.24 mg/kg and 0.32 mg/kg body weight. In some embodiments, the dosage is 0.0025 mg/kg body weight. In some embodiments, the dosage is 0.01 mg/kg body weight. In some embodiments, the dosage is 0.015 mg/kg body weight. In some embodiments, the dosage is 0.02 mg/kg body weight. In some embodiments, the dosage is 0.03 mg/kg body weight. In some embodiments, the dosage is 0.04 mg/kg body weight. In some embodiments, the dosage is 0.06 mg/kg body weight. In some embodiments, the dosage is 0.08 mg/kg body weight. In some embodiments, the dosage is 0.09 mg/kg body weight. In some embodiments, the dosage is 0.12 mg/kg body weight. In some embodiments, the dosage is 0.135 mg/kg body weight. In some embodiments, the dosage is 0.16 mg/kg body weight. In some embodiments, the dosage is 0.2025 mg/kg body weight. In some embodiments, the dosage is 0.24 mg/kg body weight. In some embodiments, the dosage is 0.32 mg/kg body weight. In some embodiments, the heterodimeric protein of the disclosure is administered by IV infusion according to these dosages.

In certain embodiments, the dosage of the combination of heterodimeric proteins may vary from between about 0.0001 mg protein/kg body weight to about 5 mg compound/kg body weight; or from about 0.001 mg/kg body weight to about 4 mg/kg body weight or from about 0.005 mg/kg body weight to about 1 mg/kg body weight or from about 0.005 mg/kg body weight to about 0.3 mg/kg body weight or from about 0.005 mg/kg body weight to about 0.2 mg/kg body weight or from about 0.005 mg/kg body weight to about 0.02 mg/kg body weight. In some embodiments, this dose may be about 0.0001, about 0.0003, about 0.0005, about 0.001, about 0.003, about 0.005, about 0.008, about 0.01, about 0.015, about 0.02, about 0.03, about 0.05, about 0.08, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.15, about 1.2, about 1.25, about 1.3, about 1.35, about 1.4, about 1.45, about 1.5, about 1.6, about 1.7, about 1.8, about 1.9, about 2, about 2.5, about 3, about 3.5, about 4, about 4.5, or about 5 mg/kg body weight. In some embodiments, the dose is about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.10 mg/kg, about 0.12 mg/kg, about 0.16 mg/kg, about 0.20 mg/kg, about 0.24 mg/kg and about 0.32 mg/kg body weight. In some embodiments, the dosage is about 0.0025 mg/kg body weight. In some embodiments, the dosage is about 0.01 mg/kg body weight. In some embodiments, the dosage is about 0.015 mg/kg body weight. In some embodiments, the dosage is about 0.02 mg/kg body weight. In some embodiments, the dosage is about 0.03 mg/kg body weight. In some embodiments, the dosage is about 0.04 mg/kg body weight. In some embodiments, the dosage is about 0.06 mg/kg body weight. In some embodiments, the dosage is about 0.08 mg/kg body weight. In some embodiments, the dosage is about 0.12 mg/kg body weight. In some embodiments, the dosage is about 0.16 mg/kg body weight. In some embodiments, the dosage is about 0.24 mg/kg body weight. In some embodiments, the dosage is about 0.32 mg/kg body weight. In some embodiments, the combination of heterodimeric proteins of the disclosure is administered by IV infusion according to these dosages.

In certain embodiments, the dosage of the combination of heterodimeric proteins may vary from between 0.0001 mg protein/kg body weight to 5 mg compound/kg body weight; or from 0.001 mg/kg body weight to 4 mg/kg body weight or from 0.005 mg/kg body weight to 1 mg/kg body weight or from 0.005 mg/kg body weight to 0.3 mg/kg body weight or from 0.005 mg/kg body weight to 0.2 mg/kg body weight or from 0.005 mg/kg body weight to 0.02 mg/kg body weight. In some embodiments, this dose may be 0.0001, 0.0003, 0.0005, 0.001, 0.003, 0.005, 0.008, 0.01, 0.015, 0.02, 0.03, 0.05, 0.08, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.15, 1.2, 1.25, 1.3, 1.35, 1.4, 1.45, 1.5, 1.6, 1.7, 1.8, 1.9, 2, 2.5, 3, 3.5, 4, 4.5, or 5 mg/kg body weight. In some embodiments, the dose is 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.12 mg/kg, 0.16 mg/kg, 0.20 mg/kg, 0.24 mg/kg and 0.32 mg/kg body weight. In some embodiments, the dosage is 0.0025 mg/kg body weight. In some embodiments, the dosage is 0.01 mg/kg body weight. In some embodiments, the dosage is 0.015 mg/kg body weight. In some embodiments, the dosage is 0.02 mg/kg body weight. In some embodiments, the dosage is 0.03 mg/kg body weight. In some embodiments, the dosage is 0.04 mg/kg body weight. In some embodiments, the dosage is 0.06 mg/kg body weight. In some embodiments, the dosage is 0.08 mg/kg body weight. In some embodiments, the dosage is 0.12 mg/kg body weight. In some embodiments, the dosage is 0.16 mg/kg body weight. In some embodiments, the dosage is 0.24 mg/kg body weight. In some embodiments, the dosage is 0.32 mg/kg body weight. In some embodiments, the combination of heterodimeric proteins of the disclosure is administered by IV infusion according to these dosages.

In some embodiments, the heterodimeric protein of the disclosure, or a combination thereof, is administered daily, i.e., every 24 hours. In some embodiments, the heterodimeric protein or a combination thereof is administered weekly, i.e., once per week (Q1W). In some embodiments, the heterodimeric protein or a combination thereof is administered once every two weeks, i.e., once every 14 days (Q2W). In some embodiments, the heterodimeric protein or a combination thereof is administered once every three weeks, i.e., once every 21 days (Q3W). In some embodiments, the heterodimeric protein or a combination thereof is administered once every four weeks, i.e., once every 28 days (Q4W). In some embodiments, the heterodimeric protein or a combination thereof is administered once every five weeks (Q5W). In some embodiments, the heterodimeric protein or a combination thereof is administered once every six weeks (Q6W). In some embodiments, the heterodimeric protein or a combination thereof is administered once every seven weeks (Q7W). In some embodiments, the heterodimeric protein or a combination thereof is administered once every eight weeks (Q8W). In some embodiments, the heterodimeric protein or a combination thereof is administered once every nine weeks (Q9W). In some embodiments, the heterodimeric protein or a combination thereof is administered once every ten weeks (Q10W). In some embodiments, the heterodimeric protein or a combination thereof is administered once every eleven weeks (Q11W). In some embodiments, the heterodimeric protein or a combination thereof is administered once every twelve weeks (Q12W). In some embodiments, the heterodimeric protein or a combination thereof is administered once every month. In some embodiments, the heterodimeric protein or a combination thereof is administered once every two months. In some embodiments, the heterodimeric protein or a combination thereof is administered once every three months. In some embodiments, the heterodimeric protein or a combination thereof is administered once every four months. In some embodiments, the heterodimeric protein or a combination thereof is administered once every five months. In some embodiments, the heterodimeric protein or a combination thereof is administered once every six months. In some embodiments, the heterodimeric protein or a combination thereof is administered once every seven months. In some embodiments, the heterodimeric protein or a combination thereof is administered once every eight months. In some embodiments, the heterodimeric protein or a combination thereof is administered once every nine months. In some embodiments, the heterodimeric protein or a combination thereof is administered once every ten months. In some embodiments, the heterodimeric protein or a combination thereof is administered once every eleven months. In some embodiments, the heterodimeric protein or a combination thereof is administered once every twelve months. In some embodiments, the heterodimeric protein or a combination thereof is administered once every year. In some embodiments, the heterodimeric protein or a combination thereof of the disclosure is administered by IV infusion according to the frequency disclosed herein.

In some embodiments, the heterodimeric protein is administered at a dose of at least 0.02 mg/kg Q2W and the number of NK cells increases at least 20-fold as compared to the number of NK cells prior to administration after two doses. In some embodiments, the heterodimeric protein is administered at a dose of at least 0.02 mg/kg Q2W and the number of NK cells increases at least 20-fold as compared to the number of NK cells prior to administration after three doses. In some embodiments, the heterodimeric protein is administered at a dose of at least 0.02 mg/kg Q2W and the number of NK cells increases at least 20-fold as compared to the number of NK cells prior to administration after four doses. In some embodiments, the heterodimeric protein is administered at a dose of at least 0.02 mg/kg Q2W and the number of NK cells increases at least 20-fold as compared to the number of NK cells prior to administration after five doses. In some embodiments, the heterodimeric protein is administered at a dose of at least 0.02 mg/kg Q2W and the number of NK cells increases at least 30-fold as compared to the number of NK cells prior to administration after two doses. In some embodiments, the heterodimeric protein is administered at a dose of at least 0.02 mg/kg Q2W and the number of NK cells increases at least 30-fold as compared to the number of NK cells prior to administration after three doses. In some embodiments, the heterodimeric protein is administered at a dose of at least 0.02 mg/kg Q2W and the number of NK cells increases at least 30-fold as compared to the number of NK cells prior to administration after four doses. In some embodiments, the heterodimeric protein is administered at a dose of at least 0.02 mg/kg Q2W and the number of NK cells increases at least 30-fold as compared to the number of NK cells prior to administration after five doses. In some embodiments, the heterodimeric protein is administered at a dose of at least 0.02 mg/kg Q2W and the number of NK cells increases at least 40-fold as compared to the number of NK cells prior to administration after two doses. In some embodiments, the heterodimeric protein is administered at a dose of at least 0.02 mg/kg Q2W and the number of NK cells increases at least 40-fold as compared to the number of NK cells prior to administration after three doses. In some embodiments, the heterodimeric protein is administered at a dose of at least 0.02 mg/kg Q2W and the number of NK cells increases at least 40-fold as compared to the number of NK cells prior to administration after four doses. In some embodiments, the heterodimeric protein is administered at a dose of at least 0.02 mg/kg Q2W and the number of NK cells increases at least 40-fold as compared to the number of NK cells prior to administration after five doses. In some embodiments, the heterodimeric protein is administered at a dose of at least 0.02 mg/kg Q2W and the number of NK cells increases at least 50-fold as compared to the number of NK cells prior to administration after two doses. In some embodiments, the heterodimeric protein is administered at a dose of at least 0.02 mg/kg Q2W and the number of NK cells increases at least 50-fold as compared to the number of NK cells prior to administration after three doses. In some embodiments, the heterodimeric protein is administered at a dose of at least 0.02 mg/kg Q2W and the number of NK cells increases at least 50-fold as compared to the number of NK cells prior to administration after four doses. In some embodiments, the heterodimeric protein is administered at a dose of at least 0.02 mg/kg Q2W and the number of NK cells increases at least 50-fold as compared to the number of NK cells prior to administration after five doses. In some embodiments, the heterodimeric protein is administered at a dose of at least 0.02 mg/kg Q2W and the number of NK cells increases at least 100-fold as compared to the number of NK cells prior to administration after two doses. In some embodiments, the heterodimeric protein is administered at a dose of at least 0.02 mg/kg Q2W and the number of NK cells increases at least 100-fold as compared to the number of NK cells prior to administration after three doses. In some embodiments, the heterodimeric protein is administered at a dose of at least 0.02 mg/kg Q2W and the number of NK cells increases at least 100-fold as compared to the number of NK cells prior to administration after four doses. In some embodiments, the heterodimeric protein is administered at a dose of at least 0.02 mg/kg Q2W and the number of NK cells increases at least 100-fold as compared to the number of NK cells prior to administration after five doses. In some embodiments, the heterodimeric protein is administered at a dose of at least 0.02 mg/kg Q2W and the number of NK cells increases at least 200-fold as compared to the number of NK cells prior to administration after two doses. In some embodiments, the heterodimeric protein is administered at a dose of at least 0.02 mg/kg Q2W and the number of NK cells increases at least 200-fold as compared to the number of NK cells prior to administration after three doses. In some embodiments, the heterodimeric protein is administered at a dose of at least 0.02 mg/kg Q2W and the number of NK cells increases at least 200-fold as compared to the number of NK cells prior to administration after four doses. In some embodiments, the heterodimeric protein is administered at a dose of at least 0.02 mg/kg Q2W and the number of NK cells increases at least 200-fold as compared to the number of NK cells prior to administration after five doses.

In some embodiments, the heterodimeric protein is administered at a dose of at least 0.04 mg/kg Q2W and the number of NK cells increases at least 20-fold as compared to the number of NK cells prior to administration after two doses. In some embodiments, the heterodimeric protein is administered at a dose of at least 0.04 mg/kg Q2W and the number of NK cells increases at least 20-fold as compared to the number of NK cells prior to administration after three doses. In some embodiments, the heterodimeric protein is administered at a dose of at least 0.04 mg/kg Q2W and the number of NK cells increases at least 20-fold as compared to the number of NK cells prior to administration after four doses. In some embodiments, the heterodimeric protein is administered at a dose of at least 0.04 mg/kg Q2W and the number of NK cells increases at least 20-fold as compared to the number of NK cells prior to administration after five doses. In some embodiments, the heterodimeric protein is administered at a dose of at least 0.04 mg/kg Q2W and the number of NK cells increases at least 30-fold as compared to the number of NK cells prior to administration after two doses. In some embodiments, the heterodimeric protein is administered at a dose of at least 0.04 mg/kg Q2W and the number of NK cells increases at least 30-fold as compared to the number of NK cells prior to administration after three doses. In some embodiments, the heterodimeric protein is administered at a dose of at least 0.04 mg/kg Q2W and the number of NK cells increases at least 30-fold as compared to the number of NK cells prior to administration after four doses. In some embodiments, the heterodimeric protein is administered at a dose of at least 0.04 mg/kg Q2W and the number of NK cells increases at least 30-fold as compared to the number of NK cells prior to administration after five doses. In some embodiments, the heterodimeric protein is administered at a dose of at least 0.04 mg/kg Q2W and the number of NK cells increases at least 40-fold as compared to the number of NY cells prior to administration after two doses. In some embodiments, the heterodimeric protein is administered at a dose of at least 0.04 mg/kg Q2W and the number of NK cells increases at least 40-fold as compared to the number of NK cells prior to administration after three doses. In some embodiments, the heterodimeric protein is administered at a dose of at least 0.04 mg/kg Q2W and the number of NK cells increases at least 40-fold as compared to the number of NK cells prior to administration after four doses. In some embodiments, the heterodimeric protein is administered at a dose of at least 0.04 mg/kg Q2W and the number of NK cells increases at least 40-fold as compared to the number of NK cells prior to administration after five doses. In some embodiments, the heterodimeric protein is administered at a dose of at least 0.04 mg/kg Q2W and the number of NK cells increases at least 50-fold as compared to the number of NK cells prior to administration after two doses. In some embodiments, the heterodimeric protein is administered at a dose of at least 0.04 mg/kg Q2W and the number of NK cells increases at least 50-fold as compared to the number of NK cells prior to administration after three doses. In some embodiments, the heterodimeric protein is administered at a dose of at least 0.04 mg/kg Q2W and the number of NK cells increases at least 50-fold as compared to the number of NK cells prior to administration after four doses. In some embodiments, the heterodimeric protein is administered at a dose of at least 0.04 mg/kg Q2W and the number of NK cells increases at least 50-fold as compared to the number of NK cells prior to administration after five doses. In some embodiments, the heterodimeric protein is administered at a dose of at least 0.04 mg/kg Q2W and the number of NK cells increases at least 100-fold as compared to the number of NK cells prior to administration after two doses. In some embodiments, the heterodimeric protein is administered at a dose of at least 0.04 mg/kg Q2W and the number of NK cells increases at least 100-fold as compared to the number of NK cells prior to administration after three doses. In some embodiments, the heterodimeric protein is administered at a dose of at least 0.04 mg/kg Q2W and the number of NK cells increases at least 100-fold as compared to the number of NK cells prior to administration after four doses. In some embodiments, the heterodimeric protein is administered at a dose of at least 0.04 mg/kg Q2W and the number of NK cells increases at least 100-fold as compared to the number of NK cells prior to administration after five doses. In some embodiments, the heterodimeric protein is administered at a dose of at least 0.04 mg/kg Q2W and the number of NK cells increases at least 200-fold as compared to the number of NK cells prior to administration after two doses. In some embodiments, the heterodimeric protein is administered at a dose of at least 0.04 mg/kg Q2W and the number of NK cells increases at least 200-fold as compared to the number of NK cells prior to administration after three doses. In some embodiments, the heterodimeric protein is administered at a dose of at least 0.04 mg/kg Q2W and the number of NK cells increases at least 200-fold as compared to the number of NK cells prior to administration after four doses. In some embodiments, the heterodimeric protein is administered at a dose of at least 0.04 mg/kg Q2W and the number of NK cells increases at least 200-fold as compared to the number of NK cells prior to administration after five doses.

In some embodiments, the heterodimeric protein is administered at a dose of at least 0.06 mg/kg Q2W and the number of NK cells increases at least 20-fold as compared to the number of NK cells prior to administration after two doses. In some embodiments, the heterodimeric protein is administered at a dose of at least 0.06 mg/kg Q2W and the number of NK cells increases at least 20-fold as compared to the number of NK cells prior to administration after three doses. In some embodiments, the heterodimeric protein is administered at a dose of at least 0.06 mg/kg Q2W and the number of NK cells increases at least 20-fold as compared to the number of NK cells prior to administration after four doses. In some embodiments, the heterodimeric protein is administered at a dose of at least 0.06 mg/kg Q2W and the number of NK cells increases at least 20-fold as compared to the number of NK cells prior to administration after five doses. In some embodiments, the heterodimeric protein is administered at a dose of at least 0.06 mg/kg Q2W and the number of NK cells increases at least 30-fold as compared to the number of NK cells prior to administration after two doses. In some embodiments, the heterodimeric protein is administered at a dose of at least 0.06 mg/kg Q2W and the number of NK cells increases at least 30-fold as compared to the number of NK cells prior to administration after three doses. In some embodiments, the heterodimeric protein is administered at a dose of at least 0.06 mg/kg Q2W and the number of NK cells increases at least 30-fold as compared to the number of NK cells prior to administration after four doses. In some embodiments, the heterodimeric protein is administered at a dose of at least 0.06 mg/kg Q2W and the number of NK cells increases at least 30-fold as compared to the number of NK cells prior to administration after five doses. In some embodiments, the heterodimeric protein is administered at a dose of at least 0.06 mg/kg Q2W and the number of NK cells increases at least 40-fold as compared to the number of NK cells prior to administration after two doses. In some embodiments, the heterodimeric protein is administered at a dose of at least 0.06 mg/kg Q2W and the number of NK cells increases at least 40-fold as compared to the number of NK cells prior to administration after three doses. In some embodiments, the heterodimeric protein is administered at a dose of at least 0.06 mg/kg Q2W and the number of NK cells increases at least 40-fold as compared to the number of NK cells prior to administration after four doses. In some embodiments, the heterodimeric protein is administered at a dose of at least 0.06 mg/kg Q2W and the number of NK cells increases at least 40-fold as compared to the number of NK cells prior to administration after five doses. In some embodiments, the heterodimeric protein is administered at a dose of at least 0.06 mg/kg Q2W and the number of NK cells increases at least 50-fold as compared to the number of NK cells prior to administration after two doses. In some embodiments, the heterodimeric protein is administered at a dose of at least 0.06 mg/kg Q2W and the number of NK cells increases at least 50-fold as compared to the number of NK cells prior to administration after three doses. In some embodiments, the heterodimeric protein is administered at a dose of at least 0.06 mg/kg Q2W and the number of NK cells increases at least 50-fold as compared to the number of NK cells prior to administration after four doses. In some embodiments, the heterodimeric protein is administered at a dose of at least 0.06 mg/kg Q2W and the number of NK cells increases at least 50-fold as compared to the number of NK cells prior to administration after five doses. In some embodiments, the heterodimeric protein is administered at a dose of at least 0.06 mg/kg Q2W and the number of NK cells increases at least 100-fold as compared to the number of NK cells prior to administration after two doses. In some embodiments, the heterodimeric protein is administered at a dose of at least 0.06 mg/kg Q2W and the number of NK cells increases at least 100-fold as compared to the number of NK cells prior to administration after three doses. In some embodiments, the heterodimeric protein is administered at a dose of at least 0.06 mg/kg Q2W and the number of NK cells increases at least 100-fold as compared to the number of NK cells prior to administration after four doses. In some embodiments, the heterodimeric protein is administered at a dose of at least 0.06 mg/kg Q2W and the number of NK cells increases at least 100-fold as compared to the number of NK cells prior to administration after five doses. In some embodiments, the heterodimeric protein is administered at a dose of at least 0.06 mg/kg Q2W and the number of NK cells increases at least 200-fold as compared to the number of NK cells prior to administration after two doses. In some embodiments, the heterodimeric protein is administered at a dose of at least 0.06 mg/kg Q2W and the number of NK cells increases at least 200-fold as compared to the number of NK cells prior to administration after three doses. In some embodiments, the heterodimeric protein is administered at a dose of at least 0.06 mg/kg Q2W and the number of NK cells increases at least 200-fold as compared to the number of NK cells prior to administration after four doses. In some embodiments, the heterodimeric protein is administered at a dose of at least 0.06 mg/kg Q2W and the number of NK cells increases at least 200-fold as compared to the number of NK cells prior to administration after five doses.

In some embodiments, the heterodimeric protein is administered at a dose of at least 0.09 mg/kg Q2W and the number of NK cells increases at least 20-fold as compared to the number of NK cells prior to administration after two doses. In some embodiments, the heterodimeric protein is administered at a dose of at least 0.09 mg/kg Q2W and the number of NK cells increases at least 20-fold as compared to the number of NK cells prior to administration after three doses. In some embodiments, the heterodimeric protein is administered at a dose of at least 0.09 mg/kg Q2W and the number of NK cells increases at least 20-fold as compared to the number of NK cells prior to administration after four doses. In some embodiments, the heterodimeric protein is administered at a dose of at least 0.09 mg/kg Q2W and the number of NK cells increases at least 20-fold as compared to the number of NY cells prior to administration after five doses. In some embodiments, the heterodimeric protein is administered at a dose of at least 0.09 mg/kg Q2W and the number of NK cells increases at least 30-fold as compared to the number of NK cells prior to administration after two doses. In some embodiments, the heterodimeric protein is administered at a dose of at least 0.09 mg/kg Q2W and the number of NK cells increases at least 30-fold as compared to the number of NK cells prior to administration after three doses. In some embodiments, the heterodimeric protein is administered at a dose of at least 0.09 mg/kg Q2W and the number of NK cells increases at least 30-fold as compared to the number of NK cells prior to administration after four doses. In some embodiments, the heterodimeric protein is administered at a dose of at least 0.09 mg/kg Q2W and the number of NK cells increases at least 30-fold as compared to the number of NK cells prior to administration after five doses. In some embodiments, the heterodimeric protein is administered at a dose of at least 0.09 mg/kg Q2W and the number of NK cells increases at least 40-fold as compared to the number of NK cells prior to administration after two doses. In some embodiments, the heterodimeric protein is administered at a dose of at least 0.09 mg/kg Q2W and the number of NK cells increases at least 40-fold as compared to the number of NK cells prior to administration after three doses. In some embodiments, the heterodimeric protein is administered at a dose of at least 0.09 mg/kg Q2W and the number of NK cells increases at least 40-fold as compared to the number of NK cells prior to administration after four doses. In some embodiments, the heterodimeric protein is administered at a dose of at least 0.09 mg/kg Q2W and the number of NK cells increases at least 40-fold as compared to the number of NK cells prior to administration after five doses. In some embodiments, the heterodimeric protein is administered at a dose of at least 0.09 mg/kg Q2W and the number of NK cells increases at least 50-fold as compared to the number of NK cells prior to administration after two doses. In some embodiments, the heterodimeric protein is administered at a dose of at least 0.09 mg/kg Q2W and the number of NK cells increases at least 50-fold as compared to the number of NY cells prior to administration after three doses. In some embodiments, the heterodimeric protein is administered at a dose of at least 0.09 mg/kg Q2W and the number of NK cells increases at least 50-fold as compared to the number of NK cells prior to administration after four doses. In some embodiments, the heterodimeric protein is administered at a dose of at least 0.09 mg/kg Q2W and the number of NK cells increases at least 50-fold as compared to the number of NK cells prior to administration after five doses. In some embodiments, the heterodimeric protein is administered at a dose of at least 0.09 mg/kg Q2W and the number of NK cells increases at least 100-fold as compared to the number of NK cells prior to administration after two doses. In some embodiments, the heterodimeric protein is administered at a dose of at least 0.09 mg/kg Q2W and the number of NK cells increases at least 100-fold as compared to the number of NK cells prior to administration after three doses. In some embodiments, the heterodimeric protein is administered at a dose of at least 0.09 mg/kg Q2W and the number of NK cells increases at least 100-fold as compared to the number of NK cells prior to administration after four doses. In some embodiments, the heterodimeric protein is administered at a dose of at least 0.09 mg/kg Q2W and the number of NK cells increases at least 100-fold as compared to the number of NK cells prior to administration after five doses. In some embodiments, the heterodimeric protein is administered at a dose of at least 0.09 mg/kg Q2W and the number of NK cells increases at least 200-fold as compared to the number of NK cells prior to administration after two doses. In some embodiments, the heterodimeric protein is administered at a dose of at least 0.09 mg/kg Q2W and the number of NK cells increases at least 200-fold as compared to the number of NK cells prior to administration after three doses. In some embodiments, the heterodimeric protein is administered at a dose of at least 0.09 mg/kg Q2W and the number of NK cells increases at least 200-fold as compared to the number of NK cells prior to administration after four doses. In some embodiments, the heterodimeric protein is administered at a dose of at least 0.09 mg/kg Q2W and the number of NK cells increases at least 200-fold as compared to the number of NK cells prior to administration after five doses.

In some embodiments, the heterodimeric protein is administered at a dose of at least 0.12 mg/kg Q2W and the number of NK cells increases at least 20-fold as compared to the number of NK cells prior to administration after two doses. In some embodiments, the heterodimeric protein is administered at a dose of at least 0.12 mg/kg Q2W and the number of NK cells increases at least 20-fold as compared to the number of NK cells prior to administration after three doses. In some embodiments, the heterodimeric protein is administered at a dose of at least 0.12 mg/kg Q2W and the number of NK cells increases at least 20-fold as compared to the number of NK cells prior to administration after four doses. In some embodiments, the heterodimeric protein is administered at a dose of at least 0.12 mg/kg Q2W and the number of NK cells increases at least 20-fold as compared to the number of NK cells prior to administration after five doses. In some embodiments, the heterodimeric protein is administered at a dose of at least 0.12 mg/kg Q2W and the number of NK cells increases at least 30-fold as compared to the number of NK cells prior to administration after two doses. In some embodiments, the heterodimeric protein is administered at a dose of at least 0.12 mg/kg Q2W and the number of NK cells increases at least 30-fold as compared to the number of NK cells prior to administration after three doses. In some embodiments, the heterodimeric protein is administered at a dose of at least 0.12 mg/kg Q2W and the number of NK cells increases at least 30-fold as compared to the number of NK cells prior to administration after four doses. In some embodiments, the heterodimeric protein is administered at a dose of at least 0.12 mg/kg Q2W and the number of NK cells increases at least 30-fold as compared to the number of NK cells prior to administration after five doses. In some embodiments, the heterodimeric protein is administered at a dose of at least 0.12 mg/kg Q2W and the number of NK cells increases at least 40-fold as compared to the number of NK cells prior to administration after two doses. In some embodiments, the heterodimeric protein is administered at a dose of at least 0.12 mg/kg Q2W and the number of NK cells increases at least 40-fold as compared to the number of NK cells prior to administration after three doses. In some embodiments, the heterodimeric protein is administered at a dose of at least 0.12 mg/kg Q2W and the number of NK cells increases at least 40-fold as compared to the number of NK cells prior to administration after four doses. In some embodiments, the heterodimeric protein is administered at a dose of at least 0.12 mg/kg Q2W and the number of NK cells increases at least 40-fold as compared to the number of NK cells prior to administration after five doses. In some embodiments, the heterodimeric protein is administered at a dose of at least 0.12 mg/kg Q2W and the number of NK cells increases at least 50-fold as compared to the number of NK cells prior to administration after two doses. In some embodiments, the heterodimeric protein is administered at a dose of at least 0.12 mg/kg Q2W and the number of NK cells increases at least 50-fold as compared to the number of NK cells prior to administration after three doses. In some embodiments, the heterodimeric protein is administered at a dose of at least 0.12 mg/kg Q2W and the number of NK cells increases at least 50-fold as compared to the number of NK cells prior to administration after four doses. In some embodiments, the heterodimeric protein is administered at a dose of at least 0.12 mg/kg Q2W and the number of NK cells increases at least 50-fold as compared to the number of NK cells prior to administration after five doses. In some embodiments, the heterodimeric protein is administered at a dose of at least 0.12 mg/kg Q2W and the number of NK cells increases at least 100-fold as compared to the number of NK cells prior to administration after two doses. In some embodiments, the heterodimeric protein is administered at a dose of at least 0.12 mg/kg Q2W and the number of NK cells increases at least 100-fold as compared to the number of NK cells prior to administration after three doses. In some embodiments, the heterodimeric protein is administered at a dose of at least 0.12 mg/kg Q2W and the number of NK cells increases at least 100-fold as compared to the number of NK cells prior to administration after four doses. In some embodiments, the heterodimeric protein is administered at a dose of at least 0.12 mg/kg Q2W and the number of NK cells increases at least 100-fold as compared to the number of NK cells prior to administration after five doses. In some embodiments, the heterodimeric protein is administered at a dose of at least 0.12 mg/kg Q2W and the number of NK cells increases at least 200-fold as compared to the number of NK cells prior to administration after two doses. In some embodiments, the heterodimeric protein is administered at a dose of at least 0.12 mg/kg Q2W and the number of NK cells increases at least 200-fold as compared to the number of NK cells prior to administration after three doses. In some embodiments, the heterodimeric protein is administered at a dose of at least 0.12 mg/kg Q2W and the number of NK cells increases at least 200-fold as compared to the number of NK cells prior to administration after four doses. In some embodiments, the heterodimeric protein is administered at a dose of at least 0.12 mg/kg Q2W and the number of NK cells increases at least 200-fold as compared to the number of NK cells prior to administration after five doses.

In some embodiments, the combination of heterodimeric proteins is administered at a dose of at least 0.02 mg/kg Q2W and the number of NK cells increases at least 20-fold as compared to the number of NK cells prior to administration after two doses. In some embodiments, the combination of heterodimeric proteins is administered at a dose of at least 0.02 mg/kg Q2W and the number of NNK cells increases at least 20-fold as compared to the number of NK cells prior to administration after three doses. In some embodiments, the combination of heterodimeric proteins is administered at a dose of at least 0.02 mg/kg Q2W and the number of NK cells increases at least 20-fold as compared to the number of NK cells prior to administration after four doses. In some embodiments, the combination of heterodimeric proteins is administered at a dose of at least 0.02 mg/kg Q2W and the number of NK cells increases at least 20-fold as compared to the number of NK cells prior to administration after five doses. In some embodiments, the combination of heterodimeric proteins is administered at a dose of at least 0.02 mg/kg Q2W and the number of NK cells increases at least 30-fold as compared to the number of NK cells prior to administration after two doses. In some embodiments, the combination of heterodimeric proteins is administered at a dose of at least 0.02 mg/kg Q2W and the number of NK cells increases at least 30-fold as compared to the number of NK cells prior to administration after three doses. In some embodiments, the combination of heterodimeric proteins is administered at a dose of at least 0.02 mg/kg Q2W and the number of NK cells increases at least 30-fold as compared to the number of NK cells prior to administration after four doses. In some embodiments, the combination of heterodimeric proteins is administered at a dose of at least 0.02 mg/kg Q2W and the number of NK cells increases at least 30-fold as compared to the number of NK cells prior to administration after five doses. In some embodiments, the combination of heterodimeric proteins is administered at a dose of at least 0.02 mg/kg Q2W and the number of NK cells increases at least 40-fold as compared to the number of NK cells prior to administration after two doses. In some embodiments, the combination of heterodimeric proteins is administered at a dose of at least 0.02 mg/kg Q2W and the number of NK cells increases at least 40-fold as compared to the number of NK cells prior to administration after three doses. In some embodiments, the combination of heterodimeric proteins is administered at a dose of at least 0.02 mg/kg Q2W and the number of NK cells increases at least 40-fold as compared to the number of NK cells prior to administration after four doses. In some embodiments, the combination of heterodimeric proteins is administered at a dose of at least 0.02 mg/kg Q2W and the number of NK cells increases at least 40-fold as compared to the number of NK cells prior to administration after five doses. In some embodiments, the combination of heterodimeric proteins is administered at a dose of at least 0.02 mg/kg Q2W and the number of NK cells increases at least 50-fold as compared to the number of NK cells prior to administration after two doses. In some embodiments, the combination of heterodimeric proteins is administered at a dose of at least 0.02 mg/kg Q2W and the number of NK cells increases at least 50-fold as compared to the number of NK cells prior to administration after three doses. In some embodiments, the combination of heterodimeric proteins is administered at a dose of at least 0.02 mg/kg Q2W and the number of NK cells increases at least 50-fold as compared to the number of NK cells prior to administration after four doses. In some embodiments, the combination of heterodimeric proteins is administered at a dose of at least 0.02 mg/kg Q2W and the number of NK cells increases at least 50-fold as compared to the number of NK cells prior to administration after five doses. In some embodiments, the combination of heterodimeric proteins is administered at a dose of at least 0.02 mg/kg Q2W and the number of NK cells increases at least 100-fold as compared to the number of NK cells prior to administration after two doses. In some embodiments, the combination of heterodimeric proteins is administered at a dose of at least 0.02 mg/kg Q2W and the number of NK cells increases at least 100-fold as compared to the number of NK cells prior to administration after three doses. In some embodiments, the combination of heterodimeric proteins is administered at a dose of at least 0.02 mg/kg Q2W and the number of NK cells increases at least 100-fold as compared to the number of NK cells prior to administration after four doses. In some embodiments, the combination of heterodimeric proteins is administered at a dose of at least 0.02 mg/kg Q2W and the number of NK cells increases at least 100-fold as compared to the number of NK cells prior to administration after five doses. In some embodiments, the combination of heterodimeric proteins is administered at a dose of at least 0.02 mg/kg Q2W and the number of NK cells increases at least 200-fold as compared to the number of NK cells prior to administration after two doses. In some embodiments, the combination of heterodimeric proteins is administered at a dose of at least 0.02 mg/kg Q2W and the number of NK cells increases at least 200-fold as compared to the number of NK cells prior to administration after three doses. In some embodiments, the combination of heterodimeric proteins is administered at a dose of at least 0.02 mg/kg Q2W and the number of NK cells increases at least 200-fold as compared to the number of NK cells prior to administration after four doses. In some embodiments, the combination of heterodimeric proteins is administered at a dose of at least 0.02 mg/kg Q2W and the number of NK cells increases at least 200-fold as compared to the number of NK cells prior to administration after five doses.

In some embodiments, the combination of heterodimeric proteins is administered at a dose of at least 0.04 mg/kg Q2W and the number of NK cells increases at least 20-fold as compared to the number of NK cells prior to administration after two doses. In some embodiments, the combination of heterodimeric proteins is administered at a dose of at least 0.04 mg/kg Q2W and the number of NK cells increases at least 20-fold as compared to the number of NK cells prior to administration after three doses. In some embodiments, the combination of heterodimeric proteins is administered at a dose of at least 0.04 mg/kg Q2W and the number of NK cells increases at least 20-fold as compared to the number of NK cells prior to administration after four doses. In some embodiments, the combination of heterodimeric proteins is administered at a dose of at least 0.04 mg/kg Q2W and the number of NK cells increases at least 20-fold as compared to the number of NK cells prior to administration after five doses. In some embodiments, the combination of heterodimeric proteins is administered at a dose of at least 0.04 mg/kg Q2W and the number of NK cells increases at least 30-fold as compared to the number of NK cells prior to administration after two doses. In some embodiments, the combination of heterodimeric proteins is administered at a dose of at least 0.04 mg/kg Q2W and the number of NK cells increases at least 30-fold as compared to the number of NK cells prior to administration after three doses. In some embodiments, the combination of heterodimeric proteins is administered at a dose of at least 0.04 mg/kg Q2W and the number of NK cells increases at least 30-fold as compared to the number of NK cells prior to administration after four doses. In some embodiments, the combination of heterodimeric proteins is administered at a dose of at least 0.04 mg/kg Q2W and the number of NK cells increases at least 30-fold as compared to the number of NK cells prior to administration after five doses. In some embodiments, the combination of heterodimeric proteins is administered at a dose of at least 0.04 mg/kg Q2W and the number of NK cells increases at least 40-fold as compared to the number of NK cells prior to administration after two doses. In some embodiments, the combination of heterodimeric proteins is administered at a dose of at least 0.04 mg/kg Q2W and the number of NK cells increases at least 40-fold as compared to the number of NK cells prior to administration after three doses. In some embodiments, the combination of heterodimeric proteins is administered at a dose of at least 0.04 mg/kg Q2W and the number of NK cells increases at least 40-fold as compared to the number of NK cells prior to administration after four doses. In some embodiments, the combination of heterodimeric proteins is administered at a dose of at least 0.04 mg/kg Q2W and the number of NK cells increases at least 40-fold as compared to the number of NK cells prior to administration after five doses. In some embodiments, the combination of heterodimeric proteins is administered at a dose of at least 0.04 mg/kg Q2W and the number of NK cells increases at least 50-fold as compared to the number of NK cells prior to administration after two doses. In some embodiments, the combination of heterodimeric proteins is administered at a dose of at least 0.04 mg/kg Q2W and the number of NK cells increases at least 50-fold as compared to the number of NK cells prior to administration after three doses. In some embodiments, the combination of heterodimeric proteins is administered at a dose of at least 0.04 mg/kg Q2W and the number of NK cells increases at least 50-fold as compared to the number of NK cells prior to administration after four doses. In some embodiments, the combination of heterodimeric proteins is administered at a dose of at least 0.04 mg/kg Q2W and the number of NK cells increases at least 50-fold as compared to the number of NK cells prior to administration after five doses. In some embodiments, the combination of heterodimeric proteins is administered at a dose of at least 0.04 mg/kg Q2W and the number of NK cells increases at least 100-fold as compared to the number of NK cells prior to administration after two doses. In some embodiments, the combination of heterodimeric proteins is administered at a dose of at least 0.04 mg/kg Q2W and the number of NK cells increases at least 100-fold as compared to the number of NK cells prior to administration after three doses. In some embodiments, the combination of heterodimeric proteins is administered at a dose of at least 0.04 mg/kg Q2W and the number of NK cells increases at least 100-fold as compared to the number of NK cells prior to administration after four doses. In some embodiments, the combination of heterodimeric proteins is administered at a dose of at least 0.04 mg/kg Q2W and the number of NK cells increases at least 100-fold as compared to the number of NK cells prior to administration after five doses. In some embodiments, the combination of heterodimeric proteins is administered at a dose of at least 0.04 mg/kg Q2W and the number of NK cells increases at least 200-fold as compared to the number of NK cells prior to administration after two doses. In some embodiments, the combination of heterodimeric proteins is administered at a dose of at least 0.04 mg/kg Q2W and the number of NK cells increases at least 200-fold as compared to the number of NK cells prior to administration after three doses. In some embodiments, the combination of heterodimeric proteins is administered at a dose of at least 0.04 mg/kg Q2W and the number of NK cells increases at least 200-fold as compared to the number of NK cells prior to administration after four doses. In some embodiments, the combination of heterodimeric proteins is administered at a dose of at least 0.04 mg/kg Q2W and the number of NK cells increases at least 200-fold as compared to the number of NK cells prior to administration after five doses.

In some embodiments, the combination of heterodimeric proteins is administered at a dose of at least 0.06 mg/kg Q2W and the number of NK cells increases at least 20-fold as compared to the number of NK cells prior to administration after two doses. In some embodiments, the combination of heterodimeric proteins is administered at a dose of at least 0.06 mg/kg Q2W and the number of NK cells increases at least 20-fold as compared to the number of NK cells prior to administration after three doses. In some embodiments, the combination of heterodimeric proteins is administered at a dose of at least 0.06 mg/kg Q2W and the number of NK cells increases at least 20-fold as compared to the number of NK cells prior to administration after four doses. In some embodiments, the combination of heterodimeric proteins is administered at a dose of at least 0.06 mg/kg Q2W and the number of NK cells increases at least 20-fold as compared to the number of NK cells prior to administration after five doses. In some embodiments, the combination of heterodimeric proteins is administered at a dose of at least 0.06 mg/kg Q2W and the number of NK cells increases at least 30-fold as compared to the number of NK cells prior to administration after two doses. In some embodiments, the combination of heterodimeric proteins is administered at a dose of at least 0.06 mg/kg Q2W and the number of NK cells increases at least 30-fold as compared to the number of NK cells prior to administration after three doses. In some embodiments, the combination of heterodimeric proteins is administered at a dose of at least 0.06 mg/kg Q2W and the number of NK cells increases at least 30-fold as compared to the number of NK cells prior to administration after four doses. In some embodiments, the combination of heterodimeric proteins is administered at a dose of at least 0.06 mg/kg Q2W and the number of NK cells increases at least 30-fold as compared to the number of NK cells prior to administration after five doses. In some embodiments, the combination of heterodimeric proteins is administered at a dose of at least 0.06 mg/kg Q2W and the number of NK cells increases at least 40-fold as compared to the number of NK cells prior to administration after two doses. In some embodiments, the combination of heterodimeric proteins is administered at a dose of at least 0.06 mg/kg Q2W and the number of NK cells increases at least 40-fold as compared to the number of NK cells prior to administration after three doses. In some embodiments, the combination of heterodimeric proteins is administered at a dose of at least 0.06 mg/kg Q2W and the number of NK cells increases at least 40-fold as compared to the number of NK cells prior to administration after four doses. In some embodiments, the combination of heterodimeric proteins is administered at a dose of at least 0.06 mg/kg Q2W and the number of NK cells increases at least 40-fold as compared to the number of NK cells prior to administration after five doses. In some embodiments, the combination of heterodimeric proteins is administered at a dose of at least 0.06 mg/kg Q2W and the number of NK cells increases at least 50-fold as compared to the number of NK cells prior to administration after two doses. In some embodiments, the combination of heterodimeric proteins is administered at a dose of at least 0.06 mg/kg Q2W and the number of NK cells increases at least 50-fold as compared to the number of NK cells prior to administration after three doses. In some embodiments, the combination of heterodimeric proteins is administered at a dose of at least 0.06 mg/kg Q2W and the number of NK cells increases at least 50-fold as compared to the number of NK cells prior to administration after four doses. In some embodiments, the combination of heterodimeric proteins is administered at a dose of at least 0.06 mg/kg Q2W and the number of NK cells increases at least 50-fold as compared to the number of NK cells prior to administration after five doses. In some embodiments, the combination of heterodimeric proteins is administered at a dose of at least 0.06 mg/kg Q2W and the number of NK cells increases at least 100-fold as compared to the number of NK cells prior to administration after two doses. In some embodiments, the combination of heterodimeric proteins is administered at a dose of at least 0.06 mg/kg Q2W and the number of NK cells increases at least 100-fold as compared to the number of NK cells prior to administration after three doses. In some embodiments, the combination of heterodimeric proteins is administered at a dose of at least 0.06 mg/kg Q2W and the number of NK cells increases at least 100-fold as compared to the number of NK cells prior to administration after four doses. In some embodiments, the combination of heterodimeric proteins is administered at a dose of at least 0.06 mg/kg Q2W and the number of NK cells increases at least 100-fold as compared to the number of NK cells prior to administration after five doses. In some embodiments, the combination of heterodimeric proteins is administered at a dose of at least 0.06 mg/kg Q2W and the number of NK cells increases at least 200-fold as compared to the number of NK cells prior to administration after two doses. In some embodiments, the combination of heterodimeric proteins is administered at a dose of at least 0.06 mg/kg Q2W and the number of NK cells increases at least 200-fold as compared to the number of NK cells prior to administration after three doses. In some embodiments, the combination of heterodimeric proteins is administered at a dose of at least 0.06 mg/kg Q2W and the number of NK cells increases at least 200-fold as compared to the number of NK cells prior to administration after four doses. In some embodiments, the combination of heterodimeric proteins is administered at a dose of at least 0.06 mg/kg Q2W and the number of NK cells increases at least 200-fold as compared to the number of NK cells prior to administration after five doses.

In some embodiments, the combination of heterodimeric proteins is administered at a dose of at least 0.09 mg/kg Q2W and the number of NK cells increases at least 20-fold as compared to the number of NK cells prior to administration after two doses. In some embodiments, the combination of heterodimeric proteins is administered at a dose of at least 0.09 mg/kg Q2W and the number of NK cells increases at least 20-fold as compared to the number of NK cells prior to administration after three doses. In some embodiments, the combination of heterodimeric proteins is administered at a dose of at least 0.09 mg/kg Q2W and the number of NK cells increases at least 20-fold as compared to the number of NK cells prior to administration after four doses. In some embodiments, the combination of heterodimeric proteins is administered at a dose of at least 0.09 mg/kg Q2W and the number of NK cells increases at least 20-fold as compared to the number of NK cells prior to administration after five doses. In some embodiments, the combination of heterodimeric proteins is administered at a dose of at least 0.09 mg/kg Q2W and the number of NK cells increases at least 30-fold as compared to the number of NK cells prior to administration after two doses. In some embodiments, the combination of heterodimeric proteins is administered at a dose of at least 0.09 mg/kg Q2W and the number of NK cells increases at least 30-fold as compared to the number of NK cells prior to administration after three doses. In some embodiments, the combination of heterodimeric proteins is administered at a dose of at least 0.09 mg/kg Q2W and the number of NK cells increases at least 30-fold as compared to the number of NK cells prior to administration after four doses. In some embodiments, the combination of heterodimeric proteins is administered at a dose of at least 0.09 mg/kg Q2W and the number of NK cells increases at least 30-fold as compared to the number of NK cells prior to administration after five doses. In some embodiments, the combination of heterodimeric proteins is administered at a dose of at least 0.09 mg/kg Q2W and the number of NK cells increases at least 40-fold as compared to the number of NK cells prior to administration after two doses. In some embodiments, the combination of heterodimeric proteins is administered at a dose of at least 0.09 mg/kg Q2W and the number of NK cells increases at least 40-fold as compared to the number of NK cells prior to administration after three doses. In some embodiments, the combination of heterodimeric proteins is administered at a dose of at least 0.09 mg/kg Q2W and the number of NK cells increases at least 40-fold as compared to the number of NK cells prior to administration after four doses. In some embodiments, the combination of heterodimeric proteins is administered at a dose of at least 0.09 mg/kg Q2W and the number of NK cells increases at least 40-fold as compared to the number of NK cells prior to administration after five doses. In some embodiments, the combination of heterodimeric proteins is administered at a dose of at least 0.09 mg/kg Q2W and the number of NK cells increases at least 50-fold as compared to the number of NK cells prior to administration after two doses. In some embodiments, the combination of heterodimeric proteins is administered at a dose of at least 0.09 mg/kg Q2W and the number of NK cells increases at least 50-fold as compared to the number of NK cells prior to administration after three doses. In some embodiments, the combination of heterodimeric proteins is administered at a dose of at least 0.09 mg/kg Q2W and the number of NK cells increases at least 50-fold as compared to the number of NK cells prior to administration after four doses. In some embodiments, the combination of heterodimeric proteins is administered at a dose of at least 0.09 mg/kg Q2W and the number of NK cells increases at least 50-fold as compared to the number of NK cells prior to administration after five doses. In some embodiments, the combination of heterodimeric proteins is administered at a dose of at least 0.09 mg/kg Q2W and the number of NK cells increases at least 100-fold as compared to the number of NK cells prior to administration after two doses. In some embodiments, the combination of heterodimeric proteins is administered at a dose of at least 0.09 mg/kg Q2W and the number of NK cells increases at least 100-fold as compared to the number of NK cells prior to administration after three doses. In some embodiments, the combination of heterodimeric proteins is administered at a dose of at least 0.09 mg/kg Q2W and the number of NK cells increases at least 100-fold as compared to the number of NK cells prior to administration after four doses. In some embodiments, the combination of heterodimeric proteins is administered at a dose of at least 0.09 mg/kg Q2W and the number of NK cells increases at least 100-fold as compared to the number of NK cells prior to administration after five doses. In some embodiments, the combination of heterodimeric proteins is administered at a dose of at least 0.09 mg/kg Q2W and the number of NK cells increases at least 200-fold as compared to the number of NK cells prior to administration after two doses. In some embodiments, the combination of heterodimeric proteins is administered at a dose of at least 0.09 mg/kg Q2W and the number of NK cells increases at least 200-fold as compared to the number of NK cells prior to administration after three doses. In some embodiments, the combination of heterodimeric proteins is administered at a dose of at least 0.09 mg/kg Q2W and the number of NK cells increases at least 200-fold as compared to the number of NK cells prior to administration after four doses. In some embodiments, the combination of heterodimeric proteins is administered at a dose of at least 0.09 mg/kg Q2W and the number of NK cells increases at least 200-fold as compared to the number of NK cells prior to administration after five doses.

In some embodiments, the combination of heterodimeric proteins is administered at a dose of at least 0.12 mg/kg Q2W and the number of NK cells increases at least 20-fold as compared to the number of NK cells prior to administration after two doses. In some embodiments, the combination of heterodimeric proteins is administered at a dose of at least 0.12 mg/kg Q2W and the number of NK cells increases at least 20-fold as compared to the number of NK cells prior to administration after three doses. In some embodiments, the combination of heterodimeric proteins is administered at a dose of at least 0.12 mg/kg Q2W and the number of NK cells increases at least 20-fold as compared to the number of NK cells prior to administration after four doses. In some embodiments, the combination of heterodimeric proteins is administered at a dose of at least 0.12 mg/kg Q2W and the number of NK cells increases at least 20-fold as compared to the number of NK cells prior to administration after five doses. In some embodiments, the combination of heterodimeric proteins is administered at a dose of at least 0.12 mg/kg Q2W and the number of NK cells increases at least 30-fold as compared to the number of NK cells prior to administration after two doses. In some embodiments, the combination of heterodimeric proteins is administered at a dose of at least 0.12 mg/kg Q2W and the number of NK cells increases at least 30-fold as compared to the number of NK cells prior to administration after three doses. In some embodiments, the combination of heterodimeric proteins is administered at a dose of at least 0.12 mg/kg Q2W and the number of NK cells increases at least 30-fold as compared to the number of NK cells prior to administration after four doses. In some embodiments, the combination of heterodimeric proteins is administered at a dose of at least 0.12 mg/kg Q2W and the number of NK cells increases at least 30-fold as compared to the number of NK cells prior to administration after five doses. In some embodiments, the combination of heterodimeric proteins is administered at a dose of at least 0.12 mg/kg Q2W and the number of NK cells increases at least 40-fold as compared to the number of NK cells prior to administration after two doses. In some embodiments, the combination of heterodimeric proteins is administered at a dose of at least 0.12 mg/kg Q2W and the number of NK cells increases at least 40-fold as compared to the number of NK cells prior to administration after three doses. In some embodiments, the combination of heterodimeric proteins is administered at a dose of at least 0.12 mg/kg Q2W and the number of NK cells increases at least 40-fold as compared to the number of NK cells prior to administration after four doses. In some embodiments, the combination of heterodimeric proteins is administered at a dose of at least 0.12 mg/kg Q2W and the number of NK cells increases at least 40-fold as compared to the number of NK cells prior to administration after five doses. In some embodiments, the combination of heterodimeric proteins is administered at a dose of at least 0.12 mg/kg Q2W and the number of NK cells increases at least 50-fold as compared to the number of NK cells prior to administration after two doses. In some embodiments, the combination of heterodimeric proteins is administered at a dose of at least 0.12 mg/kg Q2W and the number of NK cells increases at least 50-fold as compared to the number of NK cells prior to administration after three doses. In some embodiments, the combination of heterodimeric proteins is administered at a dose of at least 0.12 mg/kg Q2W and the number of NK cells increases at least 50-fold as compared to the number of NK cells prior to administration after four doses. In some embodiments, the combination of heterodimeric proteins is administered at a dose of at least 0.12 mg/kg Q2W and the number of NK cells increases at least 50-fold as compared to the number of NK cells prior to administration after five doses. In some embodiments, the combination of heterodimeric proteins is administered at a dose of at least 0.12 mg/kg Q2W and the number of NK cells increases at least 100-fold as compared to the number of NK cells prior to administration after two doses. In some embodiments, the combination of heterodimeric proteins is administered at a dose of at least 0.12 mg/kg Q2W and the number of NK cells increases at least 100-fold as compared to the number of NK cells prior to administration after three doses. In some embodiments, the combination of heterodimeric proteins is administered at a dose of at least 0.12 mg/kg Q2W and the number of NK cells increases at least 100-fold as compared to the number of NK cells prior to administration after four doses. In some embodiments, the combination of heterodimeric proteins is administered at a dose of at least 0.12 mg/kg Q2W and the number of NK cells increases at least 100-fold as compared to the number of NK cells prior to administration after five doses. In some embodiments, the combination of heterodimeric proteins is administered at a dose of at least 0.12 mg/kg Q2W and the number of NK cells increases at least 200-fold as compared to the number of NK cells prior to administration after two doses. In some embodiments, the combination of heterodimeric proteins is administered at a dose of at least 0.12 mg/kg Q2W and the number of NK cells increases at least 200-fold as compared to the number of NK cells prior to administration after three doses. In some embodiments, the combination of heterodimeric proteins is administered at a dose of at least 0.12 mg/kg Q2W and the number of NK cells increases at least 200-fold as compared to the number of NK cells prior to administration after four doses. In some embodiments, the combination of heterodimeric proteins is administered at a dose of at least 0.12 mg/kg Q2W and the number of NK cells increases at least 200-fold as compared to the number of NK cells prior to administration after five doses.

In some embodiments, prior to administration of the plurality of doses of the heterodimeric protein, the subject has not been previously administered an agent for the treatment of the condition. In some embodiments, the subject is currently being administered a checkpoint inhibitor, i.e., the subject is receiving the checkpoint inhibitor with the heterodimeric protein of the disclosure, either as part of the same or a different treatment regimen. In some embodiments, prior to administration of the plurality of doses of the heterodimeric protein, the subject has previously been administered a checkpoint inhibitor. In some embodiments, the checkpoint inhibitor targets PD-1. In some embodiments, the checkpoint inhibitor targets PD-L1. In some embodiments, the checkpoint inhibitor targets CTLA-4. In some embodiments, the checkpoint inhibitor that targets PD-1 is an anti-PD-1 antibody. Antibodies which specifically bind to PD-1 are known in the art and have been described, for example, in Naidoo et al. Ann Oncol. 2015; 26(12): 2375-2391, Philips et al. Int Immunol. 2015; 27(1):39-46, Tunger et al. J Clin Med. 2019; 8(10) and Sunshine et al. Curr Opin Pharmacol. 2015; 32-8; and U.S. Pat. Nos. 8,008,449, 8,168,757, US 20110008369, US 20130017199, US 20130022595, and in WO2006121168, WO20091154335, WO2012145493, WO2013014668, WO2009101611, EP2262837, and EP2504028. Examples of anti-PD-1 antibodies include, but are not limited to, nivolumab (BMS-936558; MDX-1106), pembrolizumab (Trade name Keytruda formerly lambrolizumab; also known as Merck 3475 and SCH-900475), pidilizumab (CT-011), cemiplimab, spartalizumab (PDR001), camrelizumab (SHR1210), sintilimab (IBI308), tislelizumab (BGB-A317), toripalimab (JS 001), and AMP-514 (Amplimmune). Nivolumab is an anti-PD-1 antibody described in WO2006/121168. Pembrolizumab is an anti-PD-1 antibody described in WO2009/114335 and Hamid et al. (2013). New England Journal of Medicine 369 (2): 134-44. Pidilizumab is a humanized IgG-1κ monoclonal antibody that binds to PD-1. Pidilizumab and other humanized anti-PD1 monoclonal antibodies are disclosed in WO2009/101611. Other anti-PD-1 antibodies include AMP 514, among others, e.g., anti-PD-1 antibodies disclosed in U.S. Pat. No. 8,609,089, US 2010028330 and/or US 20120114649. In some embodiments, the anti-PD-1 antibody is nivolumab. In some embodiments, the checkpoint inhibitor that targets PD-1 is a fusion protein, e.g. AMP-224. AMP-224 (Amplimmune) is a PD-L2 Fc fusion soluble receptor that blocks the interaction between PD-1 and B7-H1 and is disclosed in WO2010/027827 and WO2011/066342.

In some embodiments, the checkpoint inhibitor that targets PD-L1 is an anti-PD-L1 antibody. Antibodies which specifically bind to PD-L1 are known in the art and have been described, for example, in Naidoo et al. Ann Oncol. 2015 December; 26(12): 2375-2391, Philips et al. Int Immunol. 2015 January; 27(1):39-46, Tunger et al. J Clin Med. 2019 Sep. 25; 8(10), Sunshine et al. Curr Opin Pharmacol. 2015:32-8 and U.S. Pat. No. 7,943,743 and U.S. Publication No. 20120039906. Examples of anti-PD-L1 antibodies include, but are not limited to, BMS-936559 (also known as MDX-1105), BMS-39886, atezolizumab (MDPL3280A; Tecentriq), avelumab (MSB-0010718C; Bavencio), durvalumab (MEDI4736; Imfinzi), envafolimab (KN035), and cosibelimab (CK-301; Checkpoint Therapeutics). BMS-936559 is an anti-PD-L1 antibody described in WO2007/005874. Atezolizumab is a humanized monoclonal antibody with a human Fc optimized IgG1 that binds to PD-L1. BMS-39886 is an anti-PD-L1 antibody described in Brahmer J R et al. N Engl J Med 2012; 366: 2455-2465. In some embodiments, the anti-PD-L1 antibody is atezolizumab.

In some embodiments, the checkpoint inhibitor that targets CTLA-4 is an anti-CTLA-4 antibody. Antibodies which specifically bind to CTLA-4 are known in the art and have been described, for example, in Callahan M K et al. Semin Oncol. 2010; 37(5):473-484. Examples of anti-CTLA-4 antibodies include, but are not limited to, ipilimumab and tremelimumab. Both ipilimumab and tremelimumab are fully human antibodies against CTLA-4. Ipilimumab (also known as MDX-010 or Yervoy; Bristol-Myers Squibb, Princeton, NJ) is an IgG1 with a plasma half-life of 12-14 days (Hodi, F. S et al. The New England Journal of Medicine. 2010; 363 (8): 711-723). Tremelimumab (also known as CP-675,206 or ticilimumab; Pfizer, New York, NY) is an IgG2 with a plasma half-life of approximately 22 days (Reuben, J M et al. Cancer. 2006; 106 (11): 2437-44).

Method of Treatment with IL15-IL15Rα Heterodimeric Fc-Fusion Proteins and PD-L1/PD-1 Inhibitor as Combination Therapy

Another aspect of the present disclosure provides a method of treating a solid tumor as disclosed herein in a subject in need thereof, the method comprising administering to the subject (a) a plurality of doses of a heterodimeric protein, wherein each dose comprises an effective amount of any heterodimeric protein (i.e., IL15-IL15Rα heterodimeric Fc-fusion protein) disclosed herein or combinations thereof and (b) an agent targeting the PD-L1/PD-1 axis; wherein the administration of the plurality of doses of the heterodimeric protein and the agent targeting the PD-L1/PD-1 axis is sufficient to increase the number of NK cells at least 20-fold as compared to the number of NK cells prior to said administration. Another aspect of the present disclosure provides a method of treating a solid tumor as disclosed herein in a subject in need thereof, the method comprising administering to the subject (a) a plurality of doses of a heterodimeric protein, wherein each dose comprises an effective amount of any heterodimeric protein (i.e., IL15-IL15Rα heterodimeric Fc-fusion protein) disclosed herein or combinations thereof and (b) an agent targeting the PD-L1/PD-1 axis; wherein the administration of the plurality of doses of the heterodimeric protein and the agent targeting the PD-L1/PD-1 axis results in accumulation of NK cells in the subject.

Another aspect of the present disclosure provides a method for inducing the proliferation of NK cells as disclosed herein in a subject in need thereof, the method comprising administering to the subject (a) a plurality of doses of a heterodimeric protein, wherein each dose comprises an effective amount of any heterodimeric protein (i.e., IL15-IL15Rα heterodimeric Fc-fusion protein) disclosed herein or combinations thereof and (b) an agent targeting the PD-L1/PD-1 axis; wherein the administration of the plurality of doses of the heterodimeric protein and the agent targeting the PD-L1/PD-1 axis is sufficient to increase the number of NK cells at least 20-fold as compared to the number of NK cells prior to said administration. Another aspect of the present disclosure provides a method for inducing the proliferation of NK cells as disclosed herein in a subject in need thereof, the method comprising administering to the subject (a) a plurality of doses of a heterodimeric protein, wherein each dose comprises an effective amount of any heterodimeric protein (i.e., IL15-IL15Rα heterodimeric Fc-fusion protein) disclosed herein or combinations thereof and (b) an agent targeting the PD-L1/PD-1 axis; wherein the administration of the plurality of doses of the heterodimeric protein and the agent targeting the PD-L1/PD-1 axis results in accumulation of NK cells in the subject.

In any of the above methods, the heterodimeric protein may be administered according to any of the herein disclosed methods. The heterodimeric protein may be administered in any of the herein disclosed compositions. For example, in some embodiments, the administration of the plurality of doses of the heterodimeric protein is sufficient to increase the number of NK cells at least 20-fold as compared to the number of NK cells prior to said administration. In some embodiments, the administration of the plurality of doses of the heterodimeric protein results in accumulation of NK cells in the subject. In some embodiments, a plurality of doses of the agent targeting the PD-L1/PD-1 axis is administered to the subject. In some embodiments, the subject is a human subject.

In some embodiments, the number of NK cells increases at least 30-fold, 40-fold, 50-fold, 60-fold, 70-fold, 80-fold, 90-fold, 100-fold, 150-fold, 200-fold, 250-fold, or 300-fold relative to the number of NK cells prior to administration. In some embodiments, the number of NK cells increases at least 30-fold relative to the number of NK cells prior to administration. In some embodiments, the number of NK cells increases at least 40-fold relative to the number of NK cells prior to administration. In some embodiments, the number of NK cells increases at least 50-fold relative to the number of NK cells prior to administration. In some embodiments, the number of NIK cells increases at least 60-fold relative to the number of NK cells prior to administration. In some embodiments, the number of NK cells increases at least 70-fold relative to the number of NK cells prior to administration. In some embodiments, the number of NK cells increases at least 80-fold relative to the number of NK cells prior to administration. In some embodiments, the number of NK cells increases at least 90-fold relative to the number of NK cells prior to administration. In some embodiments, the number of NK cells increases at least 100-fold relative to the number of NK cells prior to administration. In some embodiments, the number of NK cells increases at least 110-fold relative to the number of NK cells prior to administration. In some embodiments, the number of NK cells increases at least 120-fold relative to the number of NK cells prior to administration. In some embodiments, the number of NK cells increases at least 130-fold relative to the number of NK cells prior to administration. In some embodiments, the number of NK cells increases at least 140-fold relative to the number of NK cells prior to administration. In some embodiments, the number of NK cells increases at least 150-fold relative to the number of NK cells prior to administration. In some embodiments, the number of NK cells increases at least 160-fold relative to the number of NK cells prior to administration. In some embodiments, the number of NK cells increases at least 170-fold relative to the number of NK cells prior to administration. In some embodiments, the number of NK cells increases at least 180-fold relative to the number of NK cells prior to administration. In some embodiments, the number of NK cells increases at least 190-fold relative to the number of NK cells prior to administration. In some embodiments, the number of NK cells increases at least 200-fold relative to the number of NK cells prior to administration. In some embodiments, the number of NK cells increases at least 210-fold relative to the number of NK cells prior to administration. In some embodiments, the number of NK cells increases at least 220-fold relative to the number of NK cells prior to administration. In some embodiments, the number of NK cells increases at least 230-fold relative to the number of NK cells prior to administration. In some embodiments, the number of NK cells increases at least 240-fold relative to the number of NK cells prior to administration. In some embodiments, the number of NK cells increases at least 250-fold relative to the number of NK cells prior to administration. In some embodiments, the number of NK cells increases at least 260-fold relative to the number of NK cells prior to administration. In some embodiments, the number of NK cells increases at least 270-fold relative to the number of NK cells prior to administration. In some embodiments, the number of NK cells increases at least 280-fold relative to the number of NK cells prior to administration. In some embodiments, the number of NK cells increases at least 290-fold relative to the number of NK cells prior to administration. In some embodiments, the number of NK cells increases at least 300-fold relative to the number of NK cells prior to administration.

In some embodiments, the accumulation is for at least one cycle. In some embodiments, the accumulation is for at least two cycles. In some embodiments, the accumulation is for at least three cycles. In some embodiments, the accumulation is for at least four cycles. In some embodiments, the accumulation is for at least five cycles. In some embodiments, the accumulation is for at least six cycles. In some embodiments, the accumulation is for at least seven cycles. In some embodiments, the accumulation is for at least eight cycles. In some embodiments, the accumulation is for at least nine cycles. In some embodiments, the accumulation is for at least ten cycles. In some embodiments, the accumulation is for at least eleven cycles. In some embodiments, the accumulation is for at least twelve cycles. In some embodiments, the accumulation is for at least thirteen cycles. In some embodiments, the accumulation is for at least fourteen cycles. In some embodiments, the accumulation is for at least fifteen cycles. In some embodiments, the accumulation is for at least sixteen cycles. In some embodiments, the accumulation is for at least seventeen cycles. In some embodiments, the accumulation is for at least eighteen cycles. In some embodiments, the accumulation is for at least nineteen cycles. In some embodiments, the accumulation is for at least twenty cycles. In some embodiments, the accumulation is for at least twenty-one cycles. In some embodiments, the accumulation is for at least twenty-two cycles. In some embodiments, the accumulation is for at least twenty-three cycles. In some embodiments, the accumulation is for at least twenty-four cycles. In some embodiments, the accumulation is for at least twenty-five cycles. In some embodiments, the accumulation is for at least twenty-six cycles.

In some embodiments, the accumulation is for no more than one cycle. In some embodiments, the accumulation is for no more than two cycles. In some embodiments, the accumulation is for no more than three cycles. In some embodiments, the accumulation is for no more than four cycles. In some embodiments, the accumulation is for no more than five cycles. In some embodiments, the accumulation is for no more than six cycles. In some embodiments, the accumulation is for no more than seven cycles. In some embodiments, the accumulation is for no more than eight cycles. In some embodiments, the accumulation is for no more than nine cycles. In some embodiments, the accumulation is for no more than ten cycles. In some embodiments, the accumulation is for no more than eleven cycles. In some embodiments, the accumulation is for no more than twelve cycles. In some embodiments, the accumulation is for no more than thirteen cycles. In some embodiments, the accumulation is for no more than fourteen cycles. In some embodiments, the accumulation is for no more than fifteen cycles. In some embodiments, the accumulation is for no more than sixteen cycles. In some embodiments, the accumulation is for no more than seventeen cycles. In some embodiments, the accumulation is for no more than eighteen cycles. In some embodiments, the accumulation is for no more than nineteen cycles. In some embodiments, the accumulation is for no more than twenty cycles. In some embodiments, the accumulation is for no more than twenty-one cycles. In some embodiments, the accumulation is for no more than twenty-two cycles. In some embodiments, the accumulation is for no more than twenty-three cycles. In some embodiments, the accumulation is for no more than twenty-four cycles. In some embodiments, the accumulation is for no more than twenty-five cycles. In some embodiments, the accumulation is for no more than twenty-six cycles.

In some embodiments, the plurality of doses comprises at least two doses. In some embodiments, the plurality of doses comprises at least three doses. In some embodiments, the plurality of doses comprises at least four doses. In some embodiments, the plurality of doses comprises at least five doses. In some embodiments, the plurality of doses comprises at least six doses. In some embodiments, the plurality of doses comprises at least seven doses. In some embodiments, the plurality of doses comprises at least eight doses. In some embodiments, the plurality of doses comprises at least nine doses. In some embodiments, the plurality of doses comprises at least ten doses. In some embodiments, the plurality of doses comprises at least eleven doses. In some embodiments, the plurality of doses comprises at least twelve doses. In some embodiments, the plurality of doses comprises at least thirteen doses. In some embodiments, the plurality of doses comprises at least fourteen doses. In some embodiments, the plurality of doses comprises at least fifteen doses. In some embodiments, the plurality of doses comprises at least sixteen doses. In some embodiments, the plurality of doses comprises at least seventeen doses. In some embodiments, the plurality of doses comprises at least eighteen doses. In some embodiments, the plurality of doses comprises at least nineteen doses. In some embodiments, the plurality of doses comprises at least twenty doses. In some embodiments, the plurality of doses comprises at least twenty-one doses. In some embodiments, the plurality of doses comprises at least twenty-two doses. In some embodiments, the plurality of doses comprises at least twenty-three doses. In some embodiments, the plurality of doses comprises at least twenty-four doses. In some embodiments, the plurality of doses comprises at least twenty-five doses. In some embodiments, the plurality of doses comprises at least twenty-six doses.

In some embodiments, the plurality of doses comprises no more than two doses. In some embodiments, the plurality of doses comprises no more than three doses. In some embodiments, the plurality of doses comprises no more than four doses. In some embodiments, the plurality of doses comprises no more than five doses. In some embodiments, the plurality of doses comprises no more than six doses. In some embodiments, the plurality of doses comprises no more than seven doses. In some embodiments, the plurality of doses comprises no more than eight doses. In some embodiments, the plurality of doses comprises no more than nine doses. In some embodiments, the plurality of doses comprises no more than ten doses. In some embodiments, the plurality of doses comprises no more than eleven doses. In some embodiments, the plurality of doses comprises no more than twelve doses. In some embodiments, the plurality of doses comprises no more than thirteen doses. In some embodiments, the plurality of doses comprises no more than fourteen doses. In some embodiments, the plurality of doses comprises no more than fifteen doses. In some embodiments, the plurality of doses comprises no more than sixteen doses. In some embodiments, the plurality of doses comprises no more than seventeen doses. In some embodiments, the plurality of doses comprises no more than eighteen doses. In some embodiments, the plurality of doses comprises no more than nineteen doses. In some embodiments, the plurality of doses comprises no more than twenty doses. In some embodiments, the plurality of doses comprises no more than twenty-one doses. In some embodiments, the plurality of doses comprises no more than twenty-two doses. In some embodiments, the plurality of doses comprises no more than twenty-three doses. In some embodiments, the plurality of doses comprises no more than twenty-four doses. In some embodiments, the plurality of doses comprises no more than twenty-five doses. In some embodiments, the plurality of doses comprises no more than twenty-six doses. In some embodiments, the plurality of doses comprises no more than fifty-two doses. In some embodiments, the plurality of doses comprises no more than seventy-eight doses. In some embodiments, the plurality of doses comprises no more than one hundred four doses. In some embodiments, the plurality of doses comprises no more than one hundred thirty doses.

In some embodiments, the number of NK cells increases at least 20-fold relative to the number of NK cells prior to administration after two doses. In some embodiments, the number of NK cells increases at least 20-fold relative to the number of NK cells prior to administration after three doses. In some embodiments, the number of NK cells increases at least 20-fold relative to the number of NK cells prior to administration after four doses. In some embodiments, the number of NK cells increases at least 20-fold relative to the number of NK cells prior to administration after five doses. In some embodiments, the number of NK cells increases at least 20-fold relative to the number of NK cells prior to administration after six doses. In some embodiments, the number of NK cells increases at least 20-fold relative to the number of NK cells prior to administration after seven doses. In some embodiments, the number of NK cells increases at least 20-fold relative to the number of NK cells prior to administration after eight doses. In some embodiments, the number of NK cells increases at least 20-fold relative to the number of NK cells prior to administration after nine doses. In some embodiments, the number of NK cells increases at least 20-fold relative to the number of NK cells prior to administration after ten doses.

In some embodiments, the number of NK cells increases at least 50-fold relative to the number of NK cells prior to administration after two doses. In some embodiments, the number of NK cells increases at least 50-fold relative to the number of NK cells prior to administration after three doses. In some embodiments, the number of NK cells increases at least 50-fold relative to the number of NK cells prior to administration after four doses. In some embodiments, the number of NK cells increases at least 50-fold relative to the number of NK cells prior to administration after five doses. In some embodiments, the number of NK cells increases at least 50-fold relative to the number of NK cells prior to administration after six doses. In some embodiments, the number of NK cells increases at least 50-fold relative to the number of NK cells prior to administration after seven doses. In some embodiments, the number of NK cells increases at least 50-fold relative to the number of NK cells prior to administration after eight doses. In some embodiments, the number of NK cells increases at least 50-fold relative to the number of NK cells prior to administration after nine doses. In some embodiments, the number of NK cells increases at least 50-fold relative to the number of NK cells prior to administration after ten doses.

In some embodiments, the number of NK cells increases at least 100-fold relative to the number of NK cells prior to administration after two doses. In some embodiments, the number of NK cells increases at least 100-fold relative to the number of NK cells prior to administration after three doses. In some embodiments, the number of NK cells increases at least 100-fold relative to the number of NK cells prior to administration after four doses. In some embodiments, the number of NK cells increases at least 100-fold relative to the number of NK cells prior to administration after five doses. In some embodiments, the number of NK cells increases at least 100-fold relative to the number of NK cells prior to administration after six doses. In some embodiments, the number of NK cells increases at least 100-fold relative to the number of NK cells prior to administration after seven doses. In some embodiments, the number of NK cells increases at least 100-fold relative to the number of NK cells prior to administration after eight doses. In some embodiments, the number of NK cells increases at least 100-fold relative to the number of NK cells prior to administration after nine doses. In some embodiments, the number of NK cells increases at least 100-fold relative to the number of NK cells prior to administration after ten doses.

In some embodiments, the number of NK cells increases at least 20-fold relative to the number of NK cells prior to administration after no more than no more than two doses. In some embodiments, the number of NK cells increases at least 20-fold relative to the number of NK cells prior to administration after no more than no more than three doses. In some embodiments, the number of NK cells increases at least 20-fold relative to the number of NK cells prior to administration after no more than four doses. In some embodiments, the number of NK cells increases at least 20-fold relative to the number of NK cells prior to administration after no more than five doses. In some embodiments, the number of NK cells increases at least 20-fold relative to the number of NK cells prior to administration after no more than six doses. In some embodiments, the number of NK cells increases at least 20-fold relative to the number of NK cells prior to administration after no more than seven doses. In some embodiments, the number of NK cells increases at least 20-fold relative to the number of NK cells prior to administration after no more than eight doses. In some embodiments, the number of NK cells increases at least 20-fold relative to the number of NK cells prior to administration after no more than nine doses. In some embodiments, the number of NK cells increases at least 20-fold relative to the number of NK cells prior to administration after no more than ten doses.

In some embodiments, the number of NK cells increases at least 50-fold relative to the number of NK cells prior to administration after no more than two doses. In some embodiments, the number of NK cells increases at least 50-fold relative to the number of NK cells prior to administration after no more than three doses. In some embodiments, the number of NK cells increases at least 50-fold relative to the number of NK cells prior to administration after no more than four doses. In some embodiments, the number of NK cells increases at least 50-fold relative to the number of NK cells prior to administration after no more than five doses. In some embodiments, the number of NK cells increases at least 50-fold relative to the number of NK cells prior to administration after no more than six doses. In some embodiments, the number of NK cells increases at least 50-fold relative to the number of NK cells prior to administration after no more than seven doses. In some embodiments, the number of NK cells increases at least 50-fold relative to the number of NK cells prior to administration after no more than eight doses. In some embodiments, the number of NK cells increases at least 50-fold relative to the number of NK cells prior to administration after no more than nine doses. In some embodiments, the number of NK cells increases at least 50-fold relative to the number of NK cells prior to administration after no more than ten doses.

In some embodiments, the number of NK cells increases at least 100-fold relative to the number of NK cells prior to administration after no more than two doses. In some embodiments, the number of NK cells increases at least 100-fold relative to the number of NK cells prior to administration after no more than three doses. In some embodiments, the number of NK cells increases at least 100-fold relative to the number of NK cells prior to administration after no more than four doses. In some embodiments, the number of NK cells increases at least 100-fold relative to the number of NK cells prior to administration after no more than five doses. In some embodiments, the number of NK cells increases at least 100-fold relative to the number of NK cells prior to administration after no more than six doses. In some embodiments, the number of NK cells increases at least 100-fold relative to the number of NK cells prior to administration after no more than seven doses. In some embodiments, the number of NK cells increases at least 100-fold relative to the number of NK cells prior to administration after no more than eight doses. In some embodiments, the number of NK cells increases at least 100-fold relative to the number of NK cells prior to administration after no more than nine doses. In some embodiments, the number of NK cells increases at least 100-fold relative to the number of NK cells prior to administration after no more than ten doses.

In some embodiments, the effective amount of the heterodimeric protein of the disclosure is an amount sufficient to increase the number of NK cells at least 20-fold relative to the number of NK cells prior to administration after two doses. In some embodiments, the effective amount of the heterodimeric protein of the disclosure is an amount sufficient to increase the number of NK cells at least 20-fold relative to the number of NK cells prior to administration after three doses. In some embodiments, the effective amount of the heterodimeric protein of the disclosure is an amount sufficient to increase the number of NK cells at least 20-fold relative to the number of NK cells prior to administration after four doses. In some embodiments, the effective amount of the heterodimeric protein of the disclosure is an amount sufficient to increase the number of NK cells at least 20-fold relative to the number of NK cells prior to administration after five doses. In some embodiments, the effective amount of the heterodimeric protein of the disclosure is an amount sufficient to increase the number of NK cells at least 20-fold relative to the number of NK cells prior to administration after six doses. In some embodiments, the effective amount of the heterodimeric protein of the disclosure is an amount sufficient to increase the number of NK cells at least 20-fold relative to the number of NY cells prior to administration after seven doses. In some embodiments, the effective amount of the heterodimeric protein of the disclosure is an amount sufficient to increase the number of NK cells at least 20-fold relative to the number of NK cells prior to administration after eight doses. In some embodiments, the effective amount of the heterodimeric protein of the disclosure is an amount sufficient to increase the number of NK cells at least 20-fold relative to the number of NK cells prior to administration after nine doses. In some embodiments, the effective amount of the heterodimeric protein of the disclosure is an amount sufficient to increase the number of NK cells at least 20-fold relative to the number of NK cells prior to administration after ten doses.

In some embodiments, the effective amount of the heterodimeric protein of the disclosure is an amount sufficient to increase the number of NK cells at least 50-fold relative to the number of NK cells prior to administration after two doses. In some embodiments, the effective amount of the heterodimeric protein of the disclosure is an amount sufficient to increase the number of NK cells at least 50-fold relative to the number of NK cells prior to administration after three doses. In some embodiments, the effective amount of the heterodimeric protein of the disclosure is an amount sufficient to increase the number of NK cells at least 50-fold relative to the number of NK cells prior to administration after four doses. In some embodiments, the effective amount of the heterodimeric protein of the disclosure is an amount sufficient to increase the number of NK cells at least 50-fold relative to the number of NK cells prior to administration after five doses. In some embodiments, the effective amount of the heterodimeric protein of the disclosure is an amount sufficient to increase the number of NK cells at least 50-fold relative to the number of NK cells prior to administration after six doses. In some embodiments, the effective amount of the heterodimeric protein of the disclosure is an amount sufficient to increase the number of NK cells at least 50-fold relative to the number of NK cells prior to administration after seven doses. In some embodiments, the effective amount of the heterodimeric protein of the disclosure is an amount sufficient to increase the number of NK cells at least 50-fold relative to the number of NK cells prior to administration after eight doses. In some embodiments, the effective amount of the heterodimeric protein of the disclosure is an amount sufficient to increase the number of NK cells at least 50-fold relative to the number of NK cells prior to administration after nine doses. In some embodiments, the effective amount of the heterodimeric protein of the disclosure is an amount sufficient to increase the number of NK cells at least 50-fold relative to the number of NK cells prior to administration after ten doses.

In some embodiments, the effective amount of the heterodimeric protein of the disclosure is an amount sufficient to increase the number of NK cells at least 100-fold relative to the number of NK cells prior to administration after two doses. In some embodiments, the effective amount of the heterodimeric protein of the disclosure is an amount sufficient to increase the number of NK cells at least 100-fold relative to the number of NK cells prior to administration after three doses. In some embodiments, the effective amount of the heterodimeric protein of the disclosure is an amount sufficient to increase the number of NK cells at least 100-fold relative to the number of NK cells prior to administration after four doses. In some embodiments, the effective amount of the heterodimeric protein of the disclosure is an amount sufficient to increase the number of NK cells at least 100-fold relative to the number of NK cells prior to administration after five doses. In some embodiments, the effective amount of the heterodimeric protein of the disclosure is an amount sufficient to increase the number of NK cells at least 100-fold relative to the number of NK cells prior to administration after six doses. In some embodiments, the effective amount of the heterodimeric protein of the disclosure is an amount sufficient to increase the number of NK cells at least 100-fold relative to the number of NK cells prior to administration after seven doses. In some embodiments, the effective amount of the heterodimeric protein of the disclosure is an amount sufficient to increase the number of NK cells at least 100-fold relative to the number of NK cells prior to administration after eight doses. In some embodiments, the effective amount of the heterodimeric protein of the disclosure is an amount sufficient to increase the number of NK cells at least 100-fold relative to the number of NK cells prior to administration after nine doses. In some embodiments, the effective amount of the heterodimeric protein of the disclosure is an amount sufficient to increase the number of NK cells at least 100-fold relative to the number of NK cells prior to administration after ten doses.

In some embodiments, the effective amount of the heterodimeric protein of the disclosure is an amount sufficient to increase the number of NK cells at least 20-fold relative to the number of NK cells prior to administration after no more than no more than two doses. In some embodiments, the effective amount of the heterodimeric protein of the disclosure is an amount sufficient to increase the number of NK cells at least 20-fold relative to the number of NK cells prior to administration after no more than no more than three doses. In some embodiments, the effective amount of the heterodimeric protein of the disclosure is an amount sufficient to increase the number of NK cells at least 20-fold relative to the number of NK cells prior to administration after no more than four doses. In some embodiments, the effective amount of the heterodimeric protein of the disclosure is an amount sufficient to increase the number of NK cells at least 20-fold relative to the number of NK cells prior to administration after no more than five doses. In some embodiments, the effective amount of the heterodimeric protein of the disclosure is an amount sufficient to increase the number of NK cells at least 20-fold relative to the number of NK cells prior to administration after no more than six doses. In some embodiments, the effective amount of the heterodimeric protein of the disclosure is an amount sufficient to increase the number of NK cells at least 20-fold relative to the number of NK cells prior to administration after no more than seven doses. In some embodiments, the effective amount of the heterodimeric protein of the disclosure is an amount sufficient to increase the number of NK cells at least 20-fold relative to the number of NK cells prior to administration after no more than eight doses. In some embodiments, the effective amount of the heterodimeric protein of the disclosure is an amount sufficient to increase the number of NK cells at least 20-fold relative to the number of NK cells prior to administration after no more than nine doses. In some embodiments, the effective amount of the heterodimeric protein of the disclosure is an amount sufficient to increase the number of NK cells at least 20-fold relative to the number of NK cells prior to administration after no more than ten doses.

In some embodiments, the effective amount of the heterodimeric protein of the disclosure is an amount sufficient to increase the number of NK cells at least 50-fold relative to the number of NK cells prior to administration after no more than two doses. In some embodiments, the effective amount of the heterodimeric protein of the disclosure is an amount sufficient to increase the number of NK cells at least 50-fold relative to the number of NK cells prior to administration after no more than three doses. In some embodiments, the effective amount of the heterodimeric protein of the disclosure is an amount sufficient to increase the number of NK cells at least 50-fold relative to the number of NK cells prior to administration after no more than four doses. In some embodiments, the effective amount of the heterodimeric protein of the disclosure is an amount sufficient to increase the number of NK cells at least 50-fold relative to the number of NK cells prior to administration after no more than five doses. In some embodiments, the effective amount of the heterodimeric protein of the disclosure is an amount sufficient to increase the number of NK cells at least 50-fold relative to the number of NK cells prior to administration after no more than six doses. In some embodiments, the effective amount of the heterodimeric protein of the disclosure is an amount sufficient to increase the number of NK cells at least 50-fold relative to the number of NK cells prior to administration after no more than seven doses. In some embodiments, the effective amount of the heterodimeric protein of the disclosure is an amount sufficient to increase the number of NK cells at least 50-fold relative to the number of NK cells prior to administration after no more than eight doses. In some embodiments, the effective amount of the heterodimeric protein of the disclosure is an amount sufficient to increase the number of NK cells at least 50-fold relative to the number of NK cells prior to administration after no more than nine doses. In some embodiments, the effective amount of the heterodimeric protein of the disclosure is an amount sufficient to increase the number of NK cells at least 50-fold relative to the number of NK cells prior to administration after no more than ten doses.

In some embodiments, the effective amount of the heterodimeric protein of the disclosure is an amount sufficient to increase the number of NK cells at least 100-fold relative to the number of NK cells prior to administration after no more than two doses. In some embodiments, the effective amount of the heterodimeric protein of the disclosure is an amount sufficient to increase the number of NK cells at least 100-fold relative to the number of NK cells prior to administration after no more than three doses. In some embodiments, the effective amount of the heterodimeric protein of the disclosure is an amount sufficient to increase the number of NK cells at least 100-fold relative to the number of NK cells prior to administration after no more than four doses. In some embodiments, the effective amount of the heterodimeric protein of the disclosure is an amount sufficient to increase the number of NK cells at least 100-fold relative to the number of NK cells prior to administration after no more than five doses. In some embodiments, the effective amount of the heterodimeric protein of the disclosure is an amount sufficient to increase the number of NK cells at least 100-fold relative to the number of NK cells prior to administration after no more than six doses. In some embodiments, the effective amount of the heterodimeric protein of the disclosure is an amount sufficient to increase the number of NK cells at least 100-fold relative to the number of NK cells prior to administration after no more than seven doses. In some embodiments, the effective amount of the heterodimeric protein of the disclosure is an amount sufficient to increase the number of NK cells at least 100-fold relative to the number of NK cells prior to administration after no more than eight doses. In some embodiments, the effective amount of the heterodimeric protein of the disclosure is an amount sufficient to increase the number of NK cells at least 100-fold relative to the number of NK cells prior to administration after no more than nine doses. In some embodiments, the effective amount of the heterodimeric protein of the disclosure is an amount sufficient to increase the number of NK cells at least 100-fold relative to the number of NK cells prior to administration after no more than ten doses.

In some embodiments, the heterodimeric protein is administered at a dose of at least 0.02 mg/kg Q2W and the number of NK cells increases at least 20-fold as compared to the number of NK cells prior to administration after two doses. In some embodiments, the heterodimeric protein is administered at a dose of at least 0.02 mg/kg Q2W and the number of NK cells increases at least 20-fold as compared to the number of NK cells prior to administration after three doses. In some embodiments, the heterodimeric protein is administered at a dose of at least 0.02 mg/kg Q2W and the number of NK cells increases at least 20-fold as compared to the number of NK cells prior to administration after four doses. In some embodiments, the heterodimeric protein is administered at a dose of at least 0.02 mg/kg Q2W and the number of NK cells increases at least 20-fold as compared to the number of NK cells prior to administration after five doses. In some embodiments, the heterodimeric protein is administered at a dose of at least 0.02 mg/kg Q2W and the number of NK cells increases at least 30-fold as compared to the number of NK cells prior to administration after two doses. In some embodiments, the heterodimeric protein is administered at a dose of at least 0.02 mg/kg Q2W and the number of NK cells increases at least 30-fold as compared to the number of NK cells prior to administration after three doses. In some embodiments, the heterodimeric protein is administered at a dose of at least 0.02 mg/kg Q2W and the number of NK cells increases at least 30-fold as compared to the number of NK cells prior to administration after four doses. In some embodiments, the heterodimeric protein is administered at a dose of at least 0.02 mg/kg Q2W and the number of NK cells increases at least 30-fold as compared to the number of NK cells prior to administration after five doses. In some embodiments, the heterodimeric protein is administered at a dose of at least 0.02 mg/kg Q2W and the number of NK cells increases at least 40-fold as compared to the number of NK cells prior to administration after two doses. In some embodiments, the heterodimeric protein is administered at a dose of at least 0.02 mg/kg Q2W and the number of NK cells increases at least 40-fold as compared to the number of NK cells prior to administration after three doses. In some embodiments, the heterodimeric protein is administered at a dose of at least 0.02 mg/kg Q2W and the number of NK cells increases at least 40-fold as compared to the number of NK cells prior to administration after four doses. In some embodiments, the heterodimeric protein is administered at a dose of at least 0.02 mg/kg Q2W and the number of NK cells increases at least 40-fold as compared to the number of NK cells prior to administration after five doses. In some embodiments, the heterodimeric protein is administered at a dose of at least 0.02 mg/kg Q2W and the number of NK cells increases at least 50-fold as compared to the number of NK cells prior to administration after two doses. In some embodiments, the heterodimeric protein is administered at a dose of at least 0.02 mg/kg Q2W and the number of NK cells increases at least 50-fold as compared to the number of NK cells prior to administration after three doses. In some embodiments, the heterodimeric protein is administered at a dose of at least 0.02 mg/kg Q2W and the number of NK cells increases at least 50-fold as compared to the number of NK cells prior to administration after four doses. In some embodiments, the heterodimeric protein is administered at a dose of at least 0.02 mg/kg Q2W and the number of NK cells increases at least 50-fold as compared to the number of NK cells prior to administration after five doses. In some embodiments, the heterodimeric protein is administered at a dose of at least 0.02 mg/kg Q2W and the number of NK cells increases at least 100-fold as compared to the number of NK cells prior to administration after two doses. In some embodiments, the heterodimeric protein is administered at a dose of at least 0.02 mg/kg Q2W and the number of NK cells increases at least 100-fold as compared to the number of NK cells prior to administration after three doses. In some embodiments, the heterodimeric protein is administered at a dose of at least 0.02 mg/kg Q2W and the number of NK cells increases at least 100-fold as compared to the number of NK cells prior to administration after four doses. In some embodiments, the heterodimeric protein is administered at a dose of at least 0.02 mg/kg Q2W and the number of NK cells increases at least 100-fold as compared to the number of NK cells prior to administration after five doses. In some embodiments, the heterodimeric protein is administered at a dose of at least 0.02 mg/kg Q2W and the number of NK cells increases at least 200-fold as compared to the number of NK cells prior to administration after two doses. In some embodiments, the heterodimeric protein is administered at a dose of at least 0.02 mg/kg Q2W and the number of NK cells increases at least 200-fold as compared to the number of NK cells prior to administration after three doses. In some embodiments, the heterodimeric protein is administered at a dose of at least 0.02 mg/kg Q2W and the number of NK cells increases at least 200-fold as compared to the number of NK cells prior to administration after four doses. In some embodiments, the heterodimeric protein is administered at a dose of at least 0.02 mg/kg Q2W and the number of NK cells increases at least 200-fold as compared to the number of NK cells prior to administration after five doses.

In some embodiments, the heterodimeric protein is administered at a dose of at least 0.04 mg/kg Q2W and the number of NK cells increases at least 20-fold as compared to the number of NK cells prior to administration after two doses. In some embodiments, the heterodimeric protein is administered at a dose of at least 0.04 mg/kg Q2W and the number of NK cells increases at least 20-fold as compared to the number of NK cells prior to administration after three doses. In some embodiments, the heterodimeric protein is administered at a dose of at least 0.04 mg/kg Q2W and the number of NK cells increases at least 20-fold as compared to the number of NK cells prior to administration after four doses. In some embodiments, the heterodimeric protein is administered at a dose of at least 0.04 mg/kg Q2W and the number of NK cells increases at least 20-fold as compared to the number of NK cells prior to administration after five doses. In some embodiments, the heterodimeric protein is administered at a dose of at least 0.04 mg/kg Q2W and the number of NK cells increases at least 30-fold as compared to the number of NK cells prior to administration after two doses. In some embodiments, the heterodimeric protein is administered at a dose of at least 0.04 mg/kg Q2W and the number of NK cells increases at least 30-fold as compared to the number of NK cells prior to administration after three doses. In some embodiments, the heterodimeric protein is administered at a dose of at least 0.04 mg/kg Q2W and the number of NK cells increases at least 30-fold as compared to the number of NK cells prior to administration after four doses. In some embodiments, the heterodimeric protein is administered at a dose of at least 0.04 mg/kg Q2W and the number of NK cells increases at least 30-fold as compared to the number of NK cells prior to administration after five doses. In some embodiments, the heterodimeric protein is administered at a dose of at least 0.04 mg/kg Q2W and the number of NK cells increases at least 40-fold as compared to the number of NK cells prior to administration after two doses. In some embodiments, the heterodimeric protein is administered at a dose of at least 0.04 mg/kg Q2W and the number of NK cells increases at least 40-fold as compared to the number of NK cells prior to administration after three doses. In some embodiments, the heterodimeric protein is administered at a dose of at least 0.04 mg/kg Q2W and the number of NK cells increases at least 40-fold as compared to the number of NK cells prior to administration after four doses. In some embodiments, the heterodimeric protein is administered at a dose of at least 0.04 mg/kg Q2W and the number of NK cells increases at least 40-fold as compared to the number of NK cells prior to administration after five doses. In some embodiments, the heterodimeric protein is administered at a dose of at least 0.04 mg/kg Q2W and the number of NK cells increases at least 50-fold as compared to the number of NK cells prior to administration after two doses. In some embodiments, the heterodimeric protein is administered at a dose of at least 0.04 mg/kg Q2W and the number of NK cells increases at least 50-fold as compared to the number of NK cells prior to administration after three doses. In some embodiments, the heterodimeric protein is administered at a dose of at least 0.04 mg/kg Q2W and the number of NK cells increases at least 50-fold as compared to the number of NK cells prior to administration after four doses. In some embodiments, the heterodimeric protein is administered at a dose of at least 0.04 mg/kg Q2W and the number of NK cells increases at least 50-fold as compared to the number of NK cells prior to administration after five doses. In some embodiments, the heterodimeric protein is administered at a dose of at least 0.04 mg/kg Q2W and the number of NK cells increases at least 100-fold as compared to the number of NK cells prior to administration after two doses. In some embodiments, the heterodimeric protein is administered at a dose of at least 0.04 mg/kg Q2W and the number of NK cells increases at least 100-fold as compared to the number of NK cells prior to administration after three doses. In some embodiments, the heterodimeric protein is administered at a dose of at least 0.04 mg/kg Q2W and the number of NK cells increases at least 100-fold as compared to the number of NK cells prior to administration after four doses. In some embodiments, the heterodimeric protein is administered at a dose of at least 0.04 mg/kg Q2W and the number of NK cells increases at least 100-fold as compared to the number of NK cells prior to administration after five doses. In some embodiments, the heterodimeric protein is administered at a dose of at least 0.04 mg/kg Q2W and the number of NK cells increases at least 200-fold as compared to the number of NK cells prior to administration after two doses. In some embodiments, the heterodimeric protein is administered at a dose of at least 0.04 mg/kg Q2W and the number of NK cells increases at least 200-fold as compared to the number of NK cells prior to administration after three doses. In some embodiments, the heterodimeric protein is administered at a dose of at least 0.04 mg/kg Q2W and the number of NK cells increases at least 200-fold as compared to the number of NK cells prior to administration after four doses. In some embodiments, the heterodimeric protein is administered at a dose of at least 0.04 mg/kg Q2W and the number of NK cells increases at least 200-fold as compared to the number of NK cells prior to administration after five doses.

In some embodiments, the heterodimeric protein is administered at a dose of at least 0.06 mg/kg Q2W and the number of NK cells increases at least 20-fold as compared to the number of NK cells prior to administration after two doses. In some embodiments, the heterodimeric protein is administered at a dose of at least 0.06 mg/kg Q2W and the number of NK cells increases at least 20-fold as compared to the number of NK cells prior to administration after three doses. In some embodiments, the heterodimeric protein is administered at a dose of at least 0.06 mg/kg Q2W and the number of NK cells increases at least 20-fold as compared to the number of NK cells prior to administration after four doses. In some embodiments, the heterodimeric protein is administered at a dose of at least 0.06 mg/kg Q2W and the number of NK cells increases at least 20-fold as compared to the number of NK cells prior to administration after five doses. In some embodiments, the heterodimeric protein is administered at a dose of at least 0.06 mg/kg Q2W and the number of NK cells increases at least 30-fold as compared to the number of NK cells prior to administration after two doses. In some embodiments, the heterodimeric protein is administered at a dose of at least 0.06 mg/kg Q2W and the number of NK cells increases at least 30-fold as compared to the number of NK cells prior to administration after three doses. In some embodiments, the heterodimeric protein is administered at a dose of at least 0.06 mg/kg Q2W and the number of NK cells increases at least 30-fold as compared to the number of NK cells prior to administration after four doses. In some embodiments, the heterodimeric protein is administered at a dose of at least 0.06 mg/kg Q2W and the number of NK cells increases at least 30-fold as compared to the number of NK cells prior to administration after five doses. In some embodiments, the heterodimeric protein is administered at a dose of at least 0.06 mg/kg Q2W and the number of NK cells increases at least 40-fold as compared to the number of NK cells prior to administration after two doses. In some embodiments, the heterodimeric protein is administered at a dose of at least 0.06 mg/kg Q2W and the number of NK cells increases at least 40-fold as compared to the number of NK cells prior to administration after three doses. In some embodiments, the heterodimeric protein is administered at a dose of at least 0.06 mg/kg Q2W and the number of NK cells increases at least 40-fold as compared to the number of NK cells prior to administration after four doses. In some embodiments, the heterodimeric protein is administered at a dose of at least 0.06 mg/kg Q2W and the number of NK cells increases at least 40-fold as compared to the number of NK cells prior to administration after five doses. In some embodiments, the heterodimeric protein is administered at a dose of at least 0.06 mg/kg Q2W and the number of NK cells increases at least 50-fold as compared to the number of NK cells prior to administration after two doses. In some embodiments, the heterodimeric protein is administered at a dose of at least 0.06 mg/kg Q2W and the number of NK cells increases at least 50-fold as compared to the number of NK cells prior to administration after three doses. In some embodiments, the heterodimeric protein is administered at a dose of at least 0.06 mg/kg Q2W and the number of NK cells increases at least 50-fold as compared to the number of NK cells prior to administration after four doses. In some embodiments, the heterodimeric protein is administered at a dose of at least 0.06 mg/kg Q2W and the number of NK cells increases at least 50-fold as compared to the number of NK cells prior to administration after five doses. In some embodiments, the heterodimeric protein is administered at a dose of at least 0.06 mg/kg Q2W and the number of NK cells increases at least 100-fold as compared to the number of NK cells prior to administration after two doses. In some embodiments, the heterodimeric protein is administered at a dose of at least 0.06 mg/kg Q2W and the number of NK cells increases at least 100-fold as compared to the number of NK cells prior to administration after three doses. In some embodiments, the heterodimeric protein is administered at a dose of at least 0.06 mg/kg Q2W and the number of NK cells increases at least 100-fold as compared to the number of NK cells prior to administration after four doses. In some embodiments, the heterodimeric protein is administered at a dose of at least 0.06 mg/kg Q2W and the number of NK cells increases at least 100-fold as compared to the number of NK cells prior to administration after five doses. In some embodiments, the heterodimeric protein is administered at a dose of at least 0.06 mg/kg Q2W and the number of NK cells increases at least 200-fold as compared to the number of NK cells prior to administration after two doses. In some embodiments, the heterodimeric protein is administered at a dose of at least 0.06 mg/kg Q2W and the number of NK cells increases at least 200-fold as compared to the number of NK cells prior to administration after three doses. In some embodiments, the heterodimeric protein is administered at a dose of at least 0.06 mg/kg Q2W and the number of NK cells increases at least 200-fold as compared to the number of NK cells prior to administration after four doses. In some embodiments, the heterodimeric protein is administered at a dose of at least 0.06 mg/kg Q2W and the number of NK cells increases at least 200-fold as compared to the number of NK cells prior to administration after five doses.

In some embodiments, the heterodimeric protein is administered at a dose of at least 0.09 mg/kg Q2W and the number of NK cells increases at least 20-fold as compared to the number of NK cells prior to administration after two doses. In some embodiments, the heterodimeric protein is administered at a dose of at least 0.09 mg/kg Q2W and the number of NK cells increases at least 20-fold as compared to the number of NK cells prior to administration after three doses. In some embodiments, the heterodimeric protein is administered at a dose of at least 0.09 mg/kg Q2W and the number of NK cells increases at least 20-fold as compared to the number of NK cells prior to administration after four doses. In some embodiments, the heterodimeric protein is administered at a dose of at least 0.09 mg/kg Q2W and the number of NK cells increases at least 20-fold as compared to the number of NK cells prior to administration after five doses. In some embodiments, the heterodimeric protein is administered at a dose of at least 0.09 mg/kg Q2W and the number of NK cells increases at least 30-fold as compared to the number of NK cells prior to administration after two doses. In some embodiments, the heterodimeric protein is administered at a dose of at least 0.09 mg/kg Q2W and the number of NK cells increases at least 30-fold as compared to the number of NK cells prior to administration after three doses. In some embodiments, the heterodimeric protein is administered at a dose of at least 0.09 mg/kg Q2W and the number of NK cells increases at least 30-fold as compared to the number of NK cells prior to administration after four doses. In some embodiments, the heterodimeric protein is administered at a dose of at least 0.09 mg/kg Q2W and the number of NK cells increases at least 30-fold as compared to the number of NK cells prior to administration after five doses. In some embodiments, the heterodimeric protein is administered at a dose of at least 0.09 mg/kg Q2W and the number of NK cells increases at least 40-fold as compared to the number of NK cells prior to administration after two doses. In some embodiments, the heterodimeric protein is administered at a dose of at least 0.09 mg/kg Q2W and the number of NK cells increases at least 40-fold as compared to the number of NK cells prior to administration after three doses. In some embodiments, the heterodimeric protein is administered at a dose of at least 0.09 mg/kg Q2W and the number of NK cells increases at least 40-fold as compared to the number of NK cells prior to administration after four doses. In some embodiments, the heterodimeric protein is administered at a dose of at least 0.09 mg/kg Q2W and the number of NK cells increases at least 40-fold as compared to the number of NK cells prior to administration after five doses. In some embodiments, the heterodimeric protein is administered at a dose of at least 0.09 mg/kg Q2W and the number of NK cells increases at least 50-fold as compared to the number of NK cells prior to administration after two doses. In some embodiments, the heterodimeric protein is administered at a dose of at least 0.09 mg/kg Q2W and the number of NK cells increases at least 50-fold as compared to the number of NK cells prior to administration after three doses. In some embodiments, the heterodimeric protein is administered at a dose of at least 0.09 mg/kg Q2W and the number of NK cells increases at least 50-fold as compared to the number of NK cells prior to administration after four doses. In some embodiments, the heterodimeric protein is administered at a dose of at least 0.09 mg/kg Q2W and the number of NK cells increases at least 50-fold as compared to the number of NE cells prior to administration after five doses. In some embodiments, the heterodimeric protein is administered at a dose of at least 0.09 mg/kg Q2W and the number of NK cells increases at least 100-fold as compared to the number of NK cells prior to administration after two doses. In some embodiments, the heterodimeric protein is administered at a dose of at least 0.09 mg/kg Q2W and the number of NK cells increases at least 100-fold as compared to the number of NK cells prior to administration after three doses. In some embodiments, the heterodimeric protein is administered at a dose of at least 0.09 mg/kg Q2W and the number of NK cells increases at least 100-fold as compared to the number of NK cells prior to administration after four doses. In some embodiments, the heterodimeric protein is administered at a dose of at least 0.09 mg/kg Q2W and the number of NK cells increases at least 100-fold as compared to the number of NK cells prior to administration after five doses. In some embodiments, the heterodimeric protein is administered at a dose of at least 0.09 mg/kg Q2W and the number of NK cells increases at least 200-fold as compared to the number of NK cells prior to administration after two doses. In some embodiments, the heterodimeric protein is administered at a dose of at least 0.09 mg/kg Q2W and the number of NK cells increases at least 200-fold as compared to the number of NK cells prior to administration after three doses. In some embodiments, the heterodimeric protein is administered at a dose of at least 0.09 mg/kg Q2W and the number of NK cells increases at least 200-fold as compared to the number of NK cells prior to administration after four doses. In some embodiments, the heterodimeric protein is administered at a dose of at least 0.09 mg/kg Q2W and the number of NK cells increases at least 200-fold as compared to the number of NK cells prior to administration after five doses.

In some embodiments, the heterodimeric protein is administered at a dose of at least 0.12 mg/kg Q2W and the number of NK cells increases at least 20-fold as compared to the number of NK cells prior to administration after two doses. In some embodiments, the heterodimeric protein is administered at a dose of at least 0.12 mg/kg Q2W and the number of NK cells increases at least 20-fold as compared to the number of NK cells prior to administration after three doses. In some embodiments, the heterodimeric protein is administered at a dose of at least 0.12 mg/kg Q2W and the number of NK cells increases at least 20-fold as compared to the number of NK cells prior to administration after four doses. In some embodiments, the heterodimeric protein is administered at a dose of at least 0.12 mg/kg Q2W and the number of NK cells increases at least 20-fold as compared to the number of NK cells prior to administration after five doses. In some embodiments, the heterodimeric protein is administered at a dose of at least 0.12 mg/kg Q2W and the number of NK cells increases at least 30-fold as compared to the number of NK cells prior to administration after two doses. In some embodiments, the heterodimeric protein is administered at a dose of at least 0.12 mg/kg Q2W and the number of NK cells increases at least 30-fold as compared to the number of NK cells prior to administration after three doses. In some embodiments, the heterodimeric protein is administered at a dose of at least 0.12 mg/kg Q2W and the number of NK cells increases at least 30-fold as compared to the number of NK cells prior to administration after four doses. In some embodiments, the heterodimeric protein is administered at a dose of at least 0.12 mg/kg Q2W and the number of NK cells increases at least 30-fold as compared to the number of NK cells prior to administration after five doses. In some embodiments, the heterodimeric protein is administered at a dose of at least 0.12 mg/kg Q2W and the number of NK cells increases at least 40-fold as compared to the number of NY cells prior to administration after two doses. In some embodiments, the heterodimeric protein is administered at a dose of at least 0.12 mg/kg Q2W and the number of NK cells increases at least 40-fold as compared to the number of NK cells prior to administration after three doses. In some embodiments, the heterodimeric protein is administered at a dose of at least 0.12 mg/kg Q2W and the number of NK cells increases at least 40-fold as compared to the number of NK cells prior to administration after four doses. In some embodiments, the heterodimeric protein is administered at a dose of at least 0.12 mg/kg Q2W and the number of NK cells increases at least 40-fold as compared to the number of NK cells prior to administration after five doses. In some embodiments, the heterodimeric protein is administered at a dose of at least 0.12 mg/kg Q2W and the number of NK cells increases at least 50-fold as compared to the number of NK cells prior to administration after two doses. In some embodiments, the heterodimeric protein is administered at a dose of at least 0.12 mg/kg Q2W and the number of NK cells increases at least 50-fold as compared to the number of NK cells prior to administration after three doses. In some embodiments, the heterodimeric protein is administered at a dose of at least 0.12 mg/kg Q2W and the number of NK cells increases at least 50-fold as compared to the number of NK cells prior to administration after four doses. In some embodiments, the heterodimeric protein is administered at a dose of at least 0.12 mg/kg Q2W and the number of NK cells increases at least 50-fold as compared to the number of NK cells prior to administration after five doses. In some embodiments, the heterodimeric protein is administered at a dose of at least 0.12 mg/kg Q2W and the number of NK cells increases at least 100-fold as compared to the number of NK cells prior to administration after two doses. In some embodiments, the heterodimeric protein is administered at a dose of at least 0.12 mg/kg Q2W and the number of NK cells increases at least 100-fold as compared to the number of NK cells prior to administration after three doses. In some embodiments, the heterodimeric protein is administered at a dose of at least 0.12 mg/kg Q2W and the number of NK cells increases at least 100-fold as compared to the number of NK cells prior to administration after four doses. In some embodiments, the heterodimeric protein is administered at a dose of at least 0.12 mg/kg Q2W and the number of NK cells increases at least 100-fold as compared to the number of NK cells prior to administration after five doses. In some embodiments, the heterodimeric protein is administered at a dose of at least 0.12 mg/kg Q2W and the number of NK cells increases at least 200-fold as compared to the number of NK cells prior to administration after two doses. In some embodiments, the heterodimeric protein is administered at a dose of at least 0.12 mg/kg Q2W and the number of NK cells increases at least 200-fold as compared to the number of NK cells prior to administration after three doses. In some embodiments, the heterodimeric protein is administered at a dose of at least 0.12 mg/kg Q2W and the number of NK cells increases at least 200-fold as compared to the number of NK cells prior to administration after four doses. In some embodiments, the heterodimeric protein is administered at a dose of at least 0.12 mg/kg Q2W and the number of NK cells increases at least 200-fold as compared to the number of NK cells prior to administration after five doses.

In some embodiments, the combination of heterodimeric proteins is administered at a dose of at least 0.02 mg/kg Q2W and the number of NK cells increases at least 20-fold as compared to the number of NK cells prior to administration after two doses. In some embodiments, the combination of heterodimeric proteins is administered at a dose of at least 0.02 mg/kg Q2W and the number of NK cells increases at least 20-fold as compared to the number of NK cells prior to administration after three doses. In some embodiments, the combination of heterodimeric proteins is administered at a dose of at least 0.02 mg/kg Q2W and the number of NK cells increases at least 20-fold as compared to the number of NK cells prior to administration after four doses. In some embodiments, the combination of heterodimeric proteins is administered at a dose of at least 0.02 mg/kg Q2W and the number of NK cells increases at least 20-fold as compared to the number of NK cells prior to administration after five doses. In some embodiments, the combination of heterodimeric proteins is administered at a dose of at least 0.02 mg/kg Q2W and the number of NK cells increases at least 30-fold as compared to the number of NK cells prior to administration after two doses. In some embodiments, the combination of heterodimeric proteins is administered at a dose of at least 0.02 mg/kg Q2W and the number of NK cells increases at least 30-fold as compared to the number of NK cells prior to administration after three doses. In some embodiments, the combination of heterodimeric proteins is administered at a dose of at least 0.02 mg/kg Q2W and the number of NK cells increases at least 30-fold as compared to the number of NK cells prior to administration after four doses. In some embodiments, the combination of heterodimeric proteins is administered at a dose of at least 0.02 mg/kg Q2W and the number of NK cells increases at least 30-fold as compared to the number of NK cells prior to administration after five doses. In some embodiments, the combination of heterodimeric proteins is administered at a dose of at least 0.02 mg/kg Q2W and the number of NK cells increases at least 40-fold as compared to the number of NK cells prior to administration after two doses. In some embodiments, the combination of heterodimeric proteins is administered at a dose of at least 0.02 mg/kg Q2W and the number of NK cells increases at least 40-fold as compared to the number of NK cells prior to administration after three doses. In some embodiments, the combination of heterodimeric proteins is administered at a dose of at least 0.02 mg/kg Q2W and the number of NK cells increases at least 40-fold as compared to the number of NK cells prior to administration after four doses. In some embodiments, the combination of heterodimeric proteins is administered at a dose of at least 0.02 mg/kg Q2W and the number of NK cells increases at least 40-fold as compared to the number of NK cells prior to administration after five doses. In some embodiments, the combination of heterodimeric proteins is administered at a dose of at least 0.02 mg/kg Q2W and the number of NK cells increases at least 50-fold as compared to the number of NK cells prior to administration after two doses. In some embodiments, the combination of heterodimeric proteins is administered at a dose of at least 0.02 mg/kg Q2W and the number of NK cells increases at least 50-fold as compared to the number of NK cells prior to administration after three doses. In some embodiments, the combination of heterodimeric proteins is administered at a dose of at least 0.02 mg/kg Q2W and the number of NK cells increases at least 50-fold as compared to the number of NK cells prior to administration after four doses. In some embodiments, the combination of heterodimeric proteins is administered at a dose of at least 0.02 mg/kg Q2W and the number of NK cells increases at least 50-fold as compared to the number of NK cells prior to administration after five doses. In some embodiments, the combination of heterodimeric proteins is administered at a dose of at least 0.02 mg/kg Q2W and the number of NK cells increases at least 100-fold as compared to the number of NK cells prior to administration after two doses. In some embodiments, the combination of heterodimeric proteins is administered at a dose of at least 0.02 mg/kg Q2W and the number of NK cells increases at least 100-fold as compared to the number of NK cells prior to administration after three doses. In some embodiments, the combination of heterodimeric proteins is administered at a dose of at least 0.02 mg/kg Q2W and the number of NK cells increases at least 100-fold as compared to the number of NK cells prior to administration after four doses. In some embodiments, the combination of heterodimeric proteins is administered at a dose of at least 0.02 mg/kg Q2W and the number of NK cells increases at least 100-fold as compared to the number of NK cells prior to administration after five doses. In some embodiments, the combination of heterodimeric proteins is administered at a dose of at least 0.02 mg/kg Q2W and the number of NK cells increases at least 200-fold as compared to the number of NK cells prior to administration after two doses. In some embodiments, the combination of heterodimeric proteins is administered at a dose of at least 0.02 mg/kg Q2W and the number of NK cells increases at least 200-fold as compared to the number of NK cells prior to administration after three doses. In some embodiments, the combination of heterodimeric proteins is administered at a dose of at least 0.02 mg/kg Q2W and the number of NK cells increases at least 200-fold as compared to the number of NK cells prior to administration after four doses. In some embodiments, the combination of heterodimeric proteins is administered at a dose of at least 0.02 mg/kg Q2W and the number of NK cells increases at least 200-fold as compared to the number of NK cells prior to administration after five doses.

In some embodiments, the combination of heterodimeric proteins is administered at a dose of at least 0.04 mg/kg Q2W and the number of NK cells increases at least 20-fold as compared to the number of NK cells prior to administration after two doses. In some embodiments, the combination of heterodimeric proteins is administered at a dose of at least 0.04 mg/kg Q2W and the number of NK cells increases at least 20-fold as compared to the number of NK cells prior to administration after three doses. In some embodiments, the combination of heterodimeric proteins is administered at a dose of at least 0.04 mg/kg Q2W and the number of NK cells increases at least 20-fold as compared to the number of NK cells prior to administration after four doses. In some embodiments, the combination of heterodimeric proteins is administered at a dose of at least 0.04 mg/kg Q2W and the number of NK cells increases at least 20-fold as compared to the number of NK cells prior to administration after five doses. In some embodiments, the combination of heterodimeric proteins is administered at a dose of at least 0.04 mg/kg Q2W and the number of NK cells increases at least 30-fold as compared to the number of NK cells prior to administration after two doses. In some embodiments, the combination of heterodimeric proteins is administered at a dose of at least 0.04 mg/kg Q2W and the number of NK cells increases at least 30-fold as compared to the number of NK cells prior to administration after three doses. In some embodiments, the combination of heterodimeric proteins is administered at a dose of at least 0.04 mg/kg Q2W and the number of NK cells increases at least 30-fold as compared to the number of NK cells prior to administration after four doses. In some embodiments, the combination of heterodimeric proteins is administered at a dose of at least 0.04 mg/kg Q2W and the number of NK cells increases at least 30-fold as compared to the number of NK cells prior to administration after five doses. In some embodiments, the combination of heterodimeric proteins is administered at a dose of at least 0.04 mg/kg Q2W and the number of NK cells increases at least 40-fold as compared to the number of NK cells prior to administration after two doses. In some embodiments, the combination of heterodimeric proteins is administered at a dose of at least 0.04 mg/kg Q2W and the number of NK cells increases at least 40-fold as compared to the number of NK cells prior to administration after three doses. In some embodiments, the combination of heterodimeric proteins is administered at a dose of at least 0.04 mg/kg Q2W and the number of NK cells increases at least 40-fold as compared to the number of NK cells prior to administration after four doses. In some embodiments, the combination of heterodimeric proteins is administered at a dose of at least 0.04 mg/kg Q2W and the number of NK cells increases at least 40-fold as compared to the number of NK cells prior to administration after five doses. In some embodiments, the combination of heterodimeric proteins is administered at a dose of at least 0.04 mg/kg Q2W and the number of NK cells increases at least 50-fold as compared to the number of NK cells prior to administration after two doses. In some embodiments, the combination of heterodimeric proteins is administered at a dose of at least 0.04 mg/kg Q2W and the number of NK cells increases at least 50-fold as compared to the number of NK cells prior to administration after three doses. In some embodiments, the combination of heterodimeric proteins is administered at a dose of at least 0.04 mg/kg Q2W and the number of NK cells increases at least 50-fold as compared to the number of NK cells prior to administration after four doses. In some embodiments, the combination of heterodimeric proteins is administered at a dose of at least 0.04 mg/kg Q2W and the number of NK cells increases at least 50-fold as compared to the number of NK cells prior to administration after five doses. In some embodiments, the combination of heterodimeric proteins is administered at a dose of at least 0.04 mg/kg Q2W and the number of NK cells increases at least 100-fold as compared to the number of NK cells prior to administration after two doses. In some embodiments, the combination of heterodimeric proteins is administered at a dose of at least 0.04 mg/kg Q2W and the number of NK cells increases at least 100-fold as compared to the number of NK cells prior to administration after three doses. In some embodiments, the combination of heterodimeric proteins is administered at a dose of at least 0.04 mg/kg Q2W and the number of NK cells increases at least 100-fold as compared to the number of NK cells prior to administration after four doses. In some embodiments, the combination of heterodimeric proteins is administered at a dose of at least 0.04 mg/kg Q2W and the number of NK cells increases at least 100-fold as compared to the number of NK cells prior to administration after five doses. In some embodiments, the combination of heterodimeric proteins is administered at a dose of at least 0.04 mg/kg Q2W and the number of NK cells increases at least 200-fold as compared to the number of NK cells prior to administration after two doses. In some embodiments, the combination of heterodimeric proteins is administered at a dose of at least 0.04 mg/kg Q2W and the number of NK cells increases at least 200-fold as compared to the number of NK cells prior to administration after three doses. In some embodiments, the combination of heterodimeric proteins is administered at a dose of at least 0.04 mg/kg Q2W and the number of NK cells increases at least 200-fold as compared to the number of NK cells prior to administration after four doses. In some embodiments, the combination of heterodimeric proteins is administered at a dose of at least 0.04 mg/kg Q2W and the number of NK cells increases at least 200-fold as compared to the number of NK cells prior to administration after five doses.

In some embodiments, the combination of heterodimeric proteins is administered at a dose of at least 0.06 mg/kg Q2W and the number of NK cells increases at least 20-fold as compared to the number of NK cells prior to administration after two doses. In some embodiments, the combination of heterodimeric proteins is administered at a dose of at least 0.06 mg/kg Q2W and the number of NK cells increases at least 20-fold as compared to the number of NK cells prior to administration after three doses. In some embodiments, the combination of heterodimeric proteins is administered at a dose of at least 0.06 mg/kg Q2W and the number of NK cells increases at least 20-fold as compared to the number of NK cells prior to administration after four doses. In some embodiments, the combination of heterodimeric proteins is administered at a dose of at least 0.06 mg/kg Q2W and the number of NK cells increases at least 20-fold as compared to the number of NK cells prior to administration after five doses. In some embodiments, the combination of heterodimeric proteins is administered at a dose of at least 0.06 mg/kg Q2W and the number of NK cells increases at least 30-fold as compared to the number of NK cells prior to administration after two doses. In some embodiments, the combination of heterodimeric proteins is administered at a dose of at least 0.06 mg/kg Q2W and the number of NK cells increases at least 30-fold as compared to the number of NK cells prior to administration after three doses. In some embodiments, the combination of heterodimeric proteins is administered at a dose of at least 0.06 mg/kg Q2W and the number of NK cells increases at least 30-fold as compared to the number of NK cells prior to administration after four doses. In some embodiments, the combination of heterodimeric proteins is administered at a dose of at least 0.06 mg/kg Q2W and the number of NK cells increases at least 30-fold as compared to the number of NK cells prior to administration after five doses. In some embodiments, the combination of heterodimeric proteins is administered at a dose of at least 0.06 mg/kg Q2W and the number of NK cells increases at least 40-fold as compared to the number of NK cells prior to administration after two doses. In some embodiments, the combination of heterodimeric proteins is administered at a dose of at least 0.06 mg/kg Q2W and the number of NK cells increases at least 40-fold as compared to the number of NK cells prior to administration after three doses. In some embodiments, the combination of heterodimeric proteins is administered at a dose of at least 0.06 mg/kg Q2W and the number of NK cells increases at least 40-fold as compared to the number of NK cells prior to administration after four doses. In some embodiments, the combination of heterodimeric proteins is administered at a dose of at least 0.06 mg/kg Q2W and the number of NK cells increases at least 40-fold as compared to the number of NK cells prior to administration after five doses. In some embodiments, the combination of heterodimeric proteins is administered at a dose of at least 0.06 mg/kg Q2W and the number of NK cells increases at least 50-fold as compared to the number of NK cells prior to administration after two doses. In some embodiments, the combination of heterodimeric proteins is administered at a dose of at least 0.06 mg/kg Q2W and the number of NK cells increases at least 50-fold as compared to the number of NK cells prior to administration after three doses. In some embodiments, the combination of heterodimeric proteins is administered at a dose of at least 0.06 mg/kg Q2W and the number of NK cells increases at least 50-fold as compared to the number of NK cells prior to administration after four doses. In some embodiments, the combination of heterodimeric proteins is administered at a dose of at least 0.06 mg/kg Q2W and the number of NK cells increases at least 50-fold as compared to the number of NK cells prior to administration after five doses. In some embodiments, the combination of heterodimeric proteins is administered at a dose of at least 0.06 mg/kg Q2W and the number of NK cells increases at least 100-fold as compared to the number of NK cells prior to administration after two doses. In some embodiments, the combination of heterodimeric proteins is administered at a dose of at least 0.06 mg/kg Q2W and the number of NK cells increases at least 100-fold as compared to the number of NK cells prior to administration after three doses. In some embodiments, the combination of heterodimeric proteins is administered at a dose of at least 0.06 mg/kg Q2W and the number of NK cells increases at least 100-fold as compared to the number of NK cells prior to administration after four doses. In some embodiments, the combination of heterodimeric proteins is administered at a dose of at least 0.06 mg/kg Q2W and the number of NK cells increases at least 100-fold as compared to the number of NK cells prior to administration after five doses. In some embodiments, the combination of heterodimeric proteins is administered at a dose of at least 0.06 mg/kg Q2W and the number of NK cells increases at least 200-fold as compared to the number of NK cells prior to administration after two doses. In some embodiments, the combination of heterodimeric proteins is administered at a dose of at least 0.06 mg/kg Q2W and the number of NK cells increases at least 200-fold as compared to the number of NK cells prior to administration after three doses. In some embodiments, the combination of heterodimeric proteins is administered at a dose of at least 0.06 mg/kg Q2W and the number of NK cells increases at least 200-fold as compared to the number of NK cells prior to administration after four doses. In some embodiments, the combination of heterodimeric proteins is administered at a dose of at least 0.06 mg/kg Q2W and the number of NK cells increases at least 200-fold as compared to the number of NK cells prior to administration after five doses.

In some embodiments, the combination of heterodimeric proteins is administered at a dose of at least 0.09 mg/kg Q2W and the number of NK cells increases at least 20-fold as compared to the number of NK cells prior to administration after two doses. In some embodiments, the combination of heterodimeric proteins is administered at a dose of at least 0.09 mg/kg Q2W and the number of NK cells increases at least 20-fold as compared to the number of NK cells prior to administration after three doses. In some embodiments, the combination of heterodimeric proteins is administered at a dose of at least 0.09 mg/kg Q2W and the number of NK cells increases at least 20-fold as compared to the number of NK cells prior to administration after four doses. In some embodiments, the combination of heterodimeric proteins is administered at a dose of at least 0.09 mg/kg Q2W and the number of NK cells increases at least 20-fold as compared to the number of NK cells prior to administration after five doses. In some embodiments, the combination of heterodimeric proteins is administered at a dose of at least 0.09 mg/kg Q2W and the number of NK cells increases at least 30-fold as compared to the number of NK cells prior to administration after two doses. In some embodiments, the combination of heterodimeric proteins is administered at a dose of at least 0.09 mg/kg Q2W and the number of NK cells increases at least 30-fold as compared to the number of NK cells prior to administration after three doses. In some embodiments, the combination of heterodimeric proteins is administered at a dose of at least 0.09 mg/kg Q2W and the number of NK cells increases at least 30-fold as compared to the number of NK cells prior to administration after four doses. In some embodiments, the combination of heterodimeric proteins is administered at a dose of at least 0.09 mg/kg Q2W and the number of NK cells increases at least 30-fold as compared to the number of NK cells prior to administration after five doses. In some embodiments, the combination of heterodimeric proteins is administered at a dose of at least 0.09 mg/kg Q2W and the number of NK cells increases at least 40-fold as compared to the number of NK cells prior to administration after two doses. In some embodiments, the combination of heterodimeric proteins is administered at a dose of at least 0.09 mg/kg Q2W and the number of NK cells increases at least 40-fold as compared to the number of NK cells prior to administration after three doses. In some embodiments, the combination of heterodimeric proteins is administered at a dose of at least 0.09 mg/kg Q2W and the number of NK cells increases at least 40-fold as compared to the number of NK cells prior to administration after four doses. In some embodiments, the combination of heterodimeric proteins is administered at a dose of at least 0.09 mg/kg Q2W and the number of NK cells increases at least 40-fold as compared to the number of NK cells prior to administration after five doses. In some embodiments, the combination of heterodimeric proteins is administered at a dose of at least 0.09 mg/kg Q2W and the number of NK cells increases at least 50-fold as compared to the number of NK cells prior to administration after two doses. In some embodiments, the combination of heterodimeric proteins is administered at a dose of at least 0.09 mg/kg Q2W and the number of NK cells increases at least 50-fold as compared to the number of NK cells prior to administration after three doses. In some embodiments, the combination of heterodimeric proteins is administered at a dose of at least 0.09 mg/kg Q2W and the number of NK cells increases at least 50-fold as compared to the number of NK cells prior to administration after four doses. In some embodiments, the combination of heterodimeric proteins is administered at a dose of at least 0.09 mg/kg Q2W and the number of NK cells increases at least 50-fold as compared to the number of NK cells prior to administration after five doses. In some embodiments, the combination of heterodimeric proteins is administered at a dose of at least 0.09 mg/kg Q2W and the number of NK cells increases at least 100-fold as compared to the number of NK cells prior to administration after two doses. In some embodiments, the combination of heterodimeric proteins is administered at a dose of at least 0.09 mg/kg Q2W and the number of NK cells increases at least 100-fold as compared to the number of NK cells prior to administration after three doses. In some embodiments, the combination of heterodimeric proteins is administered at a dose of at least 0.09 mg/kg Q2W and the number of NK cells increases at least 100-fold as compared to the number of NK cells prior to administration after four doses. In some embodiments, the combination of heterodimeric proteins is administered at a dose of at least 0.09 mg/kg Q2W and the number of NK cells increases at least 100-fold as compared to the number of NK cells prior to administration after five doses. In some embodiments, the combination of heterodimeric proteins is administered at a dose of at least 0.09 mg/kg Q2W and the number of NK cells increases at least 200-fold as compared to the number of NK cells prior to administration after two doses. In some embodiments, the combination of heterodimeric proteins is administered at a dose of at least 0.09 mg/kg Q2W and the number of NK cells increases at least 200-fold as compared to the number of NK cells prior to administration after three doses. In some embodiments, the combination of heterodimeric proteins is administered at a dose of at least 0.09 mg/kg Q2W and the number of NK cells increases at least 200-fold as compared to the number of NK cells prior to administration after four doses. In some embodiments, the combination of heterodimeric proteins is administered at a dose of at least 0.09 mg/kg Q2W and the number of NK cells increases at least 200-fold as compared to the number of NK cells prior to administration after five doses.

In some embodiments, the combination of heterodimeric proteins is administered at a dose of at least 0.12 mg/kg Q2W and the number of NK cells increases at least 20-fold as compared to the number of NK cells prior to administration after two doses. In some embodiments, the combination of heterodimeric proteins is administered at a dose of at least 0.12 mg/kg Q2W and the number of NK cells increases at least 20-fold as compared to the number of NK cells prior to administration after three doses. In some embodiments, the combination of heterodimeric proteins is administered at a dose of at least 0.12 mg/kg Q2W and the number of NK cells increases at least 20-fold as compared to the number of NK cells prior to administration after four doses. In some embodiments, the combination of heterodimeric proteins is administered at a dose of at least 0.12 mg/kg Q2W and the number of NK cells increases at least 20-fold as compared to the number of NK cells prior to administration after five doses. In some embodiments, the combination of heterodimeric proteins is administered at a dose of at least 0.12 mg/kg Q2W and the number of NK cells increases at least 30-fold as compared to the number of NK cells prior to administration after two doses. In some embodiments, the combination of heterodimeric proteins is administered at a dose of at least 0.12 mg/kg Q2W and the number of NK cells increases at least 30-fold as compared to the number of NK cells prior to administration after three doses. In some embodiments, the combination of heterodimeric proteins is administered at a dose of at least 0.12 mg/kg Q2W and the number of NK cells increases at least 30-fold as compared to the number of NK cells prior to administration after four doses. In some embodiments, the combination of heterodimeric proteins is administered at a dose of at least 0.12 mg/kg Q2W and the number of NK cells increases at least 30-fold as compared to the number of NK cells prior to administration after five doses. In some embodiments, the combination of heterodimeric proteins is administered at a dose of at least 0.12 mg/kg Q2W and the number of NK cells increases at least 40-fold as compared to the number of NK cells prior to administration after two doses. In some embodiments, the combination of heterodimeric proteins is administered at a dose of at least 0.12 mg/kg Q2W and the number of NK cells increases at least 40-fold as compared to the number of NK cells prior to administration after three doses. In some embodiments, the combination of heterodimeric proteins is administered at a dose of at least 0.12 mg/kg Q2W and the number of NK cells increases at least 40-fold as compared to the number of NK cells prior to administration after four doses. In some embodiments, the combination of heterodimeric proteins is administered at a dose of at least 0.12 mg/kg Q2W and the number of NK cells increases at least 40-fold as compared to the number of NK cells prior to administration after five doses. In some embodiments, the combination of heterodimeric proteins is administered at a dose of at least 0.12 mg/kg Q2W and the number of NK cells increases at least 50-fold as compared to the number of NK cells prior to administration after two doses. In some embodiments, the combination of heterodimeric proteins is administered at a dose of at least 0.12 mg/kg Q2W and the number of NK cells increases at least 50-fold as compared to the number of NK cells prior to administration after three doses. In some embodiments, the combination of heterodimeric proteins is administered at a dose of at least 0.12 mg/kg Q2W and the number of NK cells increases at least 50-fold as compared to the number of NK cells prior to administration after four doses. In some embodiments, the combination of heterodimeric proteins is administered at a dose of at least 0.12 mg/kg Q2W and the number of NK cells increases at least 50-fold as compared to the number of NK cells prior to administration after five doses. In some embodiments, the combination of heterodimeric proteins is administered at a dose of at least 0.12 mg/kg Q2W and the number of NK cells increases at least 100-fold as compared to the number of NK cells prior to administration after two doses. In some embodiments, the combination of heterodimeric proteins is administered at a dose of at least 0.12 mg/kg Q2W and the number of NK cells increases at least 100-fold as compared to the number of NK cells prior to administration after three doses. In some embodiments, the combination of heterodimeric proteins is administered at a dose of at least 0.12 mg/kg Q2W and the number of NK cells increases at least 100-fold as compared to the number of NK cells prior to administration after four doses. In some embodiments, the combination of heterodimeric proteins is administered at a dose of at least 0.12 mg/kg Q2W and the number of NK cells increases at least 100-fold as compared to the number of NK cells prior to administration after five doses. In some embodiments, the combination of heterodimeric proteins is administered at a dose of at least 0.12 mg/kg Q2W and the number of NK cells increases at least 200-fold as compared to the number of NK cells prior to administration after two doses. In some embodiments, the combination of heterodimeric proteins is administered at a dose of at least 0.12 mg/kg Q2W and the number of NK cells increases at least 200-fold as compared to the number of NK cells prior to administration after three doses. In some embodiments, the combination of heterodimeric proteins is administered at a dose of at least 0.12 mg/kg Q2W and the number of NK cells increases at least 200-fold as compared to the number of NK cells prior to administration after four doses. In some embodiments, the combination of heterodimeric proteins is administered at a dose of at least 0.12 mg/kg Q2W and the number of NK cells increases at least 200-fold as compared to the number of NK cells prior to administration after five doses.

In some embodiments, the NK cells are CD16+ NK cells. In some embodiments, the NK cells are CD16+/CD56+ NK cells. In some embodiments, the NK cells are CD16+/CD56− NK cells. In some embodiments, the NK cells are CD16+/CD56ₗₒ NK cells. In some embodiments, the NK cells are CD16+/CD56_{intermediate} NK cells. In some embodiments, the NK cells are CD16+/CD56ₕᵢ NK cells. In some embodiments, the NK cells are CD3−/CD16+/CD56+ NK cells. In some embodiments, the NK cells are CD3−/CD16+/CD56− NK cells. In some embodiments, the NK cells are CD3−/CD16+/CD56ₗₒ NK cells. In some embodiments, the NK cells are CD3−/CD16+/CD56_{intermediate} NK cells. In some embodiments, the NK cells are CD3−/CD16+/CD56ₕᵢ NK cells.

In some embodiments, the first monomer comprises an amino acid sequence encoded by a polynucleotide that encodes the amino acid sequence set forth in SEQ ID NO: 9. In some embodiments, the first monomer comprises an amino acid sequence encoded by a polynucleotide that encodes the amino acid sequence set forth in SEQ ID NO: 57. In some embodiments, the second monomer comprises an amino acid sequence encoded by a polynucleotide that encodes the amino acid sequence set forth in SEQ ID NO: 10. In some embodiments, the second monomer comprises an amino acid sequence encoded by a polynucleotide that encodes the amino acid sequence set forth in SEQ ID NO: 58. In some embodiments, the second monomer comprises an amino acid sequence encoded by a polynucleotide that encodes the amino acid sequence set forth in SEQ ID NO: 16. In some embodiments, the second monomer comprises an amino acid sequence encoded by a polynucleotide that encodes the amino acid sequence set forth in SEQ ID NO: 59.

In some embodiments, the first monomer comprises the amino acid sequence set forth in SEQ ID NO: 9. In some embodiments, the first monomer comprises the amino acid sequence set forth in SEQ ID NO: 57. In some embodiments, the second monomer comprises the amino acid sequence set forth in SEQ ID NO: 10. In some embodiments, the second monomer comprises the amino acid sequence set forth in SEQ ID NO: 58. In some embodiments, the second monomer comprises the amino acid sequence set forth in SEQ ID NO: 16. In some embodiments, the second monomer comprises the amino acid sequence set forth in SEQ ID NO: 59. In some embodiments, the heterodimeric protein is XENP24306, XENP32803, or a combination thereof. In some embodiments, the heterodimeric protein is XENP24306 with a C-terminal lysine, XENP24306 without a C-terminal lysine, XENP32803 with a C-terminal lysine, XENP32803 without a C-terminal lysine, or any combination thereof.

In some embodiments, the first monomer lacks a C-terminal lysine. In some embodiments, the second monomer comprises a C-terminal lysine. In some embodiments, the second monomer lacks a C-terminal lysine. In some embodiments of any of the methods disclosed herein, a combination of XENP24306 with a C-terminal lysine and XENP24306 without a C-terminal lysine. In some embodiments of any of the methods disclosed herein, the heterodimeric protein is XENP32803 with a C-terminal lysine and XENP32803 without a C-terminal lysine. In some embodiments of any of the methods disclosed herein, a combination of (1) XENP24306 with a C-terminal lysine and XENP24306 without a C-terminal lysine; and (2) XENP32803 with a C-terminal lysine and XENP32803 without a C-terminal lysine are used. In some embodiments of any of the methods disclosed herein, a combination of (1) XENP24306 with a C-terminal lysine; and (2) XENP32803 with a C-terminal lysine are used. In some embodiments of any of the methods disclosed herein, a combination of (1) XENP24306 with a C-terminal lysine; and (2) XENP32803 without a C-terminal lysine are used. In some embodiments of any of the methods disclosed herein, a combination of (1) XENP24306 with a C-terminal lysine; and (2) XENP32803 with a C-terminal lysine and XENP32803 without a C-terminal lysine are used. In some embodiments of any of the methods disclosed herein, a combination of (1) XENP24306 without a C-terminal lysine; and (2) XENP32803 with a C-terminal lysine are used. In some embodiments of any of the methods disclosed herein, a combination of (1) XENP24306 without a C-terminal lysine; and (2) XENP32803 without a C-terminal lysine are used. In some embodiments of any of the methods disclosed herein, a combination of (1) XENP24306 without a C-terminal lysine; and (2) XENP32803 with a C-terminal lysine and XENP32803 without a C-terminal lysine are used.

In some embodiments, the first monomer comprises an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1. In some embodiments, the second monomer comprises an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1. In some embodiments, the first Fc domain comprises amino acid substitutions L368D and K370S; the second Fc domain comprises amino acid substitutions S364K and E357Q; and each of said first and second Fc domains further comprises amino acid substitutions C220S, E233P, L234V, L235A, G236 del, S267K, M428L and N434S, according to EU numbering. In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, the first monomer comprises the amino acid sequence set forth in SEQ ID NO: 9, and the second monomer comprises the amino acid sequence set forth in SEQ ID NO: 10. In some embodiments, the first monomer comprises the amino acid sequence set forth in SEQ ID NO: 57, and the second monomer comprises the amino acid sequence set forth in SEQ ID NO: 10. In some embodiments, the first monomer comprises the amino acid sequence set forth in SEQ ID NO: 9, and the second monomer comprises the amino acid sequence set forth in SEQ ID NO: 58. In some embodiments, the first monomer comprises the amino acid sequence set forth in SEQ ID NO: 57, and the second monomer comprises the amino acid sequence set forth in SEQ ID NO: 58. In some embodiments, the first monomer comprises the amino acid sequence set forth in SEQ ID NO: 9, and the second monomer comprises the amino acid sequence set forth in SEQ ID NO: 16. In some embodiments, the first monomer comprises the amino acid sequence set forth in SEQ ID NO: 57, and the second monomer comprises the amino acid sequence set forth in SEQ ID NO: 16. In some embodiments, the first monomer comprises the amino acid sequence set forth in SEQ ID NO: 9, and the second monomer comprises the amino acid sequence set forth in SEQ ID NO: 59. In some embodiments, the first monomer comprises the amino acid sequence set forth in SEQ ID NO: 57, and the second monomer comprises the amino acid sequence set forth in SEQ ID NO: 59.

In some embodiments, two or more of the heterodimeric proteins as disclosed herein are administered to the subject. In some embodiments, three or more of the heterodimeric proteins as disclosed herein are administered to the subject. In some embodiments, four or more of the heterodimeric proteins as disclosed herein are administered to the subject. In some embodiments, five or more of the heterodimeric proteins as disclosed herein are administered to the subject.

In some embodiments, a combination of a first heterodimeric protein and a second heterodimeric protein is administered to the subject. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 9, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 9, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 59. In some embodiments, the first heterodimeric protein comprises a first monomer comprising the amino acid sequence set forth in SEQ ID NO. 9, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 16. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 9, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 10; and a second heterodimeric protein comprises a first monomer comprising the amino acid sequence set forth in SEQ ID NO: 57, and a second monomer comprising the amino acid sequence set forth in SEQ ID NO: 59. In some embodiments, the first heterodimeric protein comprises a first monomer comprising the amino acid sequence set forth in SEQ ID NO: 57, and a second monomer comprising the amino acid sequence set forth in SEQ ID NO: 58; and a second heterodimeric protein comprises a first monomer comprising the amino acid sequence set forth in SEQ ID NO: 57, and a second monomer comprising the amino acid sequence set forth in SEQ ID NO: 59.

Programmed death-ligand-1 (PD-L1) is a cell-surface protein that is broadly expressed by tumor cells and tumor-infiltrating immune cells in many human cancers. Overexpression of PD-L1 has been associated with poor prognosis in patients with some cancers. PD-L1 binds to PD-1 and B7.1, two known receptors whose expression on activated T cells is sustained in states of chronic stimulation, such as chronic infection or cancer. Ligation of PD-L1 with PD-1 or B7.1 inhibits T cell proliferation, cytokine production, and cytolytic activity, which leads to a functional inactivation or inhibition of T cells. Aberrant expression of PD-L1 on tumor cells has been reported to impede antitumor immunity resulting in immune evasion. Interruption of the PD-L1/PD-1 and PD-L1/B7.1 pathways is an attractive strategy for reinvigorating tumor-specific T cell immunity, and indeed, multiple inhibitors of PD-L1 or PD-1 have demonstrated clinical efficacy or promising antitumor activity in a wide range of tumor types, including melanoma, RCC, NSCLC, SCLC, urothelial bladder cancer, HNSCC, ovarian cancer, and TNBC. The evidenced benefit has led to approvals of multiple anti-PD-L1 antibodies (e.g., atezolizumab, avelumab, and durvalumab), and anti-PD-1 antibodies (e.g., nivolumab, pembrolizumab, and cemiplimab-rwlc) in select indications to date.

In some embodiments, the agent targeting the PD-L1/PD-1 axis is an inhibitor of PD-1. In some embodiments, the agent targeting the PD-L1/PD-1 axis is an inhibitor of PD-L1.

In some embodiments, the inhibitor of PD-1 is an anti-PD-1 antibody. Antibodies which specifically bind to PD-1 are known in the art and have been described, for example, in Naidoo et al. Ann Oncol. 2015; 26(12): 2375-2391, Philips et al. Int Immunol. 2015; 27(1):39-46, Tunger et al. J Clin Med. 2019; 8(10) and Sunshine et al. Curr Opin Pharmacol. 2015; 32-8; and U.S. Pat. Nos. 8,008,449, 8,168,757, US 20110008369, US 20130017199, US 20130022595, and in WO2006121168, WO20091154335, WO2012145493, WO2013014668, WO2009101611, EP2262837, and EP2504028. Examples of anti-PD-1 antibodies include, but are not limited to, nivolumab (BMS-936558; MDX-1106), pembrolizumab (Trade name Keytruda formerly lambrolizumab; also known as Merck 3475 and SCH-900475), pidilizumab (CT-011), cemiplimab, spartalizumab (PDR001), camrelizumab (SHR1210), sintilimab (IB1308), tislelizumab (BGB-A317), toripalimab (JS 001), and AMP-514 (Amplimmune). Nivolumab is an anti-PD-1 antibody described in WO2006/121168. Pembrolizumab is an anti-PD-1 antibody described in WO2009/114335 and Hamid et al. (2013). New England Journal of Medicine 369 (2): 134-44. Pidilizumab is a humanized IgG-1κ monoclonal antibody that binds to PD-1. Pidilizumab and other humanized anti-PD1 monoclonal antibodies are disclosed in WO2009/101611. Other anti-PD-1 antibodies include AMP 514, among others, e.g., anti-PD-1 antibodies disclosed in U.S. Pat. No. 8,609,089, US 2010028330 and/or US 20120114649. In some embodiments, the anti-PD-1 antibody is nivolumab. In some embodiments, the checkpoint inhibitor that targets PD-1 is a fusion protein, e.g. AMP-224. AMP-224 (Amplimmune) is a PD-L2 Fc fusion soluble receptor that blocks the interaction between PD-1 and B7-H1 and is disclosed in WO2010/027827 and WO2011/066342. In some embodiments, the anti-PD-1 antibody is administered in combination with XENP24306. In some embodiments, the anti-PD-1 antibody is administered in combination with XENP32803. In some embodiments, the anti-PD-1 antibody is administered in combination with XENP24306 and XENP32803. In some embodiments, nivolumab is administered in combination with XENP24306. In some embodiments, nivolumab is administered in combination with XENP32803. In some embodiments, nivolumab is administered in combination with XENP24306 and XENP32803.

In some embodiments, the inhibitor of PD-L1 is an anti-PD-L1 antibody. Antibodies which specifically bind to PD-L1 are known in the art and have been described, for example, in Naidoo et al. Ann Oncol. 2015 December; 26(12): 2375-2391, Philips et al. Int Immunol. 2015 January; 27(1):39-46, Tunger et al. J Clin Med. 2019 Sep. 25; 8(10), Sunshine et al. Curr Opin Pharmacol. 2015:32-8 and U.S. Pat. No. 7,943,743 and U.S Publication No. 20120039906. Examples of anti-PD-L1 antibodies include, but are not limited to, BMS-936559 (also known as MDX-1105), BMS-39886, atezolizumab (MDPL3280A; Tecentriq), avelumab (MSB0010718C; Bavencio), durvalumab (MEDI4736; Imfinzi), envafolimab (KN035), and cosibelimab (CK-301 Checkpoint Therapeutics). BMS-936559 is an anti-PD-L1 antibody described in WO2007/005874. Atezolizumab is a humanized monoclonal antibody with a human Fc optimized IgG1 that binds to PD-L1. BMS-39886 is an anti-PD-L1 antibody described in Brahmer J R et al. N Engl J Med 2012; 366: 2455-2465. In some embodiments, the anti-PD-L1 antibody is atezolizumab. In some embodiments, the anti-PD-L1 antibody is administered in combination with XENP24306. In some embodiments, the anti-PD-L1 antibody is administered in combination with XENP32803. In some embodiments, the anti-PD-L1 antibody is administered in combination with XENP24306 and XENP32803. In some embodiments, atezolizumab is administered in combination with XENP24306. In some embodiments, atezolizumab is administered in combination with XENP32803. In some embodiments, atezolizumab is administered in combination with XENP24306 and XENP32803.

The heterodimeric protein (or combination thereof) may be administered parenterally. In some embodiments, the parenteral administration is intravenous. In some embodiments, the heterodimeric protein is administered intravenously. In some embodiments, the heterodimeric protein is administered as a composition comprising a pharmaceutically acceptable buffer. Suitable carriers and their formulations are described, for example, in Remington's Pharmaceutical Sciences by E. W. Martin. In some embodiments, the heterodimeric protein is provided in a dosage form that is suitable for parenteral (e.g. intravenous) administration.

The agent targeting the PD-L1/PD-1 axis may be administered parenterally. In some embodiments, the parenteral administration is intravenous. In some embodiments, the parenteral administration is subcutaneous. In some embodiments, the agent targeting the PD-L1/PD-1 axis is administered intravenously. In some embodiments, the agent targeting the PD-L1/PD-1 axis is administered subcutaneously. In some embodiments, the agent targeting the PD-L1/PD-1 axis is administered as a composition comprising a pharmaceutically acceptable buffer. Suitable carriers and their formulations are described, for example, in Remington's Pharmaceutical Sciences by E. W. Martin. In some embodiments, the agent targeting the PD-L1/PD-1 axis is provided in a dosage form that is suitable for parenteral (e.g. intravenous or subcutaneous) administration.

The amount of the agent targeting the PD-L1/PD-1 axis to be administered in combination with the heterodimeric protein of the disclosure (or combinations thereof) varies depending upon the manner of administration, the age and body weight of the patient, and the clinical symptoms of the cancer to be treated. In some embodiments, the anti-PD-1 antibody or anti-PD-L1 antibody is administered at its approved dosage. A physician will be able to determine the adequate dosage to administer in combination with the protein of the disclosure. In some embodiments, the agent targeting the PD-L1/PD-1 axis is administered using an approved dosage regimen. In certain embodiments, the dosage may vary from between about 0.5 mg protein/kg body weight to about 100 mg compound/kg body weight; or from about 1 mg protein/kg body weight to about 100 mg compound/kg body weight; or from about 2 mg protein/kg body weight to about 50 mg compound/kg body weight; or from about 2.5 mg protein/kg body weight to about 10 mg compound/kg body weight or from about 3 mg protein/kg body weight to about 5 mg compound/kg body weight. In some embodiments, this dose may be about 0.1, about 0.3, about 0.5, about 1, about 3, about 5, about 7.5, about 10, about 15, about 25, about 50, about 75, about 100 mg/kg body weight. In some embodiments, the dosage of the anti-PD-1 antibody is 3 mg/kg. In some embodiments, the dosage of nivolumab is about 3 mg/kg. In some embodiments, the dosage of nivolumab is about 3 mg/kg every two weeks. In some embodiments, the dosage of nivolumab is about 1 mg/kg. In some embodiments, the dosage of nivolumab is about 240 mg. In some embodiments, the dosage of nivolumab is about 480 mg. In some embodiments, the dosage of nivolumab is about 240 mg every two weeks. In some embodiments, the dosage of nivolumab is about 480 mg every four weeks. In some embodiments, the dosage of the anti-PD-L1 antibody is about 3 mg/kg. In some embodiments, the dosage of the anti-PD-L1 antibody is about 840 mg. In some embodiments, the dosage of atezolizumab is about 840 mg. In some embodiments, the dosage of atezolizumab is about 1200 mg. In some embodiments, the dosage of atezolizumab is about 1680 mg. In some embodiments, the dosage of atezolizumab is about 840 mg every 2 weeks. In some embodiments, the dosage of atezolizumab is about 1200 mg every 3 weeks. In some embodiments, the dosage of atezolizumab is about 1680 mg every 4 weeks. In some embodiments, the dosage of pembrolizumab is about 200 mg. In some embodiments, the dosage of pembrolizumab is about 200 mg every three weeks. In some embodiments, the dosage of pembrolizumab is about 200 mg every two weeks. In some embodiments, the dosage of pembrolizumab is about 200 mg every week.

In certain embodiments, the dosage may vary from between 0.5 mg protein/kg body weight to 100 mg compound/kg body weight; or from 1 mg protein/kg body weight to 100 mg compound/kg body weight; or from 2 mg protein/kg body weight to 50 mg compound/kg body weight; or from 2.5 mg protein/kg body weight to 10 mg compound/kg body weight or from 3 mg protein/kg body weight to 5 mg compound/kg body weight. In some embodiments, this dose may be 0.1, 0.3, 0.5, 1, 3, 5, 7.5, 10, 15, 25, 50, 75, 100 mg/kg body weight. In some embodiments, the dosage of the anti-PD-1 antibody is 3 mg/kg. In some embodiments, the dosage of nivolumab is 3 mg/kg. In some embodiments, the dosage of nivolumab is 3 mg/kg every two weeks. In some embodiments, the dosage of nivolumab is 1 mg/kg. In some embodiments, the dosage of nivolumab is 240 mg. In some embodiments, the dosage of nivolumab is 480 mg. In some embodiments, the dosage of nivolumab is 240 mg every two weeks. In some embodiments, the dosage of nivolumab is 480 mg every four weeks. In some embodiments, the dosage of the anti-PD-L1 antibody is 3 mg/kg. In some embodiments, the dosage of the anti-PD-L1 antibody is 840 mg. In some embodiments, the dosage of atezolizumab is 840 mg. In some embodiments, the dosage of atezolizumab is 1200 mg. In some embodiments, the dosage of atezolizumab is 1680 mg. In some embodiments, the dosage of atezolizumab is 840 mg every 2 weeks. In some embodiments, the dosage of atezolizumab is 1200 mg every 3 weeks. In some embodiments, the dosage of atezolizumab is 1680 mg every 4 weeks. In some embodiments, the dosage of pembrolizumab is 200 mg. In some embodiments, the dosage of pembrolizumab is 200 mg every three weeks. In some embodiments, the dosage of pembrolizumab is 200 mg every two weeks. In some embodiments, the dosage of pembrolizumab is 200 mg every week.

The heterodimeric proteins disclosed herein, or combinations thereof, may be administered simultaneously or sequentially with an agent targeting the PD-L1/PD-1 axis (such as an anti-PD1 or anti-PD-L1 antibody). In some embodiments, the agent targeting the PD-L1/PD-1 axis is administered after administering the heterodimeric protein. In some embodiments, the agent targeting the PD-L1/PD-1 axis is administered before administering the heterodimeric protein. In some embodiments, the heterodimeric proteins disclosed herein or combinations thereof and the agent targeting the PD-L1/PD-1 axis (such as an anti-PD1 or anti-PD-L1 antibody) are administered in the same composition. In some embodiments, the heterodimeric proteins disclosed herein, or combinations thereof, are administered in a different composition than the agent targeting the PD-L1/PD-1 axis (such as an anti-PD1 or anti-PD-L1 antibody).

In some embodiments, the treatment using the agent targeting the PD-L1/PD-1 axis is an established therapy for the cancer and addition of the heterodimeric protein treatment to the regimen improves the therapeutic benefit to the patients. Such improvement could be measured as increased responses on a per patient basis or increased responses in the patient population. The heterodimeric proteins disclosed herein or combinations thereof and the agent targeting the PD-L1/PD-1 axis may synergize. In some embodiments, the heterodimeric proteins disclosed herein, or combinations thereof, may be administered at a dosage less than its therapeutically effective dose when administered as a monotherapy. In some embodiments, the agent targeting the PD-L1/PD-1 axis may be administered at a dosage less than its therapeutically effective dose when administered as a monotherapy.

In some embodiments, the agent targeting the PD-L1/PD-1 axis is administered by IV infusion. In some embodiments, the agent targeting the PD-L1/PD-1 axis is administered by IV infusion at a fixed dose on Day 1 of each 14-day cycle in combination with the heterodimeric protein of the disclosure. In some embodiments, atezolizumab is administered at a dose of about 840 mg on day 1 of each 14-day cycle in combination with the heterodimeric protein of the disclosure. In some embodiments, atezolizumab is administered at a dose of 840 mg on day 1 of each 14-day cycle in combination with the heterodimeric protein of the disclosure. In some embodiments, atezolizumab is administered using the approved dosage regimen. In some embodiments, nivolumab is administered using the approved dosage regimen. In some embodiments, pembrolizumab is administered using the approved dosage regimen.

In some embodiments, prior to administration of the plurality of doses of the heterodimeric protein, the subject has not been previously administered an agent to treat the solid tumor. In some embodiments, the subject is currently being administered a checkpoint inhibitor, i.e., the subject is receiving the checkpoint inhibitor along with the heterodimeric protein of the disclosure, either as part of the same or a different treatment regimen. In some embodiments, prior to administration of the plurality of doses of the heterodimeric protein, the subject has previously been administered a checkpoint inhibitor. In some embodiments, the checkpoint inhibitor targets PD-1. In some embodiments, the checkpoint inhibitor targets PD-L1. In some embodiments, the checkpoint inhibitor targets CTLA-4.

Examples of solid tumors to be treated by the combination of the heterodimeric proteins of the disclosure and an agent targeting the PD-L1/PD-1 axis (such as an anti-PD1 or anti-PD-L1 antibody) include, but are not limited, to carcinomas, lymphomas, blastomas and sarcomas. More particular examples of such solid tumors include squamous cell cancer, cutaneous squamous cell cancer (cSCC), small-cell lung carcinoma (SCLC), non-small cell lung cancer (NSCLC), gastrointestinal cancer, gastric cancer (GC), pancreatic cancer, glioblastoma, cervical cancer, ovarian cancer, liver cancer, bladder cancer, liposarcoma, soft-tissue sarcoma, urothelial carcinoma (UCC), ureter and renal pelvis, multiple myeloma, osteosarcoma, hepatoma, melanoma, stomach cancer, breast cancer, colon cancer, colorectal cancer, endometrial carcinoma, salivary gland carcinoma, renal cell carcinoma (RCC), liver cancer, esophageal cancer, prostate cancer, vulval cancer, thyroid cancer, hepatic carcinoma, Merkel cell carcinoma (MCC), germ cell cancer, micro-satellite instability-high (MSI-H) cancer and head and neck cancer. In some embodiments, the solid tumor is a locally advanced, recurrent, or metastatic incurable solid tumor. In some embodiments, the solid tumor is selected from the group consisting of melanoma, NSCLC, head and neck squamous cell carcinoma (HNSCC), triple-negative breast cancer (TNBC), UCC, RCC, SCLC, GC, MCC, cSCC and MSI-H cancers. In some embodiments, the solid tumor is selected from melanoma, renal cell carcinoma (RCC), NSCLC, head and neck squamous cell carcinoma (HNSCC), and triple negative breast cancer. In some embodiments, the solid tumor is selected from melanoma, RCC, NSCLC, HNSCC and TNBC. In some embodiments, the solid tumor is selected from melanoma, RCC, and NSCLC. In some embodiments, the solid tumor is selected from melanoma, NSCLC, HNSCC and TNBC. In some embodiments, the solid tumor is melanoma. In some embodiments, the solid tumor is RCC. In some embodiments, the cancer is NSCLC. In some embodiments, the solid tumor is HNSCC. In some embodiments, the solid tumor is TNBC. In some embodiments, the solid tumor is a solid tumor for which standard therapy does not exist, has proven to be ineffective or intolerable, or is considered inappropriate, or for whom a clinical trial of an investigational agent is a recognized standard of care.

A combination therapy could also provide improved responses at lower or less frequent doses of the agent targeting the PD-L1/PD-1 axis (such as an anti-PD1 or anti-PD-L1 antibody) resulting in a better tolerated treatment regimen. For example, the combined therapy of the heterodimeric protein(s) and an agent targeting the PD-L1/PD-1 axis (such as an anti-PD1 or anti-PD-L1 antibody) could provide enhanced clinical activity through various mechanisms, including augmented ADCC, ADCP, and/or NK cell, T cell, neutrophil or monocytic cell levels or immune responses.

### Exemplary Embodiments

Particular embodiments of the disclosure are set forth in the following numbered embodiments:
1. A method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising the amino acid sequence set forth in SEQ ID NO: 9; and (ii) a second monomer comprising the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.
2. A method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising an amino acid sequence encoded by a polynucleotide encoding the amino acid sequence set forth in SEQ ID NO: 9; and (ii) a second monomer comprising an amino acid sequence encoded by a polynucleotide encoding the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.
3. A method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein is sufficient to increase the number of NK cells at least 20-fold as compared to the number of NK cells prior to said administration.
4. The method according to embodiment 3, wherein the first Fc domain of the heterodimeric protein comprises the amino acid sequence set forth in SEQ ID NO: 6.
5. The method according to embodiment 3 or 4, wherein the second Fc domain of the heterodimeric protein comprises the amino acid sequence set forth in SEQ ID NO: 7.
6. The method according to embodiment 3 or 4, wherein the second Fc domain of the heterodimeric protein comprises the amino acid sequence set forth in SEQ ID NO: 8.
7. A method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.
8. A method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising an amino acid sequence encoded by a polynucleotide encoding the amino acid sequence set forth in SEQ ID NO: 9 and (ii) a second monomer comprising an amino acid sequence encoded by a polynucleotide encoding the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.
9. A method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein is sufficient to increase the number of NK cells at least 20-fold as compared to the number of NK cells prior to said administration.
10. The method according to embodiment 9, wherein the first Fc domain of the heterodimeric protein comprises the amino acid sequence set forth in SEQ ID NO: 6.
11. The method according to embodiment 9 or 10, wherein the second Fc domain of the heterodimeric protein comprises the amino acid sequence set forth in SEQ ID NO: 7.
12. The method according to embodiment 9 or 10, wherein the second Fc domain of the heterodimeric protein comprises the amino acid sequence set forth in SEQ ID NO: 8.
13. The method according to any one of embodiments 7-12, wherein the subject is suffering from a solid tumor.
14. The method according to any one of embodiments 1-13, wherein the number of NK cells increases at least 30-fold, 40-fold, 50-fold, 60-fold, 70-fold, 80-fold, 90-fold, 100-fold, 150-fold, 200-fold, 250-fold, or 300-fold relative to the number of NK cells prior to administration.
15. The method according to any one of embodiments 1-13, wherein the heterodimeric protein is administered at a dose of at least 0.04 mg/kg Q2W and the number of NK cells increases at least 20-fold after two doses.
16. The method according to any one of embodiments 1-14, wherein the heterodimeric protein is administered at a dose of at least 0.06 mg/kg Q2W and the number of NK cells increases at least 50-fold after two doses.
17. The method according to any one of embodiments 1-14, wherein the heterodimeric protein is administered at a dose of at least 0.06 mg/kg Q2W and the number of NK cells increases at least 100-fold after three doses.
18. A method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein results in accumulation of NK cells in the subject.
19. A method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising an amino acid sequence encoded by a polynucleotide encoding the amino acid sequence set forth in SEQ ID NO: 9; and (ii) a second monomer comprising an amino acid sequence encoded by a polynucleotide encoding the amino acid sequence set forth in SEQ ID NO: 10 or 16; wherein the administration of the plurality of doses of the heterodimeric protein results in accumulation of NK cells in the subject.
20. A method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein results in accumulation of NK cells in the subject.
21. The method according to embodiment 20, wherein the first Fc domain of the heterodimeric protein comprises the amino acid sequence set forth in SEQ ID NO: 6.
22. The method according to embodiment 20 or 21, wherein the second Fc domain of the heterodimeric protein comprises the amino acid sequence set forth in SEQ ID NO: 7.
23. The method according to embodiment 20 or 21, wherein the second Fc domain of the heterodimeric protein comprises the amino acid sequence set forth in SEQ ID NO: 8.
24. A method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising the amino acid sequence set forth in SEQ TD NO: 9 and (ii) a second monomer comprising the amino acid sequence set forth in SEQ ID NO: 10 or 16; wherein the administration of the plurality of doses of the heterodimeric protein results in accumulation of NK cells in the subject.
25. A method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising an amino acid sequence encoded by a polynucleotide encoding the amino acid sequence set forth in SEQ ID NO: 9; and (ii) a second monomer comprising an amino acid sequence encoded by a polynucleotide encoding the amino acid sequence set forth in SEQ ID NO: 10 or 16; wherein the administration of the plurality of doses of the heterodimeric protein results in accumulation of NK cells in the subject.
26. A method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein results in accumulation of NK cells in the subject.
27. The method according to embodiment 26, wherein the first Fc domain of the heterodimeric protein comprises the amino acid sequence set forth in SEQ ID NO: 6.
28. The method according to embodiment 26 or 27, wherein the second Fc domain of the heterodimeric protein comprises the amino acid sequence set forth in SEQ ID NO: 7.
29. The method according to embodiment 26 or 27, wherein the second Fc domain of the heterodimeric protein comprises the amino acid sequence set forth in SEQ ID NO: 8.
30. The method according to any one of embodiments 24-29, wherein the subject is suffering from a solid tumor.
31. The method according to any one of embodiments 18-30, wherein the accumulation is for at least one cycle.
32. The method according to embodiment 31, wherein the accumulation is for at least two cycles, at least three cycles, or at least four cycles.
33. The method according to any one of embodiments 1-32, wherein the NK cells are CD16+ NK cells.
34. The method according to any one of embodiments 1-33, wherein the first monomer comprises the amino acid sequence set forth in SEQ ID NO: 57.
35. The method according to any one of embodiments 1-34, wherein the second monomer comprises the amino acid sequence set forth in SEQ ID NO: 10.
36. The method according to any one of embodiments 1-34, wherein the second monomer comprises the amino acid sequence set forth in SEQ ID NO: 58.
37. The method according to any one of embodiments 1-34, wherein the second monomer comprises the amino acid sequence set forth in SEQ ID NO: 16.
38. The method according to any one of embodiments 1-34, wherein the second monomer comprises the amino acid sequence set forth in SEQ ID NO: 59.
39. The method according to any one of embodiments 1-38, wherein said heterodimeric protein is XENP24306 with a C-terminal lysine, XENP24306 without a C-terminal lysine, XENP32803 with a C-terminal lysine, XENP32803 without a C-terminal lysine, or any combination thereof.
40. The method according to any one of embodiments 1-39, wherein a combination of a first heterodimeric protein and a second heterodimeric protein is administered to the subject.
41. The method according to embodiment 40, wherein the first heterodimeric protein comprises a first monomer comprising the amino acid sequence set forth in SEQ ID NO: 9, and a second monomer comprising the amino acid sequence set forth in SEQ ID NO: 10; and the second heterodimeric protein comprises a first monomer comprising the amino acid sequence set forth in SEQ ID NO: 9, and a second monomer comprising the amino acid sequence set forth in SEQ ID NO: 16.
42. The method according to embodiment 40 or 41, wherein said first and second heterodimeric proteins are administered simultaneously.
43. The method according to embodiment 40 or 41, wherein said first and second heterodimeric proteins are administered sequentially.
44. The method according to any one of embodiments 1-6, 13-23 and 30-43, wherein said solid tumor is locally advanced, recurrent or metastatic.
45. The method according to any one of embodiments 1-6, 13-23 and 30-43, wherein said solid tumor is selected from the group consisting of squamous cell cancer, cutaneous squamous cell cancer, small-cell lung cancer, non-small cell lung cancer, gastrointestinal cancer, gastric cancer, pancreatic cancer, glioblastoma, cervical cancer, ovarian cancer, liver cancer, bladder cancer, liposarcoma, soft-tissue sarcoma, urothelial carcinoma, ureter and renal pelvis, multiple myeloma, osteosarcoma, hepatoma, melanoma, stomach cancer, breast cancer, colon cancer, colorectal cancer, endometrial carcinoma, salivary gland carcinoma, renal cell carcinoma, esophageal cancer, prostate cancer, vulval cancer, thyroid cancer, hepatic carcinoma, Merkel cell carcinoma, germ cell cancer, micro-satellite instability-high cancer and head and neck squamous cell carcinoma.
46. The method according to embodiment 45, wherein said solid tumor is selected from the group consisting of melanoma, renal cell carcinoma, non-small cell lung cancer, head and neck squamous cell carcinoma, and triple negative breast cancer.
47. The method according to embodiment 45, wherein said solid tumor is selected from the group consisting of melanoma, renal cell carcinoma, and non-small cell lung cancer.
48. The method according to embodiment 45, wherein said solid tumor is selected from the group consisting of melanoma, non-small cell lung cancer, head and neck squamous cell carcinoma, and triple negative breast cancer.
49. The method according to any one of embodiments 1-6, 13-23 and 30-48, wherein prior to administration of the plurality of doses of the heterodimeric protein, the subject has not been previously administered an agent to treat the solid tumor.
50. The method according to any one of embodiments 1-6, 13-23 and 30-48, wherein the subject is currently being administered a checkpoint inhibitor.
51. The method according to any one of embodiments 1-6, 13-23 and 30-48, wherein prior to administration of the plurality of doses of the heterodimeric protein, the subject has previously been administered a checkpoint inhibitor.
52. The method according to embodiment 50 or 51, wherein the checkpoint inhibitor targets PD-1.
53. The method according to embodiment 50 or 51, wherein the checkpoint inhibitor targets PD-L1.
54. The method according to embodiment 50 or 51, wherein the checkpoint inhibitor targets CTLA-4.
55. The method according to any one of embodiments 1-14 and 18-54, wherein said heterodimeric protein or combination of heterodimeric proteins is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.09 mg/kg, about 0.1 mg/kg, about 0.12 mg/kg, about 0.16 mg/kg, about 0.2 mg/kg, about 0.24 mg/kg and about 0.32 mg/kg body weight.
56. The method according to embodiment 55, wherein said heterodimeric protein or combination of heterodimeric proteins is administered at a dose selected from the group consisting of about 0.01 mg/kg, about 0.02 mg/kg, about 0.04 mg/kg, about 0.06 mg/kg, about 0.09 mg/kg, and about 0.12 mg/kg body weight.
57. The method according to any one of embodiments 1-14 and 18-56, wherein said heterodimeric protein is administered at a frequency selected from the group consisting of Q1W, Q2W, Q3W, Q4W, Q5W and Q6W.
58. The method according to embodiment 57, wherein said heterodimeric protein is administered at a frequency of Q2W.
59. The method according any one of embodiments 1-58, wherein said method further comprises administering to the subject an agent targeting the PD-L1/PD-1 axis.
60. The method according to embodiment 59, wherein said agent targeting the PD-L1/PD-1 axis is an anti-PD-1 antibody.
61. The method according to embodiment 60, wherein the anti-PD-1 antibody is selected from the group consisting of nivolumab, pembrolizumab, pidilizumab, cemiplimab, spartalizumab, camrelizumab, sintilimab, tislelizumab, toripalimab, and AMP-514.
62. The method according to embodiment 59, wherein said agent targeting the PD-L1/PD-1 axis is an anti-PD-L1 antibody.
63. The method according to embodiment 62, wherein the anti-PD-L1 antibody is selected from the group consisting of avelumab, durvalumab, atezolizumab, BMS-936559, BMS-39886, envafolimab, and cosibelimab.

### EXAMPLES

### Example 1: Non-Clinical Pharmacology of XmAb24306

As detailed below, a combination of IL15/IL15Rα heterodimeric proteins (XENP24306 (˜82%) and XENP32803 (˜18%) (“XENP24306+XENP32803”)) was evaluated in multiple in vitro and in vivo studies to characterize non-clinical pharmacology properties. In vitro studies demonstrated that the combination of IL15/IL15Rα heterodimeric proteins showed binding to human and cynomolgus IL-2/IL-15βγ receptor complex (CD122/CD132), had activity in human and cynomolgus CD8⁺ T cells and NK cells, but was inactive in rodent cells (mouse and rat). XENP24306+XENP32803 showed increased neonatal Fc receptor (FcRn) binding (at pH 6.0) but had no effector function in terms of mediating antibody-dependent cellular cytotoxicity (ADCC) or complement-dependent cytotoxicity (CDC). Both in vitro and in vivo studies showed that XENP24306+XENP32803 preferably expanded CD8⁺ T cells and NK cells, with modest impact on expansion of CD4⁺ T-helper lymphocytes, while having minimal impact on expansion of the Treg population and cytokine release syndrome (CRS)-associated cytokines.

### In Vitro Studies

The IL-15 component of XENP24306 and XENP32803 comprises three amino acid substitutions (D30N, E64Q, and N65D). These substitutions result in reduced potency of IL-15. The binding affinity XENP24306+XENP32803 to human and cynomolgus monkey IL-2/IL-15βγ receptor complex (CD122/CD132) was determined with surface plasmon resonance. Similar binding kinetics and affinities were observed between the two species, establishing the relevancy of cynomolgus monkey as a preclinical animal species for pharmacology and toxicity studies.

XENP24306 and XENP32803 are effectorless, demonstrated by lack of binding to FcγR and human complement component 1q (C1q), and are not expected to induce target-cell killing via ADCC or CDC mechanisms. Specifically, the Fc region XENP24306 and XENP32803 was engineered to remove binding to human, cynomolgus monkey, and mouse FcγR; no binding interactions were detected with the Bio-Layer Interferometry (BLT) method. Binding of XENP24306+XENP32803 to human C1q, a critical component of the C1 complex that initiates the complement system, was also assessed using BLI, and no binding was observed.

Furthermore, the Fc regions of XENP24306 and XENP32803 were engineered to enhance binding to FcRn at a lower pH (6.0) with the goal of extending the half-life of XmAb24306. Binding interactions with human, cynomolgus monkey, and mouse FcRn were determined with the BLI method, and affinities of XENP24306+XENP32803 for these receptors were significantly enhanced at pH 6.0, the physiologically relevant pH for endosome trafficking.

XENP24306+XENP32803-species selectivity was evaluated using a phospho-STAT5 assay. Binding of IL-15/IL-15Rα receptor complex to CD122/CD132-expressing lymphocytes led to activation of the Janus kinase signal transducer and activator of transcription signaling pathway, which resulted in phosphorylation of STAT5 and subsequent cell proliferation. XENP24306+XENP32803 did not induce phosphorylation of STAT5 in mouse or rat CD8⁺ T cells, which thereby precluded use of rodents for toxicity studies or the use of syngeneic mouse models for evaluation of XENP24306+XENP32803 for antitumor efficacy.

Potency of XENP24306+XENP32803 was assessed in in vitro cell proliferation assays. Human CD8⁺ T cells and NK cells showed strong proliferative responses to XENP24306+XENP32803 treatment. Among these two target cell populations, XENP24306+XENP32803 showed relatively higher potency for NK-cell (half maximal effective concentration [EC₅₀]: 1.2 μg/mL) than CD8⁻ T cell (EC₅₀: 12.7 g/mL) proliferation (FIGS. 1A and 1B). In addition to CD8⁺ T cell and NK-cell proliferation, XENP24306+XENP32803 also induced IFNγ production in human PBMCs. XENP24306+XENP32803 also promoted NK-cell (EC₅₀: 0.5 μg/mL) and CD8⁺ T cell (EC₅₀: 3.8 μg/mL) proliferation in cynomolgus monkey PBMCs, which validated cynomolgus monkey as a nonclinical animal species for pharmacology and toxicity studies.

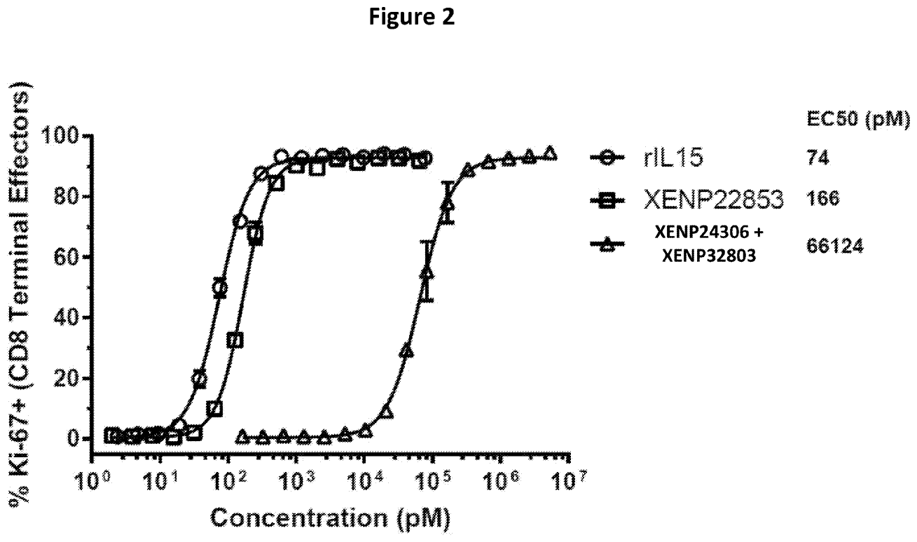

XENP24306 and XENP32803 are potency-reduced, recombinant human IL-15s, designed as IL-15/IL-15Rα heterodimer Fc fusion proteins. Approximately 900-fold lower potency was observed for XENP24306+XENP32803 than recombinant wild-type IL-15 and approximately 400-fold lower potency than recombinant wild-type IL-15 (rIL15) of similar format (wild-type IL-15/wild-type IL-15Rα heterodimer Fc fusion; named as XENP22853; SEQ ID NO: 11 (wild-type IL-15-Fc first monomer) and SEQ ID NO: 7 (IL-15Rα-Fc second monomer)), as shown on CD8⁻ terminal effector T cells (FIG. 2). XENP24306+XENP32803 potency was assessed on different human immune cell subsets. Specifically, Human PBMC were treated with increasing concentrations of XENP24306+XENP32803, recombinant wild-type IL15, or wild-type IL-15/wild-type IL-15Rα heterodimer Fc fusion (XENP22853) for 4 days and assayed by flow cytometry for proliferation through intracellular staining for the cell cycle protein Ki67. FIG. 2 shows results for CD8⁺ terminal effector T cells defined by gating for CD3⁺CD8⁺ CD45RA⁺ CCR7⁻ CD28⁻ CD95⁺ population. Curve fits were generated using the least squares method. EC₅₀ values were determined by non-linear regression analysis using agonist versus response and a variable-slope (four-parameter) equation. XENP24306+XENP32803 enhanced activation of effector memory CD8⁻ and CD4⁺ T cells and NK cells as indicated by increased frequencies of these cell subsets expressing the cell proliferation marker Ki67 and cell activation markers CD69 and CD25. XmAb24306 had minimal effects on naïve CD8⁺ or CD4⁺ T cells.

Two additional in vitro toxicity studies were performed (1) an assessment of the binding profile of XENP24306+XENP32803 using a human plasma membrane protein cell array and (2) an assessment of cytokine release induced by XENP24306+XENP32803, which compared the ability of soluble and immobilized XENP24306+XENP32803 to induce cytokine production. Data from multiple experiments using an optimized concentration of XENP24306+XENP32803 (20 μg/mL) showed that there were no convincing off-target binding interactions identified for XENP24306+XENP32803. Potential risk of Cytokine release syndrome (CRS) with XENP24306+XENP32803 was investigated using unstimulated human PBMCs in vitro. To evaluate the potential for XENP24306+XENP32803 to induce production of cytokines associated with CRS, in vitro stimulation of human PBMCs was performed at 10 and 20 μg/mL (43-fold and 87-fold higher than predicted Cmax (0.23 μg/mL) in blood at the recommended FIH dose (0.01 mg/kg)) concentrations of XENP24306+XENP32803. Both immobilized and soluble formats of XENP24306+XENP32803 induced IFNγ production. The magnitude of IFNγ induction with XmAb24306 (9- to 14-fold compared to vehicle control) was multi-fold lower than observed with an anti-CD28 antibody (393-fold compared to vehicle control) or anti-CD3 antibody (1605-fold compared to vehicle control), used as positive controls. No induction of any other cytokines such as IL-1β, IL-2, IL-4, IL-6, IL-8, IL-10, IL-12p70, IL-13, or TNF was observed. XENP24306+XENP32803 did not induce inflammatory cytokines that were known to be involved in CRS, such as IL-6 and TNF, which indicates that the risk of XENP24306+XENP32803 inducing CRS is low.

### In Vivo Studies

Immune responses were assessed in cynomolgus monkeys following single or repeat doses of XENP24306+XENP32803. No apparent elevation of inflammatory cytokines, such as IL-6, tumor necrosis factor-α (TNFα), and IFNγ was observed following IV doses of XENP24306+XENP32803. Transient elevation of other cytokines and chemokines, such as IP-10, MCP-1 (monocyte chemoattractant protein-1), MIP-1α (macrophage inflammatory protein-1α), MIP-1β (macrophage inflammatory protein-1β), TARC (Thymus and Activation Regulated Chemokine), and eotaxin was observed, indicative of PD activity. Peak serum concentrations of these cytokines and chemokines were reached within 1 day of administration and returned to pretreatment levels by Day 15. Soluble CD25 serum concentrations peaked around Day 4 after treatment and returned to pretreatment levels by Day 15.

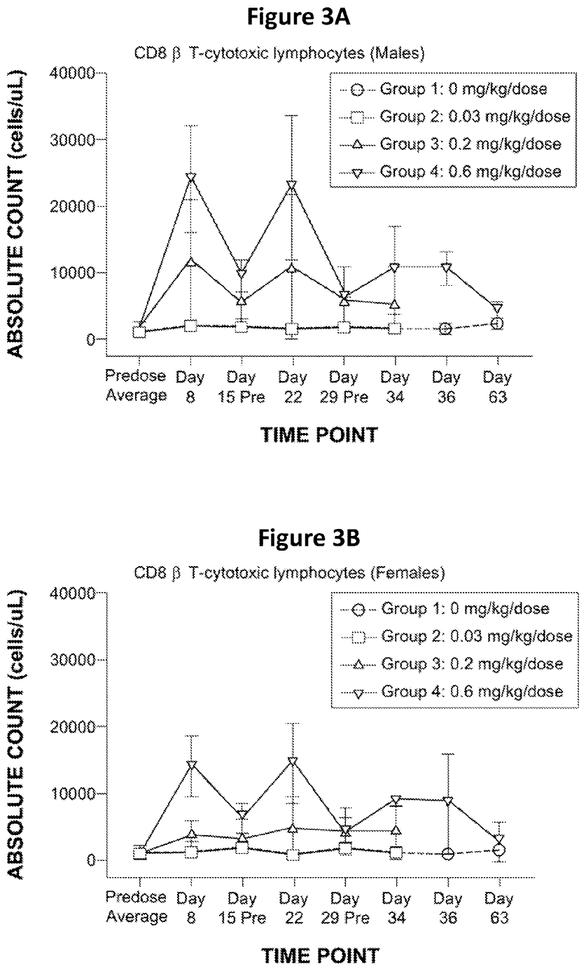

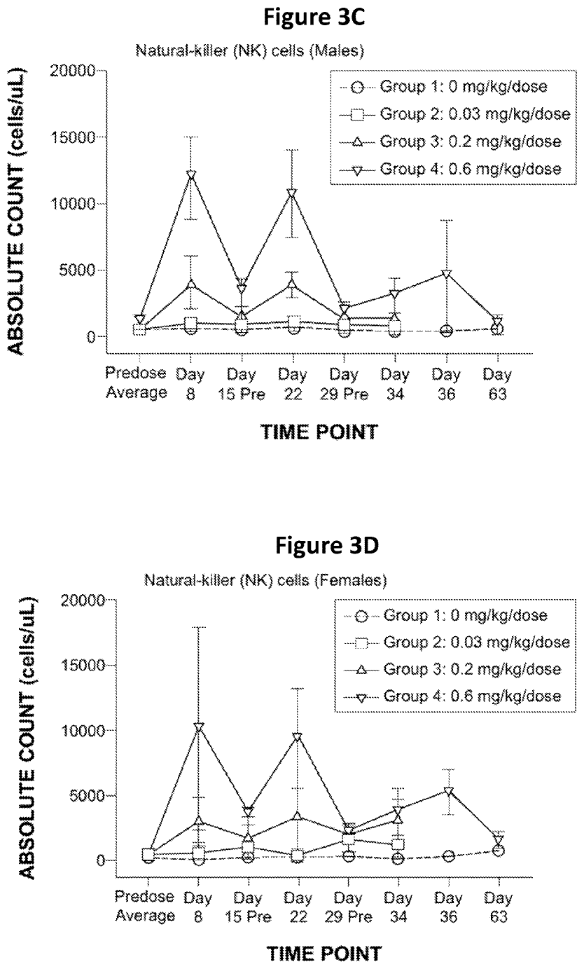

XENP24306+XENP32803 treatment expanded CD8⁺ T cell and NK-cell numbers in peripheral blood, validating the targeting of expected immune cell populations. Following an initial decrease in blood lymphocytes, likely due to margination, CD8⁺ T cells and NK cells exhibited dose-dependent expansion over pretreatment levels. Peak response in blood was achieved a week after dosing, and cell counts appeared to return close to pretreatment levels 2 weeks later. CD8⁺ memory T cell subsets, including central and effector memory, terminal effector, and stem cell memory cells were expanded, but naive CD8⁺ T cells were not. CD4⁺ T cells, Tregs, B cells, and granulocytes showed either minimal expansion or were not responsive to XENP24306+XENP32803. A transient and dose-dependent increase in frequencies of Ki67 expression (cell proliferation marker) was also observed among these target cell populations consistent with expansion of absolute cell numbers. Repeat dosing of XENP24306+XENP32803 (0.03, 0.2, and 0.6 mg/kg, Q2W) showed transient elevations in cytokine and chemokine responses after each dose. Responses to XENP24306+XENP32803 were dose-dependent, and changes were reversible with cytokines, chemokines, and sCD25 levels. The repeat-dose toxicity study demonstrated that CD8⁺ T cell and NK-cell expansion (approximately 6-fold at mid dose and 14-17 fold at high dose) in peripheral blood was transient after each dose with lower peak counts observed following repeated XENP24306+XENP32803 treatment (FIG. 3). Peripheral CD8⁺ T cell and NK-cell numbers returned to pretreatment levels after a 4-week recovery period.

The ability of XENP24306+XENP32803 to enhance leukocyte proliferation and effector activity was tested in a repeat dose study in a mouse graft-versus-host-disease (GVHD) model. XENP24306+XENP32803 (at four dose levels of 0.01, 0.03, 0.1, or 0.3 mg/kg, dosed on Days 0, 7, 14, and 21) was evaluated in non-obese diabetic/severe combined immunodeficient gamma (NSG) mice engrafted with human PBMCs, as a single agent. This study monitored an immune response against the mouse host that was measurable by clinical signs of GVHD (i.e., body weight loss and mortality), and immune monitoring assessments, such as elevations in peripheral human CD8⁻ T cell and NK-cell counts and serum IFNγ concentrations. Dose-dependent, GVHD-inducing activity was observed with significant body weight loss seen in mice treated with 0.3 mg/kg XENP24306+XENP32803, while significant elevations in CD8⁻ T cell and NK-cell counts and serum IFNγ concentrations were detected at lower doses. Time (Day 7, 14, 21) and dose-dependent increases in CD8⁺ T cell and NK-cell counts were observed. Expansion of CD4-T cells was only observed on Day 14 at the two highest dose levels tested. The minimum pharmacologically active dose manifested by increased expansion of NK cells was 0.01 mg/kg, whereas higher doses were required to demonstrate significant enhancements of CD8⁺ T cells and serum IFNγ. Thus, XENP24306+XENP32803 promoted proliferation and effector enhancement of CD8⁻ T cells and NK cells that contributed to GVHD.

XENP24306+XENP32803 (at three dose levels of 0.1, 0.3, or 1.0 mg/kg, dosed on Days 0, 7, 14 and 21) was evaluated for antitumor efficacy in mouse, as a single agent. NSG mice engrafted with MCF-7 human breast cancer cells and human PBMCs were used to determine if XENP24306+XENP32803 promoted antitumor responses. Significant antitumor activity, as indicated by reduced tumor growth, was observed at all XENP24306+XENP32803 dose levels (0.1, 0.3, and 1.0 mg/kg) when given as a single agent. Time- and dose-dependent elevations in peripheral CD8⁺ T cell, CD4⁺ T cell, and NK-cell counts and serum IFNγ concentrations were measured, demonstrating that XENP24306+XENP32803 promoted antitumor responses.

### Example 2: Pharmacokinetics and Drug Metabolism in Animals

A combination of XENP24306 (˜82%) and XENP32803 (˜18%) (“XENP24306+XENP32803”) binds to human and cynomolgus monkey IL-2/IL-15βγ heterodimeric receptor complex with comparable affinities and is active on both human and cynomolgus monkey CD8⁺ T cells and NK cells. Therefore, pharmacokinetics (PK) of XENP24306+XENP32803 were investigated in cynomolgus monkeys to support dose selection for Good Laboratory Practice (GLP) toxicity studies and to support selection of dose and dose regimen in the first-in-human (FIH) study. To support GLP toxicity studies, an electrochemiluminescent assay was developed and validated to quantify XENP24306+XENP32803 in cynomolgus monkey serum samples. Goat anti-human IL-15Rα antibody was used as capture, while mouse anti-human/primate IL-15 biotinylated antibody and sulfo-tagged streptavidin were used as primary and secondary detection reagents. The lower limit of quantification (LLOQ) was 30.0 ng/mL.

A time-resolved fluorescence method was developed to quantify XENP24306+XENP32803 concentrations in non-GLP PK/PD studies in cynomolgus monkey serum samples. The LLOQ in this assay was 1.4 ng/mL.

### Single-Dose Pharmacokinetics in Cynomolgus Monkeys

A preliminary pilot study designed to assess efficacy and to help define the max tolerated dose for GLP study design was conducted. Single-dose pharmacokinetics of XENP24306+XENP32803 were characterized in two, independent PK/PD studies in cynomolgus monkeys at 3.0 mg/kg in males and at 0.6 mg/kg in females. XENP24306+XENP32803 demonstrated a multiphasic profile with a mean Clearance (CL) of 66.4 mL/day/kg and mean volume of distribution at steady state (Vₛₛ) of 107 mL/kg following a single, 3.0 mg/kg IV administration to male cynomolgus monkeys. Mean Cₘₐₓ and exposure (area under the concentration-time curve from Time 0 to infinity [AUC_{0-∞}]) was 69.6 μg/mL and 45.4 day·μg/mL, respectively. Following a single IV administration of 0.6 mg/kg XENP24306+XENP32803 to female cynomolgus monkeys, the mean Cₘₐₓ was 11.9 μg/mL, exposure (AUC_{0-∞}) was 11.7 day·μg/mL, CL was 52.6 mL/day/kg, and Vₛₛ was 89.0 mL/kg. See Table 3.

TABLE 3
Summary (Mean ± SD) Pharmacokinetic parameters for
XENP24306 + XENP32803 following a single, intravenous
3.0 mg/kg dose in male cynomolgus monkeys and a single,
intravenous 0.6 mg/kg dose in female cynomolgus monkeys

| | 3.0 mg/kg | 0.6 mg/kg |
|---|---|---|
| PK Parameter | (male; n = 3) | (female; n = 3) |
| Cₘₐₓ(μg/mL) | 69.6 ± 5.03 | 11.9 ± 0.618 |
| AUC_{0-∞} (day · μg/mL) | 45.4^{a} | 11.7 ± 2.1 |
| CL (mL/day/kg) | 66.4^{a} | 52.6 ± 8.81 |
| Vₛₛ(mL/kg) | 107^{a} | 89.0 ± 4.58 |

^{a}Mean of 2 animals, therefore no SD reported.
The 3.0 mg/kg dose was not well tolerated.

### Repeat-Dose Pharmacokinetics in Cynomolgus Monkeys

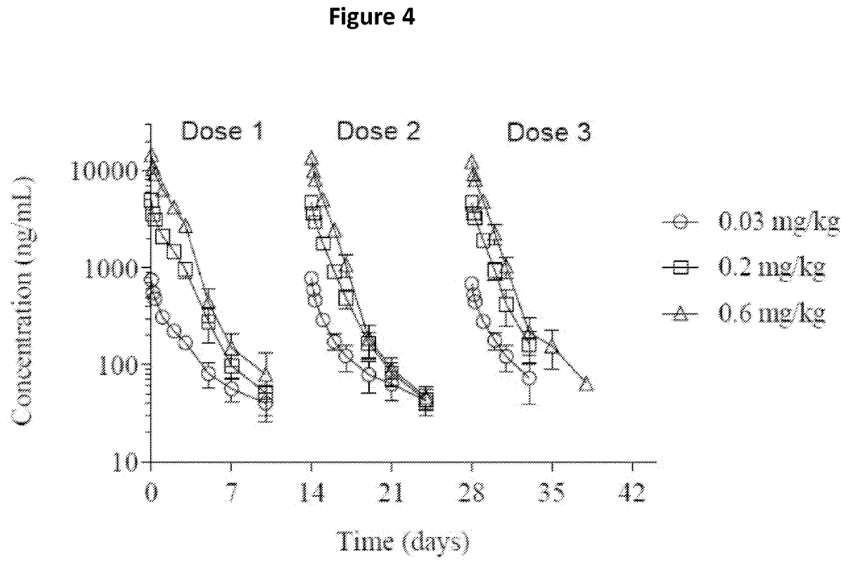

The toxicokinetics (TK) of XENP24306+XENP32803 were characterized in a 5-week, GLP, repeat-dose, toxicity study in cynomolgus monkeys. Three dose levels (0.03, 0.2, and 0.6 mg/kg XENP24306+XENP32803) were given at 14-day intervals for a total of 3 doses. Systemic exposure was confirmed in all animals and there were no sex differences observed in XENP24306+XENP32803 exposure in cynomolgus monkeys (FIG. 4). The Cₘₐₓ was dose-proportional after the first dose. There was a slight trend for decreasing Cₘₐₓ with repeated dosing; however, the ranges (mean±SD) were overlapping for Cₘₐₓ after the first, second, and third doses. The AUC₀₋₁₄ was slightly less than dose-proportional after the first dose. In addition to this, exposure (AUC) decreased with repeated XENP24306+XENP32803 dosing, particularly at the 0.2 mg/kg dose (from 7.74 to 5.96 day·μg g/mL, 22% decrease) and the 0.6 mg/kg dose (from 21.1 to 14.9 day·μg/mL, 30% decrease; Table 4). This decrease in systemic exposure (AUC) upon repeated dosing might be attributed to an increase in TMDD as a result of increased target-cell population. The XENP24306+XENP32803 CL after the first dose ranged from 18 to 28 mL/day/kg, and the Vₛₛ was in the range of 52 to 86 mL/kg. The higher-than-normal IgG clearances (<10 mL/day/kg for a typical IgG) of XENP24306+XENP32803 observed in these studies were likely a consequence of TMDD. Time-varying, non-linear PK behavior was observed for XENP24306+XENP32803 across dose levels as indicated by increased CL with increased dose after the first dose and a further less-than-dose-proportional increase in AUC₀₋₁₄ after repeat dosing. A similar PK behavior is expected for XENP24306+XENP32803 in humans. Increased target-cell population in response to XENP24306+XENP32803 dosing was expected to increase the TMDD effect leading to time varying pharmacokinetics, as observed in this study. No accumulation was observed following repeated administration as indicated by decreasing AUC values, with an AUC ratio of 0.704- to 0.991-fold between the first and second doses (Table 4)

TABLE 4
Group mean (±SD) toxicokinetic parameters (males and
females combined) for XENP24306 + XENP32803 in cynomolgus
monkeys following Q2W (every 2 weeks) intravenous dosing.

| | Group 2 | Group 3 | Group 4 |
|---|---|---|---|
| Toxicokinetic Parameter | (0.03 mg/kg) | (0.2 mg/kg) | (0.6 mg/kg) |
| Cₘₐₓ, first dose (μg/mL) | 0.750 ± 0.0410 | 5.03 ± 0.851 | 14.7 ± 1.73 |
| Cₘₐₓ, second dose (μg/mL) | 0.776 ± 0.0415 | 4.73 ± 0.455 | 13.6 ± 1.88 |
| Cₘₐₓ, third dose (μg/mL) | 0.687 ± 0.0510 | 4.75 ± 0.555 | 12.4 ± 1.58 |
| AUC₀₋₁₄, first dose | 1.56 ± 0.148 | 7.74 ± 0.960 | 21.1 ± 1.21 |
| (day · μg/mL) | | | |
| AUC₀₋₁₄, second dose | 1.55 ± 0.247 | 5.96 ± 0.489 | 14.9 ± 1.36 |
| (day · μg/mL) | | | |
| CL, first dose (mL/day/kg) | 17.9 ± 2.22 | 26.0 ± 3.09 | 28.4 ± 1.61 |
| Vss, first dose (mL/kg) | 86.2 ± 6.31 | 56.1 ± 5.72 | 52.3 ± 6.98 |

### Example 3: Pharmacodynamic Effects

### Effect on Cytokines, Chemokines and Soluble CD25

Cytokines were assessed following single-dose 0.6 or 3.0 mg/kg of a combination of IL15/IL15Rα heterodimeric proteins (XENP24306 (˜82%) and XENP32803 (˜18%) (“XENP24306+XENP32803”)) in two, independent, cynomolgus monkey PK/PD studies). At both the 0.6 mg/kg and 3.0 mg/kg XENP24306+XENP32803 dose, elevations of serum markers as well as cytokines and chemokines peaked within 8 to 16 hours following dosing and generally returned to pretreatment levels by day 15. Serum markers that were elevated following XENP24306+XENP32803 treatment included eotaxin, eotaxin-3, IL-8, IP-10, MCP-1, MCP-4, MDC, MIP-1α, MIP-1β, and TARC. Increased expression of these cytokines and chemokines may further contribute to the lymphocyte expansions induced by XENP24306+XENP32803.

In two, independent, PK/PD studies, sCD25/IL-2Rα was assessed following a single dose of 0.6 or 3.0 mg/kg XENP24306+XENP32803. At both the 0.6 mg/kg and 3.0 mg/kg XENP24306+XENP32803 dose groups, the pattern for sCD25 showed gradual increases 3 to 4 days following dosing, which aligned with CD25 expression on T cells.

### Effect on Lymphocytes

After a single dose of 0.6 mg/kg or 3.0 mg/kg XENP24306+XENP32803, lymphocytes were mildly-to-moderately decreased until 3 days following dosing. This was followed by a variable, dose-dependent, moderate-to-marked increase that peaked 7 to 9 days after dosing. Lymphocytes were subsequently recovered or partially recovered towards pretreatment levels by end of study. Monocytes tended to mirror lymphocytes, but to a much lesser degree. Blood smear examination performed on the 0.6 mg/kg-dose animals noted that many of the lymphocytes were atypical/reactive.

### Mononuclear Cell Infiltration

Following single-dose 0.6 mg/kg XENP24306+XENP32803, minimal-to-mild mononuclear cell infiltration was observed in the sinusoids of the liver. At single-dose 3.0 mg/kg XENP24306+XENP32803, mononuclear-cell infiltrates were noted in the liver, kidneys, lung, jejunum, urinary bladder, and skin.

### Example 4: Repeat-Dose Toxicity

Two, repeat-dose, GLP studies were conducted: (1) a 5-week toxicity study with a 4-week recovery period described in this Example and (2) a dedicated cardiovascular safety pharmacology study described in Example 5.

The 5-week, repeat-dose, GLP toxicity study was conducted in male and female cynomolgus monkeys to evaluate toxicity, pharmacology, and TK of a combination of IL15/IL15Rα heterodimeric proteins (XENP24306 (˜82%) and XENP32803 (˜18%) (“XENP24306+XENP32803”)). Animals either received vehicle (control group) or were dosed with 0.03, 0.2, or 0.6 mg/kg XENP24306+XENP32803 via IV bolus on Days 1, 15, and 29, and underwent necropsy on Day 34 (main study cohort) or Day 64 (recovery cohort; control and 0.6 mg/kg XmAb24306). The 30-day recovery period was designed to assess reversibility or persistence of any XENP24306+XENP32803-related effects.

Assessment of toxicity was based on clinical observations, body weight, qualitative food evaluation, ophthalmology, ECG, clinical pathology parameters (hematology, coagulation, clinical chemistry, urinalysis, and urine chemistry), bioanalytical and TK parameters, ADA, cytokines, flow cytometry analyses, gross necropsy findings, organ weights, and histopathologic examinations.

TK analysis confirmed systemic exposure of XENP24306+XENP32803 at all dose levels tested. There were no differences in exposure between sexes. The Cₘₐₓ was dose proportional after the first dose. The AUC₀₋₁₄ after the first dose increased with dose, but was slightly less than dose proportional, and exposure (AUC) decreased upon repeated dosing. XENP24306+XENP32803 appeared to have non-linear kinetics in cynomolgus monkeys due to TMDD at the dose levels tested (Example 2).

All findings in the repeat-dose GLP toxicity study were consistent with the expected pharmacologic response of T cell and NK-cell expansion and activation with an associated pro-inflammatory response. The NOAEL defined from the dedicated repeat-dose, GLP toxicity study was determined to be 0.03 mg/kg XENP24306+XENP32803. Corresponding safety margins of the proposed XENP24306+XENP32803 FIH dose of 0.01 mg/kg IV Q2W to the NOAEL are described in Example 5.

### Example 5: Safety Pharmacology

A single, dedicated, GLP safety pharmacology study was performed in telemetry-instrumented male cynomolgus monkeys (four per group, including a vehicle control group) to assess the potential effects of a combination of IL15/IL15Rα heterodimeric proteins (XENP24306 (˜82%) and XENP32803 (˜18%) (“XENP24306+XENP32803”)) on the cardiovascular system. XENP24306+XENP32803 was administered at 0.03, 0.2, and 0.6 mg/kg (same doses as in the GLP toxicity study) by IV bolus injection on Days 1 and 15, and animals returned to the colony on Day 23. The following parameters and end points were evaluated: clinical signs, food consumption (qualitative evaluation), body weight, cardiovascular evaluation (systolic, diastolic, and MAP, heart rate, and ECG (including qualitative evaluation, and measurements of the RR-, PR-, QRS-, and QT-intervals and derived heart rate-corrected QT [QTca] interval), body temperature, serum albumin concentrations, and XENP24306+XENP32803 exposure and ADA incidence.

XENP24306+XENP32803 was clinically well tolerated at all doses (0.03, 0.2, and 0.6 mg/kg) with all animals surviving the study period and no veterinary intervention required. No adverse clinical signs, test article-related changes in food consumption, body weight changes, or ECG abnormalities were observed at any dose. ECGs were considered qualitatively normal for the cynomolgus monkey with no treatment-related changes in PR-, QRS-, or QTca-intervals.

Systemic exposure of XENP24306+XENP32803 was demonstrated at all dose levels. No treatment-related changes in body weight or qualitative food consumption occurred during the study.

Based on the totality of findings from GLP studies in cynomolgus monkeys, the no-observed-adverse-effect level (NOAEL) dose was considered to be 0.03 mg/kg XENP24306+XENP32803. Due to the immune agonist properties of XENP24306+XENP32803, determination of the FIH dose was based on a minimum anticipated biological effect level (MABEL) approach. A dose of 0.01 mg/kg XENP24306+XENP32803, IV, as a single agent is proposed as the FIH dose for XENP24306+XENP32803. This FIH dose is based on EC₂₀ (0.23 μg/mL; geometric mean of 20 donors) and was derived using in vitro NK-cell (CD3⁻ CD56⁺) proliferation (percent of cells that express Ki67) in human PBMCs, the most sensitive in vitro assay with XENP24306+XENP32803. See FIG. 1. The recommended FIH dose of 0.01 mg/kg XENP24306+XENP32803 is anticipated to be safe and is expected to provide minimal biological effect with minimal risk for treatment-mediated reactions in humans. Cₘₐₓ of XENP24306+XENP32803 administered IV in humans at the recommended FIH dose (i.e., at 0.01 mg/kg) is not expected to exceed this EC₂₀ level. The starting dose of 0.01 mg/kg XENP24306+XENP32803 in humans has a three-fold safety margin to the NOAEL dose (0.03 mg/kg XENP24306+XENP32803, Q2W) in the 5-week, GLP toxicity study in cynomolgus monkeys. Cₘₐₓ of XENP24306+XENP32803 administered IV in humans at 0.01 mg/kg XENP24306+XENP32803 is expected to be 3.3-fold below the observed Cₘₐₓ (0.75±0.04 μg/mL; first dose) at the NOAEL dose in cynomolgus monkeys. See Table 5. Furthermore, AUC at 0.01 mg/kg XENP24306+XENP32803 in humans is expected to be 1.8-fold below the AUC observed at the NOAEL dose in cynomolgus monkeys (Table 5). In summary, the observed Cₘₐₓ and AUC at the NOAEL of XENP24306+XENP32803 in a relevant nonclinical GLP toxicity model (cynomolgus monkeys) further support the MABEL-based starting dose of 0.01 mg/kg XENP24306+XENP32803 IV and provide sufficient safety margins (Table 5) for the study.

The dosing frequency of XENP24306+XENP32803 in humans is Q2W and is supported by the 5-week, cynomolgus monkey, GLP toxicity study, where XENP24306+XENP32803 was generally well tolerated when given Q2W with no significant, acute toxicities. Peak, peripheral PD response (target-cell expansion such as NK and CD8⁺ T cells) was achieved a week after dosing and these peripheral target cell counts were declining toward their baseline by end of 2 weeks, following XENP24306+XENP32803 administration. Furthermore, cytokines and chemokines indicative of PD activity peaked between 8 to 16 hours following dosing and returned to baseline within 14 days of dosing (See Example 3). Therefore, an initial dosing frequency of Q2W is considered appropriate in the monotherapy dose escalation study with XENP24306+XENP32803 with the dose-limiting toxicity observation period encompassing the first cycle of study treatment.

TABLE 5
Non-clinical safety margin estimates for XENP24306 + XENP32803
at proposed FIH dose: dose, AUC, and Cₘₐₓ based exposure multiples
for the recommended starting dose of XENP24306 + XENP32803
(0.01 mg/kg, Q2W) versus NOAEL (0.03 mg/kg, Q2W) in the 5-
Week, GLP, Toxicity Study in Cynomolgus Monkeys

| | Cₘₐₓ | AUC | Dose |
|---|---|---|---|
| | (μg/mL) | (day · μg/mL)^{a} | (mg/kg) |
| Starting dose in human: 0.01 mg/kg | 0.23 | 0.86 | 0.01 |
| Anticipated values | | | |
| NOAEL in cynomolgus monkey: | 0.75 | 1.56 | 0.03 |
| 0.03 mg/kg | | | |
| Observed values | | | |
| Safety margins | 3.3x | 1.8x | 3x |

AUC = area under the concentration-time curve; Cmax = maximum observed serum concentration; GLP = Good Laboratory Practice; IV = intravenous; NOAEL = no-observed-adverse-effect level; Q2W = every 2 weeks.
^{a}AUCₕᵤₘₐₙ is predicted AUC₀₋₁₄ (i.e., dose/scaled human clearance) and AUC_{cyno} is observed AUC₀₋₁₄ after the first dose at NOAEL (0.03 mg/kg) in the 5-week, GLP toxicity study in cynomolgus monkeys.
Scaled human clearance = 11.6 mL/day/kg.

### Example 6: Monotherapy, Open-Label, Multicenter, Global, Dose-Escalation Study of a Combination of IL15/IL15Rα Heterodimeric Proteins

A monotherapy, open-label, multicenter, global, dose-escalation study to evaluate the safety, tolerability pharmacokinetics and activity of a combination of IL15/IL15Rα heterodimeric proteins (XENP24306 (˜82%) and XENP32803 (˜18%) (“XENP24306+XENP32803”)) will be conducted.

The study consists of a screening period of up to 28 days, a treatment period, and a minimum follow-up period of 90 days after treatment.

Patients will be enrolled in two stages: a dose-escalation stage and an expansion stage.

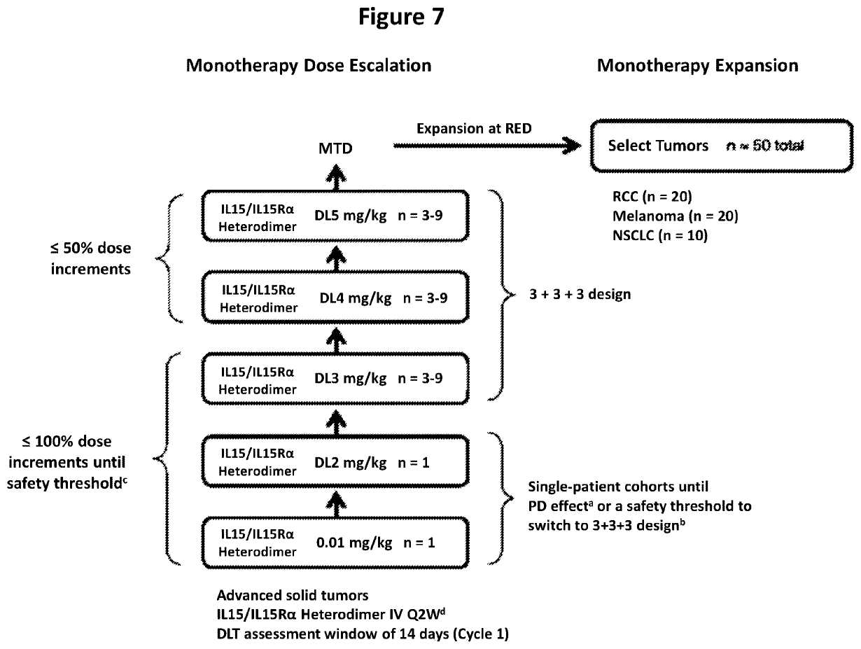

Approximately 21-54 patients with locally advanced, recurrent, or metastatic incurable solid tumors will be enrolled in the dose-escalation stage study. The initial dose of XENP24306+XENP32803 will be 0.01 mg/kg Q2W. XENP24306+XENP32803 will be administered by IV infusion. The XENP24306+XENP32803 dose will be increased by up to 100% of the preceding dose level for each successive cohort, until a safety threshold (defined as a dose-limiting toxicity (DLT) in 1 patient or a Grade 2 major organ adverse event not attributable to another clearly identifiable cause in at least 2 patients during the DLT assessment window in a given cohort) is observed. Subsequently, cohorts of 3-9 patients each will be evaluated at escalating dose levels following a 3+3+3 design to determine the maximum tolerated dose (MTD) or maximum administered dose (MAD) for single-agent XENP24306+XENP32803. FIG. 7.

Patients in this study will be initially assessed for eligibility during the screening period (lasting ≤28 days). Following confirmation of eligibility, patients will receive 0.01 mg/kg of XENP24306+XENP32803 by IV infusion on the first day of every 14-day cycle (Q2W). XENP24306+XENP32803 PK will be assessed. Patients will be evaluated weekly by physical examination and blood collections for routine hematologic and metabolic laboratory assessments for the first eight cycles of XENP24306+XENP32803 treatment during dose escalation, the first two cycles during expansion, and less frequently thereafter. Tumor assessment will occur at baseline and after initiation of study.

Patients enrolling into cleared cohorts of monotherapy dose-escalation cohorts (i.e., backfill cohorts) must have one of the following PD-L1-selected tumor types: melanoma, non-small cell lung cancer (NSCLC), head and neck squamous cell carcinoma (HNSCC), triple-negative breast cancer (TNBC) urothelial carcinoma (UCC), renal cell carcinoma (RCC), small cell lung carcinoma (SCLC), GC, Merkel cell carcinoma (MCC), cutaneous squamous cell carcinoma (cSCC), microsatellite instability-high (MSI-H) cancers.

Approximately 185-240 patients with locally advanced, recurrent, or metastatic incurable malignancies that have progressed after available standard therapy; or for whom standard therapy has proven to be ineffective or intolerable, or is considered inappropriate; or for whom a clinical trial of an investigational agent is a recognized standard of care will be enrolled in the expansion cohorts of the study. This expansion stage will consist of defined cohorts of patients to better characterize the safety, pharmacokinetics, PD activity, and preliminary anti-tumor activity of XENP24306+XENP32803 as a single agent. XENP24306+XENP32803 will be administered by IV infusion in the expansion stage. A provisional XENP24306+XENP32803 recommended expansion dose (RED) will be proposed at or below the MTD/MAD established in dose escalation. Once the RED of XENP24306+XENP32803 has been proposed, additional patients will be enrolled in the expansion stage and treated at the RED.

All patients will be closely monitored for adverse events throughout the study and for at least 90 days after the final dose of study treatment or until initiation of another systemic anti-cancer therapy, whichever occurs first. Adverse events will be graded according to NCI CTCAE v5.0.

To characterize the pharmacokinetics, immunogenicity response, and PD properties of XENP24306+XENP32803 as a single agent, blood samples will be taken at various timepoints before and after dosing.

Patients will undergo tumor assessments at screening (baseline) and at regular intervals during the study, which will be measured by Response Evaluation Criteria in Solid Tumors (RECIST) v.1. A modified RECIST v1.1 for immune-based therapeutics (iRECIST) will also be used in this study to better characterize the different patterns of responses associated with cancer immunotherapy (CIT) and to allow a better understanding of the preliminary activity profile of XENP24306+XENP32803. iRECIST is intended to supplement standard RECIST v1.1 in this study to allow the investigators to make an integrated assessment of benefit and risk for patients.

The activity objective for this study is to make a preliminary assessment of the activity of XENP24306+XENP32803 when administered as a single agent on the basis of the following endpoints:
Serum concentration of XENP24306+XENP32803;
Percentage of participants with adverse events;
Objective response rate (ORR), defined as the proportion of patients with a complete response (CR) or partial response (PR);
Duration of response (DOR), defined as the time from the first occurrence of a documented objective response to disease progression or death from any cause (whichever occurs first);
Progression-free survival (PFS) after enrollment, defined as the time from enrollment to the first occurrence of disease progression or death from any cause (whichever occurs first); and
Overall survival (OS) after enrollment, defined as the time from enrollment to death from any cause.

The safety objective for this study is to evaluate the safety of XENP24306+XENP32803 when administered as a single agent based on the incidence and severity of adverse events and on changes from baseline in targeted vital signs, or clinical laboratory test results or ECGs parameters.

The pharmacokinetic (PK) objective for this study is to characterize the XENP24306+XENP32803 PK profile when administered as a single agent on the basis of serum concentration of XENP24306+XENP32803 at specified timepoints.

The immunogenicity objective for this study is to evaluate the immune response to XENP24306+XENP32803 when administered as a single agent (Ia) on the basis of ADAs to XENP24306+XENP32803 at baseline and incidence of ADAs to XENP24306+XENP32803 during the study.

### Example 7: Monotherapy, Open-Label, Multicenter, Global, Dose-Escalation Study of XENP24306

A monotherapy, open-label, multicenter, global, dose-escalation study to evaluate the safety, tolerability pharmacokinetics and activity of XENP24306 will be conducted.

The study consists of a screening period of up to 28 days, a treatment period, and a minimum follow-up period of 90 days after treatment.

Patients will be enrolled in two stages: a dose-escalation stage and an expansion stage.

Approximately 21-54 patients with locally advanced, recurrent, or metastatic incurable solid tumors will be enrolled in the dose-escalation stage study. The initial dose of XENP24306 will be 0.01 mg/kg Q2W. XENP24306 will be administered by IV infusion. The XENP24306 dose will be increased by up to 100% of the preceding dose level for each successive cohort, until a safety threshold (defined as a dose-limiting toxicity (DLT) in 1 patient or a Grade ≥2 major organ adverse event not attributable to another clearly identifiable cause in at least 2 patients during the DLT assessment window in a given cohort) is observed. Subsequently, cohorts of 3-9 patients each will be evaluated at escalating dose levels following a 3+3+3 design to determine the maximum tolerated dose (MTD) or maximum administered dose (MAD) for single-agent XENP24306. FIG. 7.

Patients in this study will be initially assessed for eligibility during the screening period (lasting ≤28 days). Following confirmation of eligibility, patients will receive 0.01 mg/kg of XENP24306 by IV infusion on the first day of every 14-day cycle (Q2W). XENP24306 PK will be assessed. Patients will be evaluated weekly by physical examination and blood collections for routine hematologic and metabolic laboratory assessments for the first eight cycles of XENP24306 treatment during dose escalation, the first two cycles during expansion, and less frequently thereafter. Tumor assessment will occur at baseline and after initiation of study.

Patients enrolling into cleared cohorts of monotherapy dose-escalation cohorts (i.e., backfill cohorts) must have one of the following PD-L1-selected tumor types: melanoma, non-small cell lung cancer (NSCLC), head and neck squamous cell carcinoma (HNSCC), triple-negative breast cancer (TNBC) urothelial carcinoma (UCC), renal cell carcinoma (RCC), small cell lung carcinoma (SCLC), GC, Merkel cell carcinoma (MCC), cutaneous squamous cell carcinoma (cSCC), microsatellite instability-high (MSI-H) cancers.

Approximately 185-240 patients with locally advanced, recurrent, or metastatic incurable malignancies that have progressed after available standard therapy; or for whom standard therapy has proven to be ineffective or intolerable, or is considered inappropriate; or for whom a clinical trial of an investigational agent is a recognized standard of care will be enrolled in the expansion cohorts of the study. This expansion stage will consist of defined cohorts of patients to better characterize the safety, pharmacokinetics, PD activity, and preliminary anti-tumor activity of XENP24306 as a single agent. XENP24306 will be administered by IV infusion in the expansion stage. A provisional XENP24306 recommended expansion dose (RED) will be proposed at or below the MTD/MAD established in dose escalation. Once the RED of XENP24306 has been proposed, additional patients will be enrolled in the expansion stage and treated at the RED.

All patients will be closely monitored for adverse events throughout the study and for at least 90 days after the final dose of study treatment or until initiation of another systemic anti-cancer therapy, whichever occurs first. Adverse events will be graded according to NCI CTCAE v5.0.

To characterize the pharmacokinetics, immunogenicity response, and PD properties of XENP24306 as a single agent, blood samples will be taken at various timepoints before and after dosing.

Patients will undergo tumor assessments at screening (baseline) and at regular intervals during the study, which will be measured by Response Evaluation Criteria in Solid Tumors (RECIST) v.1. A modified RECIST v1.1 for immune-based therapeutics (iRECIST) will also be used in this study to better characterize the different patterns of responses associated with cancer immunotherapy (CIT) and to allow a better understanding of the preliminary activity profile of XENP24306. iRECIST is intended to supplement standard RECIST v1.1 in this study to allow the investigators to make an integrated assessment of benefit and risk for patients.

The activity objective for this study is to make a preliminary assessment of the activity of XENP24306 when administered as a single agent on the basis of the following endpoints:
Serum concentration of XENP24306;
Percentage of participants with adverse events;
Objective response rate (ORR), defined as the proportion of patients with a complete response (CR) or partial response (PR);
Duration of response (DOR), defined as the time from the first occurrence of a documented objective response to disease progression or death from any cause (whichever occurs first);
Progression-free survival (PFS) after enrollment, defined as the time from enrollment to the first occurrence of disease progression or death from any cause (whichever occurs first); and
Overall survival (OS) after enrollment, defined as the time from enrollment to death from any cause.

The safety objective for this study is to evaluate the safety of XENP24306 when administered as a single agent based on the incidence and severity of adverse events and on changes from baseline in targeted vital signs, or clinical laboratory test results or ECGs parameters.

The pharmacokinetic (PK) objective for this study is to characterize the XENP24306 PK profile when administered as a single agent on the basis of serum concentration of XENP24306 at specified timepoints.

The immunogenicity objective for this study is to evaluate the immune response to XENP24306 when administered as a single agent (Ia) on the basis of ADAs to XENP24306 at baseline and incidence of ADAs to XENP24306 during the study.

### Example 8: Monotherapy, Open-Label, Multicenter, Global, Dose-Escalation Study of XENP32803

A monotherapy, open-label, multicenter, global, dose-escalation study to evaluate the safety, tolerability pharmacokinetics and activity of XENP32803 will be conducted.

The study consists of a screening period of up to 28 days, a treatment period, and a minimum follow-up period of 90 days after treatment.

Patients will be enrolled in two stages: a dose-escalation stage and an expansion stage.

Approximately 21-54 patients with locally advanced, recurrent, or metastatic incurable solid tumors will be enrolled in the dose-escalation stage study. The initial dose of XENP32803 will be 0.01 mg/kg Q2W. XENP32803 will be administered by IV infusion. The XENP32803 dose will be increased by up to 100% of the preceding dose level for each successive cohort, until a safety threshold (defined as a dose-limiting toxicity (DLT) in 1 patient or a Grade ≥2 major organ adverse event not attributable to another clearly identifiable cause in at least 2 patients during the DLT assessment window in a given cohort) is observed. Subsequently, cohorts of 3-9 patients each will be evaluated at escalating dose levels following a 3+3+3 design to determine the maximum tolerated dose (MTD) or maximum administered dose (MAD) for single-agent XENP32803. FIG. 7.

Patients in this study will be initially assessed for eligibility during the screening period (lasting ≤28 days). Following confirmation of eligibility, patients will receive 0.01 mg/kg of XENP32803 by IV infusion on the first day of every 14-day cycle (Q2W). XENP32803 PK will be assessed. Patients will be evaluated weekly by physical examination and blood collections for routine hematologic and metabolic laboratory assessments for the first eight cycles of XENP32803 treatment during dose escalation, the first two cycles during expansion, and less frequently thereafter. Tumor assessment will occur at baseline and after initiation of study.

Patients enrolling into cleared cohorts of monotherapy dose-escalation cohorts (i.e., backfill cohorts) must have one of the following PD-L1-selected tumor types: melanoma, non-small cell lung cancer (NSCLC), head and neck squamous cell carcinoma (HNSCC), triple-negative breast cancer (TNBC) urothelial carcinoma (UCC), renal cell carcinoma (RCC), small cell lung carcinoma (SCLC), GC, Merkel cell carcinoma (MCC), cutaneous squamous cell carcinoma (cSCC), microsatellite instability-high (MSI-H) cancers.

Approximately 185-240 patients with locally advanced, recurrent, or metastatic incurable malignancies that have progressed after available standard therapy; or for whom standard therapy has proven to be ineffective or intolerable, or is considered inappropriate; or for whom a clinical trial of an investigational agent is a recognized standard of care will be enrolled in the expansion cohorts of the study. This expansion stage will consist of defined cohorts of patients to better characterize the safety, pharmacokinetics, PD activity, and preliminary anti-tumor activity of XENP32803 as a single agent. XENP32803 will be administered by IV infusion in the expansion stage. A provisional XENP32803 recommended expansion dose (RED) will be proposed at or below the MTD/MAD established in dose escalation. Once the RED of XENP32803 has been proposed, additional patients will be enrolled in the expansion stage and treated at the RED.

All patients will be closely monitored for adverse events throughout the study and for at least 90 days after the final dose of study treatment or until initiation of another systemic anti-cancer therapy, whichever occurs first. Adverse events will be graded according to NCI CTCAE v5.0.

To characterize the pharmacokinetics, immunogenicity response, and PD properties of XENP32803 as a single agent, blood samples will be taken at various timepoints before and after dosing.

Patients will undergo tumor assessments at screening (baseline) and at regular intervals during the study, which will be measured by Response Evaluation Criteria in Solid Tumors (RECIST) v1.1. A modified RECIST v1.1 for immune-based therapeutics (iRECIST) will also be used in this study to better characterize the different patterns of responses associated with cancer immunotherapy (CIT) and to allow a better understanding of the preliminary activity profile of XENP32803. iRECIST is intended to supplement standard RECIST v1.1 in this study to allow the investigators to make an integrated assessment of benefit and risk for patients.

The activity objective for this study is to make a preliminary assessment of the activity of XENP32803 when administered as a single agent on the basis of the following endpoints:
Serum concentration of XENP32803;
Percentage of participants with adverse events;
Objective response rate (ORR), defined as the proportion of patients with a complete response (CR) or partial response (PR);
Duration of response (DOR), defined as the time from the first occurrence of a documented objective response to disease progression or death from any cause (whichever occurs first);
Progression-free survival (PFS) after enrollment, defined as the time from enrollment to the first occurrence of disease progression or death from any cause (whichever occurs first); and
Overall survival (OS) after enrollment, defined as the time from enrollment to death from any cause.

The safety objective for this study is to evaluate the safety of XENP32803 when administered as a single agent based on the incidence and severity of adverse events and on changes from baseline in targeted vital signs, or clinical laboratory test results or ECGs parameters.

The pharmacokinetic (PK) objective for this study is to characterize the XENP32803 PK profile when administered as a single agent on the basis of serum concentration of XENP32803 at specified timepoints.

The immunogenicity objective for this study is to evaluate the immune response to XENP32803 when administered as a single agent (Ia) on the basis of ADAs to XENP32803 at baseline and incidence of ADAs to XENP32803 during the study.

### Example 9: Non-Clinical Pharmacology of XENP24306+XENP32803 in Combination with Anti-PD-L1/PD-1 Inhibitors. In Vivo Studies

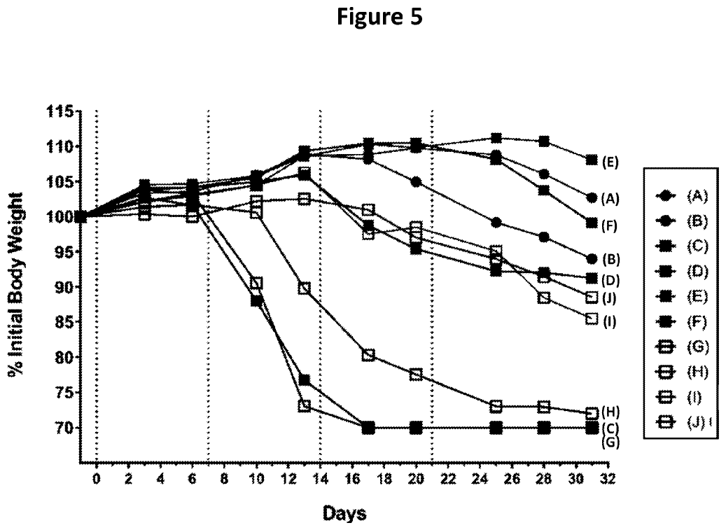

The ability of a combination of IL15/IL15Rα heterodimeric proteins (XENP24306 (˜82%) and XENP32803 (˜18%) (“XENP24306+XENP32803”)) to enhance leukocyte proliferation and effector activity was tested in a repeat dose study in a mouse graft-versus-host-disease (GVHD) model. XENP24306+XENP32803 (at four dose levels of 0.01, 0.03, 0.1, or 0.3 mg/kg, dosed on Days 0, 7, 14, and 21) was evaluated in non-obese diabetic/severe combined immunodeficient gamma (NSG) mice engrafted with human PBMCs in combination with XENP16432; an anti-PD-1 inhibitor given at a fixed dose of 3.0 mg/kg. This study monitored an immune response against the mouse host that was measurable by clinical signs of GVHD (i.e., body weight loss and mortality), and immune monitoring assessments, such as elevations in peripheral human CD8⁺ T cell and NK-cell counts and serum IFNγ concentrations. Dose-dependent, GVHD-inducing activity was observed with significant body weight loss seen in mice treated with 0.3 mg/kg XENP24306+XENP32803, while significant elevations in CD8⁻ T cell and NK-cell counts and serum IFNγ concentrations were detected at lower doses (FIG. 5). Time (Day 7, 14, 21) and dose-dependent increases in CD8⁺ T cell and NK-cell counts were observed. Expansion of CD4⁺ T cells was only observed on Day 14 at the two highest dose levels tested. The minimum pharmacologically active dose manifested by increased expansion of NK cells was 0.01 mg/kg, whereas higher doses were required to demonstrate significant enhancements of CD8⁺ T cells and serum IFNγ. Thus, XENP24306+XENP32803 promoted proliferation and effector enhancement of CD8⁺ T cells and NK cells that contributed to GVHD. Combination groups of XENP24306+XENP32803 (at doses of 0.1 and 0.3 mg/kg) with an anti-PD-1 antibody showed significantly superior GVHD-inducing activity compared with anti-PD-1 antibody alone.

This study describes the immunostimulatory activity of XENP24306+XENP32803, an IL15/IL15Rα-Fc fusion protein, on human immune cells. Importantly, this study demonstrates the benefit of combined treatment using XENP24306+XENP32803 with XENP16432/anti-PD1, an anti-PD1 bivalent antibody, to enhance immune responses over anti-PD1 treatment alone, suggesting the possibility of improving clinical benefit by combining approved anti-PD-L1 agents with XENP24306+XENP32803.

The minimum pharmacologically active dose (MPAD), revealed by increased expansion of NK cells relative to untreated control, was 0.01 mg/kg when XENP24306+XENP32803 was administered alone. Higher doses were required to demonstrate significant enhancements of T cells and serum IFNγ, as well as exacerbation of GVHD.

Combination treatment of XENP24306+XENP32803 with XENP16432/anti-PD1 also promoted significant enhancement of leukocyte numbers and IFNγ production compared to anti-PD1 single agent treatment. Notably, as leukocyte numbers expanded in response to the proliferative effects of XENP24306+XENP32803, measured trough serum concentrations of XENP24306+XENP32803 decreased, presumably due to target mediated drug disposition on a progressively expanding leukocyte population.

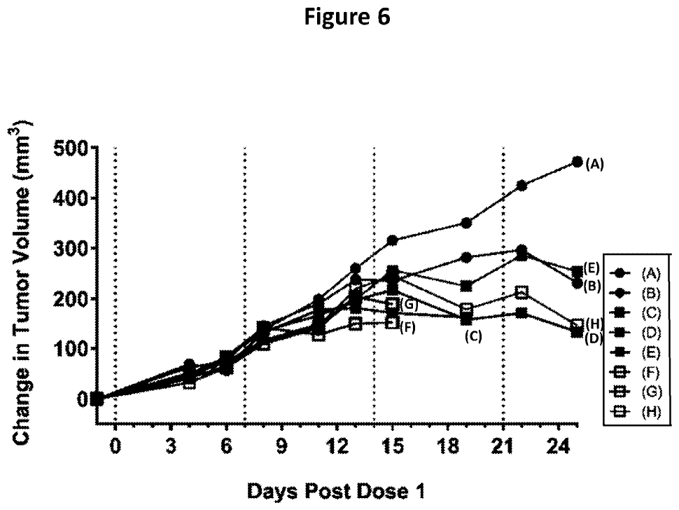

XENP24306+XENP32803 (at three dose levels of 0.1, 0.3, or 1.0 mg/kg, dosed on Days 0, 7, 14 and 21) was evaluated for antitumor efficacy in mouse, in combination with XENP16432; an anti-PD-1 inhibitor given at a fixed dose of 3.0 mg/kg. NSG mice engrafted with MCF-7 human breast cancer cells and human PBMCs were used to determine if XENP24306+XENP32803 in combination with anti-PD-1 promoted antitumor responses. Time- and dose-dependent elevations in peripheral CD8⁺ T cell, CD4⁺ T cell, and NK-cell counts and serum IFNγ concentrations were measured, demonstrating that XENP24306+XENP32803 promoted antitumor responses. FIG. 6.

Animals treated with PBS (Group A) displayed steady tumor growth through the end of the study. No animals from Group A were euthanized/found dead over the course of the study. Animals treated with XENP16432/anti-PD1 (Group B) initially displayed similar tumor growth kinetics as PBS treated animals (Group A) through Day 13. Beginning on Day 15, however,

XENP16432/anti-PD1-treated animals displayed statistically significant tumor growth inhibition in comparison to PBS-treated mice. The tumor volume reduction seen in XENP16432/anti-PD1-treated animals is consistent with a general allogeneic anti-tumor response. No XENP16432/anti-PD1-treated mice were euthanized/found dead over the course of the study. Treatment with 0.1 mg/kg XENP24306+XENP32803 (Group E) induced a significant tumor size reduction in comparison to PBS-treated animals as early as on Day 8. By Day 13, all three dose levels of XENP24306+XENP32803 (1.0, 0.3 and 0.1 mg/kg; Groups C, D and E) showed significant and dose-dependent tumor growth reductions in comparison to PBS-treated mice. Tumor volumes remained diminished through the end of the study. Single agent XENP24306+XENP32803 treatment also resulted in significant tumor growth inhibition in comparison to single agent XENP16432/anti-PD1 (Group B) treatment as early as on Day 8 for the 0.1 mg/kg XENP24306+XENP32803 treated animals (Group E). By Day 13, 1.0 mg/kg XENP24306+XENP32803 (Group C) gained significance over XENP16432/anti-PD1 with respect to tumor volume reductions, while for 0.3 mg/kg XENP24306+XENP32803 (Group D) significance over XENP16432/anti-PD1 occurred on Day 19.

In addition, compared with the anti-PD-1 (alone) treatment group, higher doses of XENP24306+XENP32803 (0.3 and 1.0 mg/kg) in combination with the anti-PD-1 inhibitor showed significantly greater reduction in tumor growth, higher peripheral CD8⁺ T cell and NK-cell expansion, and IFNγ elevation. In particular, when dosed in combination with XENP16432/anti-PD1, 0.3 and 0.1 mg/kg XENP24306+XENP32803 (Groups G and H) resulted in dose dependent and statistically significant tumor volume reductions as early as on Day 8 in comparison to both the PBS control and single agent XENP16432/anti-PD1 groups. All three combination dose groups of XENP24306+XENP32803 with XEN16432 displayed dose-dependent, statistically significant tumor size reductions in comparison to both PBS and single agent XENP16432/anti-PD1 on Day 11.

This study describes the anti-tumor activity of XENP24306+XENP32803, an IL15/IL15Rα-Fc fusion protein. Importantly, this study also demonstrates the additional benefit of combined treatment using XENP24306+XENP32803 and XENP16432, an anti-PD1 bivalent antibody, administered together to enhance anti-tumor immune responses over anti-PD1 treatment alone, suggesting the possibility of improving clinical benefit by combining approved anti-PD-L1 agents with XENP24306+XENP32803. Dose-dependent XENP24306+XENP32803 anti-tumor activity was correlated with dose-dependent increases in peripheral blood leukocyte numbers and elevations in IFNγ production.

All dose levels, including the lowest level of 0.1 mg/kg XENP24306+XENP32803, were active in this antitumor model, and all dose levels of XENP24306+XENP32803 promoted increased leukocyte expansion and IFNγ production, with the highest 1 mg/kg dose of XENP24306+XENP32803 mediating the greatest effects. Combination treatment of XENP24306+XENP32803 with XENP16432/anti-PD1 also resulted in an increased enhancement of leukocyte numbers and IFNγ production in comparison to anti-PD1 monotherapy.

### Example 10: Combination Therapy, Open-Label, Multicenter, Global, Dose-Escalation Study of XENP24306+XENP32803 in Combination with Atezolizumab

A combination therapy, open-label, multicenter, global, dose-escalation study to evaluate the safety, tolerability pharmacokinetics and activity of XENP24306 (e.g., ˜82%)+XENP32803 (e.g., ˜18%) in combination with an anti-PD-L1/PD-1 antibody such as atezolizumab will be conducted.

The study consists of a screening period of up to 28 days, a treatment period, and a minimum follow-up period of 90 days after treatment. Patients considering enrollment into combination therapy expansion cohorts with PD-L1 selected tumors can have tissue prescreening for PD-L1 status performed prior to the 28-day screening period.

Patients will be enrolled in two stages: a dose-escalation stage and an expansion stage.

Approximately 21-54 patients with locally advanced, recurrent, or metastatic incurable solid tumors will be enrolled in the dose-escalation stage for the combination therapy portion of the study. XENP24306+XENP32803 and atezolizumab will be administered by IV infusion. Following confirmation of eligibility, patients will receive XENP24306+XENP32803 in combination with atezolizumab by IV infusion on the first day of every 14-day cycle. The combination therapy starting dose of XENP24306+XENP32803 will be 0.01 mg/kg IV every two weeks. Atezolizumab will be administered by IV infusion at a fixed dose of 840 mg on Day 1 of each 14-day cycle in combination with XENP24306+XENP32803. Atezolizumab will be administered after XENP24306+XENP32803 and subsequent observation period.

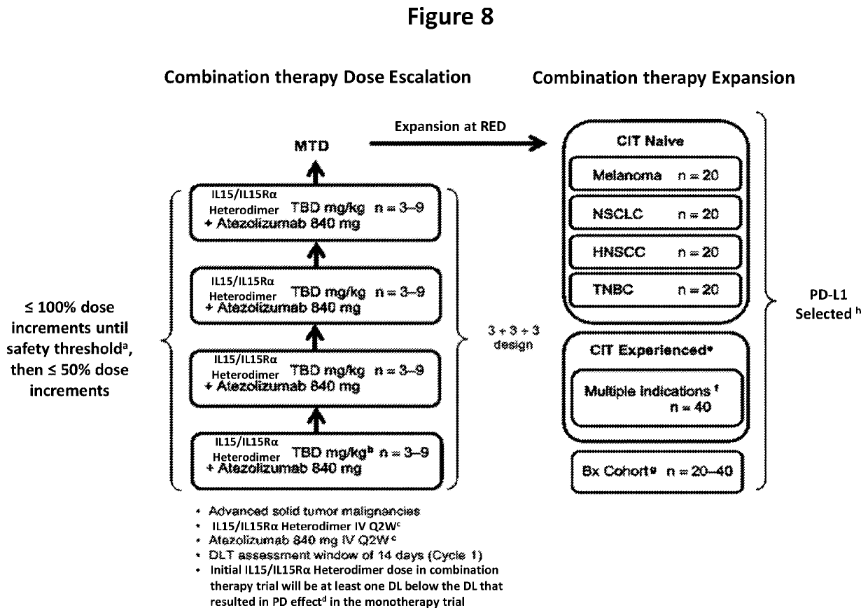

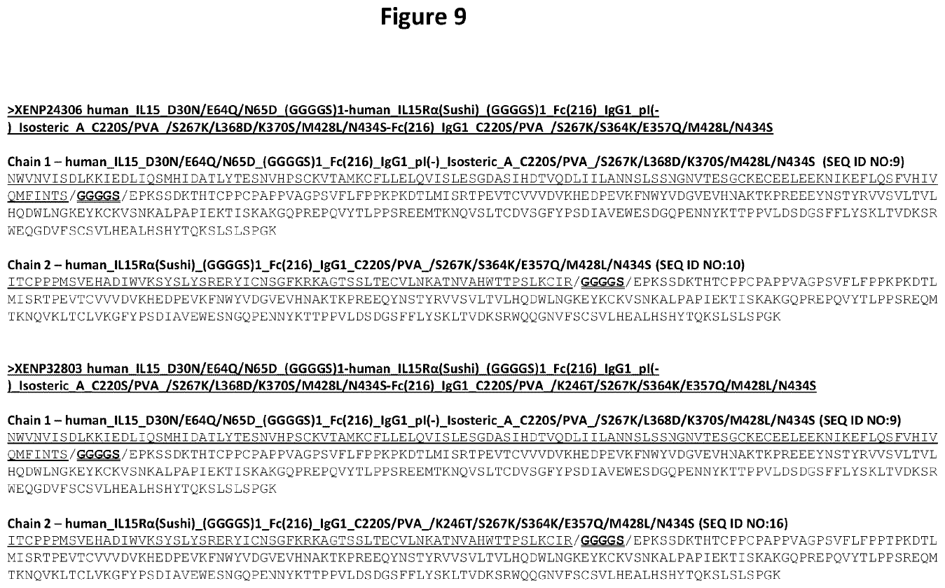

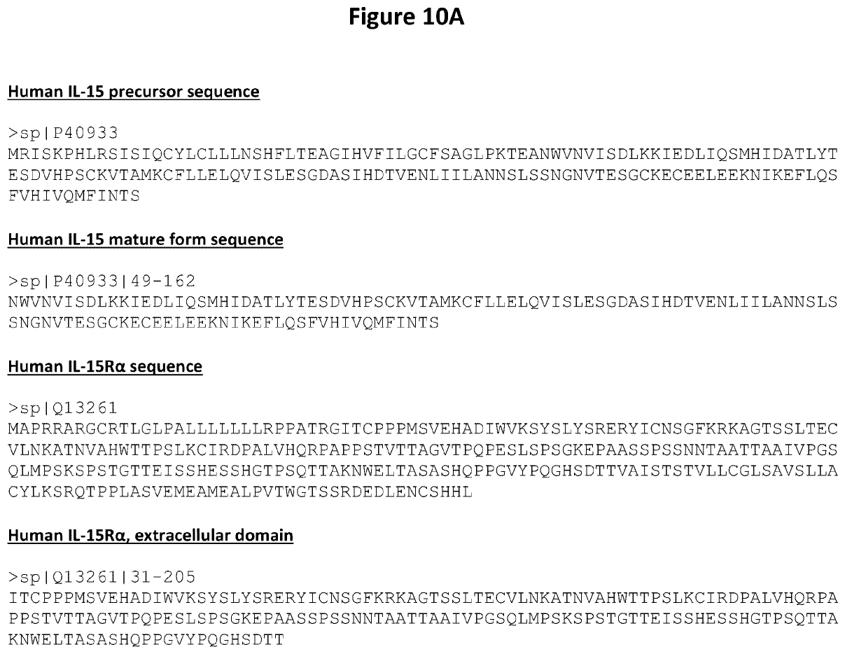

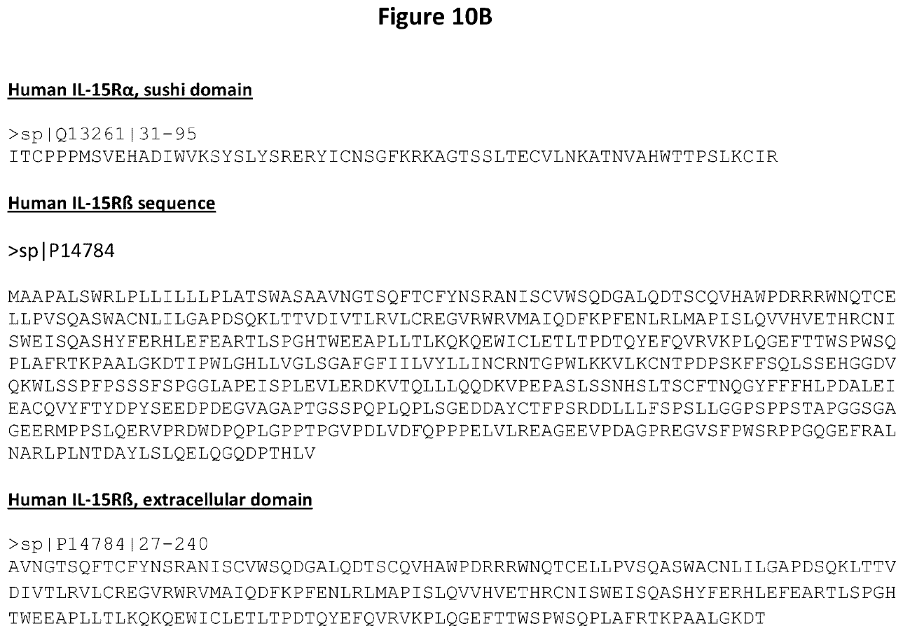

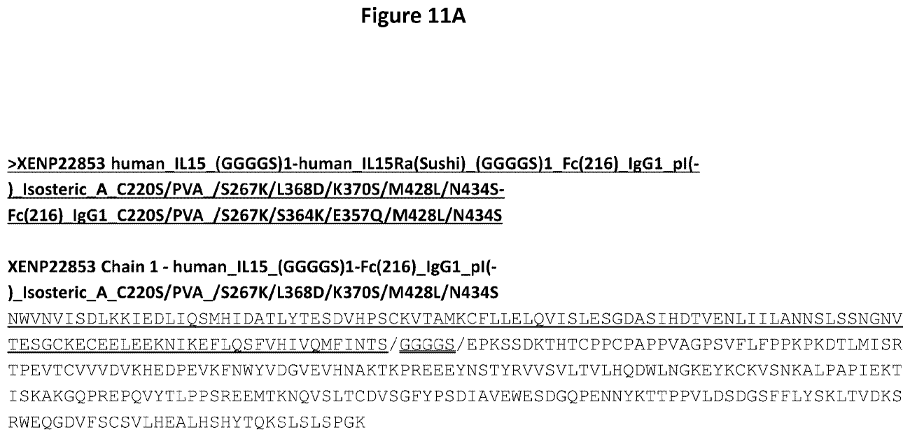

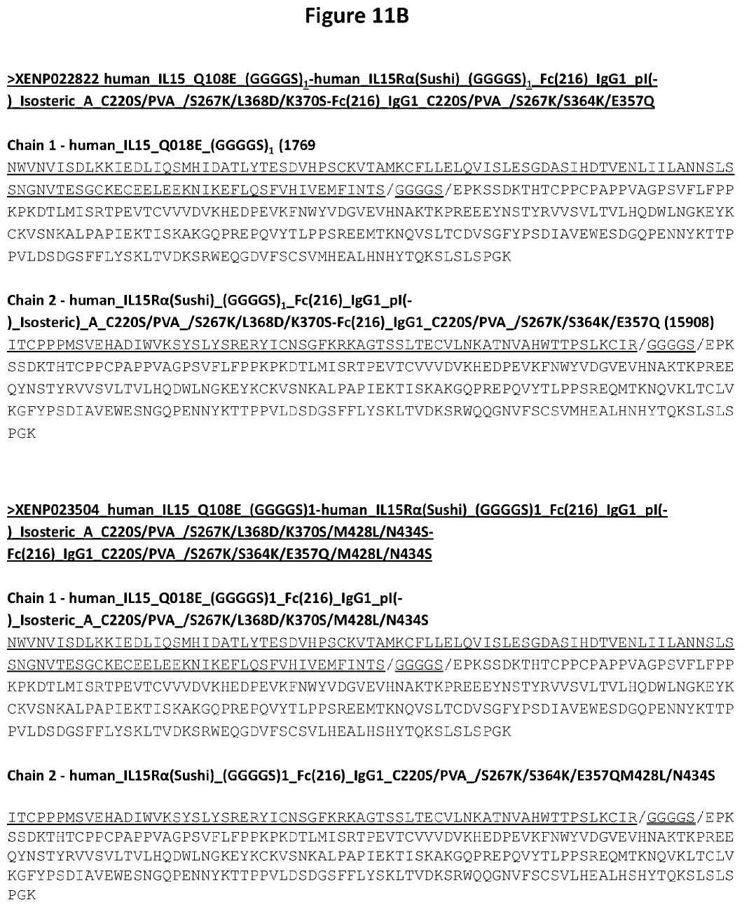

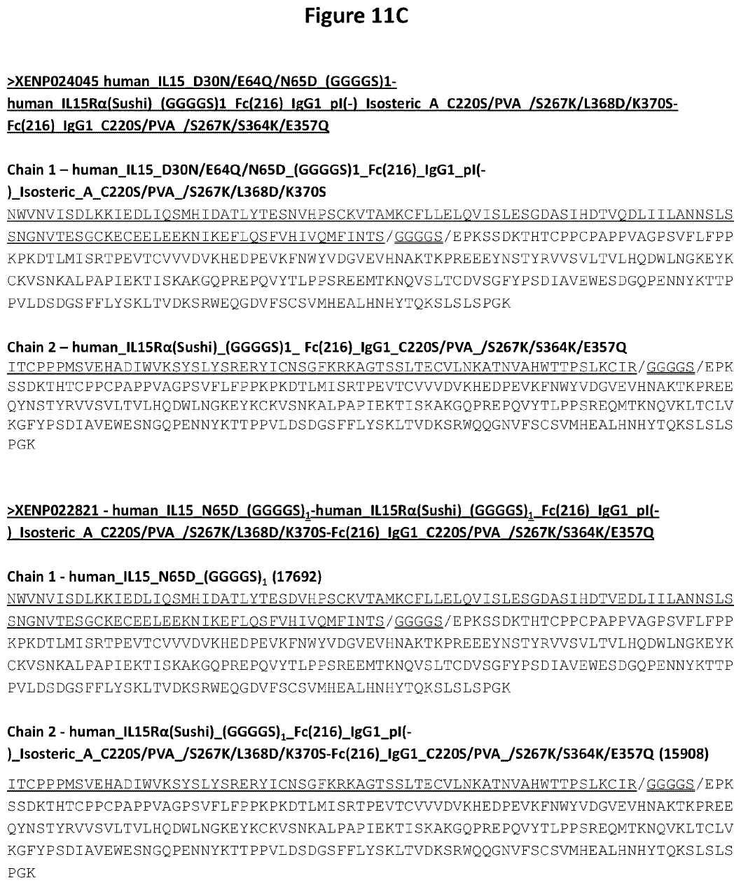

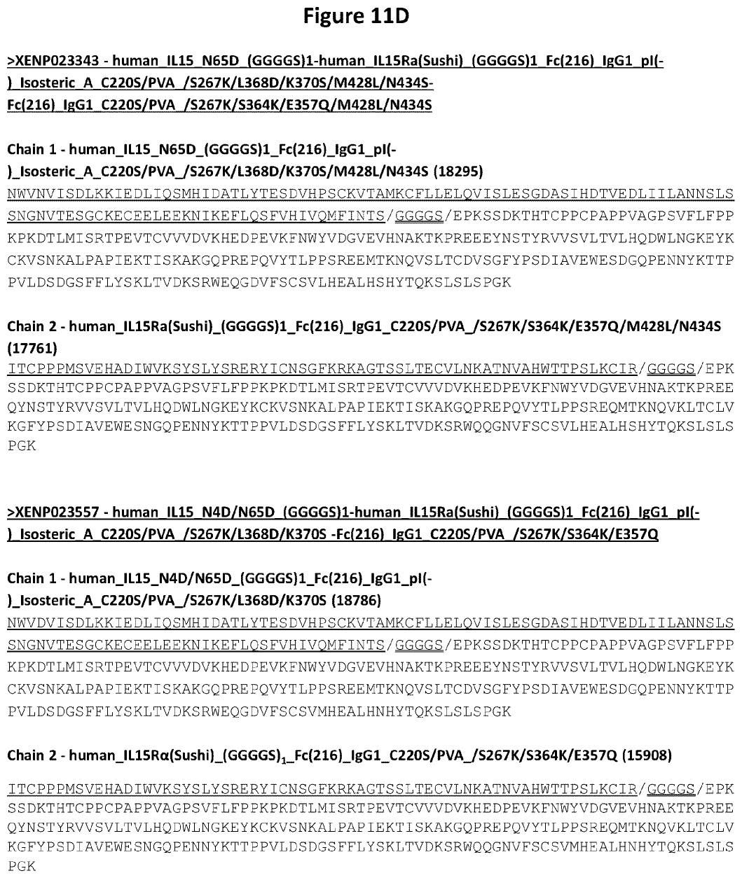

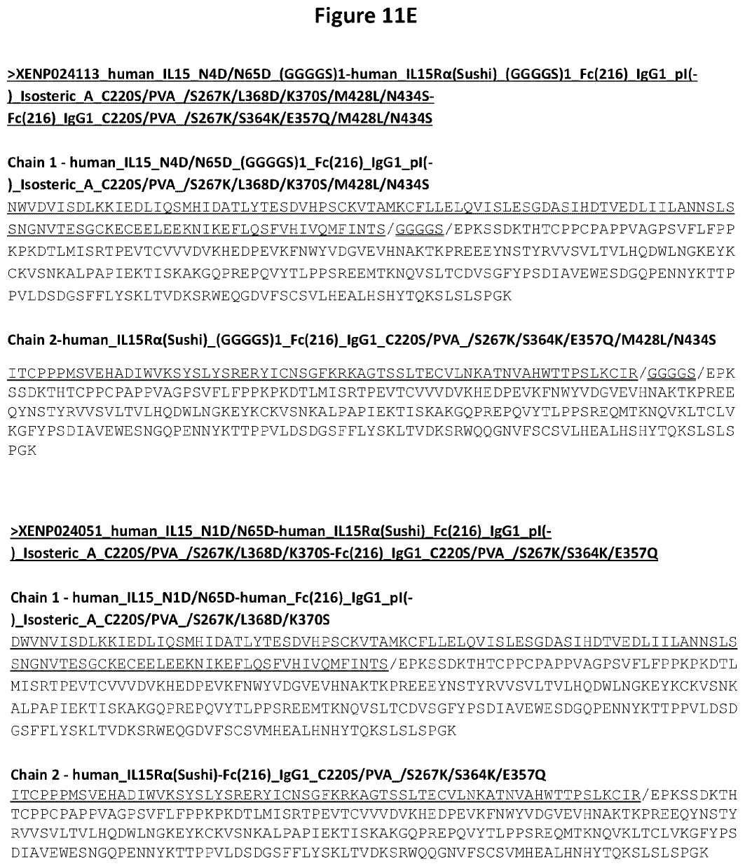

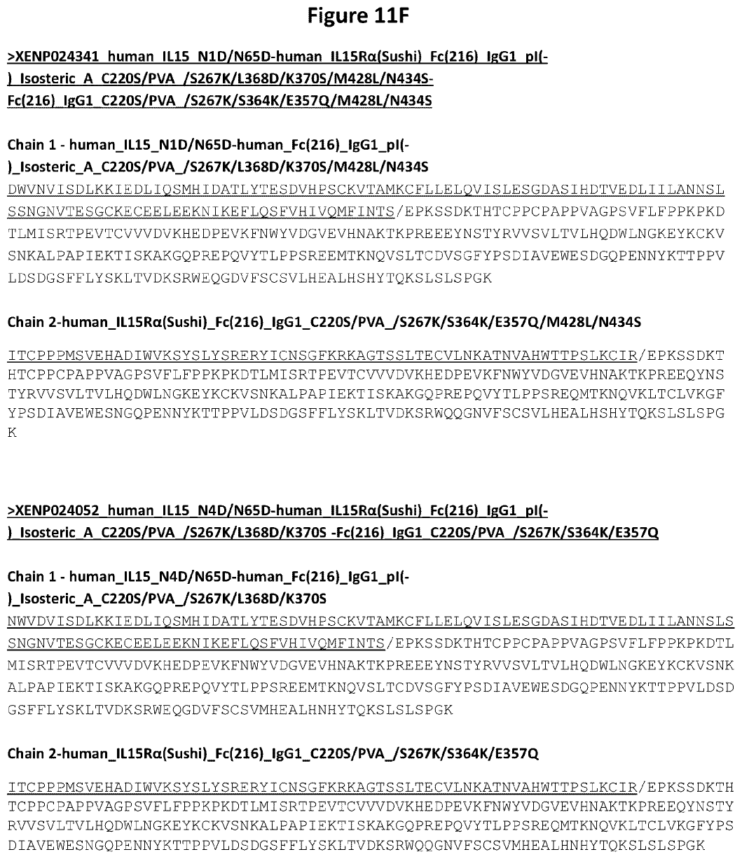

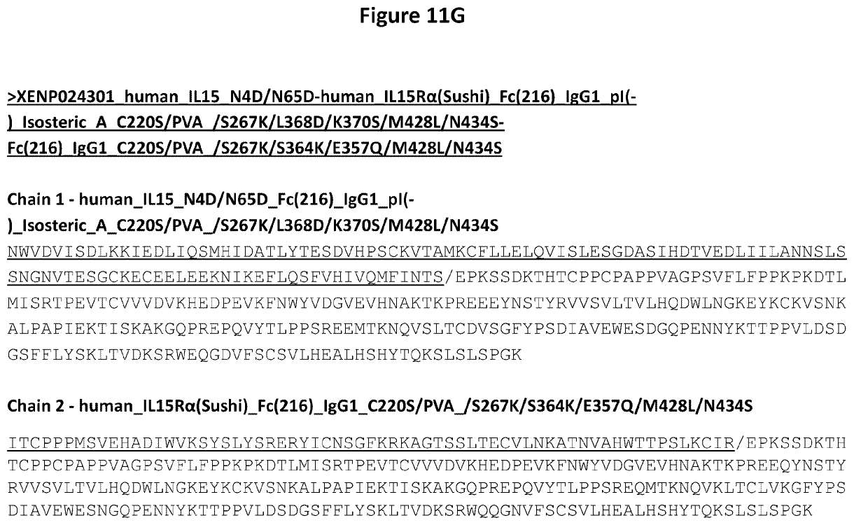

The XENP24306+XENP32803 dose will be increased by up to 100% of the preceding dose level for each successive cohort, until a safety threshold (defined as a DLT in 1 patient or a Grade ≥2 major organ adverse event not attributable to another clearly identifiable cause in at least 2 patients during the DLT assessment window in a given cohort) is observed. Subsequently, cohorts of 3-9 patients each will be evaluated at escalating dose levels following a 3+3+3 design to determine the MTD (or MAD) for XENP24306+XENP32803 in combination with atezolizumab. FIG. 8.

Patients enrolling into cleared cohorts of combination therapy dose-escalation cohorts (i.e., backfill cohorts) must meet have one of the following PD-L1-selected tumor types: melanoma, non-small cell lung cancer (NSCLC), head and neck squamous cell carcinoma (HNSCC), triple-negative breast cancer (TNBC) urothelial carcinoma (UCC), renal cell carcinoma (RCC), small cell lung carcinoma (SCLC), gastric cancer (GC), Merkel cell carcinoma (MCC), cutaneous squamous cell carcinoma (cSCC), microsatellite instability-high (MSI-H) cancers.

In total, up to approximately 225-350 patients may be enrolled in this study, at approximately 25-35 global investigative sites. Patients in this study will be initially assessed for eligibility during the screening period (lasting ≤28 days). The starting dose of XENP24306+XENP32803 in combination with atezolizumab will be no higher than one dose level below the XENP24306+XENP32803 dose demonstrating PD activity in the monotherapy portion of the study (Example 6). In the case that the initial monotherapy XENP24306+XENP32803 dose level of 0.01 mg/kg demonstrates PD activity, the XENP24306+XENP32803 starting dose will be no higher than 0.005 mg/kg in the initial atezolizumab combination cohort. XENP24306+XENP32803 and atezolizumab will be administered by IV infusion in the expansion stage. A provisional XENP24306+XENP32803 recommended expansion dose (RED) will be proposed at or below the MTD/MAD established in dose escalation.

Once the RED of XENP24306+XENP32803 in combination with atezolizumab has been proposed, additional patients will be enrolled in the expansion stage and treated at the RED.

XENP24306+XENP32803 PK will be assessed. Patients will be evaluated weekly by physical examination and blood collections for routine hematologic and metabolic laboratory assessments for the first eight cycles of XENP24306+XENP32803 in combination with atezolizumab treatment during dose escalation, the first two cycles during expansion, and less frequently thereafter. Tumor assessment will occur at baseline and after initiation of study.

All patients will be closely monitored for adverse events throughout the study and for at least 90 days after the final dose of study treatment or until initiation of another systemic anti-cancer therapy, whichever occurs first. Adverse events will be graded according to NCI CTCAE v5.0.

To characterize the pharmacokinetics, immunogenicity response, and PD properties of XENP24306+XENP32803 in combination with atezolizumab, blood samples will be taken at various timepoints before and after dosing.

Patients will undergo tumor assessments at screening (baseline) and at regular intervals during the study, which will be measured by RECIST v1.1. iRECIST will also be used in this study to better characterize the different patterns of responses associated with cancer immunotherapy (CIT) and to allow a better understanding of the preliminary activity profile of XENP24306+XENP32803 in combination with atezolizumab. iRECIST is intended to supplement standard RECIST v1.1 in this study to allow the investigators to make an integrated assessment of benefit and risk for patients.

The activity objective for this study is to make a preliminary assessment of the activity of XENP24306+XENP32803 when administered in combination with atezolizumab, on the basis of the following endpoints:
Serum concentration of XENP24306+XENP32803;
Percentage of participants with adverse events;
Objective response rate (ORR), defined as the proportion of patients with a complete response (CR) or partial response (PR) on two consecutive occasions ≥4 weeks apart;
Duration of response (DOR), defined as the time from the first occurrence of a documented objective response to disease progression or death from any cause (whichever occurs first;
Progression-free survival (PFS) after enrollment, defined as the time from enrollment to the first occurrence of disease progression or death from any cause (whichever occurs first); and
Overall survival (OS) after enrollment, defined as the time from enrollment to death from any cause.

The safety objective for this study is to evaluate the safety of XENP24306+XENP32803 when administered in combination with atezolizumab, based on the incidence and severity of adverse events and on changes from baseline in targeted vital signs, or clinical laboratory test results or ECGs parameters.

The pharmacokinetic (PK) objective for this study is to characterize the XENP24306+XENP32803 PK profile when administered in combination with atezolizumab, on the basis of serum concentration of XENP24306+XENP32803 at specified timepoints.

The immunogenicity objective for this study is to evaluate the immune response to XENP24306+XENP32803 when administered in combination with atezolizumab, on the basis of ADAs to XENP24306+XENP32803 and ADAs to XENP24306+XENP32803 and atezolizumab during the study.

### Example 11: Combination Therapy, Open-Label, Multicenter, Global, Dose-Escalation Study of XENP24306 in Combination with Atezolizumab

A combination therapy, open-label, multicenter, global, dose-escalation study to evaluate the safety, tolerability pharmacokinetics and activity of XENP24306 in combination with an anti-PD-L1/PD-1 antibody such as atezolizumab will be conducted.

The study consists of a screening period of up to 28 days, a treatment period, and a minimum follow-up period of 90 days after treatment. Patients considering enrollment into combination therapy expansion cohorts with PD-L1 selected tumors can have tissue prescreening for PD-L1 status performed prior to the 28-day screening period.

Patients will be enrolled in two stages: a dose-escalation stage and an expansion stage.

Approximately 21-54 patients with locally advanced, recurrent, or metastatic incurable solid tumors will be enrolled in the dose-escalation stage for the combination therapy portion of the study. XENP24306 and atezolizumab will be administered by IV infusion. Following confirmation of eligibility, patients will receive XENP24306 in combination with atezolizumab by IV infusion on the first day of every 14-day cycle. The combination therapy starting dose of XENP24306 will be 0.01 mg/kg IV every two weeks. Atezolizumab will be administered by IV infusion at a fixed dose of 840 mg on Day 1 of each 14-day cycle in combination with XENP24306. Atezolizumab will be administered after XENP24306 and subsequent observation period.

The XENP24306 dose will be increased by up to 100% of the preceding dose level for each successive cohort, until a safety threshold (defined as a DLT in 1 patient or a Grade ≥2 major organ adverse event not attributable to another clearly identifiable cause in at least 2 patients during the DLT assessment window in a given cohort) is observed. Subsequently, cohorts of 3-9 patients each will be evaluated at escalating dose levels following a 3+3+3 design to determine the MTD (or MAD) for XENP24306 in combination with atezolizumab. FIG. 8.

Patients enrolling into cleared cohorts of combination therapy dose-escalation cohorts (i.e., backfill cohorts) must meet have one of the following PD-L1-selected tumor types: melanoma, non-small cell lung cancer (NSCLC), head and neck squamous cell carcinoma (HNSCC), triple-negative breast cancer (TNBC) urothelial carcinoma (UCC), renal cell carcinoma (RCC), small cell lung carcinoma (SCLC), gastric cancer (GC), Merkel cell carcinoma (MCC), cutaneous squamous cell carcinoma (cSCC), microsatellite instability-high (MSI-H) cancers.

In total, up to approximately 225-350 patients may be enrolled in this study, at approximately 25-35 global investigative sites. Patients in this study will be initially assessed for eligibility during the screening period (lasting ≤28 days). The starting dose of XENP24306 in combination with atezolizumab will be no higher than one dose level below the XENP24306 dose demonstrating PD activity in the monotherapy portion of the study (Example 6). In the case that the initial monotherapy XENP24306 dose level of 0.01 mg/kg demonstrates PD activity, the XENP24306 starting dose will be no higher than 0.005 mg/kg in the initial atezolizumab combination cohort. XENP24306 and atezolizumab will be administered by IV infusion in the expansion stage. A provisional XENP24306 recommended expansion dose (RED) will be proposed at or below the MTD/MAD established in dose escalation.

Once the RED of XENP24306 in combination with atezolizumab has been proposed, additional patients will be enrolled in the expansion stage and treated at the RED.

XENP24306 PK will be assessed. Patients will be evaluated weekly by physical examination and blood collections for routine hematologic and metabolic laboratory assessments for the first eight cycles of XENP24306 in combination with atezolizumab treatment during dose escalation, the first two cycles during expansion, and less frequently thereafter. Tumor assessment will occur at baseline and after initiation of study.

All patients will be closely monitored for adverse events throughout the study and for at least 90 days after the final dose of study treatment or until initiation of another systemic anti-cancer therapy, whichever occurs first. Adverse events will be graded according to NCI CTCAE v5.0.

To characterize the pharmacokinetics, immunogenicity response, and PD properties of XENP24306 in combination with atezolizumab, blood samples will be taken at various timepoints before and after dosing.

Patients will undergo tumor assessments at screening (baseline) and at regular intervals during the study, which will be measured by RECIST v1.1. iRECIST will also be used in this study to better characterize the different patterns of responses associated with cancer immunotherapy (CIT) and to allow a better understanding of the preliminary activity profile of XENP24306 in combination with atezolizumab. iRECIST is intended to supplement standard RECIST v1.1 in this study to allow the investigators to make an integrated assessment of benefit and risk for patients.

The activity objective for this study is to make a preliminary assessment of the activity of XENP24306 when administered in combination with atezolizumab, on the basis of the following endpoints:
Serum concentration of XENP24306;
Percentage of participants with adverse events;
Objective response rate (ORR), defined as the proportion of patients with a complete response (CR) or partial response (PR) on two consecutive occasions ≥4 weeks apart;
Duration of response (DOR), defined as the time from the first occurrence of a documented objective response to disease progression or death from any cause (whichever occurs first;
Progression-free survival (PFS) after enrollment, defined as the time from enrollment to the first occurrence of disease progression or death from any cause (whichever occurs first); and
Overall survival (OS) after enrollment, defined as the time from enrollment to death from any cause.

The safety objective for this study is to evaluate the safety of XENP24306 when administered in combination with atezolizumab, based on the incidence and severity of adverse events and on changes from baseline in targeted vital signs, or clinical laboratory test results or ECGs parameters.

The pharmacokinetic (PK) objective for this study is to characterize the XENP24306 PK profile when administered in combination with atezolizumab, on the basis of serum concentration of XENP24306 at specified timepoints.

The immunogenicity objective for this study is to evaluate the immune response to XENP24306 when administered in combination with atezolizumab, on the basis of ADAs to XENP24306 and ADAs to XENP24306 and atezolizumab during the study.

### Example 12: Combination Therapy, Open-Label, Multicenter, Global, Dose-Escalation Study of XENP32803 in Combination with Atezolizumab

A combination therapy, open-label, multicenter, global, dose-escalation study to evaluate the safety, tolerability pharmacokinetics and activity of XENP32803 in combination with an anti-PD-L1/PD-1 antibody such as atezolizumab will be conducted.

The study consists of a screening period of up to 28 days, a treatment period, and a minimum follow-up period of 90 days after treatment. Patients considering enrollment into combination therapy expansion cohorts with PD-L1 selected tumors can have tissue prescreening for PD-L1 status performed prior to the 28-day screening period.

Patients will be enrolled in two stages: a dose-escalation stage and an expansion stage.

Approximately 21-54 patients with locally advanced, recurrent, or metastatic incurable solid tumors will be enrolled in the dose-escalation stage for the combination therapy portion of the study. XENP32803 and atezolizumab will be administered by IV infusion. Following confirmation of eligibility, patients will receive XENP32803 in combination with atezolizumab by IV infusion on the first day of every 14-day cycle. The combination therapy starting dose of XENP32803 will be 0.01 mg/kg IV every two weeks. Atezolizumab will be administered by IV infusion at a fixed dose of 840 mg on Day 1 of each 14-day cycle in combination with XENP32803. Atezolizumab will be administered after XENP32803 and subsequent observation period.

The XENP32803 dose will be increased by up to 100% of the preceding dose level for each successive cohort, until a safety threshold (defined as a DLT in 1 patient or a Grade ≥2 major organ adverse event not attributable to another clearly identifiable cause in at least 2 patients during the DLT assessment window in a given cohort) is observed. Subsequently, cohorts of 3-9 patients each will be evaluated at escalating dose levels following a 3+3+3 design to determine the MTD (or MAD) for XENP32803 in combination with atezolizumab. FIG. 8.

Patients enrolling into cleared cohorts of combination therapy dose-escalation cohorts (i.e., backfill cohorts) must meet have one of the following PD-L1-selected tumor types: melanoma, non-small cell lung cancer (NSCLC), head and neck squamous cell carcinoma (HNSCC), triple-negative breast cancer (TNBC) urothelial carcinoma (UCC), renal cell carcinoma (RCC), small cell lung carcinoma (SCLC), gastric cancer (GC), Merkel cell carcinoma (MCC), cutaneous squamous cell carcinoma (cSCC), microsatellite instability-high (MSI-H) cancers.

In total, up to approximately 225-350 patients may be enrolled in this study, at approximately 25-35 global investigative sites. Patients in this study will be initially assessed for eligibility during the screening period (lasting ≤28 days). The starting dose of XENP32803 in combination with atezolizumab will be no higher than one dose level below the XENP32803 dose demonstrating PD activity in the monotherapy portion of the study (Example 6). In the case that the initial monotherapy XENP32803 dose level of 0.01 mg/kg demonstrates PD activity, the XENP32803 starting dose will be no higher than 0.005 mg/kg in the initial atezolizumab combination cohort. XENP32803 and atezolizumab will be administered by IV infusion in the expansion stage. A provisional XENP32803 recommended expansion dose (RED) will be proposed at or below the MTD/MAD established in dose escalation.

Once the RED of XENP32803 in combination with atezolizumab has been proposed, additional patients will be enrolled in the expansion stage and treated at the RED.

XENP32803 PK will be assessed. Patients will be evaluated weekly by physical examination and blood collections for routine hematologic and metabolic laboratory assessments for the first eight cycles of XENP32803 in combination with atezolizumab treatment during dose escalation, the first two cycles during expansion, and less frequently thereafter. Tumor assessment will occur at baseline and after initiation of study.

All patients will be closely monitored for adverse events throughout the study and for at least 90 days after the final dose of study treatment or until initiation of another systemic anti-cancer therapy, whichever occurs first. Adverse events will be graded according to NCI CTCAE v5.0.

To characterize the pharmacokinetics, immunogenicity response, and PD properties of XENP32803 in combination with atezolizumab, blood samples will be taken at various timepoints before and after dosing.

Patients will undergo tumor assessments at screening (baseline) and at regular intervals during the study, which will be measured by RECIST v1.1. iRECIST will also be used in this study to better characterize the different patterns of responses associated with cancer immunotherapy (CIT) and to allow a better understanding of the preliminary activity profile of XENP32803 in combination with atezolizumab. iRECIST is intended to supplement standard RECIST v1.1 in this study to allow the investigators to make an integrated assessment of benefit and risk for patients.

The activity objective for this study is to make a preliminary assessment of the activity of XENP32803 when administered in combination with atezolizumab, on the basis of the following endpoints:
Serum concentration of XENP32803;
Percentage of participants with adverse events;
Objective response rate (ORR), defined as the proportion of patients with a complete response (CR) or partial response (PR) on two consecutive occasions ≥4 weeks apart;
Duration of response (DOR), defined as the time from the first occurrence of a documented objective response to disease progression or death from any cause (whichever occurs first;
Progression-free survival (PFS) after enrollment, defined as the time from enrollment to the first occurrence of disease progression or death from any cause (whichever occurs first); and
Overall survival (OS) after enrollment, defined as the time from enrollment to death from any cause.

The safety objective for this study is to evaluate the safety of XENP32803 when administered in combination with atezolizumab, based on the incidence and severity of adverse events and on changes from baseline in targeted vital signs, or clinical laboratory test results or ECGs parameters.

The pharmacokinetic (PK) objective for this study is to characterize the XENP32803 PK profile when administered in combination with atezolizumab, on the basis of serum concentration of XENP32803 at specified timepoints.

The immunogenicity objective for this study is to evaluate the immune response to XENP32803 when administered in combination with atezolizumab, on the basis of ADAs to XENP32803 and ADAs to XENP32803 and atezolizumab during the study.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### Example 13: Open-Label, Multicenter, Global, Dose-Escalation Study of a Combination of IL15/IL15Rα Heterodimeric Proteins Alone or in Combination with Atezolizumab

A monotherapy, open-label, multicenter, global, dose-escalation study to evaluate the safety, tolerability pharmacokinetics and activity of a combination of IL15/IL15Rα heterodimeric proteins (XENP24306 (˜82%) and XENP32803 (˜18%) (“XENP24306+XENP32803”)) in accordance with Example 6 and a combination therapy, open-label, multicenter, global, dose-escalation study to evaluate the safety, tolerability pharmacokinetics and activity of XENP24306+XENP32803 in combination with an anti-PD-L1/PD-1 antibody such as atezolizumab in accordance with Example 10 were conducted.

Thirteen patients suffering from a solid tumor were recruited to the study. In the dose escalation arm of the study (phase 1a), one patient received 0.01 mg/ml XENP24306+XENP32803; three patients received 0.02 mg/ml XENP24306+XENP32803; three patients received 0.04 mg/ml XENP24306+XENP32803; three patients received 0.06 mg/ml XENP24306+XENP32803; and three patients received 0.09 mg/ml XENP24306+XENP32803 by IV infusion on the first day of every 14-day cycle (Q2W). See, Example 6 and FIG. 7. Pharmacodynamic (PD) activity in these patients was monitored by the expansion of CD8⁺ T cell and/or NK cells.

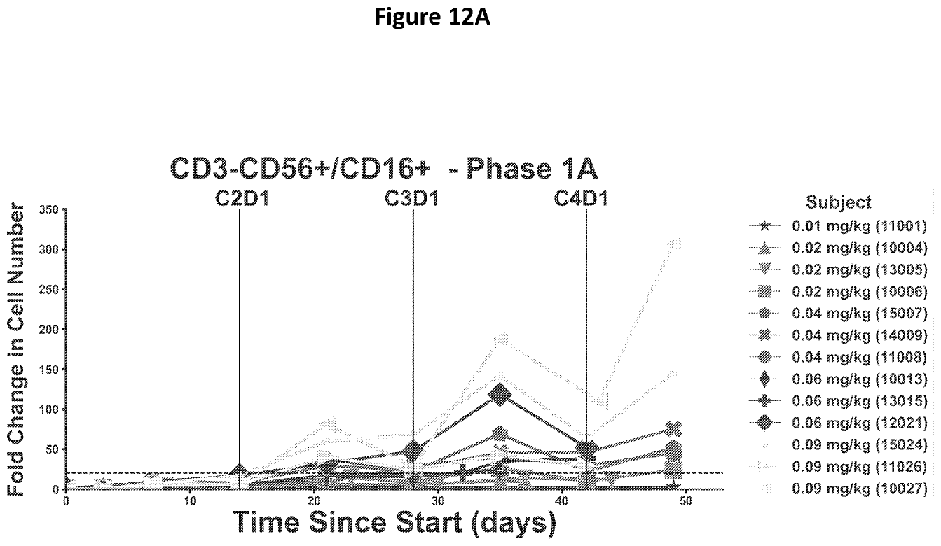

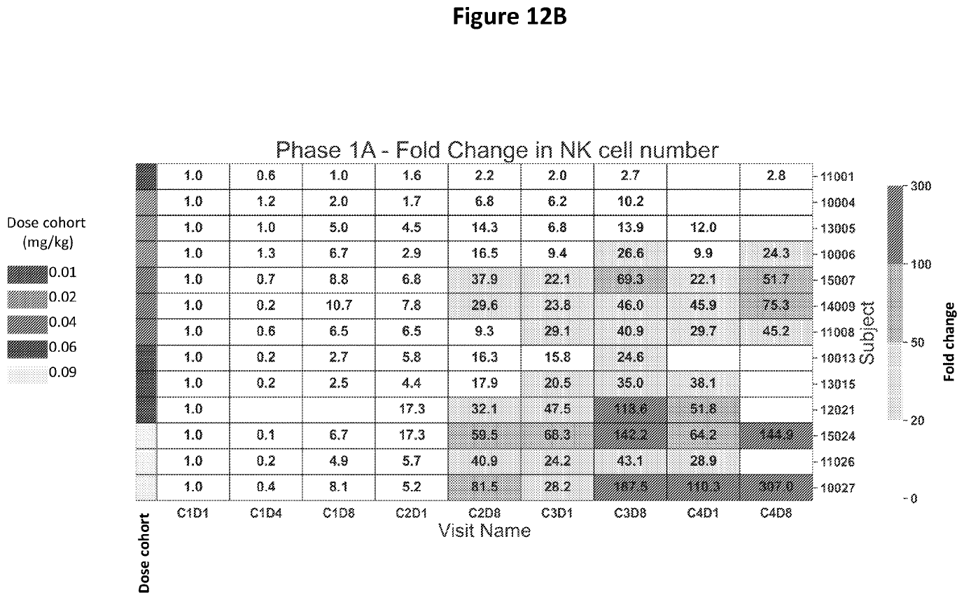

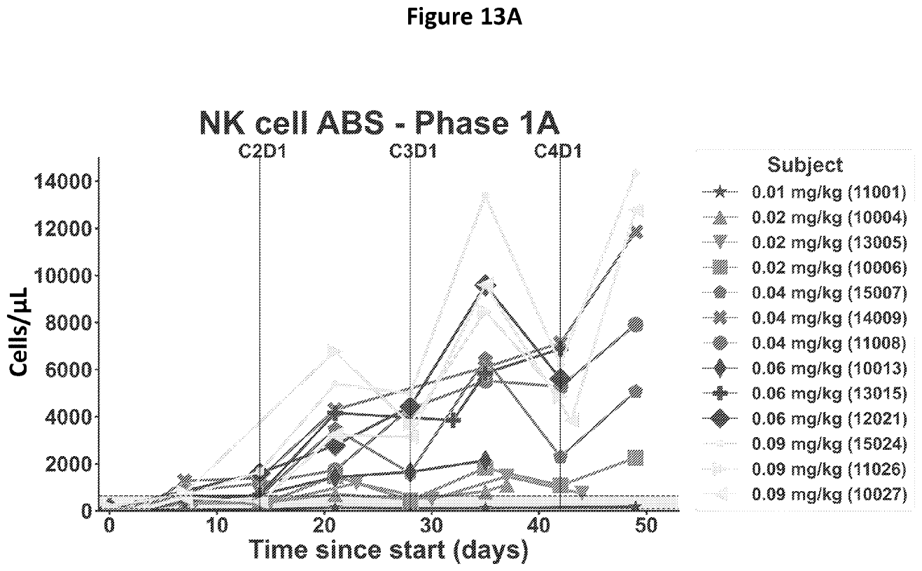

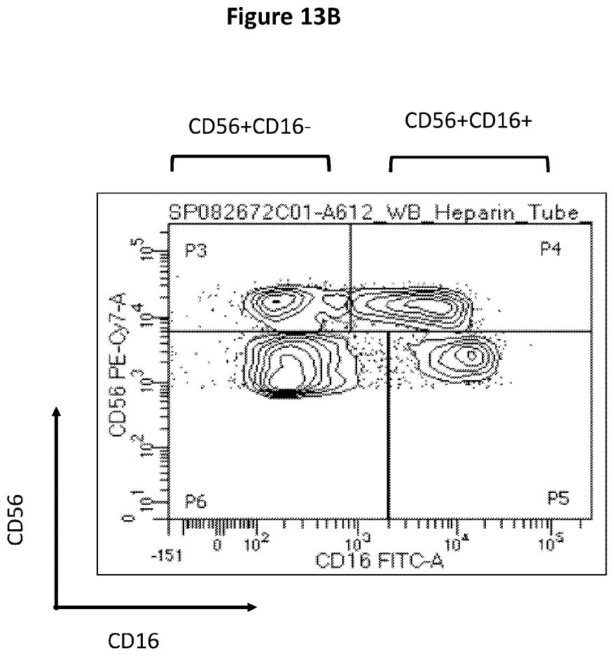

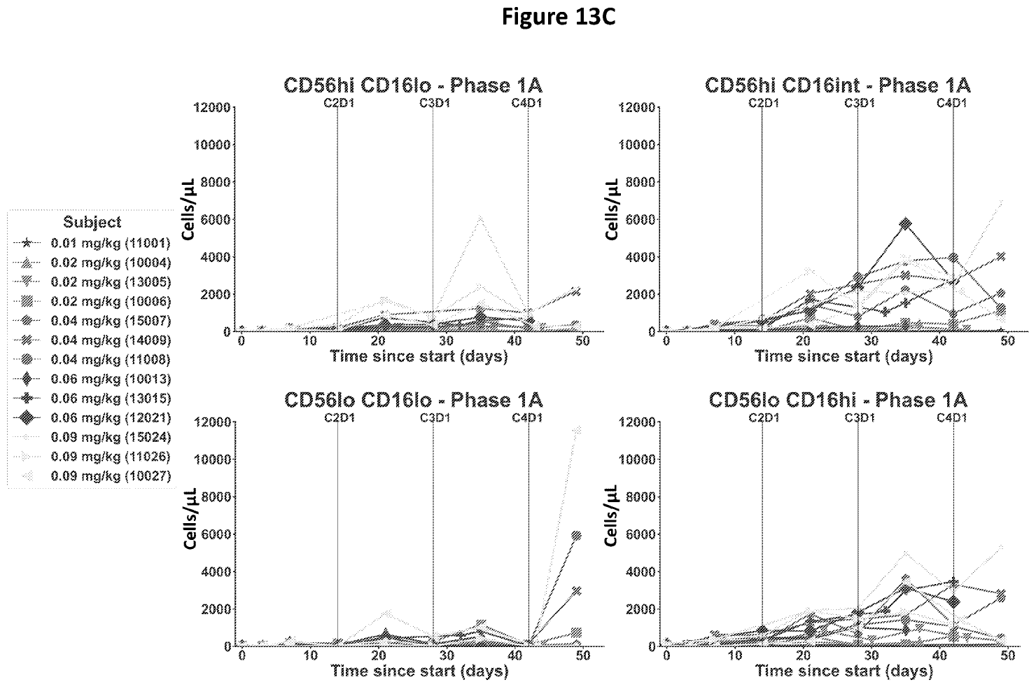

Dose dependent expansion of CD16+ NK cells (in both CD56 hi and CD56 lo subpopulations) was observed with XENP24306+XENP32803 in the phase 1a dose escalation study. See FIGS. 12A-12B and 13A-13C. Unexpectedly, greater than 20-fold increases in NK cells were observed. Additionally, a surprising cycle-to-cycle accumulation of NK cells was observed. Cf. FIGS. 3C and 3D.

The starting dose of XENP24306+XENP32803 for the combination therapy arm of the study (phase 1b) was set at 0.01 mg/kg XENP24306+XENP32803. Twelve patients suffering from a solid tumor were recruited to the study, including one patient (patient 15007) that was a crossover from the phase 1a trial. Four patients (including crossover patient 15007) received 0.01 mg/ml XENP24306+XENP32803 in combination with 840 mg atezolizumab; five patients received 0.02 mg/ml XENP24306+XENP32803 in combination with 840 mg atezolizumab; and three patients received 0.04 mg/ml XENP24306+XENP32803 in combination with 840 mg atezolizumab by IV Q2W. See, Example 10 and FIG. 8. Pharmacodynamic (PD) activity in these patients was monitored by the expansion of CD8+ T cell and/or NK cells.

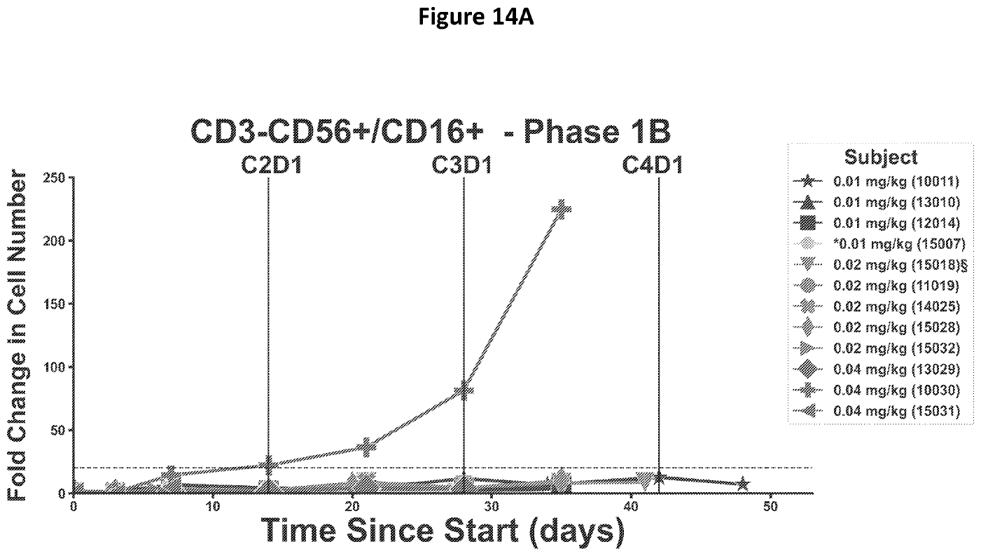

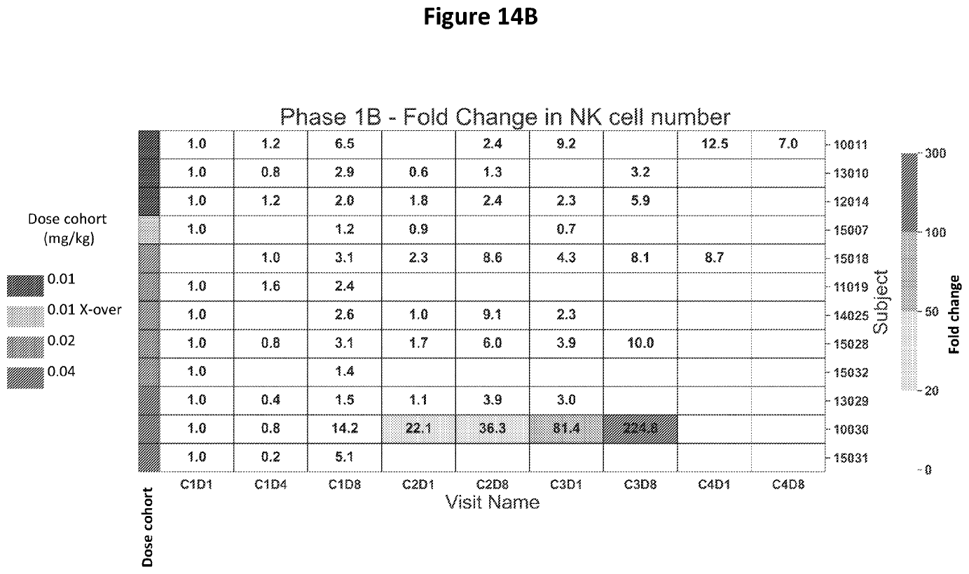

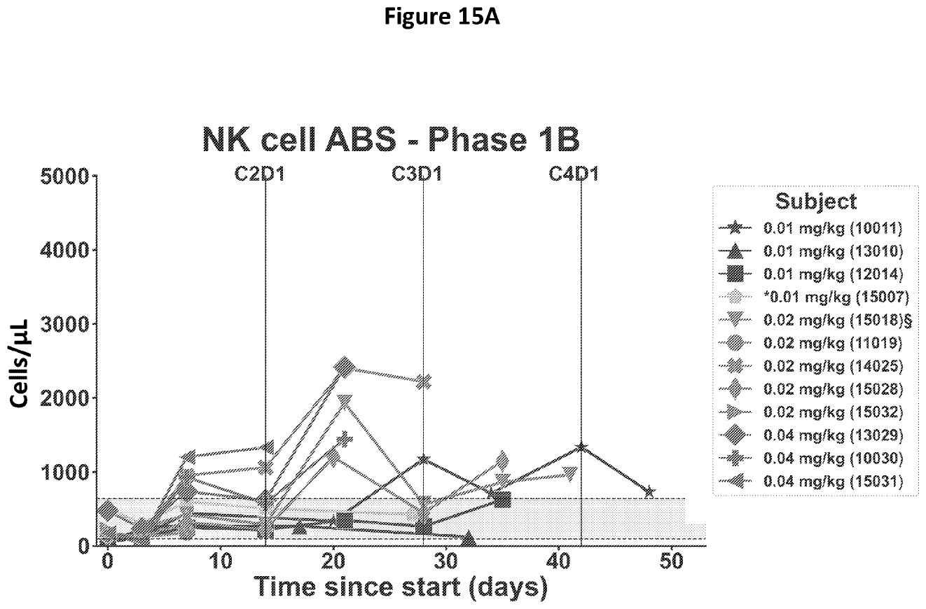

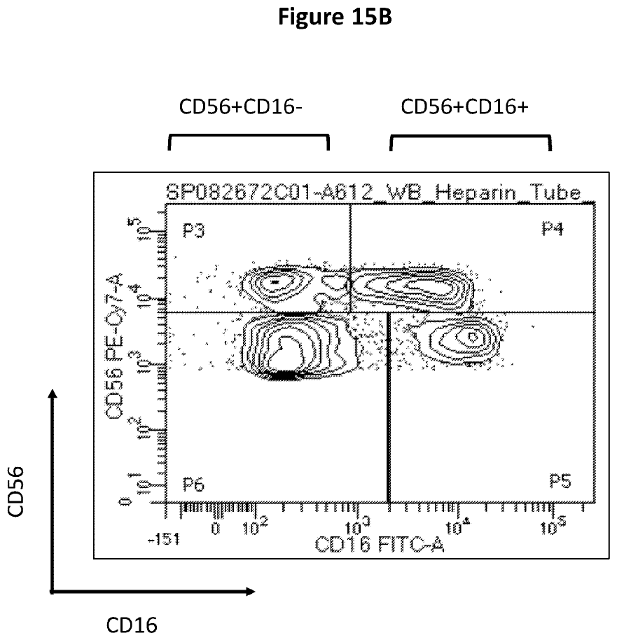

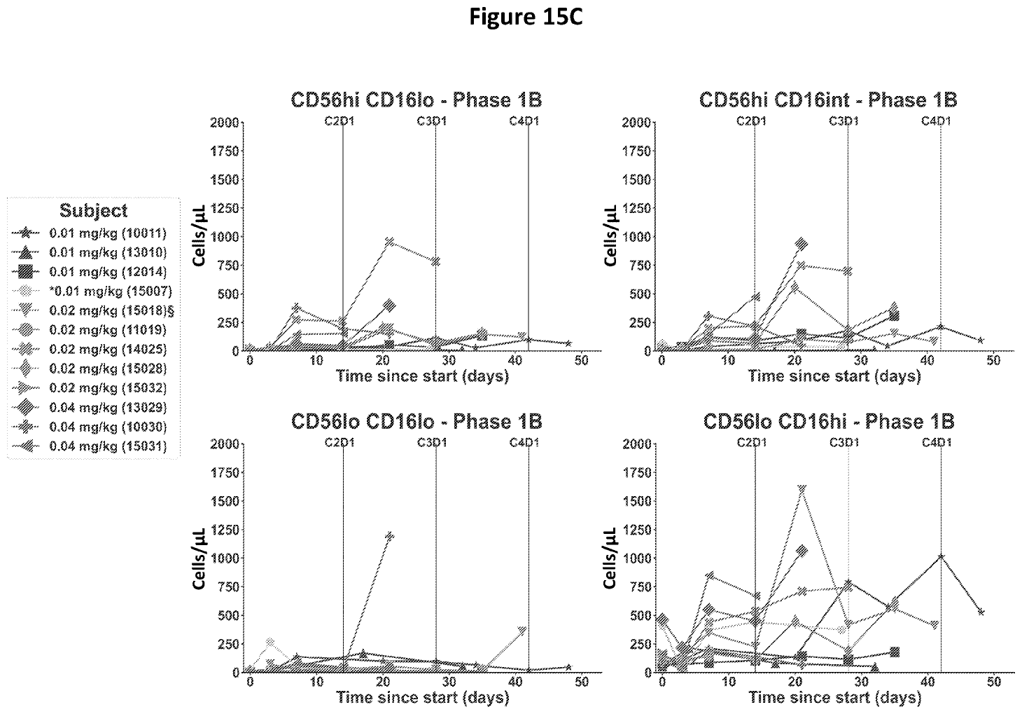

Dose dependent expansion of CD16+ NK cells (in both CD56 hi and CD56 lo subpopulations) was observed with XENP24306+XENP32803 in combination with atezolizumab in the phase 1b dose escalation study. See FIGS. 14A-14B and 15A-15C. Again, greater than 20-fold increases in NK cells were observed, as was cycle-to-cycle accumulation.

What is claimed:

1. A method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising the amino acid sequence set forth in SEQ ID NO: 9; and (ii) a second monomer comprising the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.

2. A method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising an amino acid sequence encoded by a polynucleotide encoding the amino acid sequence set forth in SEQ ID NO: 9;
and (ii) a second monomer comprising an amino acid sequence encoded by a polynucleotide encoding the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.

3. A method of treating a solid tumor in a human subject in need thereof, the method comprising administering to the subject a plurality of doses of a heterodimeric protein, wherein each dose comprises a therapeutically effective amount of the heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein is sufficient to increase the number of NK cells at least 20-fold as compared to the number of NK cells prior to said administration.

4. The method according to claim 3, wherein the first Fc domain of the heterodimeric protein comprises the amino acid sequence set forth in SEQ ID NO: 6.

5. The method according to claim 3 or 4, wherein the second Fc domain of the heterodimeric protein comprises the amino acid sequence set forth in SEQ ID NO: 7.

6. The method according to claim 3 or 4, wherein the second Fc domain of the heterodimeric protein comprises the amino acid sequence set forth in SEQ ID NO: 8.

7. A method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein is sufficient to increase the number of NK cells at least 20-fold as compared to the number of NK cells prior to said administration.

8. A method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising an amino acid sequence encoded by a polynucleotide encoding the amino acid sequence set forth in SEQ ID NO: 9 and (ii) a second monomer comprising an amino acid sequence encoded by a polynucleotide encoding the amino acid sequence set forth in SEQ ID NO: 10 or 16; wherein
the administration of the plurality of doses of the heterodimeric protein is sufficient to increase the number of NK cells at least 20-fold as compared to the number of NK cells prior to said administration.

9. A method for inducing the proliferation of NK cells in a human subject, the method comprising administering to the subject a plurality of doses of a heterodimeric protein, wherein each dose comprises an effective amount of the heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein is sufficient to increase the number of NK cells at least 20-fold as compared to the number of NK cells prior to said administration.

10. The method according to claim 9, wherein the first Fc domain of the heterodimeric protein comprises the amino acid sequence set forth in SEQ ID NO: 6.

11. The method according to claim 9 or 10, wherein the second Fc domain of the heterodimeric protein comprises the amino acid sequence set forth in SEQ ID NO: 7.

12. The method according to claim 9 or 10, wherein the second Fc domain of the heterodimeric protein comprises the amino acid sequence set forth in SEQ ID NO: 8.

13. The method according to any one of claims 7-12, wherein the subject is suffering from a solid tumor.

14. The method according to any one of claims 1-13, wherein the number of NK cells increases at least 30-fold, 40-fold, 50-fold, 60-fold, 70-fold, 80-fold, 90-fold, 100-fold, 150-fold, 200-fold, 250-fold, or 300-fold relative to the number of NK cells prior to administration.

15. The method according to any one of claims 1-13, wherein the heterodimeric protein is administered at a dose of at least 0.04 mg/kg Q2W and the number of NK cells increases at least 20-fold after two doses.

16. The method according to any one of claims 1-14, wherein the heterodimeric protein is administered at a dose of at least 0.06 mg/kg Q2W and the number of NK cells increases at least 50-fold after two doses.

17. The method according to any one of claims 1-14, wherein the heterodimeric protein is administered at a dose of at least 0.06 mg/kg Q2W and the number of NK cells increases at least 100-fold after three doses.

18. A method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein results in accumulation of NK cells in the subject.

19. A method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising an amino acid sequence encoded by a polynucleotide encoding the amino acid sequence set forth in SEQ ID NO: 9;
and (ii) a second monomer comprising an amino acid sequence encoded by a polynucleotide encoding the amino acid sequence set forth in SEQ ID NO: 10 or 16; wherein the administration of the plurality of doses of the heterodimeric protein results in accumulation of NK cells in the subject.

20. A method of treating a solid tumor in a human subject in need thereof, the method comprising administering to the subject a plurality of doses of a heterodimeric protein, wherein each dose comprises a therapeutically effective amount of a heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering;
wherein the administration of the plurality of doses of the heterodimeric protein results in accumulation of NK cells in the subject.

21. The method according to claim 20, wherein the first Fc domain of the heterodimeric protein comprises the amino acid sequence set forth in SEQ ID NO: 6.

22. The method according to claim 20 or 21, wherein the second Fc domain of the heterodimeric protein comprises the amino acid sequence set forth in SEQ ID NO: 7.

23. The method according to claim 20 or 21, wherein the second Fc domain of the heterodimeric protein comprises the amino acid sequence set forth in SEQ ID NO: 8.

24. A method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising the amino acid sequence set forth in SEQ ID NO: 9 and (ii) a second monomer comprising the amino acid sequence set forth in SEQ ID NO: 10 or 16; wherein the administration of the plurality of doses of the heterodimeric protein results in accumulation of NK cells in the subject.

25. A method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising an amino acid sequence encoded by a polynucleotide encoding the amino acid sequence set forth in SEQ ID NO: 9; and (ii) a second monomer comprising an amino acid sequence encoded by a polynucleotide encoding the amino acid sequence set forth in SEQ ID NO: 10 or 16; wherein
the administration of the plurality of doses of the heterodimeric protein results in accumulation of NK cells in the subject.

26. A method for inducing the proliferation of NK cells in a human subject, the method comprising administering to the subject a plurality of doses of a heterodimeric protein, wherein each dose comprises an effective amount of a heterodimeric protein, wherein the heterodimeric protein comprises (i) a first monomer comprising an IL-15 protein and a first Fc domain, wherein said IL-15 protein comprises the amino acid sequence set forth in SEQ ID NO: 5 and is covalently attached to the N-terminus of said first Fc domain by a first linker, wherein the first linker comprises the amino acid sequence set forth in SEQ ID NO: 39, wherein n is 1; and (ii) a second monomer comprising an IL-15Rα protein and a second Fc domain, wherein said IL-15Rα protein comprises the amino acid sequence set forth in SEQ ID NO: 4 and is covalently attached to the N-terminus of said second Fc domain by a second linker, wherein the second linker comprises the amino acid sequence set forth in SEQ ID NO: 39, wherein n is 1; wherein the first Fc domain comprises amino acid substitutions L368D and K370S; wherein the second Fc domain comprises amino acid substitutions S364K and E357Q; and wherein each of said first and second Fc domains further comprises amino acid substitutions C220S, E233P, L234V, L235A, G236 del, S267K, M428L and N434S, according to EU numbering; wherein the administration of the plurality of doses of the heterodimeric protein results in accumulation of NNK cells in the subject.

27. The method according to claim 26, wherein the first Fc domain of the heterodimeric protein comprises the amino acid sequence set forth in SEQ ID NO: 6.

28. The method according to claim 26 or 27, wherein the second Fc domain of the heterodimeric protein comprises the amino acid sequence set forth in SEQ ID NO: 7.

29. The method according to claim 26 or 27, wherein the second Fc domain of the heterodimeric protein comprises the amino acid sequence set forth in SEQ ID NO: 8.

30. The method according to any one of claims 24-29, wherein the subject is suffering from a solid tumor.

31. The method according to any one of claims 18-30, wherein the accumulation is for at least one cycle.

32. The method according to claim 31, wherein the accumulation is for at least two cycles, at least three cycles, or at least four cycles.

33. The method according to any one of claims 1-32, wherein the NK cells are CD16+ NK cells.

34. The method according to any one of claims 1-33, wherein the first monomer comprises the amino acid sequence set forth in SEQ ID NO: 57.

35. The method according to any one of claims 1-34, wherein the second monomer comprises the amino acid sequence set forth in SEQ ID NO: 10.

36. The method according to any one of claims 1-34, wherein the second monomer comprises the amino acid sequence set forth in SEQ ID NO: 58.

37. The method according to any one of claims 1-34, wherein the second monomer comprises the amino acid sequence set forth in SEQ ID NO: 16.

38. The method according to any one of claims 1-34, wherein the second monomer comprises the amino acid sequence set forth in SEQ ID NO: 59.

39. The method according to any one of claims 1-38, wherein said heterodimeric protein is XENP24306 with a C-terminal lysine, XENP24306 without a C-terminal lysine, XENP32803 with a C-terminal lysine, XENP32803 without a C-terminal lysine, or any combination thereof.

40. The method according to any one of claims 1-39, wherein a combination of a first heterodimeric protein and a second heterodimeric protein is administered to the subject.

41. The method according to claim 40, wherein the first heterodimeric protein comprises a first monomer comprising the amino acid sequence set forth in SEQ ID NO: 9, and a second monomer comprising the amino acid sequence set forth in SEQ ID NO: 10; and the second heterodimeric protein comprises a first monomer comprising the amino acid sequence set forth in SEQ ID NO: 9, and a second monomer comprising the amino acid sequence set forth in SEQ ID NO: 16.

42. The method according to claim 40 or 41, wherein said first and second heterodimeric proteins are administered simultaneously.

43. The method according to claim 40 or 41, wherein said first and second heterodimeric proteins are administered sequentially.

44. The method according to any one of claims 1-6, 13-23 and 30-43, wherein said solid tumor is locally advanced, recurrent or metastatic.

45. The method according to any one of claims 1-6, 13-23 and 30-43, wherein said solid tumor is selected from the group consisting of squamous cell cancer, cutaneous squamous cell cancer, small-cell lung cancer, non-small cell lung cancer, gastrointestinal cancer, gastric cancer, pancreatic cancer, glioblastoma, cervical cancer, ovarian cancer, liver cancer, bladder cancer, liposarcoma, soft-tissue sarcoma, urothelial carcinoma, ureter and renal pelvis, multiple myeloma, osteosarcoma, hepatoma, melanoma, stomach cancer, breast cancer, colon cancer, colorectal cancer, endometrial carcinoma, salivary gland carcinoma, renal cell carcinoma, esophageal cancer, prostate cancer, vulval cancer, thyroid cancer, hepatic carcinoma, Merkel cell carcinoma, germ cell cancer, micro-satellite instability-high cancer and head and neck squamous cell carcinoma.

46. The method according to claim 45, wherein said solid tumor is selected from the group consisting of melanoma, renal cell carcinoma, non-small cell lung cancer, head and neck squamous cell carcinoma, and triple negative breast cancer.

47. The method according to claim 45, wherein said solid tumor is selected from the group consisting of melanoma, renal cell carcinoma, and non-small cell lung cancer.

48. The method according to claim 45, wherein said solid tumor is selected from the group consisting of melanoma, non-small cell lung cancer, head and neck squamous cell carcinoma, and triple negative breast cancer.

49. The method according to any one of claims 1-6, 13-23 and 30-48, wherein prior to administration of the plurality of doses of the heterodimeric protein, the subject has not been previously administered an agent to treat the solid tumor.

50. The method according to any one of claims 1-6, 13-23 and 30-48, wherein the subject is currently being administered a checkpoint inhibitor.

51. The method according to any one of claims 1-6, 13-23 and 30-48, wherein prior to administration of the plurality of doses of the heterodimeric protein, the subject has previously been administered a checkpoint inhibitor.

52. The method according to claim 50 or 51, wherein the checkpoint inhibitor targets PD-1.

53. The method according to claim 50 or 51, wherein the checkpoint inhibitor targets PD-L1.

54. The method according to claim 50 or 51, wherein the checkpoint inhibitor targets CTLA-4.

55. The method according to any one of claims 1-14 and 18-54, wherein said heterodimeric protein or combination of heterodimeric proteins is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.09 mg/kg, about 0.1 mg/kg, about 0.12 mg/kg, about 0.16 mg/kg, about 0.2 mg/kg, about 0.24 mg/kg and about 0.32 mg/kg body weight.

56. The method according to claim 55, wherein said heterodimeric protein or combination of heterodimeric proteins is administered at a dose selected from the group consisting of about 0.01 mg/kg, about 0.02 mg/kg, about 0.04 mg/kg, about 0.06 mg/kg and about 0.09 mg/kg, and about 0.12 mg/kg body weight.

57. The method according to any one of claims 1-14 and 18-56, wherein said heterodimeric protein is administered at a frequency selected from the group consisting of Q1W, Q2W, Q3W, Q4W, Q5W and Q6W.

58. The method according to claim 57, wherein said heterodimeric protein is administered at a frequency of Q2W.

59. The method according any one of claims 1-58, wherein said method further comprises administering to the subject an agent targeting the PD-L1/PD-1 axis.

60. The method according to claim 59, wherein said agent targeting the PD-L1/PD-1 axis is an anti-PD-1 antibody.

61. The method according to claim 60, wherein the anti-PD-1 antibody is selected from the group consisting of nivolumab, pembrolizumab, pidilizumab, cemiplimab, spartalizumab, camrelizumab, sintilimab, tislelizumab, toripalimab, and AMP-514.

62. The method according to claim 59, wherein said agent targeting the PD-L1/PD-1 axis is an anti-PD-L1 antibody.

63. The method according to claim 62, wherein the anti-PD-L1 antibody is selected from the group consisting of avelumab, durvalumab, atezolizumab, BMS-936559, BMS-39886, envafolimab, and cosibelimab.
